(19) United States
(12) Patent Application Publication
(10) Pub. No.: US 2007/0014721 A1
(43) Pub. Date: Jan. 18, 2007

(54) Hydrazide conjugates as imaging agents

(76) Inventors: Thomas D. Harris, Salem, NH (US); Simon P. Robinson, Stow, MA (US); Richard R. Cesati III, Pepperell, MA (US); Padmaja Yalamanchili, Weston, MA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/476,377

(22) Filed: Jun. 28, 2006

Related U.S. Application Data
(60) Provisional application No. 60/695,496, filed on Jun. 30, 2005.

(51) Int. Cl. A61K 51/00

(52) U.S. Cl. 424/1.11; 564/147; 548/530; 546/226; 540/607

(57) ABSTRACT

The present disclosure is directed to diagnostic agents. More specifically, the disclosure is directed to compounds, diagnostic agents, compositions, and kits for detecting and/or imaging and/or monitoring a pathological disorder associated with coronary plaque, carotid plaque, aortic plaque, plaque of the arterial vessel, aneurism, vasculitis, and other diseases of the arterial wall. In addition, the disclosure is directed to methods of detecting and/or imaging and/or monitoring changes in the arterial wall, including expansive and constrictive remodeling, total vessel wall area, internal lumen size, and exterior artery perimeter.

### CROSS REFERENCE TO RELATED APPLICATION

This claims the benefit of U.S. Provisional Application No. 60/695,496 filed Jun. 30, 2005.

The present disclosure is directed to diagnostic agents. More specifically, the disclosure is directed to compounds, diagnostic agents, compositions, and kits for detecting and/or imaging and/or monitoring a pathological disorder associated with coronary plaque, carotid plaque, aortic plaque, plaque of the arterial vessel, aneurism, vasculitis, and other diseases of the arterial wall. In addition, the disclosure is directed to methods of detecting and/or imaging and/or monitoring changes in the arterial wall, including expansive and constrictive remodeling, total vessel wall area, internal lumen size, and exterior artery perimeter.

Cardiovascular diseases are the leading cause of death in the United States, accounting annually for more than one million deaths. Atherosclerosis is the major contributor to coronary heart disease and a primary cause of non-accidental death in Western countries. Considerable effort has been made in defining the etiology and potential treatment of atherosclerosis and its consequences, including myocardial infarction, angina, organ failure, and stroke. Despite this effort, there are many unanswered questions including how and when atherosclerotic lesions become vulnerable and life-threatening, the best point of intervention, and how to detect and monitor the progression of lesions.

In the last two decades, many radiotracers have been developed based on several molecules and cell types involved in atherosclerosis. In general, radiolabeled proteins and platelets have shown some clinical potential as imaging agents of atherosclerosis, but due to poor target/background and target/blood ratios, these agents are not ideal for imaging coronary or even carotid lesions. Radiolabeled peptides, antibody fragments, and metabolic tracers like FDG appear to offer new opportunities for nuclear scintigraphic techniques in the non-invasive imaging of atherothrombosis. However, a non-invasive method to diagnose and monitor various cardiovascular diseases are needed.

### BRIEF DESCRIPTION OF THE SEVERAL VIEWS OF THE DRAWING

FIG. 1 illustrates the magnetic resonance image of the abdominal aorta of an ApoE knockout mouse after administration of Example 114.

FIG. 2 illustrates the magnetic resonance image of the abdominal aorta of an ApoE knockout mouse after administration of Example 116.

FIG. 3 illustrates the magnetic resonance image of the abdominal aorta of an ApoE knockout mouse after administration of Example 113.

FIG. 4 illustrates the magnetic resonance image of the abdominal aorta of an ApoE knockout mouse after administration of gadopentetate dimeglumine.

FIG. 5 illustrates the magnetic resonance image of the abdominal aorta of an ApoE knockout mouse prior to the administration of contrast agent.

### DETAILED DESCRIPTION

In one aspect of the present disclosure is provided a compound of formula (I) 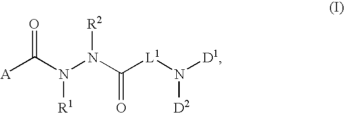
or a pharmaceutically acceptable salt thereof, wherein

A is a D-amino acid residue or a peptide consisting of a D-amino acid residue and a second D-amino acid;

D¹ and D² are independently selected from hydrogen, a chelator, and an imaging moiety;

L¹ is a linker; or

L¹ and D², together with the nitrogen atom to which they are attached, form a five- to seven-membered ring; and

R¹ and R² are independently selected from hydrogen and alkyl.

In a first embodiment of the first aspect of the present disclosure is provided a compound of formula (I), or a pharmaceutically acceptable salt thereof, wherein at least one of D¹ and D² is an imaging moiety. In a second embodiment of the first aspect of the present disclosure the imaging moiety comprises a non-metallic isotope. In a third embodiment of the first aspect of the present disclosure the non-metallic isotope is ¹⁴C, ¹³N, ¹⁸F, ¹²³I, or ¹²⁵I.

In a fourth embodiment of the first aspect of the present disclosure is provided a compound of formula (I), or a pharmaceutically aceeptable salt thereof, wherein L¹ is a linker selected from alkylene, alkenylene, arylene, heteroalkylene, arylalkylene, and heterocyclylene. In a fifth embodiment of the first aspect of the present disclosure L¹ is alkylene. In a sixth embodiment of the first aspect of the present disclosure L¹ is arylalkylene.

In a seventh embodiment of the first aspect of the present disclosure is provided a compound of formula (I), or a pharmaceutically acceptable salt thereof, wherein A is a D-amino acid residue. In an eighth embodiment of the first aspect of the present disclosure A is 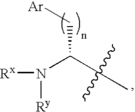
wherein

n is 0-6;

Ar is an aryl group; and

R^{x} and R^{y} are independently selected from hydrogen, alkenyl, alkoxycarbonyl, alkylcarbonyl, alkyl, aryl, and arylalkyl.

In a ninth embodiment of the first aspect of the present disclosure n is 2, Ar is phenyl, and R^{x} and R^{y} are hydrogen.

In a tenth embodiment of the first aspect of the present disclosure is provided a compound of formula (I), or a pharmaceutically acceptable salt thereof, wherein one of D¹ and D² is a hydrogen and the other is a chelator. In an eleventh embodiment of the first aspect of the present disclosure is provided a compound of formula (I), or a pharmaceutically acceptable salt thereof, wherein one of D¹ and D² is a hydrogen and the other is a chelator wherein the compound further comprises an imaging agent.

In a twelfth embodiment of the first aspect of the present disclosure is provided a compound of formula (I), or a pharmaceutically acceptable salt thereof, wherein one of D¹ and D² is hydrogen and the other is a chelator of formula (II) 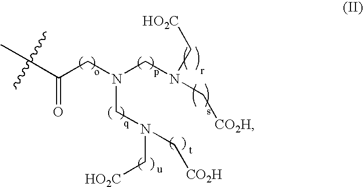
wherein

o, p, q, r, s, t, and u are each independently 1-6.

In a thirteenth embodiment of the first aspect of the present disclosure o, r, s, t, and u are each 1 and p and q are each 2.

In a second aspect of the present disclosure is provided a diagnostic agent, comprising:

a. a compound of formula (III) 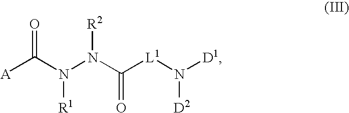
or a pharmaceutically acceptable salt thereof, wherein

A is a D-amino acid residue or a peptide consisting of a D-amino acid residue and a second D-amino acid;

D¹ and D² are independently selected from hydrogen and a chelator;

L¹ is a linker; or

L¹ and D², together with the nitrogen atom to which they are attached, form a five- to seven-membered ring; and

R¹ and R² are independently selected from hydrogen and alkyl; and

b. an imaging agent.

In a first embodiment of the second aspect of the present disclosure the imaging agent is an echogenic substance, an optical reporter, a boron neutron absorber, a paramagnetic metal ion, a ferromagnetic metal, a gamma-emitting radioisotope, a positron-emitting radioisotope, or an x-ray absorber. In a second embodiment of the second aspect of the present disclosure the imaging agent is a paramagnetic metal ion. In a third embodiment of the second aspect of the present disclosure the paramagnetic metal ion is Gd(III). In a fourth embodiment of the second aspect of the present disclosure the imaging agent is a gamma-emitting radioisotope or positron-emitting radioisotope selected from ⁹⁹mTc, ⁹⁵Tc, ¹¹¹In, ⁶²Cu, ⁶⁴Cu, ⁶⁷Ga, ⁶⁸Ga, and ¹⁵³Gd. In a fifth embodiment of the second aspect of the present disclosure the imaging agent is ⁹⁹mTc. In a sixth embodiment of the second aspect of the present disclosure the imaging agent is ¹¹¹In.

In a third aspect of the present disclosure is provided a compound which is 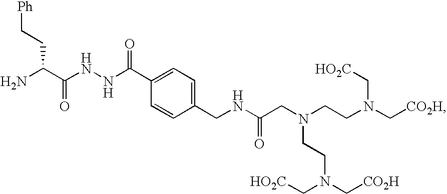
or a pharmaceutically acceptable salt thereof.

In a fourth aspect of the present disclosure is provided a compound which is 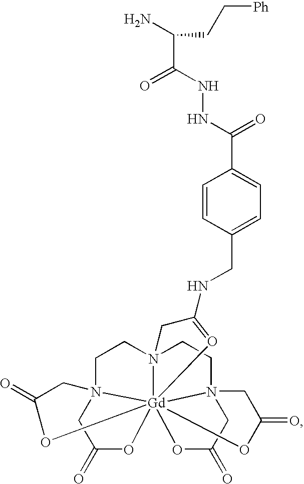
or a pharmaceutically acceptable salt thereof.

In a fifth aspect of the present disclosure is provided a compound which is 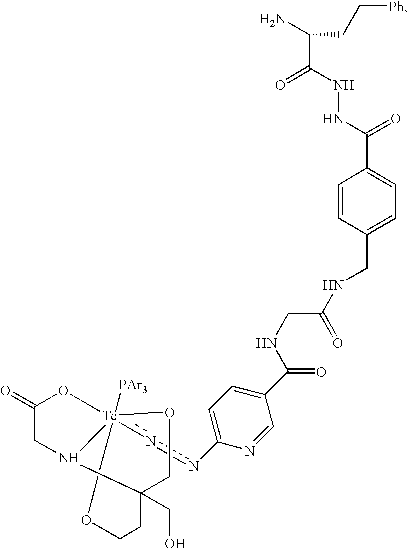
or a pharmaceutically acceptable salt thereof, wherein Ar is selected from phenyl, m-phenylsulfonic acid, or p-phenylsulfonic acid.

In a sixth aspect of the present disclosure is provided a compound which is 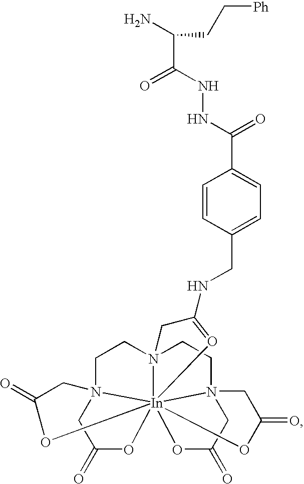
or a pharmaceutically acceptable salt thereof.

In a seventh aspect of the present disclosure is provided a composition comprising:

(a) a compound of formula (I), or a pharmaceutically acceptable salt thereof; and

(b) a pharmaceutically acceptable carrier.

In an eighth aspect of the present disclosure is provided a composition comprising:

a. a compound of formula (III) 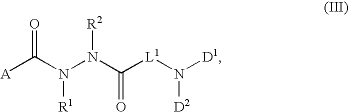
or a pharmaceutically acceptable salt thereof, wherein

A is a D-amino acid residue or a peptide consisting of a D-amino acid residue and a second D-amino acid;

D¹ and D² are independently selected from hydrogen and a chelator;

L¹ is a linker; or

L¹ and D², together with the nitrogen atom to which they are attached, form a five- to seven-membered ring; and

R¹ and R² are independently selected from hydrogen and alkyl;

b. an imaging agent; and

(c) a pharmaceutically acceptable carrier.

In a ninth aspect of the present disclosure is provided a kit for detecting, imaging, and/or monitoring changes in the arterial wall, including expansive and constrictive remodeling, total vessel wall area, internal lumen size, and exterior artery perimeter in a patient comprising:

a. a compound of formula (III) 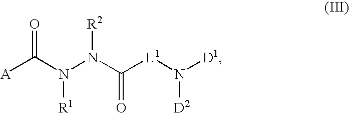
or a pharmaceutically acceptable salt thereof, wherein

A is a D-amino acid residue or a peptide consisting of a D-amino acid residue and a second D-amino acid;

D¹ and D² are independently selected from hydrogen and a chelator;

L¹ is a linker; or

L¹ and D², together with the nitrogen atom to which they are attached, form a five- to seven-membered ring; and

R¹ and R² are independently selected from hydrogen and alkyl;

b. an imaging agent;

c. a pharmaceutically acceptable carrier; and

d. instructions for preparing a composition comprising a diagnostic agent for detecting, imaging, and/or monitoring changes in the arterial wall, including expansive and constrictive remodeling, total vessel wall area, internal lumen size, and exterior artery perimeter in a patient.

In a tenth aspect of the present disclosure is provided a method of detecting, imaging, and/or monitoring changes in the arterial wall, including expansive and constrictive remodeling, total vessel wall area, internal lumen size, and exterior artery perimeter in a patient comprising the steps of:

a. administering to the patient a compound of formula (I); and

b. acquiring an image of a site of concentration of the compound in the patient by a diagnostic imaging technique.

In an eleventh aspect of the present disclosure is provided a method of detecting, imaging, and/or monitoring changes in the arterial wall, including expansive and constrictive remodeling, total vessel wall area, internal lumen size, and exterior artery perimeter in a patient comprising the steps of:

a. administering to the patient a diagnostic agent comprising:
i. a compound of formula (III) 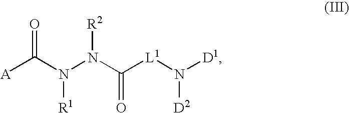
   or a pharmaceutically acceptable salt thereof, wherein

A is a D-amino acid residue or a peptide consisting of a D-amino acid residue and a second D-amino acid;

D¹ and D are independently selected from hydrogen and a chelator;

L¹ is a linker; or

L¹ and D², together with the nitrogen atom to which they are attached, form a five- to seven-membered ring; and

R¹ and R² are independently selected from hydrogen and alkyl; and
ii. an imaging agent; and

b. acquiring an image of a site of concentration of the compound in the patient by a diagnostic imaging technique.

Other aspects of the invention may include suitable combinations of embodiments and aspects disclosed herein.

Yet other aspects and embodiments may be found in the description provided herein.

Unless otherwise specifically noted herein, the terms set forth below will have the following definitions.

In some instances, the number of carbon atoms in any particular group is denoted before the recitation of the group. For example, the term “C₆₋₁₀aryl” denotes an aryl group containing from six to ten carbon atoms, and the term “C₆₋₁₀aryl-C₁₋₁₀alkyl,” refers to an aryl group of six to ten carbon atoms attached to the parent molecular moiety through an alkyl group of one to ten carbon atoms. Where these designations exist they supercede all other definitions contained herein.

As used herein, the singular forms “a”, “an”, and “the” include plural reference unless the context clearly dictates otherwise.

The term “alkenyl,” as used herein, refers to a straight or branched chain hydrocarbon of two to fourteen carbon atoms containing at least one carbon-carbon double bond.

The term “alkenylene,” as used herein, refers to a divalent group of derived from a straight or branched chain hydrocarbon containing from two to fourteen carbon atoms at least one carbon-carbon double bond.

The term “alkoxy,” as used herein refers to an alkyl group attached to the parent molecular moiety through an oxygen atom.

The term “alkoxyalkyl,” as used herein, refers to an alkoxy group attached to the parent molecular moiety through an alkyl group.

The term “alkoxycarbonyl,” as used herein, refers to an alkoxy group attached to the parent molecular moiety through a carbonyl group.

The term “alkyl,” as used herein, refers to a group derived from a straight or branched chain saturated hydrocarbon.

The term “alkylaryl,” as used herein, refers to an alkyl group attached to the parent molecular moiety through an aryl group.

The term “alkylcarbonyl,” as used herein, refers to an alkyl group attached to the parent molecular moiety through a carbonyl group.

The term “alkylene,” as used herein, refers to a divalent group derived from a straight or branched chain saturated hydrocarbon of one to fourteen carbon atoms.

As used herein, the phrase “amino acid residue” means a moiety derived from a naturally-occurring or synthetic organic compound containing an amino group (—NH₂), a carboxylic acid group (—COOH), and any of various side groups, especially any of the 20 compounds that have the basic formula NH₂CHRCOOH, and that link together by peptide bonds to form proteins or that function as chemical messengers and as intermediates in metabolism. For example, in compound X 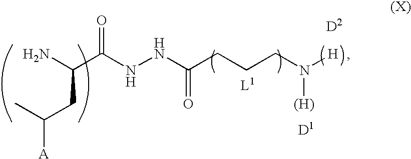
the portion of the molecule denoted as “A” is a residue of the amino acid D-leucine.

As used herein, the terms “ancillary” and “co-ligands” refers to ligands that serve to complete the coordination sphere of the radionuclide together with the chelator of the reagent. For radiopharmaceuticals comprising a binary ligand system, the radionuclide coordination sphere comprises one or more chelators from one or more reagents and one or more ancillary or co-ligands, provided that there are a total of two types of ligands or chelators. For example, a radiopharmaceutical comprised of one chelator from one reagent and two of the same ancillary or co-ligands and a radiopharmaceutical comprising two chelators from one or two reagents and one ancillary or co-ligand are both considered to comprise binary ligand systems. For radiopharmaceuticals comprising a ternary ligand system, the radionuclide coordination sphere comprises one or more chelators from one or more reagents and one or more of two different types of ancillary or co-ligands, provided that there are a total of three types of ligands or chelators. For example, a radiopharmaceutical comprised of one chelator from one reagent and two different ancillary or co-ligands is considered to comprise a ternary ligand system.

Ancillary or co-ligands useful in the preparation of radiopharmaceuticals and in diagnostic kits useful for the preparation of said radiopharmaceuticals comprise one or more oxygen, nitrogen, carbon, sulfur, phosphorus, arsenic, selenium, and tellurium donor atoms. A ligand can be a transfer ligand in the synthesis of a radiopharmaceutical and also serve as an ancillary or co-ligand in another radiopharmaceutical. Whether a ligand is termed a transfer or ancillary or co-ligand depends on whether the ligand remains in the radionuclide coordination sphere in the radiopharmaceutical, which is determined by the coordination chemistry of the radionuclide and the chelator of the reagent or reagents.

The term “aryl,” as used herein, refers to a phenyl group, or a bicyclic fused ring system wherein one or more of the rings is a phenyl group. Bicyclic fused ring systems consist of a phenyl group fused to a monocyclic cycloalkenyl group, a monocyclic cycloalkyl group, or another phenyl group. The aryl groups of the present invention can be attached to the parent molecular moiety through any substitutable carbon atom in the group. Representative examples of aryl groups include, but are not limited to, anthracenyl, azulenyl, fluorenyl, indanyl, indenyl, naphthyl, phenyl, and tetrahydronaphthyl.

The term “arylalkyl,” as used herein, refers to an aryl group attached to the parent molecular moiety through an alkyl group.

The term “arylalkylene,” as used herein, refers to a divalent arylalkyl group, where one point of attachment to the parent molecular moiety is on the aryl portion and the other is on the alkyl portion.

The term “arylene,” as used herein, refers to a divalent aryl group.

As used herein, the term “bacteriostat” means a component that inhibits the growth of bacteria in a formulation either during its storage before use of after a diagnostic kit is used to synthesize a diagnostic agent.

The term “buffer,” as used herein, refers to a substance used to maintain the pH of the reaction mixture from about 3 to about 10.

The term “carbonyl,” as used herein, refers to —(O)—.

The term “cyano,” as used herein, refers to —CN.

The term “carrier”, as used herein, refers to an adjuvant or vehicle that may be administered to a patient, together with the compounds and/or diagnostic agents of this disclosure which does not destroy the activity thereof and is non-toxic when administered in doses sufficient to deliver an effective amount of the diagnostic agent and/or compound.

The term “chelator,” as used herein, refers to the moiety or group on a molecule that binds to a metal ion through one or more donor atoms. The chelator is optionally attached to the parent molecular moiety through a linker, L². Examples of suitable L² groups include, but are not limited to, —C(O)CH₂—Ar—CH₂NHC(O)—, where Ar is an arylene group; —C(O)—; —C(O)-Het-NHNHC(O)—, where Het is heteroarylene; and —C(O)-Het-. In certain embodiments of the compounds and/or diagnostic agents of the disclosure, the chelator is a surfactant capable of forming an echogenic substance-filled lipid sphere or microbubble.

In certain other embodiments, the chelator has a formula selected from 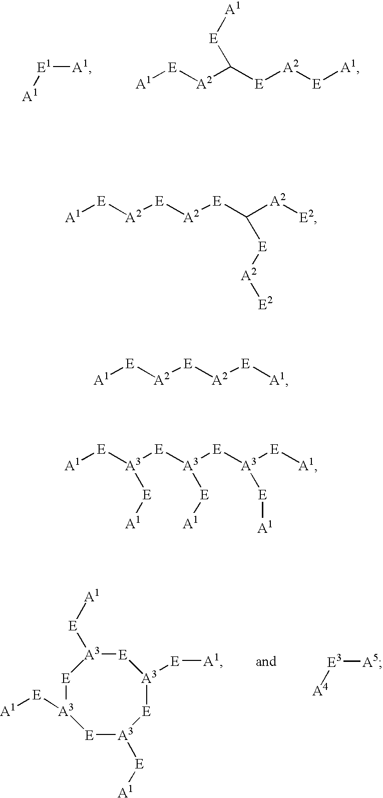
wherein

each A¹ is independently selected from —NR¹⁹R²⁰, —N(R²⁶)₂, —SH, —S(Pg), —OH, —PR¹⁹R²⁰, —P(O)R²¹R²², —CO₂H, a bond to the parent molecular moiety, and a bond to L²;

each A² is independently selected from N(R²⁶), N(R¹⁹), S, O, P(R¹⁹), and —OP(O)(R²¹)O—;

A³ is N;

A⁴ is selected from OH and OC(═O)C₁₋₂₀ alkyl;

A⁵ is OC(═O)C₁₋₂₀ alkyl;

each E is independently selected from C₁₋₁₆alkylene substituted with 0-3 R²³, C₆₋₁₀arylene substituted with 0-3 R²³, C₃₋₁₀cycloalkylene substituted with 0-3 R²³, heterocyclyl-C₁₋₁₀alkylene substituted with 0-3 R²³, C₆₋₁₀aryl-C₁₋₁₀alkylene substituted with 0-3 R²³, C₁₋₁₀alkyl-C₆₋₁₀arylene substituted with 0-3 R²³, and heterocyclylene substituted with 0-3 R²³;

E¹ is selected from a bond and E;

each E² is independently selected from C₁₋₁₆alkyl substituted with 0-3 R²³, C₆₋₁₀aryl substituted with 0-3 R²³, C₃₋₁₀cycloalkyl substituted with 0-3 R²³ heterocyclyl-C₆₋₁₀alkyl substituted with 0-3 R²³, C₆₋₁₀aryl-C₁₋₁₀alkyl substituted with 0-3 R²³, C₁₋₁₀alkyl-C₆₋₁₀aryl substituted with 0-3 R²³, and heterocyclyl substituted with 0-3 R²³;

E³ is C₁₋₁₀alkylene substituted with 1-3 R³²;

Pg is a thiol protecting group;

R²⁹ and R²⁰ are each independently selected from a bond to L², a bond to the parent molecular moiety, hydrogen, C₁₋₁₀alkyl substituted with 0-3 R²³, aryl substituted with 0-3 R²³, C₃₋₁₀cycloalkyl substituted with 0-3 R²³, heterocyclyl-C₁₋₁₀alkyl substituted with 0-3 R²³, C₆₋₁₀aryl-C₁₋₁₀alkyl substituted with 0-3 R²³, and heterocyclyl substituted with 0-3 R²³.

R²¹ and R²² are each independently selected from a bond L², a bond to the parent molecular moiety, —OH, C₁₋₁₀alkyl substituted with 0-3 R²³, aryl substituted with 0-3 R²³, C₃₋₁₀cycloalkyl substituted with 0-3 R²³, heterocyclyl-C₁₋₁₀alkyl substituted with 0-3 R²³, C₆₋₁₀aryl-C₁₋₁₀alkyl substituted with 0-3 R²³, and heterocyclyl substituted with 0-3 R²³;

each R²³ is independently selected from a bond to L², a bond to the parent molecular moiety, ═O, halo, trifluoromethyl, cyano, —CO₂R²⁴, —C(═O)R²⁴, —C(═O)N(R²⁴)₂, —CHO, —CH₂OR²⁴, —OC(═O)R²⁴, —OC(═O)OR²⁴, —OR²⁴, —OC(═O)N(R²⁴)₂, —NR²⁴C(═O)R²⁴, —NR²⁴C(═O)OR²⁴, —NR²⁴C(═O)N(R²⁴)₂, —NR²⁴SO₂N(R²⁴)₂, —NR²⁴SO₂R²⁴, —SO₃H, —SO₂R²⁴, —SR²⁴, —S(═O)R²⁴, —SO₂N(R²⁴)₂, —N(R²⁴)₂, —NHC(═S)NHR²⁴, ═NOR²⁴, NO₂, —C(═O)NHOR²⁴, —C(═O)NHNR²⁴R²⁴, —OCH₂CO₂H, 2-(1-morpholino)ethoxy, C₁₋₅alkyl, C₂₋₄alkenyl, C₃₋₆cycloalkyl, C₃₋₆cycloalkylmethyl, C₂₋₆alkoxyalkyl, aryl substituted with 0-2 R²⁴, and heterocyclyl;

each R²⁴ is independently selected from a bond to L², a bond to the parent molecular moiety, hydrogen, C₁₋₆alkyl, phenyl, benzyl, and C₁₋₆ alkoxy;

each R²⁶ is independently a coordinate bond to a metal or a hydrazine protecting group;

each R³² selected from R³⁴, ═O, —CO₂R³³, —C(═O)R³³, —C(═O)N(R³³)₂, —CH₂OR³³, —OR³³, —N(R³³)₂, and C₂-C₄ alkenyl;

each R³³ is independently selected from R³⁴, hydrogen, C₁-C₆ alkyl, phenyl, benzyl, and trifluoromethyl; and

R³⁴ is a bond to L²;

wherein at least one of A¹, R¹⁹, R²⁰, R²¹, R²², R²³, R²⁴, and R³⁴ is a bond to L² or the parent molecular moiety.

In an embodiment of the present disclosure, the chelant is of the formula:

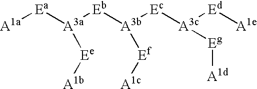

wherein

A^{1c} is a bond to L²;

A^{1a}, A^{1b}, A^{1d} and A^{1e} are each —CO₂H;

A^{3a}, A^{3b}, and A^{3c} are each N;

E^{b}, and E^{c} are C₂alkylene; and

E^{a}, E^{d}, E^{e}, E^{f}, and E^{g} are CH₂.

In another embodiment of the present disclosure the chelant is of the formula:

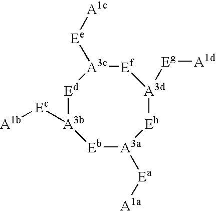

wherein:

A^{3a}, A^{3b}, A^{3c} and A^{3d} are each N;

A^{1a} is a bond to L²;

A^{1b}, A^{1c} and A^{1d} are each —CO₂H;

E^{a}, E^{c}, E^{g} and E^{e} are each CH₂; and

E^{b}, E^{d}, E^{f} and E^{h} are each C₂alkylene.

In another embodiment of the present disclosure, the chelant is of the formula:

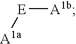

wherein

A^{1a} is —N(R²⁶)₂;

A^{1b} is NHR¹⁹;

E is a bond;

R¹⁹ is a bond to L²; and

each R²⁶ is a co-ordinate bond to a metal.

The term “cycloalkyl,” as used herein, refers to a saturated monocyclic, bicyclic, or tricyclic hydrocarbon ring system having three to fourteen carbon atoms and zero heteroatoms. Representative examples of cycloalkyl groups include, but are not limited to, cyclopropyl, cyclopentyl, bicyclo[3.1.1]heptyl, and adamantyl.

The term “cycloalkylene,” as used herein, refers to a divalent cycloalkyl group.

The term “cycloalkylmethyl,” as used herein, refers to a cycloalkyl group attached to the parent molecular moiety through a —CH₂— group.

As used herein, the term “diagnostic agent” refers to a compound that may be used to detect, image and/or monitor the presence and/or progression of a condition(s), pathological disorder(s) and/or disease(s). It should be understood that all compounds of the present invention that contain an imaging agent are diagnostic agents. For example, a compound of formula (I) wherein one of D¹ and D² is an imaging agent is a diagnostic agent.

The term “diagnostic imaging technique,” as used herein, refers to a procedure used to detect a diagnostic agent.

The terms “diagnostic kit” and “kit”, as used herein, refer to a collection of components in one or more vials that are used by the practicing end user in a clinical or pharmacy setting to synthesize diagnostic agents. The kit provides all the requisite components to synthesize and use the diagnostic agents (except those that are commonly available to the practicing end user such as water or saline for injection), such as a solution of the imaging agent or a precursor thereof, equipment for heating during the synthesis of the diagnostic agent, equipment necessary for administering the diagnostic agent to the patient such as syringes and shielding (if required), and imaging equipment.

As used herein, the phrase “donor atom” refers to the atom directly attached to a metal by a chemical bond.

The term “halo,” as used herein, refers to Br, Cl, F, or I.

The term “heteroalkylene,” as used herein, refers to an alkylene group wherein one to seven of the carbon atoms are replaced by a heteroatom selected from O, NH, and S.

The term “heterocyclyl,” as used herein, refers to a five-, six-, or seven-membered ring containing one, two, or three heteroatoms independently selected from the group consisting of nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a phenyl group, a monocyclic cycloalkenyl group, a monocyclic cycloalkyl group, or another monocyclic heterocyclyl group. The heterocyclyl groups of the present invention can be attached to the parent molecular moiety through a carbon atom or a nitrogen atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “heterocyclylalkyl,” as used herein, refers to a heterocyclyl group attached to the parent molecular moiety through an alkyl group.

The term “heterocyclylalkylene,” as used herein, refers to a divalent heterocyclylalkyl group, where one point of attachment to the parent molecular moiety is on the heterocyclyl portion and the other is on the alkyl portion.

The term “heterocyclylene,” as used herein, refers to a divalent heterocyclyl group.

The term “imaging moiety,” as used herein, refer to a portion or portions of a molecule that contain an imaging agent. The term “imaging agent,” as used herein, refers to an element or functional group in a diagnostic agent that allows for the detection, imaging, and/or monitoring of the presence and/or progression of a condition(s), pathological disorder(s), and/or disease(s). The imaging moiety may contain a linker, L³, which connects the imaging agent to the parent molecular moiety. Examples of suitable L³ groups include straight or branched chain alkylene groups,

—C(O)—, and the like.

The imaging agent may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

Suitable echogenic gases include a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include ¹¹C, ¹⁴C, ¹³N, ¹⁸F, ¹²³I, ¹²⁴I, and ¹²⁵I.

Suitable optical reporters include a fluorescent reporter and chemiluminescent groups.

Suitable radioisotopes include ⁹⁹mTc, ⁹⁵Tc, ¹¹¹In, ⁶²Cu, ⁶⁴Cu, ^{Ga}, ⁶⁸Ga, and ¹⁵³Gd. In a specific embodiment of the present disclosure suitable radioisotopes include ⁹⁹mTc, ¹¹¹In, ⁶⁸Ga, ¹⁵³Gd.

Suitable paramagnetic metal ions include: Gd(III), Dy(III), Fe(III), and Mn(II).

Suitable X-ray absorbers include: Re, Sm, Ho, Lu, Pm, Y, Bi, Pd, Gd, La, Au, Au, Yb, Dy, Cu, Rh, Ag, and Ir.

The term “linker,” as used herein, refers to a portion of a molecule that serves as a spacer between two other portions of the molecule. Linkers may also serve other functions as described herein.

As used herein, the term“lyophilization aid” means a component that has favorable physical properties for lyophilization, such as the glass transition temperature, and is added to the formulation to improve the physical properties of the combination of all the components of the formulation for lyophilization.

As used herein, the term “metallopharmaceutical” means a pharmaceutical comprising a metal. The metal is the origin of the imageable signal in diagnostic applications and the source of the cytotoxic radiation in radiotherapeutic applications.

As used herein, the phrase “pharmaceutically acceptable” refers to those compounds, diagnostic agents, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The compounds and/or diagnostic agents of the present disclosure can exist as pharmaceutically acceptable salts. The term “pharmaceutically acceptable salt,” as used herein, represents salts or zwitterionic forms of the compounds and/or diagnostic agents of the present disclosure which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use The salts can be prepared during the final isolation and purification of the compounds and/or diagnostic agents or separately by reacting a suitable nitrogen atom with a suitable acid. Representative acid addition salts include acetate, adipate, alginate, citrate, aspartate, benzoate, benzenesulfonate, bisulfate, butyrate, camphorate, camphorsulfonate; digluconate, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

Basic addition salts can be prepared during the final isolation and purification of the compounds and/or diagnostic agents by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, and N,N′-dibenzylethylenediamine. Other representative organic amines useful for the formation of base addition salts include ethylenediamine, ethanolamine, diethanolamine, meglumine, piperidine, and piperazine.

The term “radiopharmaceutical,” as used herein, refers to a metallopharmaceutical in which the metal is a radioisotope.

As used herein, the term “reagent” means a compound of this disclosure capable of direct transformation into a diagnostic agent of this disclosure. Reagents may be utilized directly for the preparation of the diagnostic agents of this disclosure or may be a component in a kit of this disclosure.

The term “reducing agent,” as used herein, refers to a compound that reacts with a radionuclide (which is typically obtained as a relatively unreactive, high oxidation state compound) to lower its oxidation state by transferring electron(s) to the radionuclide, thereby making it more reactive.

As used herein, the phrase “solubilization aid” is a component that improves the solubility of one or more other components in the medium required for the formulation.

As used herein, the phrase “stabilization aid” means a component that is added to the metallopharmaceutical or to the diagnostic kit either to stabilize the metallopharmaceutical or to prolong the shelf-life of the kit before it must be used. Stabilization aids can be antioxidants, reducing agents or radical scavengers and can provide improved stability by reacting with species that degrade other components or the metallopharmaceutical.

The term “stable”, as used herein, refers to compounds and/or diagnostic agents which possess the ability to allow manufacture and which maintain their integrity for a sufficient period of time to be useful for the purposes detailed herein. Typically, the compounds and/or diagnostic agents of the present disclosure are stable at a temperature of 40° C. or less in the absence of moisture or other chemically reactive conditions for at least a week.

The term “sterile,” as used herein, means free of or using methods to keep free of pathological microorganisms.

Asymmetric centers exist in the compounds and/or diagnostic agents of the present invention. These centers are designated by the symbols “R” or “S”, depending on the configuration of substituents around the chiral carbon atom. It should be understood that the invention encompasses all stereochemical isomeric forms of the present compounds and/or diagnostic agents, or mixtures thereof, unless otherwise specifically stated. Individual stereoisomers of compounds and/or diagnostic agents can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds and/or diagnostic agents of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and/or diagnostic agents and mixtures thereof.

When any variable occurs more than one time in any substituent or in any formula, its definition on each occurrence is independent of its definition at every other occurrence. Thus, for example, if a group is shown to be substituted with 0-2 R²³, then said group may optionally be substituted with up to two R²³, and R²³ at each occurrence is selected independently from the defined list of possible R²³. Also, by way of example, for the group —N(R²⁴)₂, each of the two R²⁴ substituents on the nitrogen is independently selected from the defined list of possible R²⁴. Combinations of substituents and/or variables are permissible only if such combinations result in stable compounds and/or diagnostic agents. When a bond to a substituent is shown to cross the bond connecting two atoms in a ring, then such substituent may be bonded to any atom on the ring.

When the imaging agent is a radioisotope, the compound may further comprise a first ancillary ligand and a second ancillary ligand capable of stabilizing the radioisotope. A large number of ligands can serve as ancillary or co-ligands, the choice of which is determined by a variety of considerations such as the ease of synthesis of the radiopharmaceutical, the chemical and physical properties of the ancillary ligand, the rate of formation, the yield, and the number of isomeric forms of the resulting radiopharmaceuticals, the ability to administer said ancillary or co-ligand to a patient without adverse physiological consequences to said patient, and the compatibility of the ligand in a lyophilized kit formulation. The charge and lipophilicity of the ancillary ligand will effect the charge and lipophilicity of the radiopharmaceuticals. For example, the use of 4,5-dihydroxy-1,3-benzenedisulfonate results in radiopharmaceuticals with an additional two anionic groups because the sulfonate groups will be anionic under physiological conditions. The use of N-alkyl substituted 3,4-hydroxypyridinones results in radiopharmaceuticals with varying degrees of lipophilicity depending on the size of the alkyl substituents.

It should also be understood that the compounds and/or diagnostic agents of this disclosure may adopt a variety of conformational and ionic forms in solution, in pharmaceutical compositions and in vivo. Although the depictions herein of specific compounds and/or diagnostic agents of this disclosure are of particular conformations and ionic forms, other conformations and ionic forms of those compounds and/or diagnostic agents are envisioned and embraced by those depictions.

Pharmaceutically acceptable carriers, adjuvants and vehicles that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, TRIS (tris(hydroxymethyl)amino-methane), partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropyle-ne-block polymers, polyethylene glycol and wool fat.

According to this disclosure, the pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceuti-cally-acceptable oils, such as olive oil or castor oil, especially in their polyoxyethylated versions. These oil solutions or suspensions may also contain a long-chain alcohol diluent or dispersant.

In some cases, depending on the dose and rate of injection, the binding sites on plasma proteins may become saturated with prodrug and activated agent. This leads to a decreased fraction of protein-bound agent and could compromise its half-life or tolerability as well as the effectiveness of the agent. In these circumstances, it is desirable to inject the prodrug agent in conjunction with a sterile albumin or plasma replacement solution. Alternatively, an apparatus/syringe can be used that contains the contrast agent and mixes it with blood drawn up into the syringe; this is then re-injected into the patient.

The compounds, diagnostic agents and pharmaceutical compositions of the present disclosure may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term “parenteral” as used herein includes subcutaneous, intravenous, intramuscular, intra-articular, intra-synovial, intrasternal, intrathecal, intrahepatic, intralesional and intracranial injection or infusion techniques.

When administered orally, the pharmaceutical compositions of this disclosure may be administered in any orally acceptable dosage form including, but not limited to, capsules, tablets, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, when administered in the form of suppositories for rectal administration, the pharmaceutical compositions of this disclosure may be prepared by mixing the agent with a suitable non-irritating excipient that is solid at room temperature but liquid at rectal temperature and therefore will melt in the rectum to release the drug. Such materials include cocoa butter, beeswax and polyethylene glycols.

As noted before, the pharmaceutical compositions of this disclosure may also be administered topically, especially when the target of treatment includes areas or organs readily accessible by topical application, including the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds and/or diagnostic agents of this disclosure include, but are not limited to, mineral oil, liquid petrolatum, white petrolatum, propylene glycol, poly-oxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, typically, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

For administration by nasal aerosol or inhalation, the pharmaceutical compositions of this disclosure are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active ingredient that may be combined with the carrier materials to produce a single dosage form will vary depending upon the host treated and the particular mode of administration. A typical preparation will contain from about 5% to about 95% active compound (w/w). Typically, such preparations contain from about 20% to about 80% active compound.

For intravenous and other types of administration, acceptable dose ranges range from about 0.001 to about 1.0 mmol/kg of body weight, with the typical dose of the active ingredient compound ranging from about 0.001 to about 0.5 mmol/kg of body weight. Even more typical is from about 0.01 to about 0.1 mmol/kg, and the most typical dose of the active ingredient compound is from about 0.0001 and to about 0.05 mmol/kg.

As the skilled artisan will appreciate, lower or higher doses than those recited above may be required. Specific dosage regimens for any particular patient will depend upon a variety of factors, including the activity of the specific compound employed, the age, body weight, general health status, sex, diet, time of administration, rate of excretion, drug combination and the judgment of the treating physician.

Another aspect of the present disclosure is diagnostic kits for the preparation of diagnostic agents for detecting, imaging, and/or monitoring a pathological disorder associated with matrix metalloproteinase activity. Diagnostic kits of the present disclosure comprise one or more vials containing the sterile, non-pyrogenic, formulation comprising a predetermined amount of a reagent of the present disclosure, and optionally other components such as one or two ancillary ligands such as tricine and 3-[bis(3-sulfophenyl)phosphine]benzenesulfonic acid (TPPTS), reducing agents, transfer ligands, buffers, lyophilization aids, stabilization aids, solubilization aids and bacteriostats.

The inclusion of one or more optional components in the formulation will frequently improve the ease of synthesis of the diagnostic agent by the practicing end user, the ease of manufacturing the kit, the shelf-life of the kit, or the stability and shelf-life of the imaging agent. The inclusion of one or two ancillary ligands is required for diagnostic kits comprising reagent comprising a hydrazine or hydrazone bonding moiety. The one or more vials that contain all or part of the formulation can independently be in the form of a sterile solution or a lyophilized solid.

Another aspect of the present disclosure is diagnostic kits for the preparation of diagnostic agents for the diagnosis of cardiovascular disorders, infectious disease, inflammatory disease and cancer. Diagnostic kits of the present disclosure contain one or more vials containing the sterile, non-pyrogenic, formulation comprising a predetermined amount of the chelant described in this disclosure, a stabilizing coligand, a reducing agent, and optionally other components such as buffers, lyophilization aids, stabilization aids, solubilization aids and bacteriostats.

The inclusion of one or more optional components in the formulation will frequently improve the ease of synthesis of the diagnostic agent by practicing end user, the ease of manufacturing the kit, the shelf-life of the kit, or the stability and shelf-life of the imaging agent. The improvement achieved by the inclusion of an optional component in the formulation must be weighed against the added complexity of the formulation and added cost to manufacture the kit. The one or more vials that contain all or part of the formulation can independently be in the form of a sterile solution or a lyophilized solid.

Buffers useful in the preparation of diagnostic agents and kits thereof include but are not limited to phosphate, citrate, sulfosalicylate, and acetate. A more complete list can be found in the United States Pharmacopeia.

Lyophilization aids useful in the preparation of diagnostic agents and kits thereof include but are not limited to mannitol, lactose, sorbitol, dextran, Ficoll, and polyvinylpyrrolidine (PVP).

Stabilization aids useful in the preparation of of diagnostic agents and kits thereof include but are not limited to ascorbic acid, cysteine, monothioglycerol, sodium bisulfite, sodium metabisulfite, gentisic acid, and inositol.

Solubilization aids useful in the preparation of diagnostic agents and kits thereof include but are not limited to ethanol, glycerin, polyethylene glycol, propylene glycol, polyoxyethylene sorbitan monooleate, sorbitan monoloeate, polysorbates, poly(oxyethylene)-poly(oxypropylene)poly(oxyethylene) block copolymers (Pluronics) and lecithin. Typical solubilizing aids are polyethylene glycol, and Pluronics copolymers.

Bacteriostats useful in the preparation of of diagnostic agents and kits thereof include but are not limited to benzyl alcohol, benzalkonium chloride, chlorbutanol, and methyl, propyl or butyl paraben.

A component in a diagnostic kit can also serve more than one function. A reducing agent can also serve as a stabilization aid, a buffer can also serve as a transfer ligand, a lyophilization aid can also serve as a transfer, ancillary or coligand and so forth.

The predetermined amounts of each component in the formulation are determined by a variety of considerations that are in some cases specific for that component and in other cases dependent on the amount of another component or the presence and amount of an optional component. In general, the minimal amount of each component is used that will give the desired effect of the formulation. The desired effect of the formulation is that the practicing end user can synthesize the diagnostic agent and have a high degree of certainty that the diagnostic agent can be injected safely into a patient and will provide diagnostic information about the disease state of that patient.

The diagnostic kits of the present disclosure can also contain written instructions for the practicing end user to follow to synthesize the diagnostic agents. These instructions may be affixed to one or more of the vials or to the container in which the vial or vials are packaged for shipping or may be a separate insert, termed the package insert.

X-ray contrast agents, ultrasound contrast agents and metallopharmaceuticals for use as magnetic resonance imaging contrast agents are provided to the end user in their final form in a formulation contained typically in one vial, as either a lyophilized solid or an aqueous solution. The end user reconstitutes the lyophilized solid with water or saline and withdraws the patient dose or simply withdraws the dose from the aqueous solution formulation as provided.

These diagnostic agents, whether for gamma scintigraphy, positron emission tomography, MRI, ultrasound or x-ray image enhancement, are useful, inter alia, to detect and monitor changes in cardiovascular diseases over time.

The compounds and/or diagnostic agents of the present disclosure can be prepared following the procedures described herein. Generally, peptides, polypeptides and peptidomimetics are elongated by deprotecting the alpha-amine of the C-terminal residue and coupling the next suitably protected amino acid through a peptide linkage using the methods described. This deprotection and coupling procedure is repeated until the desired sequence is obtained. This coupling can be performed with the constituent amino acids in a stepwise fashion, or condensation of fragments (two to several amino acids), or combination of both processes, or by solid phase peptide synthesis according to the method originally described in J. Am. Chem. Soc., 1963, 85, 2149-2154.

The peptides, polypeptides and peptidomimetics may also be synthesized using automated synthesizing equipment. In addition to the foregoing, procedures for peptide, polypeptide and peptidomimetic synthesis are described in Stewart and Young, Solid Phase Peptide Synthesis, 2nd ed, Pierce Chemical Co., Rockford, Ill. (1984); Gross, Meienhofer, Udenfriend, Eds., The Peptides: Analysis, Synthesis, Biology, Vol. 1, 2, 3, 5, and 9, Academic Press, New York, (1980-1987); Bodanszky, Peptide Chemistry: A Practical Textbook, Springer-Verlag, New York (1988); and Bodanszky et al., The Practice of Peptide Synthesis, Springer-Verlag, New York (1984).

The coupling between two amino acid derivatives, an amino acid and a peptide, polypeptide or peptidomimetic, two peptide, polypeptide or peptidomimetic fragments, or the cyclization of a peptide, polypeptide or peptidomimetic can be carried out using standard coupling procedures such as the azide method, mixed carbonic acid anhydride (isobutyl chloroformate) method, carbodiimide (dicyclohexylcarbodiimide, diisopropylcarbodiimide, or water-soluble carbodiimides) method, active ester (p-nitrophenyl ester, N-hydroxysuccinic imido ester) method, Woodward reagent K method, carbonyldiimidazole method, phosphorus reagents such as BOP-Cl, or oxidation-reduction method. Some of these methods (especially the carbodiimide) can be enhanced by the addition of 1-hydroxybenzotriazole or 1-hydroxy-7-azabenzotriazole. These coupling reactions may be performed either in solution (liquid phase) or on a solid phase, such as polystyrene or a suitable resin (vide infra).

The functional groups of the constituent amino acids or amino acid mimetics are typically protected during the coupling reactions to avoid undesired bonds being formed. The protecting groups that can be used are listed in Greene, Protective Groups in Organic Synthesis, John Wiley & Sons, New York (1981) and The Peptides: Analysis, Synthesis, Biology, Vol. 3, Academic Press, New York (1981).

The α-carboxyl group of the C-terminal residue may be protected by an ester that can be cleaved to give the carboxylic acid. These protecting groups include:
(1) alkyl esters such as methyl and t-butyl;
(2) aryl esters such as benzyl and substituted benzyl, or
(3) esters that can be cleaved by mild base treatment or mild reductive means such as trichloroethyl and phenacyl esters.

In the solid phase case, the C-terminal amino acid is attached to an insoluble carrier (usually polystyrene). These insoluble carriers contain a group that will react with the carboxyl group to form a bond which is stable to the elongation conditions but readily cleaved later. Examples include: oxime resin (DeGrado and Kaiser (1980) J. Org. Chem. 45, 1295-1300) chloro or bromomethyl resin, hydroxymethyl resin, and aminomethyl resin. Many of these resins are commercially available with the desired C-terminal amino acid already incorporated.

The α-amino group of each amino acid is typically protected. Any protecting group known in the art may be used. Examples of these are:
(1) acyl types such as formyl, trifluoroacetyl, phthalyl, and p-toluenesulfonyl;
(2) aromatic carbamate types such as benzyloxycarbonyl (Cbz) and substituted benzyloxycarbonyls, 1-(p-biphenyl)-1-methylethoxycarbonyl, and 9-fluorenyl-methyloxycarbonyl (Fmoc);
(3) aliphatic carbamate types such as tert-butyloxycarbonyl (Boc), ethoxycarbonyl, diisopropylmethoxycarbonyl, and allyloxycarbonyl;
(4) cyclic alkyl carbamate types such as cyclopentyloxycarbonyl and adamantyloxycarbonyl;
(5) alkyl types such as triphenylmethyl and benzyl;
(6) trialkylsilane such as trimethylsilane; and
(7) thiol containing types such as phenylthiocarbonyl and dithiasuccinoyl.

Typical alpha-amino protecting groups are either Boc or Fmoc. Many amino acid or amino acid mimetic derivatives suitably protected for peptide synthesis are commercially available.

The α-amino protecting group is cleaved prior to the coupling of the next amino acid. When the Boc group is used, the methods of choice are trifluoroacetic acid, neat or in dichloromethane, or HCl in dioxane. The resulting ammonium salt is then neutralized either prior to the coupling or in situ with basic solutions such as aqueous buffers, or tertiary amines in dichloromethane or dimethylformamide. When the Fmoc group is used, the reagents of choice are piperidine or substituted piperidines in dimethylformamide, but any secondary amine or aqueous basic solutions can be used. The deprotection is carried out at a temperature between 0° C. and room temperature.

Any of the amino acids or amino acid mimetics bearing side chain functionalities are typically protected during the preparation of the peptide using any of the above-identified groups. Those skilled in the art will appreciate that the selection and use of appropriate protecting groups for these side chain functionalities will depend upon the amino acid or amino acid mimetic and presence of other protecting groups in the peptide, polypeptide or peptidomimetic. The selection of such a protecting group is important in that it must not be removed during the deprotection and coupling of the α-amino group.

For example, when Boc is chosen for the α-amine protection the following protecting groups are acceptable: p-toluenesulfonyl (tosyl) moieties and nitro for arginine; benzyloxycarbonyl, substituted benzyloxycarbonyls, tosyl or trifluoroacetyl for lysine; benzyl or alkyl esters such as cyclopentyl for glutamic and aspartic acids; benzyl ethers for serine and threonine; benzyl ethers, substituted benzyl ethers or 2-bromobenzyloxycarbonyl for tyrosine; p-methylbenzyl, p-methoxybenzyl, acetamidomethyl, benzyl, or t-butylsulfonyl for cysteine; and the indole of tryptophan can either be left unprotected or protected with a formyl group.

When Fmoc is chosen for the a-amine protection usually tert-butyl based protecting groups are acceptable. For instance, Boc can be used for lysine, tert-butyl ether for serine, threonine and tyrosine, and tert-butyl ester for glutamic and aspartic acids.

Once the elongation of the peptide, polypeptide or peptidomimetic, or the elongation and cyclization of a cyclic peptide or peptidomimetic is completed all of the protecting groups are removed. For the liquid phase synthesis the protecting groups are removed in whatever manner as dictated by the choice of protecting groups. These procedures are well known to those skilled in the art.

When a solid phase synthesis is used to synthesize a cyclic peptide or peptidomimetic, the peptide or peptidomimetic should be removed from the resin without simultaneously removing protecting groups from functional groups that might interfere with the cyclization process. Thus, if the peptide or peptidomimetic is to be cyclized in solution, the cleavage conditions need to be chosen such that a free a-carboxylate and a free a-amino group are generated without simultaneously removing other protecting groups. Alternatively, the peptide or peptidomimetic may be removed from the resin by hydrazinolysis, and then coupled by the azide method. Another very convenient method involves the synthesis of peptides or peptidomimetics on an oxime resin, followed by intramolecular nucleophilic displacement from the resin, which generates a cyclic peptide or peptidomimetic (Tetrahedron Letters, 1990, 43, 6121-6124). When the oxime resin is employed, the Boc protection scheme is generally chosen. Then, a typical method for removing side chain protecting groups generally involves treatment with anhydrous HF containing additives such as dimethyl sulfide, anisole, thioanisole, or p-cresol at 0° C. The cleavage of the peptide or peptidomimetic can also be accomplished by other acid reagents such as trifluoromethanesulfonic acid/trifluoroacetic acid mixtures.

Unusual amino acids used in this disclosure can be synthesized by standard methods familiar to those skilled in the art (The Peptides: Analysis, Synthesis, Biology, Vol. 5, pp. 342-449, Academic Press, New York (1981)). N-Alkyl amino acids can be prepared using procedures described previously (Cheung et al., Can. J. Chem., 1977, 55, 906; Freidinger et al., J. Org. Chem., 1982, 48, 77).

The chelator is selected to form stable complexes with the metal ion chosen for the particular application. Chelators for diagnostic radiopharmaceuticals are selected to form stable complexes with the radioisotopes that have imageable gamma ray or positron emissions, such as ^{99m}Tc, ⁹⁵Tc, ¹¹¹In, ⁶²Cu, ⁶⁰Cu, ⁶⁴Cu, ⁶⁷Ga, ⁶⁸Ga, ⁸⁶Y.

Chelators for technetium, copper and gallium isotopes are selected from diaminedithiols, monoamine-monoamidedithiols, triamide-monothiols, monoamine-diamide-monothiols, diaminedioximes, and hydrazines. The chelators are generally tetradentate with donor atoms selected from nitrogen, oxygen and sulfur. The thiol sulfur atoms and the hydrazines may bear a protecting group which can be displaced either prior to using the reagent to synthesize a radiopharmaceutical or more often in situ during the synthesis of the radiopharmaceutical.

Exemplary thiol protecting groups include those listed in Greene and Wuts, Protective Groups in Organic Synthesis, John Wiley & Sons, New York (1991). Any thiol protecting group known in the art may be used. Examples of thiol protecting groups include, but are not limited to, the following: acetamidomethyl, benzamidomethyl, 1-ethoxyethyl, benzoyl, and triphenylmethyl.

Exemplary protecting groups for hydrazine chelators are hydrazones which can be aldehyde or ketone hydrazones having substituents selected from hydrogen, alkyl, aryl and heterocycle. Examples of hydrazones are described in U.S. Pat. No. 5,750,088.

The hydrazine chelator, when bound to a metal radionuclide, is termed a hydrazido, or diazenido group and serves as the point of attachment of the radionuclide to the remainder of the radiopharmaceutical. A diazenido group can be either terminal (only one atom of the group is bound to the radionuclide) or chelating. In order to have a chelating diazenido group at least one other atom of the group must also be bound to the radionuclide. The atoms bound to the metal are termed donor atoms.

Chelators for such metals as indium (e.g. ¹¹¹In), yttrium (e.g. ⁸⁶Y & ⁹⁰Y), and lanthanides (e.g. Eu(III), Gd(III), and Dy(III)) are selected from cyclic and acyclic polyaminocarboxylates such as DTPA, DOTA, DO3A, 2-benzyl-DOTA, alpha-(2-phenethyl) 1,4,7,10-tetraazazcyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyphenyl)-2,2′:6′,2″-terpyridine. Procedures for synthesizing these chelators that are not commercially available can be found in J. Chem. Soc. Perkin Trans., 1992, 1, 1175; Bioconjugate Chem., 1991, 2, 187; J. Nucl. Med., 1990, 31, 473; U.S. Pat. No. 5,064,956, and U.S. Pat. No. 4,859,777.

The coordination sphere of metal ion includes all the ligands or groups bound to the metal. For a transition metal complex to be stable it typically has a coordination number (number of donor atoms) comprised of an integer greater than or equal to 4 and less than or equal to 8; that is there are 4 to 8 atoms bound to the metal and it is said to have a complete coordination sphere. For a lanthanide series or actinide series metal complex, the metal typically has a coordination number (number of donor atoms) comprised of an integer greater than or equal to 4 and less than or equal to 10; that is there are 4 to 10 atoms bound to the metal and it is said to have a complete coordination sphere. The requisite coordination number for a stable metallopharmaceutical complex is determined by the identity of the element, its oxidation state, and the type of donor atoms. If the chelator does not provide all of the atoms necessary to stabilize the metal complex by completing its coordination sphere, the coordination sphere is completed by donor atoms from other ligands, termed ancillary or co-ligands, which can also be either terminal or chelating.

A large number of ligands can serve as ancillary or co-ligands, the choice of which is determined by a variety of considerations such as the ease of synthesis of the radiopharmaceutical, the chemical and physical properties of the ancillary ligand, the rate of formation, the yield, and the number of isomeric forms of the resulting radiopharmaceuticals, the ability to administer said ancillary or co-ligand to a patient without adverse physiological consequences to said patient, and the compatibility of the ligand in a lyophilized kit formulation. The charge and lipophilicity of the ancillary ligand will effect the charge and lipophilicity of the radiopharmaceuticals. For example, the use of 4,5-dihydroxy-1,3-benzene disulfonate results in radiopharmaceuticals with an additional two anionic groups because the sulfonate groups will be anionic under physiological conditions. The use of N-alkyl substituted 3,4-hydroxypyridinones results in radiopharmaceuticals with varying degrees of lipophilicity depending on the size of the alkyl substituents.

Certain technetium radiopharmaceuticals of the present disclosure are comprised of a hydrazido or diazenido chelator and an ancillary ligand, A_{L1}, or a chelator and two types of ancillary ligands A_{L1} and A_{L2}, or a tetradentate chelator comprised of two nitrogen and two sulfur atoms. Ancillary ligands A_{L1} are comprised of two or more hard donor atoms such as oxygen and amine nitrogen (sp³ hybridized). The donor atoms occupy at least two of the sites in the coordination sphere of the radionuclide metal; the ancillary ligand A_{L1} serves as one of the three ligands in the ternary ligand system. Examples of ancillary ligands A_{L1} include but are not limited to dioxygen ligands and functionalized aminocarboxylates. A large number of such ligands are available from commercial sources.

Ancillary dioxygen ligands include ligands that coordinate to the metal ion through at least two oxygen donor atoms. Examples include but are not limited to: glucoheptonate, gluconate, 2-hydroxyisobutyrate, lactate, tartrate, mannitol, glucarate, maltol, Kojic acid, 2,2-bis(hydroxymethyl)propionic acid, 4,5-dihydroxy-1,3-benzene disulfonate, or substituted or unsubstituted 1,2- or 3,4-hydroxypyridinones. (The names for the ligands in these examples refer to either the protonated or non-protonated forms of the ligands.)

Functionalized aminocarboxylates include ligands that have a combination of amine nitrogen and oxygen donor atoms. Examples include but are not limited to: iminodiacetic acid, 2,3-diaminopropionic acid, nitrilotriacetic acid, N,N′-ethylenediamine diacetic acid, N,N,N′-ethylenediamine triacetic acid, hydroxyethylethylenediamine triacetic acid, and N,N′-ethylenediamine bis-hydroxyphenylglycine. (The names for the ligands in these examples refer to either the protonated or non-protonated forms of the ligands.)

A series of functionalized aminocarboxylates are disclosed in U.S. Pat. No. 5,350,837 that result in improved rates of formation of technetium labeled hydrazino modified proteins. We have determined that certain of these aminocarboxylates result in improved yields of the radiopharmaceuticals of the present disclosure. Examples of ancillary ligands A_{L1} include functionalized aminocarboxylates that are derivatives of glycine; for example, tricine (tris(hydroxymethyl)methylglycine).

Examples of technetium diagnostic agent of the present disclosure comprise a hydrazido or diazenido chelator and two types of ancillary ligand designated A_{L1} and A_{L2}, or a diaminedithiol chelator. The second type of ancillary ligands A_{L2} comprise one or more soft donor atoms selected from phosphine phosphorus, arsine arsenic, imine nitrogen (sp² hybridized), sulfur (sp² hybridized) and carbon (sp hybridized); atoms which have p-acid character. Ligands A_{L2} can be monodentate, bidentate or tridentate; the denticity is defined by the number of donor atoms in the ligand. One of the two donor atoms in a bidentate ligand and one of the three donor atoms in a tridentate ligand must be a soft donor atom. U.S. Pat. No. 5,744,120 and U.S. Pat. No. 5,739,789 disclose radiopharmaceuticals comprising one or more ancillary or co-ligands A_{L2} that are more stable compared to radiopharmaceuticals that do not comprise one or more ancillary ligands, A_{L2}; that is, they have a minimal number of isomeric forms, the relative ratios of which do not change significantly with time, and that remain substantially intact upon dilution.

The ligands A_{L2} that comprise phosphine or arsine donor atoms are trisubstituted phosphines, trisubstituted arsines, tetrasubstituted diphosphines and tetrasubstituted diarsines. The ligands A_{L2} that comprise imine nitrogen are unsaturated or aromatic nitrogen-containing, 5 or 6-membered heterocycles. The ligands that comprise sulfur (sp² hybridized) donor atoms are thiocarbonyls, and comprise the moiety C═S. The ligands comprising carbon (sp hybridized) donor atoms are isonitriles, comprising the moiety CNR, where R is an organic radical. A large number of such ligands are available from commercial sources. Isonitriles can be synthesized as described in U.S. Pat. No. 4,452,774 and U.S. Pat. No. 4,988,827.

Examples of ancillary ligands A_{L2} are trisubstituted phosphines and unsaturated or aromatic 5 or 6 membered heterocycles.

The ancillary ligands A_{L2} may be substituted with alkyl, aryl, alkoxy, heterocyclyl, arylalkyl, alkylaryl and arylalkylaryl groups and may or may not bear functional groups comprising heteroatoms such as oxygen, nitrogen, phosphorus or sulfur. Examples of such functional groups include but are not limited to: hydroxyl, carboxyl, carboxamide, nitro, ether, ketone, amino, ammonium, sulfonate, sulfonamide, phosphonate, and phosphonamide. The functional groups may be chosen to alter the lipophilicity and water solubility of the ligands that may affect the biological properties of the radiopharmaceuticals, such as altering the distribution into non-target tissues, cells or fluids, and the mechanism and rate of elimination from the body.

Chelators for magnetic resonance imaging contrast agents are selected to form stable complexes with paramagnetic metal ions, such as Gd(III), Dy(III), Fe(III), and Mn(II), are selected from cyclic and acyclic polyaminocarboxylates such as DTPA, DOTA, DO3A, 2-benzyl-DOTA, alpha-(2-phenethyl)1,4,7,10-tetraazacyclododecane-1-acetic-4,7, 1 0-tris (methyl acetic) acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyphenyl)-2,2′:6′,2″-terpyridine.

The rate of clearance from the blood is of particular importance for cardiac imaging procedures, since the cardiac blood pool is large compared to the disease foci that one desires to image. For an effective cardiac imaging agent, the target to background ratios (disease foci-to-blood and disease foci-to-muscle) are typically greater or equal to about 1.5, typically greater or equal to about 2.0, and more typically even greater. Certain pharmaceuticals of the present disclosure have blood clearance rates that result in less than about 10% i.d./g at 2 hours post-injection, measured in a mouse model, or less than about 0.5% i.d./g at 2 hours post-injection, measured in a dog model. In one embodiment diagnostic agents of the present disclosure have blood clearance rates that result in less than about 3% i.d./g at 2 hours post-injection, measured in a mouse model, or less than about 0.05% i.d./g at 2 hours post-injection, measured in a dog model.

The diagnostic agents of the disclosure containing technetium further comprising hydrazido or diazenido chelator units can be easily prepared by admixing a salt of a radionuclide, a reagent of the present disclosure, an ancillary ligand A_{L1}, an ancillary ligand A_{L2}, and a reducing agent, in an aqueous solution at temperatures from about 0° C. to about 100° C. The diagnostic agents of the disclosure containing technetium comprising a tetradentate chelator having two nitrogen and two sulfur atoms can be easily prepared by admixing a salt of a radionuclide, a reagent of the present disclosure, and a reducing agent, in an aqueous solution at temperatures from about 0° C. to about 100° C.

When the chelator in the reagent of the present disclosure is present as a hydrazone group, then it first typically converted to a hydrazine, which may or may not be protonated, prior to complexation with the metal radionuclide. The conversion of the hydrazone group to the hydrazine can occur either prior to reaction with the radionuclide, in which case the radionuclide and the ancillary or co-ligand or ligands are combined not with the reagent but with a hydrolyzed form of the reagent bearing the chelator, or in the presence of the radionuclide in which case the reagent itself is combined with the radionuclide and the ancillary or co-ligand or ligands. In the latter case, the pH of the reaction mixture is usually neutral or acidic.

Alternatively, the diagnostic agents of the present disclosure comprising hydrazido or diazenido chelator may be prepared by first admixing a salt of a radionuclide, an ancillary ligand A_{L1}, and a reducing agent in an aqueous solution at temperatures from about 0° C. to about 100° C. to form an intermediate radionuclide complex with the ancillary ligand A_{L1} then adding a reagent of the present disclosure and an ancillary ligand A_{L2} and reacting further at temperatures from about 0° C. to about 100° C.

Alternatively, the diagnostic agents of the present disclosure comprising a hydrazido or diazenido chelator may be prepared by first admixing a salt of a radionuclide, an ancillary ligand A_{L1}, a reagent of the present disclosure, and a reducing agent in an aqueous solution at temperatures from about 0° C. to about 100° C. to form an intermediate radionuclide complex, and then adding an ancillary ligand A_{L2} and reacting further at temperatures about 0° C. to about 100° C.

The technetium radionuclides are typically in the chemical form of pertechnetate or perrhenate and a pharmaceutically acceptable cation. The pertechnetate salt form is typically sodium pertechnetate such as obtained from commercial ^{99m}Tc generators. The amount of pertechnetate used to prepare the radiopharmaceuticals of the present disclosure can range from about 0.1 mCi to about 1 Ci, or more typically from about 1 to about 200 mCi.

The amount of the reagent of the present disclosure used to prepare the technetium diagnostic agent of the present disclosure may range from about 0.01 μg to about 10 mg, or more typically from about 0.5 μg to about 200 μg. The amount used will be dictated by the amounts of the other reactants and the identity of the radiopharmaceuticals of the present disclosure to be prepared.

The amounts of the ancillary ligands A_{L1} used may range from about 0.1 mg to about 1 g, or more typically from about 1 mg to about 100 mg. The exact amount for a particular radiopharmaceutical is a function of identity of the radiopharmaceuticals of the present disclosure to be prepared, the procedure used and the amounts and identities of the other reactants. Too large an amount of A_{L1} will result in the formation of by-products comprised of technetium labeled A_{L1} without a biologically active molecule or by-products comprised of technetium labeled biologically active molecules with the ancillary ligand AL, but without the ancillary ligand A_{L2}. Too small an amount of A_{L1} will result in other by-products such as technetium labeled biologically active molecules with the ancillary ligand A_{L2} but without the ancillary ligand A_{L1}, or reduced hydrolyzed technetium, or technetium colloid.

The amounts of the ancillary ligands A_{L2} used may range from about 0.001 mg to about 1 g, or more typically from about 0.01 mg to about 10 mg. The exact amount for a particular radiopharmaceutical is a function of the identity of the radiopharmaceuticals of the present disclosure to be prepared, the procedure used and the amounts and identities of the other reactants. Too large an amount of A_{L2} will result in the formation of by-products comprised of technetium labeled A_{L2} without a biologically active molecule or by-products comprised of technetium labeled biologically active molecules with the ancillary ligand A_{L2} but without the ancillary ligand A_{L1}.

The indium, copper, gallium, and yttrium diagnostic agents of the present disclosure can be easily prepared by admixing a salt of a radionuclide and a reagent of the present disclosure, in an aqueous solution at temperatures from about 0° C. to about 100° C. These radionuclides are typically obtained as a dilute aqueous solution in a mineral acid, such as hydrochloric, nitric or sulfuric acid. The radionuclides are combined with from one to about one thousand equivalents of the reagents of the present disclosure dissolved in aqueous solution. A buffer is typically used to maintain the pH of the reaction mixture from about 3 to about 10.

The gadolinium, dysprosium, iron and manganese diagnostic agents of the present disclosure can be easily prepared by admixing a salt of the paramagnetic metal ion and a reagent of the present disclosure, in an aqueous solution at temperatures from about 0° C. to about 100° C. These paramagnetic metal ions are typically obtained as a dilute aqueous solution in a mineral acid, such as hydrochloric, nitric or sulfuric acid. The paramagnetic metal ions are combined with from one to about one thousand equivalents of the reagents of the present disclosure dissolved in aqueous solution. A buffer is typically used to maintain the pH of the reaction mixture from about 3 to about 10.

The total time of preparation will vary depending on the identity of the metal ion, the identities and amounts of the reactants and the procedure used for the preparation. The preparations may be complete, resulting in greater than about 80% yield of the radiopharmaceutical, in about 1 minute or may require more time. If higher purity metallopharmaceuticals are needed or desired, the products can be purified by any of a number of techniques well known to those skilled in the art such as liquid chromatography, solid phase extraction, solvent extraction, dialysis or ultrafiltration.

The diagnostic radiopharmaceuticals are administered by intravenous injection, usually in saline solution, at a dose of about 1 to about 100 mCi per 70 kg body weight, or typically at a dose of about 5 to about 50 mCi. Imaging is performed using known procedures.

The diagnostic agents of the disclosure containing a magnetic resonance imaging contrast component may be used in a similar manner as other MRI agents as described in U.S. Pat. No. 5,155,215; U.S. Pat. No. 5,087,440; Magn. Reson. Med., 1986, 3, 808; Radiology, 1988, 166, 835; and Radiology, 1988, 166, 693. Generally, sterile aqueous solutions of the contrast agents are administered to a patient intravenously in dosages ranging from about 0.01 to about 1.0 mmoles per kg body weight.

For use as X-ray contrast agents, the diagnostic agents of the present disclosure should generally have a heavy atom concentration of about 1 mM to about 5 M, typically about 0.1 M to about 2 M. Dosages, administered by intravenous injection, will typically range from about 0.5 mmol/kg to about 1.5 mmol/kg, typically about 0.8 mmol/kg to about 1.2 mmol/kg. Imaging is performed using known techniques, typically X-ray computed tomography.

The diagnostic agents of the disclosure containing ultrasound contrast components are administered by intravenous injection in an amount of about 10 to about 30 μL of the echogenic gas per kg body weight or by infusion at a rate of about 3 μL/kg/min. Imaging may be performed using known techniques of sonography.

Other features of the disclosure will become apparent in the course of the following descriptions of exemplary embodiments which are given for illustration of the disclosure and are not intended to be limiting thereof. The present disclosure will now be illustrated by reference to the following specific, non-limiting examples. Those skilled in the art of organic synthesis may be aware of still other synthetic routes to the disclosure compounds and/or diagnostic agents. The reagents and intermediates used herein are either commercially available or prepared according to standard literature procedures, unless otherwise described.

This disclosure is intended to encompass compounds having formula (I) when prepared by synthetic processes or by metabolic processes including those occurring in the human or animal body (in vivo) or processes occurring in vitro. For example, compounds of the present disclosure where A is a peptide consisting of a D-amino acid residue and a second D-amino acid may be generated by cleavage of a larger sequence (e.g., a peptide consisting of 3 amino acids and a D-amino acid residue) either synthetically or in vivo.

General. ¹H NMR spectra were recorded on a Bruker Avance DRX (600 MHz) spectrometer. Chemical shifts are reported in ppm from tetramethylsilane with the residual solvent resonance resulting from incomplete deuteration as the internal standard (CDCl₃: δ 7.25 ppm, C₆D₆: δ 7.16 ppm, DMSO-d₆: δ 2.50 ppm). Data are reported as follows: chemical shift, integration, multiplicity (s=singlet, d=doublet, t=triplet, q=quartet, quin=quintet, b or br=broad, m=multiplet), and coupling constants. ¹³C NMR spectra were recorded on a Bruker Avance DRX (150 MHz) with complete proton decoupling. Chemical shifts are reported in ppm from tetramethylsilane with the solvent as the internal reference (CDCl₃: δ 77.0 ppm, C₆D₆: δ 128.4 ppm, DMSO-d₆: δ 39.5 ppm). ¹⁹F NMR spectra were recorded on a Bruker Avance DRX (565 MHz); chemical shifts are reported in ppm relative to an external standard (CCl₃F; δ=0.00 ppm).

Enantiomer ratios were determined by chiral GLC analysis (Alltech Associates Chiralsil-Val column (25 m×0.25 mm)) in comparison with authentic racemic materials. Low-resolution mass spectrometry was performed on an Agilent Technologies 1100 Series LC/MS ESI-MS (positive mode). High-resolution mass spectrometry was performed on a lonspec Ultima FTMS; ESI-MS (positive mode).

Unless otherwise stated, all reactions were conducted in oven (150° C.) and flame-dried glassware under an inert atmosphere of dry nitrogen. Indicated temperatures refer to those of the reaction bath, while ambient laboratory temperature is noted as 22° C. Anhydrous solvents were obtained from Aldrich and were used as received. Amino acid derivatives and amino acid resins were obtained from Advanced Chemtech, Novabiochem, Bachem Bioscience, RSP Amino Acids, or PepTech Amino Acids. Within each experimental are footnotes concerning the preparation of certain starting materials. 4-Cyanocinnamic acid was obtained from Ryan Scientific. 4-(Aminomethyl)phenylacetic acid was obtained from Tyger Scientific. Alexa Fluor 350™ succinimidyl ester was purchased from Molecular Probes. DOTA tri-t-butyl ester was purchased from Macrocyclics. All other reagents were used as obtained from Aldrich, Fluka, Lancaster, or Strem Chemicals.

Abbreviations

Commonly accepted abbreviations for widely used reagents and solvents are used throughout the patent. The 21 natural amino acids are described using the commonly accepted three letter abbreviations. Other abbreviations are as described below.
Ahx=6-aminohexanoic acid
Aib=α-aminoisobutyric acid
Amb=4-aminomethylbenzoic acid
Bip=biphenylalanine
DCHA=N,N-dicyclohexylamine
DEA=diethylamine
DIEA=diisopropylethylamine
EDC=1-[3-(dimethylamino)propyl]-3-ethylcarbodiimide hydrochloride
EEDQ=2-ethoxy-1-ethoxycarbonyl-1,2-dihydroquinoline
HBTU=2-(1H-benzotriazol-1-yl)-1,1,3,3-tetramethyluronium hexafluorophosphate
HOAt=1-hydroxy-7-azabenzotriazole
HOBt=1-hydroxybenzotriazole
Hphe=homophenylalanine
Nal=Naphthylalanine
NLeu=N-leucine
NLys=N-Lysine
Stya=Styrylalanine
TAEA=tris(2-aminoethyl)amine
TEA=triethylamine
Tic=1,2,3,4-tetrahydroisoquinoline-3-carboxylic acid
TIS=triisopropylsilane
TMP=2,4,6-trimethylpyridine

### EXAMPLE 1

### Synthesis of 2-((1E)-2-{[5-(N-{5-[N-((2R)-2-Amino-4-methylpentanoylamino)-carbamoyl]pentyl}carbamoyl)(2-pyridyl)]amino}-2-azavinyl)benzenesulfonic Acid

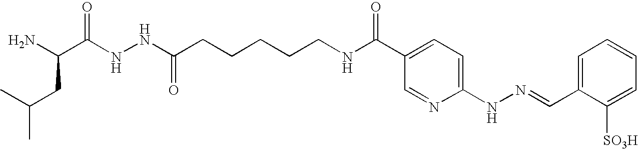

### Part A—Preparation of N-[(tert-Butoxy)carbonylamino]-6-[(fluoren-9-ylmethoxy)carbonylamino]hexanamide

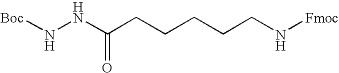

A solution of Fmoc-6-Ahx-OH (15.0 g, 42.4 mmol), HBTU (20.9 g, 55.12 mmol,), HOBt (8.44 g, 55.12 mmol), and DIEA (18.5 mL, 106 mmol) in 200 mL of DMF was treated with a solution of t-butyl carbazate (6.73 g, 50.8 mmol) and DIEA (3.7 mL, 21.2 mmol ) in 50 mL of DMF. Additional DIEA was added as necessary to maintain a pH=10. After 45 minutes the solution was concentrated under reduced pressure, and the residue was taken up in ethyl acetate. The solution was washed consecutively with 0.1 N HCl (2×250 mL), saturated NaHCO₃ (2×250 mL), and saturated NaCl (150 mL). The organic layer was dried (MgSO₄), filtered, and concentrated to give the title compound as a pale yellow solid (20.7 g, 98%). MS (ESI): 368.3 (100, M+H−Boc).

### Part B—Preparation of 6-Amino-N-[(tert-butoxy)carbonylamino]hexanamide

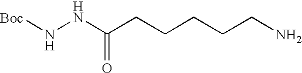

The product of Part A (1.44 g, 3.1 mmol) was treated with 20% piperidine in DMF (4.0 mL) at room temperature under nitrogen for 20 minutes. The solution was concentrated under reduced pressure and the resulting solid was purified by flash chromatography over silica gel, eluting consecutively with methanol, 100:3 methanol:TEA, and 100:6 methanol:TEA, to give the title compound as a colorless solid (0.79 g, 104%). ¹H NMR (CDCl₃): δ 4.12 (bs, 2H), 2.80-2.68 (m, 2H), 2.24 (t, J=7.3 Hz, 2H), 1.72-1.60 (m, 2H), 1.58-1.46 (m, 2H), 1.45 (s, 9H), 1.43-1.33 (m 2H). MS (ESI): 246.3 (M+H).

### Part C—Preparation of Sodium 2-[(1E)-2-Aza-2-({5-[N-(5-{N-[(tert-butoxy)-carbonylamino]carbamoyl}pentyl)carbamoyl](2-pyridyl)}amino)vinyl]benzenesulfonate

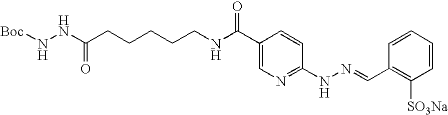

A solution of the product of Part B (0.72 g, 2.9 mmol), sodium 2-[(1E)-2-aza-2-({5-[(2,5-dioxopyrrolidinyl)oxycarbonyl](2-pyridyl)}amino)vinyl]benzenesulfonate (prepared according to the procedure described in Bioconjugate Chemistry (1999), 10(5), 808-814, 1.29 g, 2.9 mmol), HOAt (0.40 g, 2.9 mmol), and DIEA (1.02 mL, 5.9 mmol) in anhydrous DMF (10 mL) was stirred at room temperature under nitrogen. After 2 hours, additional sodium 2-[(1E)-2-aza-2-({5-[(2,5-dioxopyrrolidinyl)oxycarbonyl](2-pyridyl)}amino)vinylbenzene sulfonate (0.27 g, 0.6 mmol) and DIEA (0.1 mL, 0.6 mmol) were added and the reaction was stirred overnight. The reaction mixture was filtered and the filtrate was concentrated. The resulting residue was purified by flash chromatography over silica gel, eluting with 85:15 dichloromethane:methanol, to give the title compound as a colorless solid (0.81 g, 50%, HPLC purity >95%). ¹H NMR (DMSO-d₆): δ 11.32 (s, 1H), 9.45 (s, 1H), 9.01 (s, 1H), 8.63 (s, 1H), 8.59 (d, J=2.1 Hz, 1H), 8.34-8.23 (m, 1H), 8.08-7.97 (m, 2H), 7.78 (dd, J=1.4, 7.5 Hz, 1H), 7.40-7.18 (m, 3H), 3.28-3.17 (m, 2H), 2.07 (t, J=7.2 Hz, 2H), 1.60-1.45 (m, 4H), 1.45-1.21 (m, 11H). MS (ESI): 449.2 (M−Boc+H).

### Part D—Preparation of 2-{(1E)-2-[(5-{N-[5-(N-Aminocarbamoyl)pentyl]carbamoyl}(2-pyridyl))amino]-2-azavinyl}benzenesulfonic Acid

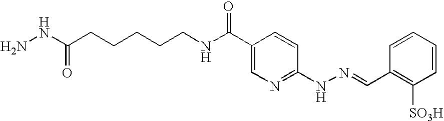

The product of Part C (0.37 g, 0.7 mmol) was treated with 50% TFA in dichloromethane (5 mL) for 10 minutes at room temperature under nitrogen. The solution was concentrated under reduced pressure and the residue was purified by HPLC on a Phenomenex Jupiter C18 column (41.4×250 mm) using a 0.9%/min gradient of 0 to 27% acetonitrile containing 0.1% TFA at a flow rate of 80 mL/min. The main product peak eluting at 18.9 minutes was lyophilized to give the title compound as a colorless solid (0.24 g, 80%). ¹H NMR (DMSO-d₆): δ 10.75 (s, 1H), 9.22 (s, 1H), 8.64-8.54 (m, 1H), 8.53 (d, J=1.8 Hz, 1H), 8.29-8.11 (m, 2H), 7.80 (dd, J=1.9, 7.0 Hz, 1H), 7.47-7.32 (m, 2H), 7.23 (d, J=9.1 Hz, 1H), 4.50 (bs, 3H), 3.26 (q, J=6.4 Hz, 2H), 2.23 (t, J=7.3 Hz, 2H), 1.66-1.45 (m, 4H), 1.40-1.22 (m, 2H). MS (ESI): 449.1 (M+H).

### Part E—Preparation of 2-((1E)-2-{[5-(N-{5-[N-((2R)-2-Amino-4-methylpentanoylamino)-carbamoyl]pentyl}carbamoyl)(2-pyridyl)]amino}-2-azavinyl)benzenesulfonic Acid

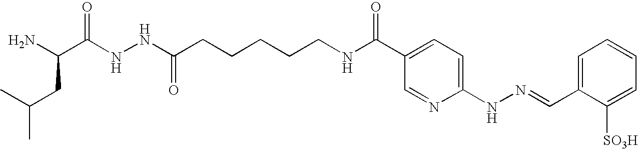

A solution of Fmoc-D-Leu-OH (16.0 mg, 0.045 mmol), the product of Part D (20.3 mg, 0.045 mmol), and HOAt (12.1 mg, 0.090 mmol) in anhydrous DMF (1 mL) was treated with collidine (37.0 μL, 0.280 mmol) and DIC (13.8 μL, 0.090 mmol), and stirred at room temperature under nitrogen for 2 hours. Then piperidine (0.25 mL) was added to the reaction, and after 10 minutes, the reaction was concentrated under reduced pressure. The resulting residue was purified by HPLC on a Phenomenex Luna column (21.2×250 mm) using a 0.9%/min gradient of 0 to 27% acetonitrile containing 0.1% TFA at a flow rate of 20 mL/min. The main product peak eluting at 25.4 minutes was lyophilized to give the title compound as a colorless solid (8.6 mg, 34%, HPLC purity 100%). ¹H NMR (DMSO-d₆): δ 10.41 (s, 1H), 10.01 (s, 1H), 9.24 (bs, 1H), 8.60 (bs, 1H), 8.54 (s, 1H), 8.31-8.16 (m, 3H), 7.81 (dd, J=1.8, 7.2 Hz, 1H), 7.46-7.37 (m, 2H), 7.22 (d, J=9.0 Hz, 1H), 3.78 (bs, 1H), 3.25 (q, J=6.0 Hz, 2H), 2.17 (t, J=7.5 Hz, 2H), 1.78-1.69 (m, 1H), 1.62-1.49 (m, 6H), 1.48-1.30 (m, 2H), 0.94-0.87 (m, 6H). MS (ESI): 562.2 (100, M+H), 1123.3 (15, 2M+H). HRMS: Calcd for C₂₅H₃₅N₇O₆S (M+H): 562.2442, Found: 562.2434.

### EXAMPLE 2

### Synthesis of 2-{(1E)-2-Aza-2-[(5-{N-[5-(N-{2-[(2-methylpropyl)amino]-acetylamino}-carbamoyl)pentyl]carbamoyl}(2-pyridyl))amino]vinyl}benzenesulfonic Acid

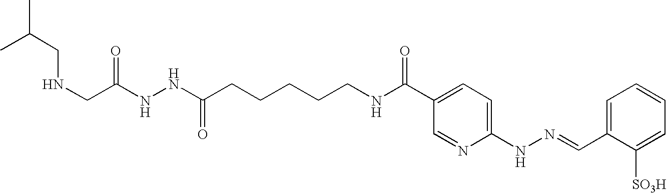

A solution of Fmoc-NLeu-OH (19.8 mg, 0.056 mmol, prepared by the procedure of Kruijtzer, J. A. W.; Hofmeyer, L. J. F.; Heerma, W.; Verslius, C.; Liskamp, R. M. J. Chem. Eur. J. 1998, 8, 1570-1580), and HOAt (7.6 mg, 0.056 mmol) in anhydrous DMF (0.5 mL) was treated with collidine (37.0 μL, 0.280 mmol) and DIC (17.4 μL, 0.112 mmol), and pre-activated for 15 minutes. The product of Example 1D was added, and the reaction was stirred at room temperature under nitrogen. After 4 hours additional product of Example 1D (25.1 mg, 0.056 mmol) and DIC (17.4 μL, 0.112 mmol) were added, and the reaction was stirred for an additional 18 hours. The solvents were removed under reduced pressure, and the residue was taken up in 20% piperidine in DMF (0.5 mL). After 30 minutes, the reaction was concentrated under reduced pressure. The resulting residue was purified by HPLC on a Phenomenex Luna column (21.2×250 mm) using a 0.9%/min gradient of 0 to 31.5% acetonitrile containing 0.1% TFA at a flow rate of 20 mL/min. The main product peak eluting at 24.7 minutes was lyophilized to give the title compound as a colorless solid (17.7 mg, 56%, HPLC purity 100%). MS (ESI): 562.2 (100, M+H), 1123.4 (10, 2M+H). HRMS: Calcd for C₂₅H₃₅N₇O₆S (M+H): 562.2442, Found: 562.2441.

### EXAMPLE 3

### Synthesis of N-{(2R)-2-[(tert-Butoxy)carbonylamino]-4-methylpentanoylamino}-6-aminohexanamide

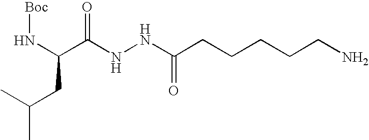

### Part A—Preparation of N-Amino-6-[(fluoren-9-ylmethoxy)carbonylamino]hexanamide, Trifluoroacetic Acid Salt

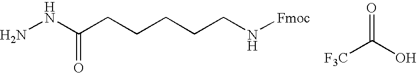

The product of Example 1A (19.5 g, 41.75 mmol) was treated with 50 mL of 50% TFA in dichloromethane for 30 minutes at ambient temperatures under nitrogen. The solution was concentrated under reduced pressure to give a pale yellow oil. The oil was dissolved in 30:70 acetonitrile:water (150 mL) and lyophilized to give an off-white solid (18.89 g, 99%). ¹H NMR (CDCl₃): δ 10.36 (s, 1H), 7.89 (d, J=7.3 Hz, 2H), 7.67 (d, J=7.7 Hz, 2H), 7.41 (t, J=7.7 Hz, 2H), 7.33 (t, J=7.3 Hz, 2H), 7.25 (t, J=6.0 Hz, 1H), 4.30 (d, J=6.6 Hz, 2H), 4.20 (t, J=6.6 Hz, 1H), 2.96 (q, J=6.0 Hz, 2H), 2.158 (t, J=7.5 Hz, 2H), 1.51 (quin, J=7.8 Hz, 2H), 1.39 (quin, J=7.8 Hz, 2H), 1.26 (m, 2H). MS (ESI): 368.2 (100, M+H).

### Part B—Preparation of N-{(2R)-2-[(tert-Butoxy)carbonylamino]-4-methylpentanoylamino}-6-[(fluoren-9-ylmethoxy)carbonylamino]hexanamide

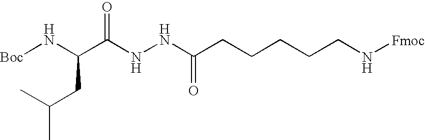

A solution of Boc-D-Leu-OH (2.99 g, 12.0 mmol), HBTU (4.55 g, 12 mmol), HOBt (1.84 g, 12.0 mmol), and DIEA (5.17 mL, 40.0 mmol) in DMF (25 mL) was stirred for 20 minutes at ambient temperatures and treated with a solution of the product of Part A (48.13 g, 10 mmol), and DIEA (2.86 mL, 20.0 mmol) in DMF (10 mL). Additional DIEA was added as necessary to maintain a pH=10. Additional HBTU (2.27 g, 5.00 mmol), and DIEA (1.29 mL, 10.0 mmol) were added after 6 hours. After 12 hours the solution was then slowly added with rapid stirring to 4 L of water. The precipitate was collected by filtration on a course fritted funnel, washed with water, and dried in a vacuum desiccator to give the title compound as a colorless solid (5.831 g, 100%). ¹H NMR (DMSO-d₆): δ 9.76 (s, 1H), 9.72 (s, 1H), 7.89 (d, J=7.5 Hz, 2H), 7.68 (d, J=7.5 Hz, 2H), 7.41 (t, J=7.4 Hz, 2H), 7.33 (t, J=7.4 Hz, 2H), 7.38-7.32 (m, 1H), 6.98-6.83 (m, 1H), 4.29 (d, J=6.9 Hz, 2H), 4.20 (t, J=6.9 Hz, 1H), 4.06-3.98 (m, 1H), 3.00-2.91 (m, 2H), 2.14-2.03 (m, 2H), 1.70-1.19 (m, 18H), 0.93-0.80 (m, 6H). MS (ESI): 481.3 (100, M+H−Boc). HRMS: Calcd for C₃₂H₄₅N₄O₆ (M+H): 581.3334, Found: 581.3342.

### Part C—Preparation of N-Amino-6-[(fluoren-9-ylmethoxy)carbonylamino]hexanamide

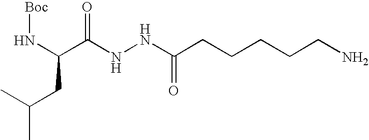

The product of Part B (0.405 g, 0.700 mmol) was dissolved in 20% piperidine in DMF (10 mL) and stirred under nitrogen for 30 minutes at ambient temperatures. The solution was concentrated under reduced pressure to give a pale yellow solid. The solid was purified by HPLC on a Phenomenex C-18 Luna column (41.4×250 mm) using a 0.9%/min gradient of 13.5 to 40.5% acetonitrile containing 0.1% TFA at a flow rate of 80 mL/min. The main product peak eluting at 15.2 minutes was lyophilized to give the title compound as an off-white, hygroscopic solid (0.289 g, 81%). MS (ESI): 359.4 (100, M+H).

A solution of the above solid, (0.298 g, 0.610 mmol) in 50:50 acetonitrile:water (10 mL) was treated with 10 g of Bio-Rad AG 3-X4 ion exchange resin (free base form) and gently mixed for 5 minutes. The solution was removed by decanting and lyophilized to produce the title compound as a white non-hydroscopic powder (0.200 g, 100%). ¹H NMR (CDCl₃): δ 5.05 (s, IH), 4.23 (s, 1H), 2.90-2.81 (m, 2H), 2.35-2.23 (m, 2H), 1.77-1.39 (m, 18H), 1.00-0.87 (m, 6H). MS (ESI): 359.4 (100, M+H); HRMS: Calcd for C₁₇H₃₅N₄O₄ (M+H): 359.2653; Found: 359.2657.

### EXAMPLE 4

### Synthesis of (2R)—N-{2-[4-(Aminomethyl)phenyl]acetylamino}-2-[(tert-butoxy)carbonylamino]-4-methylpentanamide, Trifluoroacetic Acid Salt

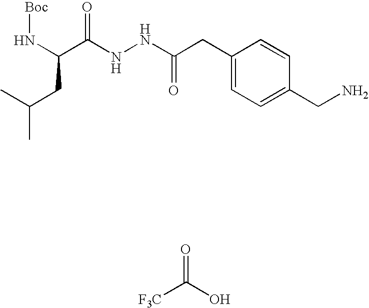

### Part A—Preparation of N-Amino-2-(4-{[(fluoren-9-ylmethoxy)carbonylamino]-methyl}phenyl)acetamide, Trifluoroacetic Acid Salt

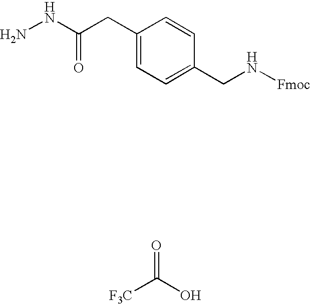

A solution of 2-(4-{[(fluoren-9-ylmethoxy)carbonylamino]methyl}phenyl)acetic acid (470 mg, 1.21 mmol) (prepared according to: Yu, C.; Taylor, J. W. Tetrahedron Letters, 1996, 11, 1731-1734), HBTU (552 mg, 1.46 mmol), and DIEA (0.212 mL, 2.42 mmol) in anhydrous DMF (0.5 mL) was stirred at ambient temperatures under nitrogen for 20 minutes, and treated with t-butyl carbazate (160 mg, 1.21 mmol). The solution was stirred for 1 hour and the solvents were removed under reduced pressure. The resulting residue was treated with 50% TFA in dichloromethane (5 mL), and stirred at room temperature for 30 minutes. The solvents were removed under reduced pressure and the residue was purified by HPLC on a Phenomenex Luna C 18 column (21.2×250 mm) using a 0.9%/min gradient of 27 to 45% acetonitrile containing 0.1% TFA at a flow rate of 20 mL/min. The main product peak eluting at 16.3 minutes was lyophilized to give the title compound as a colorless solid (487 mg, 95%, HPLC purity 100%). ¹H NMR (2:1 CD₃CN:D₂O): δ 7.80 (d, J=7.8 Hz, 2H), 7.62 (d, J=7.2 Hz, 2H), 7.48 (br s, 0.5H), 7.40 (t, J=7.2 Hz, 2H), 7.31 (t, J=7.2 Hz, 2H), 7.20 (d, J=7.8 Hz, 2H), 7.16 (d, J=7.8 Hz, 2H), 6.90 (br s, 0.5H), 4.47 (br s, 0.5H), 4.35 (d, J=6.6 Hz, 2H), 4.20 (t, J=6.3 Hz, 1H), 4.19 (s, 2H), 3.95 (s, 2H), 3.51 (s, 2H). MS (ESI): 402.3 (65, M+H), 803.4 (100, M+H).

### Part B—Preparation of N-Amino-2-(4-{[(fluoren-9-ylmethoxy)carbonylamino]-methyl}phenyl)acetamide, Trifluoroacetic Acid Salt

A solution of Boc-D-Leu-OH (28 mg, 0.12 mmol), HOAt (16 mg, 0.12 mmol) and TMP (80 μL, 0.60 mmol) in anhydrous DMF (0.5 mL) was treated with DIC (30 mg, 0.24 mmol). The reaction was stirred at ambient temperatures under nitrogen for 20 minutes, and treated with the product of Part A (48.5 mg, 0.121 mmol). The solution was stirred for 3 hours, and treated with TAEA (tris-(2-aminoethyl)amine, prepared according to the procedure described in J. Org. Chem. (1990), 55(5), 1673-5) (0.25 mL). The reaction was stirred at room temperature for 30 minutes, and concentrated under reduced pressure. The residue was purified by HPLC on a Phenomenex Luna C 18 column (21.2×250 mm) using a 0.9%/min gradient of 9 to 36% acetonitrile containing 0.1% TFA at a flow rate of 20 mL/min. The main product peak eluting at 23.9 minutes was lyophilized to give the title compound as a colorless solid (25.7 mg, 94%, HPLC purity 100%). ¹H NMR (CD₃CN): δ 8.87 (br s, 1H), 8.53 (br s, 1H), 7.80 (br s, 1H), 7.38 (d, J=8.4 Hz, 2H), 7.33 (d, J=8.4 Hz, 2H), 5.68 (br s, 1H), 4.09 (s, 2H), 3.54 (s, 2H), 1.68 (m, 1H), 1.51 (m, 2H), 1.40 (s, 9H), 0.98-0.85 (m, 6H). MS (ESI): 785.5 (100, 2M+H), 393.4 (90, M+H); HRMS: Calcd for C₂₀H₃₃N₄O₄ (M+H): 393.2496; Found: 393.2499. Chiral analysis: 99.0% D-Leu.

### EXAMPLE 5

### Synthesis of (2R)—N-amino-2-[(tert-Butoxy)carbonylamino]-4-methylpentanamide, Trifluoroacetic Acid Salt

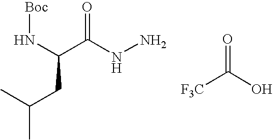

### Part A—Preparation of (2R)-2-[(tert-Butoxy)carbonylamino]-N-[(fluoren-9-ylmethoxy)carbonylamino]-4-methylpentanamide

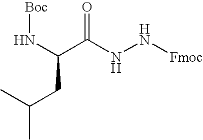

A solution of Boc-D-Leu-OH (134 mg, 0.536 mmol), HOAt (73 mg, 0.54 mmol) and TMP (0.87 mL, 2.7 mmol) in anhydrous DMF (2 mL) was treated with DIC (135 mg, 1.07 mmol), and stirred at ambient temperatures under nitrogen for 20 minutes. The solution was treated with 9-fluorenylmethyl carbazate (136 mg, 0.536 mmol) and stirred for 5 hours. The solution was diluted with dichloromethane (20 mL), washed consecutively with 10% citric acid (3×20 mL), saturated NaHCO₃ (20 mL), and saturated NaCl (20 mL), dried (MgSO₄), filtered, and concentrated to give crude title compound as a viscous oil (271 mg, 108%). MS (ESI): 368.3 (95, M+H), 490.2 (100, M+Na).

### Part B—Preparation of (2R)—N-amino-2-[(tert-Butoxy)carbonylamino]-4-methylpentanamide, Trifluoroacetic Acid Salt

The product of Part A and TAEA (1.6 mL) was dissolved in DMF (1 mL) and stirred at room temperature for 30 minutes. The DMF was removed under reduced pressure and the residue was purified by HPLC on a Phenomenex Luna C18 column (41.4×250 mm) using a 0.9%/min gradient of 18 to 45% acetonitrile containing 0.1% TFA at a flow rate of 20 mL/min. The main product peak eluting at 11.9 minutes was lyophilized to give the title compound as a colorless solid (76.7 mg, 58%, HPLC purity 100%). ¹H NMR (1:1 CD₃CN:D₂O): δ 4.03-3.93 (m, 1H), 1.63-1.52 (M, 1H), 1.52-1.49 (M, 2H), 1.35 (s, 9H), 0.90-0.81 (m, 6H). MS (ESI): 513.3 (20, 2M+Na), 268.2 (100, M+Na); HRMS: Calcd for C₁₁H₂₃NaN₃O₃ (M+Na): 268.1632; Found: 268.1635.

### EXAMPLE 6

### Synthesis of (2R)—N-[3-(2-{2-[2-(2-Aminoethoxy)ethoxy]ethoxy}ethoxy)-propanoylamino]-2-[(tert-butoxy)carbonylamino]-4-methylpentanamide, Trifluoroacetic Acid Salt

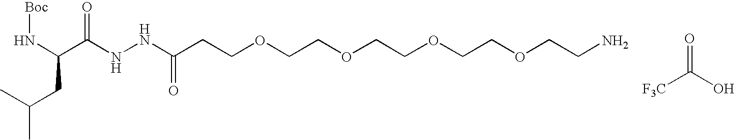

A solution of 3-{2-[2-(2-{2-[(fluoren-9-ylmethoxy)carbonylamino]ethoxy}-ethoxy)ethoxy]ethoxy}propanoic acid (ChemBioChem (2002), 3(2-3), 238-242, 43.8 mg, 0.090 mmol), HBTU (41 mg, 0.11 mmol) and DIEA (31 μL, 0.18 mmol) in anhydrous DMF (0.5 mL) was stirred at ambient temperatures under nitrogen for 20 minutes, and treated with the product of Example 5B (22 mg, 0.090 mmol). The solution was stirred for 2 hours and treated with TAEA (0.15 mL). The reaction was stirred at room temperature an additional 30 minutes, and DMF was removed under reduced pressure. The resulting residue was purified by HPLC on a Phenomenex Luna C18 column (21.2×250 mm) using a 0.9%/min gradient of 18 to 45% acetonitrile containing 0.1% TFA at a flow rate of 20 mL/min. The main product peak eluting at 14.8 minutes was lyophilized to give the title compound as a colorless solid (30 mg, 69%, HPLC purity 100%). ¹H NMR (CD₃CN) δ 4.08-3.97 (m, 1H), 3.69 (t, J=6.3 Hz, 2H), 3.64 (t, J=5.1 Hz, 2H) 3.62-3.50 (m, 12H), 3.08 (t, J=3.7 Hz, 2H), 2.48 (t, J=6.0 Hz, 2H), 1.67-1.56 (m, 1H), 1.56-1.43 (m, 2H), 1.36 (s, 9H), 0.91-0.81 (m, 6H); ¹³C NMR (CDCl₃): δ 173, 171, 161 (q, J=34.6 Hz), 156, 116 (q, J=290 Hz), 79.9, 69.1, 69.0, 69.0, 68.9, 68.9, 51.2, 39.9, 38.6, 33.3, 27.0, 23.8, 21.7, 20.1. MS (ESI): 493.4 (100, M+H); HRMS: Calcd for C₂₂H₄₅N₄O₈ (M+H): 493.3232; Found: 493.3225.

### EXAMPLE 7

### Synthesis of (2R)-2-[(tert-Butoxy)carbonylamino]-N-(hydrazinocarbonylamino)-4-methylpentanamide, Trifluoroacetic Acid Salt

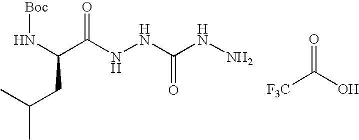

A solution of carbohydrazide (82 mg, 0.92 mmol) in DMF (1 mL) was added dropwise to a solution of Boc-DLeu-OH (57 mg, 0.23 mmol) and PyBOP (143 mg, 0.275 mmol) and DIEA (80 μL, 0.46 mmol) in DMF (1 mL). The reaction was stirred at room temperature under nitrogen for 3 hours and the solvents were removed under reduced pressure. The resulting residue was purified by HPLC on a Phenomenex Luna C18 column (21.2×250 mm) using a 0.9%/min gradient of 9 to 36% acetonitrile containing 0.1% TFA at a flow rate of 20 nL/min. The main product peak eluting at 19.5 minutes was lyophilized to give the title compound as a colorless solid (61 mg, 87%, HPLC purity 100%). ¹H NMR (1:1 CD₃CN:D₂P): δ 8.60 (bs, 1H), 7.62 (bs, 1H), 5.62 (s, 1H), 4.10 (s, 1H), 1.73-1.62 (m, 1H), 1.61-1.48 (m, 2H), 1.42 (s, 9H), 0.96-0.88 (m, 6H); ¹³C NMR (CDCl₃): δ 174, 160, 157, 52.9, 41.6, 28.8, 25.4, 23.6, 22.1. MS (ESI): 607.4 (10, 2M+H), 248.4 (100, M−tBu+H), 204.4 (60, M−Boc+H); HRMS: Calcd for C₁₂H₂₆N₅O₄ (M+H): 304.1979; Found: 304.1975.

### EXAMPLE 8

### Synthesis of N-[(2R)-2-(Dimethylamino)-4-methylpentanoylamino]-6-aminohexanamide, Bis-Acetic Acid Salt

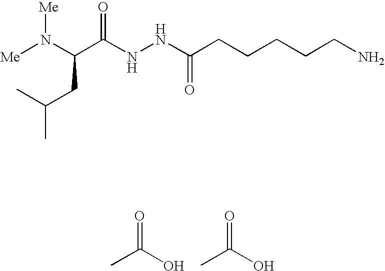

### Part A—Preparation of N-[(2R)-2-(Dimethylamino)-4-methylpentanoylamino]-6-[(fluoren-9-ylmethoxy)carbonylamino]hexanamide

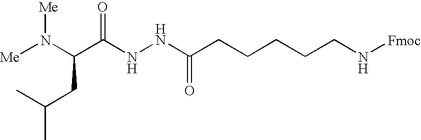

A solution of the product of Example 3B (49 mg, 0.83 mmol) in 50% TFA in dichloromethane (2 mL) was stirred at room temperature for 20 minutes and concentrated under reduced pressure to give a solid. This solid was taken up in acetonitrile (1 mL), and treated with 37% formaldehyde solution (62 μL, 8.3 mmol) and HOAc (10 μL). The reaction was stirred at room temperature under nitrogen for 1 hour and evaporated to dryness. The resulting residue was purified by HPLC on a Phenomenex Luna C18 column (21.2×250 mm) using a 0.9%/min gradient of 22.5 to 49.5% acetonitrile containing 0.1% TFA at a flow rate of 20 mL/min. The main product peak eluting at 25.1 minutes was lyophilized to give the title compound as a colorless solid (18.5 mg, 43%, HPLC purity 100%). ¹H NMR (CD₃CN) δ 9.59 (s, 1H), 8.23 (s, 1H), 7.84 (d, J=7.8 Hz, 2H), 7.66 (d, J=7.8 Hz, 2H), 7.43 (t, J=7.5 Hz, 2H), 7.35 (t, J=7.5 Hz, 2H), 5.69 (s, 1H), 4.33 (d, J=7.2 Hz, 2H), 4.23 (t, J=7.2 Hz, 1H), 3.91-3.84 (m, 1H), 3.08 (q, J=6.6 Hz, 2H), 2.88 (s, 6H), 2.21 (t, J=7.5 Hz, 2H), 1.68-1.43 (m, 7H), 1.38-1.31 (m, 2H), 0.91-0.89 (m, 6H). MS (ESI): 509.4 (100, M+H).

### Part B—Preparation of N-[(2R)-2-(Dimethylamino)-4-methylpentanoylamino]-6-aminohexanamide, Bis-Acetic Acid Salt

The product of Part A was dissolved in 20% piperidine in DMF (2 mL) and stirred at room temperature under nitrogen for 30 minutes. The solvents were removed under reduced pressure and the resulting residue was purified by HPLC on a Phenomenex Luna C18 column (21.2×250 mm) using a 0.9%/min gradient of 0 to 18% acetonitrile containing 15 mM NH₄OAc (pH=7) at a flow rate of 20 mL/min. The main product peak eluting at 12.7 minutes was lyophilized to give the title compound as a colorless solid (3.1 mg, 42%, HPLC purity 100%). ¹H NMR (CD₃CN:D₂O) δ 3.09-3.06 (m, 1H), 2.88 (t, J=7.5 Hz, 2H), 2.28 (s, 6H), 2.22 (t, J=7.2 Hz, 2H), 1.81 (s, 6H), 1.65-1.48 (m, 6H), 1.41-1.32 (m, 3H), 0.90-0.86 (m, 6H); ¹³C NMR (CDCl₃): δ 179, 174, 171, 64.6, 41.0, 39.2, 37.0, 32.9, 26.3, 25.1, 24.8, 24.2, 23.0, 22.6, 21.3. MS (ESI): 287.4 (100, M+H); HRMS: Calcd for C₁₄H₃₁N₄O₂ (M+H): 287.2442; Found: 287.2443.

### EXAMPLE 9

### Synthesis of N-{(2S)-2-[(tert-Butoxy)carbonylamino]-4,N-dimethylpentanoylamino}-6-aminohexanamide, Trifluoroacetic Acid Salt

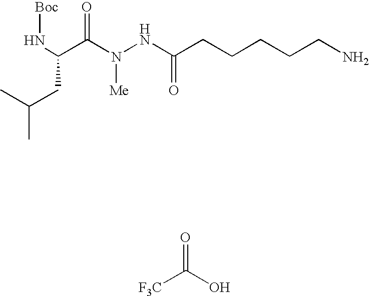

### Part A—Preparation of N-[(tert-Butoxy)-N-methylcarbonylamino]-6-[(fluoren-9-ylmethoxy)carbonylamino]hexanamide

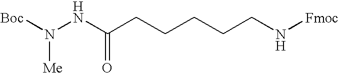

A solution of Fmoc-Ahx-OH (4.84 g, 13.7 mmol), HOBt (2.72 g, 17.8 mmol), and HBTU (6.75 g, 17.8 mmol) in anhydrous DMF (10 mL) was treated with DIEA (5.00 mL, 28.7 mmol) and stirred at room temperature for 20 minutes. The solution was treated with Boc-N(Me)-NH₂ (Malachowski, M. P.; Tie, C.; Wang, K.; Broadrup R. L. J. Org. Chem. 2002, 67, 8962-8969), and stirred under nitrogen at room temperature for 18 hours. The reaction was diluted with ethyl acetate (50 mL), washed consecutively with 10% citric acid (3×50 mL), saturated NaHCO₃ (2×50 mL), and saturated LiCl (2×25 mL), dried (MgSO₄), filtered, and concentrated under high vacuum to give the title compound as a colorless solid (5.257 g, 80%). ¹H NMR (CDCl₃) δ 7.77 (d, J=7.6 Hz, 2H), 7.59 (d, J=7.3 Hz, 2H), 7.41 (t, J=7.4 Hz, 2H), 7.32 (t, J=7.0 Hz, 2H), 4.41 (d, J=5.8 Hz, 2H), 4.23 (t, J=6.7 Hz, 1H), 3.26-3.15 (m, 2H), 3.13 (s, 3H), 2.33-2.22 (m, 2H), 1.74-1.63 (m, 2H), 1.52-1.38 (m, 13H). MS (ESI): 382.2 (100, M−Boc+H), 504.3 (15, M+Na).

### Part B—Preparation of N-{(2S)-2-[(tert-Butoxy)carbonylamino]-4,N-dimethylpentanoylamino}-6-[(fluoren-9-ylmethoxy)carbonylamino]hexanamide

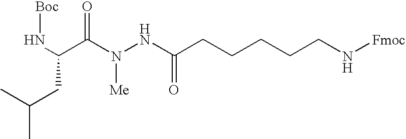

The product of Part A (122.9 mg, 0.255 mmol) was dissolved in 50:50 TFA:dichloromethane (2.0 mL) and stirred at room temperature for 20 minutes and concentrated by the use of reduced pressure. The resulting amber oil was dissolved in DMF (2.0 mL) along with Boc-Leu-OH (76.4 mg, 0.306 mmol), HBTU (116.2 mg, 0.306 mmol) and DIEA (0.089 mL, 0.510 mmol). The reaction solution was stirred at room temperature under nitrogen for 3 hours. The reaction was diluted with ethyl acetate (5.0 mL), washed consecutively with 0.1N HCl (2×5.0 mL), 10% NaHCO₃ (5.0 mL), water (5.0 mL), and saturated NaCl (5.0 ML), dried (MgSO₄), filtered, and concentrated. The crude product was purified by flash chromatography on silica gel (1:2 pentane:ethyl acetate) to give the title compound as a viscous oil (71.2 mg, 47%, HPLC purity 100%). ¹H NMR (CDCl₃): δ 8.84 (bs, 1H), 7.75 (d, J=7.8 Hz, 2H), 7.56 (d, J=7.2 Hz, 2H), 7.39 (t, J=7.5 Hz, 2H), 7.30 (t, J=7.2 Hz, 2H), 5.02-4.93 (m, 1H), 4.85 (bs, 1H), 4.61-4.51 (m, 1H), 4.44-4.35 (m, 2H), 4.25-4.17 (m, 1H), 3.20 (bs, 2H), 3.15 (s, 3H), 2.29-2.20 (m, 2H), 1.73-1.58 (m, 3H), 1.58-1.46 (m 2H), 1.46-1.34 (m, 13H), 0.89-0.83 (m, 6H); ¹³C NMR (CDCl₃): δ 174.24, 171.08, 156.51, 156.29, 143.97, 141.33, 127.67, 127.03, 125.00, 119.97, 80.34, 66.55, 60.39, 47.75, 47.30, 40.91, 40.71, 35.62, 33.94, 29.58, 28.30, 26.24, 24.55, 22.88,22.01. MS (ESI): 617.4 (100, M+Na), 495.3 (70, M−Boc+H); HRMS: Calcd for C₃₃H₄₇N₄O₆ (M+H): 595.3490; Found: 595.3495.

### Part C—Preparation of N-{(2S)-2-[(tert-Butoxy)carbonylamino]-4,N-dimethylpentanoylamino}-6-aminohexanamide, Trifluoroacetic Acid Salt

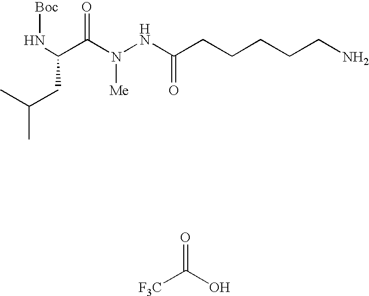

The product of Part B (35.0 mg, 0.058 mmol) was dissolved in TAEA (0.22 mL, 1.471 mmol) and DMF (0.5 mL) and stirred at room temperature under nitrogen for 30 minutes. The volatiles were removed under reduced vacuum and the resulting crude product was purified by HPLC on a Phenomenex Luna C18(2) column (21.2×250 mm) using a 0.9%/min gradient of 18 to 45% acetonitrile containing 0.1% TFA at a flow rate of 20 mL/min. The main product peak eluting at 17.7 minutes was lyophilized to give the title compound as a colorless solid (25.9 mg, 91%, HPLC purity 100%). ¹H NMR (CD₃CN): δ 9.01 (s, 1H), 7.20 (bs, 3H), 5.48 (d, J=8.4 Hz, 1H), 4.48 (bs, 1H), 3.02 (s, 3H), 2.96 (bs, 2H), 2.24 (t, J=7.2 Hz, 2H), 1.75-1.58 (m, 5H), 1.50-1.28 (m, 13H), 0.89 (d, J=6.6 Hz, 6H). MS (ESI): 373.5 (100, M+H); HRMS: Calcd for C₁₈H₃₇N₄O₄ (M+H): 373.2809; Found: 373.2810. Chiral analysis: 99.9% L-Leu.

### EXAMPLE 10

### Synthesis of 2-{4-[(N-{5-[N-((2R)-2-Amino-4-methylpentanoylamino)carbamoyl]-pentyl}carbamoyl)methyl]-1,4,7,10-tetraaza-7,10-bis(carboxymethyl)cyclododecyl}-acetic Acid

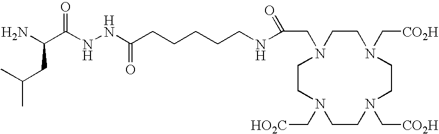

### Part A—Preparation of 6-[(tert-Butoxy)carbonylamino]-N-[(fluoren-9-ylmethoxy)-carbonylamino]hexanamide

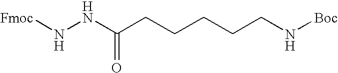

A solution of Boc-Ahx-OH (1.29 g, 5.58 mmol), HOBt (1.02 g, 6.66 mmol), HBTU (2.54 g, 6.66 mmol) and DIEA (2.44 mL, 14.0 mmol) in anhydrous DMF (10 mL) was stirred at ambient temperatures under nitrogen for 20 minutes, and treated with 9-fluorenylmethyl carbazate (1.42 g, 5.58 mmol) and DIEA (0.5 mL, 2.87 mmol). The solution was stirred for 3.5 hours, diluted with dichloromethane (30 mL), washed consecutively with 10% citric acid (50 mL), saturated NaHCO₃ (3×50 mL), and saturated NaCl (3×50 mL), dried over MgSO₄, filtered, and concentrated to give a yellow oil. The oil was purified by flash chromatography over silica gel, eluting with 2:1 ethyl acetate:hexanes to give the title compound as a colorless solid (2.061 g, 79%, HPLC purity 100%). ¹H NMR (CDCl₃): δ 7,75 (d, J=7.5 Hz, 2H), 7.58 (d, J=7.4 Hz, 2H), 7.50 (br, 1H), 7.39 (t, J=7.4 Hz, 2H), 7.30 (dt, J=7.0 Hz, 2H), 6.87 (br, 1H), 4.63 (s, 1H), 4.44 (d, J=6.9 Hz, 2H), 4.24 (t, J=7.1 Hz, 1H), 3.08 (s, 2H), 2.23 (s, 2H), 1.68 (m, 2H), 1.42 (s, 9H), 1.47-1.35 (m, 4H); ¹³C NMR (CDCl₃): δ 172.6, 171.2, 156.2, 143.5, 141.3, 127.9, 127.8, 127.1, 125.1, 124.9, 120.0, 79.2, 68.0, 60.4, 46.9, 40.2, 33.8, 29.6, 28.4, 26.0, 24.7, 21.1, 14.2. MS (ESI): 368.4(100, M−Boc+H).

### Part B—Preparation of 6-Amino-N-[(fluoren-9-ylmethoxy)carbonylamino]hexanamide, Trifluoroacetic Acid Salt

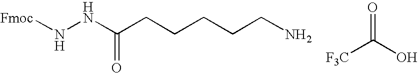

The product of Part A (0.79 g, 3.1 mmol) was treated with 50% TFA/dichloromethane (12.0 mL) at room temperature under nitrogen for 20 minutes. The solution was concentrated under reduced pressure to give a yellow oil (1.38 g). ¹H NMR (CDCl₃): δ 9.18 (s, 1H), 7.74 (d, J=7.6 Hz, 2H), 7.52 (d, J=7.6 Hz, 2H), 7.39 (t, J=7.5 Hz, 2H), 7.35 (br, 1H), 7.29 (t, J=7.2 Hz, 2H), 6.07 (br, 2H), 4.38 (d, J=7.5 Hz, 2H), 4.20 (t, J=7.5 Hz, 1H), 3.05 (m, 2H), 2.33 (m, 2H), 1.73 (m, 4H), 1.48 (m, 2H); ¹³C NMR (CDCl₃): δ 175.1, 160.1, 143.0, 141.3, 128.0, 127.2, 124.9, 120.1, 117.8, 115.9, 114.0, 112.1, 68.7, 46.6, 40.5, 32.0, 27.6, 25.1, 23.6, 14.1. MS (ESI): 368.2 (100, M+H).

### Part C—Preparation of tert-Butyl 2-(1,4,7,10-Tetraaza-4,10-bis{[(tert-butyl)-oxycarbonyl]-methyl}-7-{[N-(5-{N-[(fluoren-9-ylmethoxy)carbonylamino]-carbamoyl}pentyl)-carbamoyl]-methyl}cyclododecyl)acetate

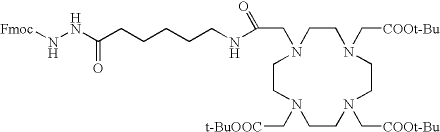

A solution of DOTA tri-t-butyl ester (0.972 g, 1.70 mmol), HBTU (0.772 g, 2.04 mmol), HOBt (0.312 g, 2.04 mmol), and DIEA (0.59 mL, 5.9 mmol) in anhydrous DMF (8.0 mL) was stirred at room temperature under nitrogen for 20 minutes. The product of Part B (1.38 g, 1.70 mmol) was added in one portion. Additional HBTU (0.772 g, 2.04 mmol) was added after 1 hour and the reaction was stirred for an additional 3 hours. The reaction mixture was quenched with 10% citric acid (20 mL) and diluted with dichloromethane (30 mL). The aqueous layer was extracted with dichloromethane (3×30 mL). The combined organic extracts were washed consecutively with 10% citric acid (30 mL), saturated NaHCO₃ (3×30 mL), and saturated NaCl (3×30 mL), dried (MgSO₄), filtered, and concentrated to give a yellow oil. The oil was purified by flash chromatography over silica gel, eluting with ethyl acetate to give the title compound as a colorless oil (0.746 g, 48%). ¹H NMR (4:1 CDCl₃:DMSO-d₆): δ 7.54 (m, 2H), 7.41 (m, 2H), 7.17 (m, 2H), 7.08 (m, 2H), 4.15 (d, 2H), 4.02 (m, 1H), 2.97 (m, 2H), 2.68-2.45 (m, 24H), 2.00 (t, 2H), 1.44 (t, 2H), 1.30-1.11 (m, 31H). MS (ESI): 461.9 (100, M+2H), 922.5 (80, M+H).

### Part D—Preparation of 2-{4-[(N-{5-[N-((2R)-2-Amino-4-methylpentanoylamino)-carbamoyl]-pentyl}carbamoyl)methyl]-1,4,7,10-tetraaza-7,10-bis(carboxymethyl)-cyclododecyl}-acetic Acid

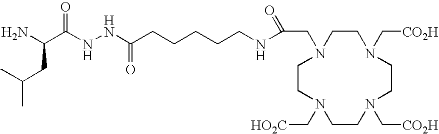

A solution of Boc-D-Leu-OH (83.0 mg, 0.330 mmol), HBTU (164 mg, 0.430 mmol), and DIEA (116 μL, 0.67 mmol) in anhydrous DMF (2 mL) was stirred at room temperature under nitrogen for 20 minutes. In a separate flask, a solution of the product of Part C (307 mg, 0.330 mmol) in 20% piperidine in DMF (5.0 mL) was stirred at room temperature under nitrogen for 45 minutes, and concentrated under reduced pressure. The resulting residue was dissolved in DMF (5 mL) and added to the solution of activated Boc-dLeu-OH. The solution was concentrated after 1 hour to give a yellow viscous oil. This oil was dissolved in TFA (2 mL), treated with TIS (20 μL), and stirred at room temperature under nitrogen for 1 hour. The solution was concentrated under reduced pressure and the residue was purified by HPLC on a Phenomenex Luna C18 column (21.2×250 mm) using a 0.9%/o/min gradient of 0 to 18% acetonitrile containing 0.1% TFA at a flow rate of 20 mL/min. The main product peak eluting at 14.4 minutes was lyophilized to give the title compound as a colorless solid (18.2 mg, 8.5%). MS (ESI): 645.5 (90, M+H), 323.3 (100, M+2H); HRMS: Calcd for C₂₈H₅₃N₈O₉ (M+H): 645.3930; Found: 645.3933. Chiral analysis: 99.4% D-Leu.

### EXAMPLE 11

### Synthesis of 2-(7-{[N-({4-[(N-{5-[N-((2R)-2-Amino-4-methylpentanoylamino)-carbamoyl]pentyl}carbamoyl)methyl]phenyl}methyl)carbamoyl]methyl}-1,4,7,10-tetraaza-4,10-bis(carboxymethyl)cyclododecyl)acetic Acid

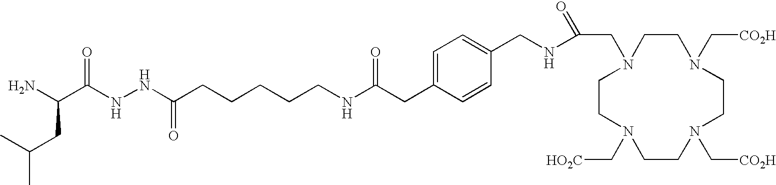

### Part A—Preparation of N-{(2R)-2-[(tert-Butoxy)carbonylamino]-4-methylpentanoylamino}-6-{2-[4-(aminomethyl)phenyl]acetylamino}hexanamide, Trifluoroacetic Acid Salt

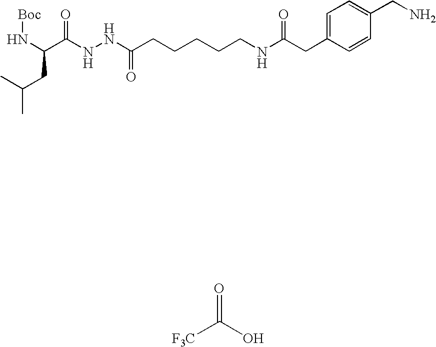

A solution of 2-(4-{[(fluoren-9-ylmethoxy)carbonylamino]methyl}phenyl)acetic acid (29.7 mg, 0.0770 mmol), HBTU (31.7 mg, 0.0840 mmol) and DIEA (24 μL, 0.14 mmol) in anhydrous DMF (0.5 mL) was stirred at ambient temperatures under nitrogen for 20 minutes, and treated with the product of Example 3 (25.0 mg, 0.070 mmol) and DIEA (10 μL, 0.057 mmol). The solution was stirred for additional 48 hours, and treated with TAEA (0.25 mL). The reaction was stirred at room temperature under nitrogen an additional 20 minutes, and concentrated under reduced pressure. The residue was purified by HPLC on a Phenomenex Luna C18 column (21.2×250 mm) using a 0.9%/min gradient of 13.5 to 36% acetonitrile containing 0.1% TFA at a flow rate of 20 mL/min. The main product peak eluting at 19.6 minutes was lyophilized to give the title compound as a colorless solid (16.3 mg, 46%, HPLC purity 95%). ¹H NMR (4:1 CD₃CN:D₂O): δ 7.35 (d, J=8.4 Hz, 2H), 7.31 (d, J=8.4 Hz, 2H), 4.15 (m, 1H), 4.05 (s, 2H), 3.48 (s, 2H), 3.11 (t, J=6.6 Hz, 2H), 2.16 (t, J=7.5 Hz, 2H), 1.68-1.60 (m, 1H), 1.54-1.51 (m, 4H), 1.42-1.44 (m, 2H), 1.38 (s, 9H), 1.30-1.22 (m, 2H), 0.89 (dd, J=7.2 Hz, 6H). MS (ESI): 506.4 (100, M+H), 406.4 (10, M−Boc+H).

### Part B—Preparation of 2-(7-{[N-({4-[(N-{5-[N-((2R)-2-Amino-4-methylpentanoylamino)-carbamoyl]pentyl}carbamoyl)methyl]phenyl}-methyl)carbamoyl]methyl}-1,4,7,10-tetraaza-4,10-bis(carboxymethyl)-cyclododecyl)acetic Acid

A solution of DOTA tri-t-butyl ester (10 mg, 0.017 mmol), HBTU (7.8 mg, 0.021 mmol) and DIEA (6.0 μL, 0.034 mmol) in anhydrous DMF (0.5 mL) was stirred at room temperature under nitrogen for 20 minutes, and treated with the product of Part A (8.7 mg, 0.017 mmol). Stirring was continued for 1 hour and the solution was concentrated under reduced pressure. The residue was dissolved in TFA (1 mL), treated with TIS (10 μL), and stirred for 4 hours. The solution was concentrated under reduced pressure and the residue was purified by HPLC on a Phenomenex Luna C18 column (21.2×250 mm) using a 0.9%/min gradient of 0 to 18% acetonitrile containing 0.1% TFA at a flow rate of 20 mL/min. The main product peak eluting at 20.5 minutes was lyophilized to give the title compound as a colorless solid (8.5 mg, 62%, HPLC purity 96%). MS (ESI): 793.5 (40, M+H), 396.9 (100, M+2H); HRMS: Calcd for C₃₇H₆₂N₉O₁₀ (M+H): 792.4620; Found: 792.462.

### EXAMPLE 12

### Synthesis of 2-{7-[(N-{[5-(N-{5-[N-((2R)-2-Amino-4-methylpentanoylamino)-carbamoyl]pentyl}carbamoyl)(2-pyridyl)]amino}carbamoyl)methyl]-1,4,7,10-tetraaza-4,10-bis(carboxymethyl)cyclododecyl}acetic Acid, Trifluoroacetic Acid Salt

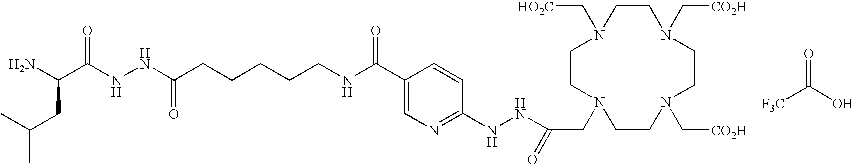

### Part A—Preparation of 6-{[2-(1,4,7,10-Tetraaza-4,7,10-tris{[(tert-butyl)oxycarbonyl]methyl}cyclododecyl)acetylamino]amino}pyridine-3-carboxylic Acid, Bis-Trifluoroacetic Acid Salt

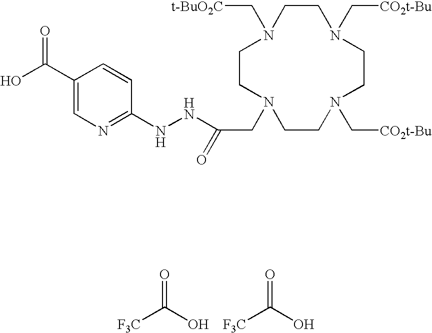

A solution of DOTA tri-t-butyl ester (225 mg, 0.393 mmol), HOBt (50 mg, 0.33 mmol), and EDC (62 mg, 0.32 mmol) in 50:50 dichloromethane:acetonitrile (2.0 mL) was stirred at ambient temperatures under nitrogen for 5 minutes, and treated with 6-hydrazinictoic acid (50 mg, 0.32 mmol). The solution was stirred for 60 hours and concentrated under reduced pressure. The residue was purified by HPLC on a Phenomenex Luna C18 column (21.2×250 mm) using a 0.9%/min gradient of 9 to 36% acetonitrile containing 0.1% TFA at a flow rate of 20 mL/min. The main product peak eluting at 32.9 minutes was lyophilized to give the title compound as an off-white solid (3.0 mg, 1.0%). MS (ESI): 708.4 (60, M+H).

### Part B—Preparation of tert-butyl 2-[10-({N-[(5-{N-[5-(N-{(2R)-2-[(tert-Butoxy)carbonylamino]-4-methylpentanoylamino}carbamoyl)pentyl]carbamoyl}(2-pyridyl))amino]carbamoyl}methyl)-1,4,7,10-tetraaza-4,7-bis {[(tert-butyl)oxycarbonyl]methyl}cyclododecyl]acetate, Trifluoroacetic Acid Salt

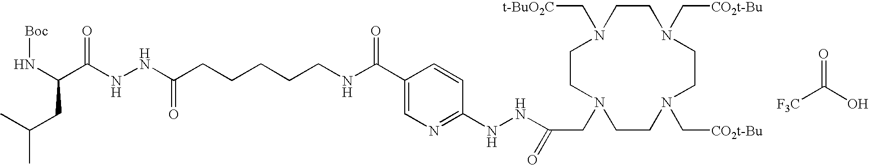

A solution of the product of Part A (3.0 mg, 3.2 μmol), HBTU (1.8 mg, 4.8 μmol), DIEA (8.1 μL, 4.8 μmol), and the product of Example 3 (2.2 mg, 4.8 μmol) in DMF (0.5 mL) was stirred at ambient temperatures for 1 hour. The solution was concentrated under reduced pressure and the residue was purified by HPLC on a Phenomenex Luna C18 column (21.2×250 mm) using a 0.9%/min gradient of 9 to 45% acetonitrile containing 0.1% TFA at a flow rate of 20 mL/min. The main product peak eluting at 24.7 minutes was lyophilized to give the title compound as a colorless solid (3.0 mg, 73%). MS (ESI): 1048.6 (60, M+H).

### Part C—Preparation of 2-{7-[(N-{[5-(N-{5-[N-((2R)-2-Amino-4-methylpentanoylamino)-carbamoyl]pentyl}carbamoyl)(2-pyridyl)]amino}-carbamoyl)methyl]-1,4,7,10-tetraaza-4,10-bis(carboxymethyl)cyclododecyl}acetic Acid, Trifluoroacetic Acid Salt

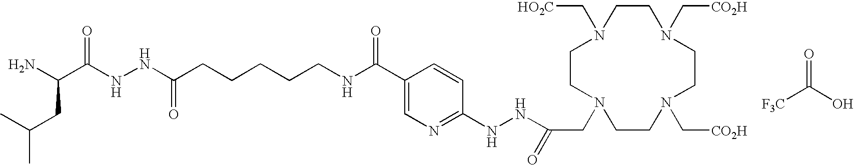

The product of Part B (3.0 mg, 2.4 μmol) was dissolved in75:25 TFA:dichloromethane (1.0 mL) and stirred for 6 hours at ambient temperatures under nitrogen. The solution was concentrated under reduced pressure and the residue was dissolved in 50:50 acetonitrile:water (5.0 mL) and lyophilized to give the title compound as a brown solid (1.0 mg, 47%). MS (ESI): 780.4 (40, M+H).

### EXAMPLE 13

### Synthesis of N-((2R)-2-{[(4-{(2S)-2-[(tert-Butoxy)carbonylamino]-4-methylpentanoylamino}phenyl)methoxy]carbonylamino}-4-methylpentanoylamino)-6-aminohexanamide, Trifluoroacetic Acid Salt

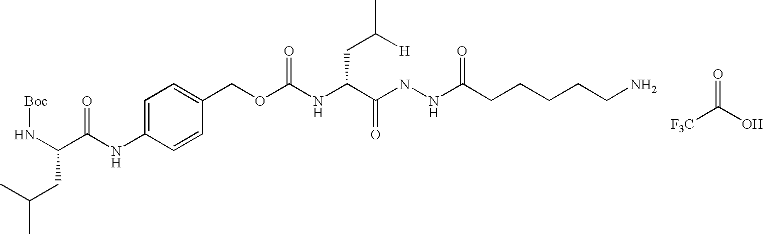

### Part A—Preparation of (2S)-2-[(tert-Butoxy)carbonylamino]-N-[4-(hydroxymethyl)-phenyl]-4-methylpentanamide

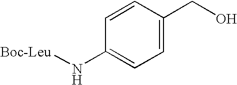

A solution of Boc-Leu-OH (2.02 g, 8.10 mmol), 4-aminobenzyl alcohol (1.00 g, 8.10 mmol), and EEDQ (2.21 g, 8.90 mmol) in 1:1 toluene:ethanol (20 mL) was stirred at room temperature under nitrogen for 4 hours. The solution was concentrated under reduced pressure and the resulting residue was purified by flash chromatography on silica gel, eluting consecutively with 1:4 ethyl acetate:hexanes, 1:2 ethyl acetate:hexanes, and 1:1 ethyl acetate:hexanes to give the title compound as a colorless solid (2.62 g, 96%). ¹H NMR (CDCl₃): δ 8.46 (s, 1H), 7.49 (d, J=8.3 Hz, 2H), 7.28 (d, J=8.3 Hz, 2H), 4.98 (s, 1H), 4.64 (s, 2H), 4.27 (s, 1H), 1.83-1.73 (m, 2H), 1.70 (s, 1H), 1.62-1.55 (m, 1H), 1.47 (s, 9H), 1.030.93 (m, 6H). MS (ESI): 237.3 (100, M−Boc+H). HRMS: Calcd for C₁₈H₂₈N₂O₄ (M+H): 337.2122; Found: 337.2118.

### Part B—Preparation of (4-{(25)-2-[(tert-Butoxy)carbonylamino]-4-methylpentanoylamino}phenyl)methyl (4-Nitrophenoxy)formate

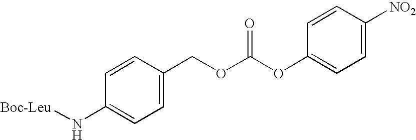

A solution of the product of Part A (1.00 g, 3.0 mmol) and 4-nitrophenyl chloroformate (0.6 g, 3.0 mmol) in anhydrous dichloromethane (10 mL) was cooled to 0° C., treated with pyridine (0.4 mL, 4.9 mmol) and stirred at ambient temperatures under nitrogen for 2 hours. The solution was diluted with dichloromethane (30 mL), washed with water (50 mL) and saturated NaCl (50 mL), dried (MgSO₄), filtered, and concentrated under reduced pressure. The resulting residue was purified by flash chromatography on silica gel, eluting with 3:1 ethyl acetate/hexanes to give the title compound as a colorless crystalline solid (1.02 g, 68%). ¹H NMR (CDCl₃): δ 8.48 (s, 1H), 8.30-8.26 (m, 2H), 7.57 (d, J=8.4 Hz, 2H), 7.42-7.36 (m, 4H), 5.25 (s, 2H), 4.92 (s, 1H), 4.24 (s, 1H), 1.85-1.70 (m, 2H), 1.62-1.53 (m, 1H), 1.48 (s, 9H), 1.02-0.95 (m, 6H); ¹³C NMR (CDCl₃): δ 170.9, 155.5, 152.4, 145.4, 138.6, 129.8, 129.7, 125.3, 121.8, 119.9, 80.8, 70.7, 53.8, 40.2, 28.3, 24.8, 22.9, 21.9. MS (ESI): 524.3 (100, M+Na). HRMS: Calcd for C₂₅H₃₁N₃O₈ (M+H): 502.2184; Found: 502.2183.

### Part C—Preparation of N-((2R)-2-{[(4-{(2S)-2-[(tert-Butoxy)carbonylamino]-4-methylpentanoylamino}phenyl)methoxy]carbonylamino}-4-methylpentanoylamino)-6-aminohexanamide, Trifluoroacetic Acid Salt

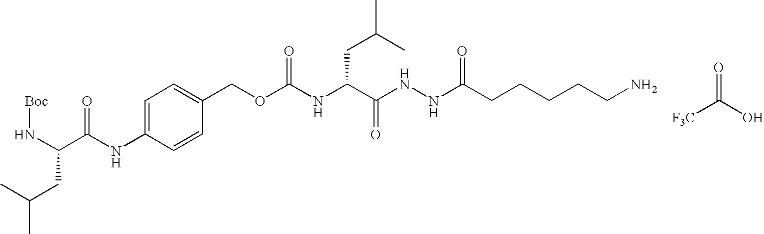

The product of Example 3, Part B (29.0 mg, 0.050 mmol) was dissolved in 50:50 TFA:dichloromethane (2.0 mL), stirred at room temperature for 20 minutes, and concentrated under reduced pressure to give a solid. This solid was taken up in anhydrous DMF (0.5 mL), and treated with DIEA (17 μL, 0.10 mmol), the product of Part B (25.0 mg, 0.050 mmol), and HOBt (7.7 mg, 0.050 mmol). The reaction was stirred at room temperature under nitrogen for 24 hours, treated with TAEA (0.2 mL), and stirred for an additional 20 minutes. The solution was concentrated under reduced pressure and the resulting residue was purified by HPLC on a Phenomenex Luna C18 column (21.2×250 mm) using a 0.9%/min gradient of 13.5 to 36% acetonitrile containing 0.1% TFA at a flow rate of 20 mL/min. The main product peak eluting at 28.8 minutes was lyophilized to give the title compound as a colorless solid (18 mg, 58%, HPLC purity 90%). MS (ESI): 621.5 (60, M+H); HRMS: Calcd for C₃₁H₅₃N₆O₇ (M+H): 621.3970; Found: 621.3977.

### EXAMPLE 14

### Synthesis of N-[(2R)-2-({[4-((2S)-2-{(2S)-2-[2-((2S)-2-[((2S)-1-Acetylpyrrolidin-2-yl)carbonylamino]-N-{4-[(tert-butoxy)carbonylamino]butyl}-4-methylpentanoylamino)-acetylamino]-4-methylpentanoylamino}-4-methylpentanoylamino)phenyl]methoxy}-carbonylamino)-4-methylpentanoylamino]-6-aminohexanamide Trifluoroacetic Acid Salt

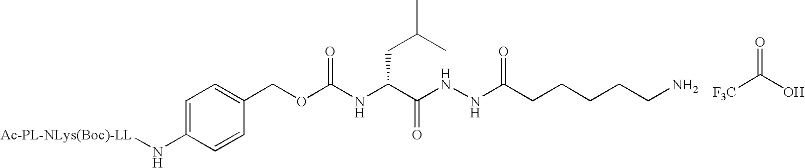

### Part A—Preparation of N-[(2R)-2-({[4-((2S)-2-Amino-4-methylpentanoylamino)phenyl]-methoxy}carbonylamino)-4-methylpentanoylamino]-6-[(fluoren-9-ylmethoxy)-carbonylamino]hexanamide, Trifluoroacetic Acid Salt

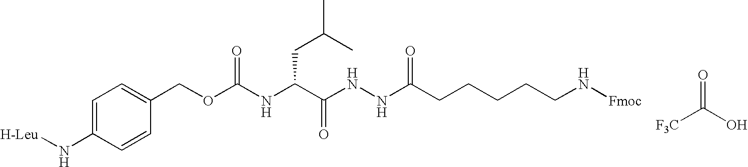

Product of Example 3B (65 mg, 0.112 mmol) was dissolved in 50:50 TFA:dichloromethane (2.0 mL), stirred at room temperature for 20 minutes, and concentrated under reduced pressure to give a solid. This solid was taken up in anhydrous DMF (0.5 mL) and treated with DIEA (39 μL, 0.224 mmol), the product of Example 13B (56.0 mg, 0.112 mmol), and HOBt (17 mg, 0.112 mmol). The reaction was stirred at room temperature under nitrogen for 24 hours, treated with 50:50 TFA:dichloromethane (2.0 mL), and stirred for an additional 30 minutes at room temperature. The solvents were removed under reduced pressure and the resulting residue was purified by HPLC on a Phenomenex Luna C18 column (21.2×250 mm) using a 0.9%/min gradient of 18 to 54% acetonitrile containing 0.1% TFA at a flow rate of 20 mL/min. The main product peak eluting at 33.3 minutes was lyophilized to give the title compound as a colorless solid (58 mg, 70%, HPLC purity 100%). ¹H NMR (CD₃CN) δ 7/78 (dJ=7.2 Hz, 2H), 7.60 (d, J=8.4 Hz, 2H), 7.49 (d, J=8.4 Hz, 2H), 7.38 (t, J=7.5 Hz, 2H), 7.34-7.27 (m, 4H), 5.09-4.93 (m, 2H), 4.29 (d, J=7.2 Hz, 2H), 4.24-4.11 (m, 2H), 3.99 (t, J=7.2 Hz, 1H), 3.04-2.94 (m, 2H), 2.21-2.11 (m, 2H), 1.77-0.99 (m, 12H), 0.96-0.77 (m, 12H). MS (ESI): 743.4 (100, M+H), 1485.6 (20, 2M+H).

### Part B—Preparation of Fmoc-PL-NLys(Boc)-L-HMPB BHA Resin

This peptide was synthesized as part of a peptide library using Irori MacroKan® reaction vessels. Fmoc-Leu-HMPB BHA resin (0.26 g/vessel, substitution level=0.54 mmol/g) was swollen by washing with DMF (8 mL/MacroKan®). The following steps were performed: (Step 1) The Fmoc group was removed using 20% piperidine in DMF (8 mL/MacroKan) for 3 minutes, followed by a second treatment for 30 minutes. (Step 2) The resin was washed thoroughly with dichloromethane (8 mL/MacroKan, 9×3 min), and DMF (8 mL/MacroKan, 3×3 min). (Step 3) Fmoc-NLys(Boc)-OH (5.0 molar equiv), HOBt (5 molar equiv), HBTU (5 molar equiv) in DMF (6 mL/MacroKan), and DIEA (5-10 molar equiv) were added to the reaction flask and the reaction was allowed to proceed for 8 hours. (Step 4) The MacroKans was washed thoroughly with DMF (8 mL/MacroKan, 3×3 min) and dichloromethane (8 mL/MacroKan, 9×3 min). (Step 5) A portion of the resin was removed and assayed for completeness of the reaction. (Step 6) Steps 3-5 were repeated as necessary to complete the coupling reaction. Steps 1-6 were repeated until the sequence Fmoc-PL-NLys(Boc)-L had been attained.

### Part C—Preparation of Ac-PL-NLys(Boc)-L-OH

The MacroKan reaction vessels from Part B were placed in a flask and the resin was swollen by washing with DMF (8 mL/MarcoKan). The Fmoc group was removed using 20% piperidine in DMF (8 mL/MacroKan) for 3 minutes, followed by a second treatment for 30 minutes. (Step 2) The resin was washed thoroughly with dichloromethane (8 mL/MacroKan, 9×3 min), and DMF (8 mL/MacroKan, 3×3 min). Acetic anhydride (5 molar equiv), DIEA (5 molar equiv), and DMF (6 mL/MacroKan) were added, and the reaction was allowed to proceed for 4 hours. The MacroKans was washed thoroughly with DMF (8 mL/MacroKan, 3×3 min) and dichloromethane (8 mL/MacroKan, 9×3 min) and dried overnight under reduced pressure.

The peptide-resin was removed from the MacroKans, placed in a sintered glass funnel, and treated with 1% TFA in dichloromethane (3 mL). After 2 minutes, the solution was filtered, by the application of pressure, directly into a solution of 10% pyridine in methanol (2 mL). The cleavage step was repeated nine times. The combined filtrates were evaporated to 5% of their volume, diluted with water (10 mL), and cooled in an ice-water bath. The resulting precipitate was collected by filtration in a sintered glass funnel, washed with water, and dried under vacuum. Purification was accomplished by HPLC on a Phenomenex Luna C18 column (21.2×250 mm) using a 0.9%/min gradient of 27 to 54% acetonitrile containing 0.1% TFA to give the title compound as a colorless solid (67.7 mg, 79%). MS (ESI): 612.5 (20, M+H), 512.4 (100, M+H−Boc).

### Part D—Preparation of N-[(2R)-2-({[4-((2S)-2-{(2S)-2-[2-((2S)-2-[((2S)-1-Acetylpyrrolidin-2-yl)carbonylamino]-N-{4-[(tert-butoxy)carbonylamino]butyl}-4-methylpentanoylamino)acetylamino]-4-methylpentanoylamino}-4-methylpentanoylamino)phenyl]methoxy}carbonylamino)-4-methylpentanoylamino]-6-[(fluoren-9-ylmethoxy)carbonylamino]hexanamide

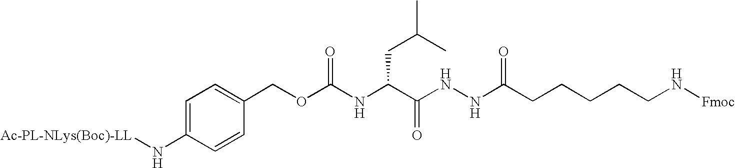

A solution of the product of Part C (19 mg, 0.031 mmol), HOAt (2.8 mg, 0.021 mmol), TMP (14 μL, 0.10 mmol), and DIC (13 μL, 0.084 mmol) in anhydrous DMF (0.5 mL) was stirred at room temperature under nitrogen for 20 minutes. The product of Part A (24 mg, 0.033 mmol) was added, and stirring was continued for 48 hours. The solution was concentrated under reduced pressure and the resulting residue was purified by HPLC on a Phenomenex Luna C18 column (21.2×250 mm) using a 0.9%/min gradient of 45 to 72% acetonitrile containing 15 mM NH₄OAc (pH=7) at a flow rate of 20 mL/min. The main product peak eluting at 20.8 minutes was lyophilized to give the title compound as a colorless solid (11.4 mg, 28%, HPLC purity 100%). MS (ESI): 1236.7 (75, M−Boc+H), 1292.7 (100, M−44+H), 1336.8 (40, M+H).

### Part E—Preparation of N-[(2R)-2-({[4-((2S)-2-{(2S)-2-[2-((2S)-2-[((2S)-1-Acetylpyrrolidin-2-yl)carbonylamino]-N-{4-[(tert-butoxy)carbonylamino]butyl}-4-methylpentanoylamino)acetylamino]-4-methylpentanoylamino}-4-methylpentanoylamino)phenyl]methoxy}-carbonylamino)-4-methylpentanoylamino]-6-aminohexanamide, Trifluoroacetic Acid Salt

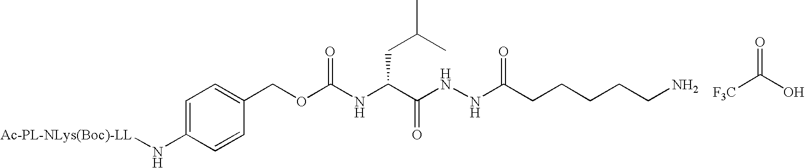

A solution of the product of Part D (13.4 mg, 0.010 mmol) in DMF (1.0 mL) was treated with TAEA (0.2 mL) and stirred at room temperature for 20 minutes. The solution was concentrated under reduced pressure and the resulting residue was purified by HPLC on a Phenomenex Luna C18 column (21.2×250 mm) using a 0.9%/min gradient of 18 to 49.5% acetonitrile containing 0.1% TFA at a flow rate of 20 mL/min. The main product peak eluting at 33.1 minutes was lyophilized to give the title compound as a colorless solid (10.0 mg, 90%, HPLC purity 100%). MS (ESI): 1114.7 (100, M+H), 507.9 (40, 2M+H); HRMS: Calcd for C₅₆H₉₆N₁₁O₁₂ (M+H): 1114.7234; Found: 1114.7223. Chiral analysis: 75.2% L-Leu.

### EXAMPLE 15

### Synthesis of N-{(2R)-2-[(tert-Butoxy)carbonylamino]-4-methylpentanoylamino}-6-(2-bromopropanoylamino)hexanamide

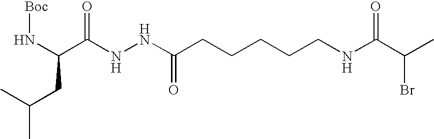

A solution of the product of Example 3, Part B (112 mg, 0.193 mmol) in 20% piperidine in DMF (1.0 mL) was stirred at room temperature under nitrogen for 20 minutes and concentrated under reduced pressure. The resulting residue was taken up in DMF (0.8 mL) and treated with TMP (51 μL, 0.38 mmol) and 2-bromopropionyl bromide (22 μL, 0.21 mmol). The solution was concentrated after 10 minutes and the resulting residue was purified by HPLC on a Phenomenex Luna C18 column (21.2×250 mm) using a 0.9%/min gradient of 27 to 49.5% acetonitrile containing 0.1% TFA at a flow rate of 20 mL/min. The main product peak eluting at 19.1 minutes was lyophilized to give the title compound as a colorless solid (26.6 mg, 28%, HPLC purity 100%). ¹H NMR (CDCl₃) δ 8.59 (s, 1H), 8.08 (s, 1H), 6.60 (s, 1H), 4.92 (d, J=7.2 Hz, 1H), 4.41 (q, J=7.2 Hz, 1H), 4.22 (s, 1H), 3.31-3.23 (m, 2H), 2.26 (t, J=7.2 Hz, 2H), 1.85 (d, J=7.2 Hz, 3H), 1.73-1.65 (m, 4H), 1.58-1.49 (m, 3H), 1.49-1.36 (m, 11H), 0.98-0.91 (m, 6H). MS (ESI): 393.1 (100, M−Boc+H), 395.2 (90. M−Boc+H).

### EXAMPLE 16

### Synthesis of N-{(2R)-2-[(tert-Butoxy)carbonylamino]-4-methylpentanoylamino}-6-(2-fluoropropanoylamino)hexanamide

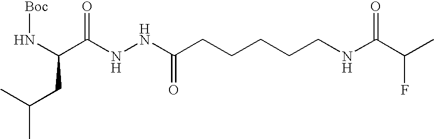 Method A

A solution of the product of Example 3 (9.6 mg, 0.027 mmol), sodium 2-fluoropropionate (2.5 mg, 0.027 mmol), HBTU (10 mg, 0.032 mmol), and DIEA (7.7 μL, 0.54 mmol) in DMF (0.5 mL) was stirred at room temperature under nitrogen for 10 minutes. The solvents were removed under reduced pressure and the resulting residue was purified by HPLC on a Phenomenex Luna C18 column (21.2×250 mm) using a 0.9%/min gradient of 18 to 45% acetonitrile containing 0.1% TFA at a flow rate of 20 mL/min. The main product peak eluting at 24.2 minutes was lyophilized to give the title compound as a colorless solid (8.5 mg, 73%, HPLC purity 100%). ¹H NMR (CD₃CN) δ 9.19 (s, 1H), 8.50 (m, 1H), 6.48 (s, 1H), 5.14-5.05 (m, 1H), 4.96 (dd, J₁=6.8 Hz, J₂=49.2 Hz, 1H), 4.26 (s, 1H), 3.34-3.22 (m, 2H), 1.73-1.61 (m, 4H), 1.59-1.50 (m, 6H), 1.47-1.34 (m, 11H), 0.97-0.89 (m, 6H). MS (ESI): 455.3 (10, M+Na), 333.3 (100, M−Boc+H); HRMS: Calcd for C₂₀H₃₇FN₄O₅ (M+H): 433.2821; Found: 433.2824.

Method B

A solution of KF (3.9 μL of a 0.1 mg/μL solution in water, 0.007 mmol) and Kryptofix® (5.0 mg, 0.013 mmol) in acetonitrile (0.5 mL) was evaporated under a stream of nitrogen while warming the flask at 90° C. This azeotropic drying step was repeated five times and the resulting residue was dried further under high vacuum for 15 minutes. The residue was dissolved in a solution of the product of Example 15 (1.1 mg, 0.0020 mmol) in 0.25 mL of anhydrous acetonitrile. The solution was stirred at 90° C. under nitrogen for 5 minutes. Analysis by LC/MS indicated a conversion of ˜15% of the bromide to the title compound.

### EXAMPLE 17

### Synthesis of N-{(2R)-2-[(tert-Butoxy)carbonylamino]-4-methylpentanoylamino}-6-[(2,5-dinitrophenyl)carbonylamino]hexanamide

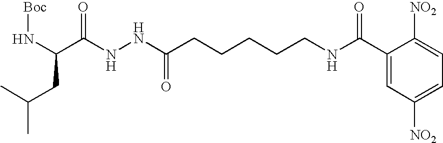

A solution of 2,5-dinitrobenzoic acid (0.212 g, 1.00 mmol), oxalyl chloride (96 μL, 1.1 mmol), and DMF ( 10 μL) in dichloromethane (10 mL), was stirred at ambient temperatures for 45 minutes. The solution was concentrated under reduced pressure resulting in a yellow oil (225 mg). This oil (80 mg, 0.344 mmol) was dissolved in dichloromethane (2.0 mL) along with the product of Example 3 (62 mg, 0.17 mmol) and DIEA (60 μL, 0.34 mmol). The solution was stirred at ambient temperatures for 2 hours, and concentrated under vacuum to produce an orange solid. The compound was purified by HPLC on a Phenomenex Luna C18 column (21.2×250 mm) using a 0.9%/min gradient of 27 to 54% acetonitrile containing 0.1% TFA at a flow rate of 20 mL/min. The main product peak eluting at 25.0 minutes was lyophilized to give the title compound as an off-white solid (25 mg, 27%). ¹H NMR (CDCl₃/CD₃CN) δ 8.38 (s, 1H), 8.27-8.22 (m, 2H), 8.01-7.90 (m, 2H), 7.10 (s, 1H), 4.98 (m, 1H), 3.25 (q, J=6.0 Hz, 2H), 2.10 (t, J=7.2 Hz, 2H), 1.58-1.23 (m, 18H), 0.81-0.74 (m, 6H). MS (ESI): 451.4 (100, M+H−Boc).

### EXAMPLE 18

### Synthesis of N-{(2R)-2-[(tert-Butoxy)carbonylamino]-4-methylpentanoylamino}-6-[(2-fluoro-5-nitrophenyl)carbonylamino]hexanamide

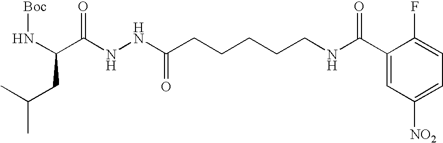

A solution of the product from Example 17 (8.0 mg, 0.014 mmol), KF (2.5 mg, 0.044 mmol), and Kryptofix® (16 mg, 0.044 mmol) in acetonitrile (1.0 mL), was evaporated under a stream of nitrogen while warming the flask at 75° C. This azeotropic drying step was repeated five times and the resulting residue was dried further under high vacuum for 15 minutes. The residue was dissolved in acetonitrile (1.0 mL) and heated at 85° C. for 5 minutes, and concentrated under reduced pressure to give an orange/red oil. The oil was purified by HPLC on a Phenomenex Luna C18 column (21.2×250 mm) using a 0.9%/min gradient of 27 to 54% acetonitrile containing 0.1% TFA at a flow rate of 20 mL/min. The main product peak eluting at 24.6 minutes was lyophilized to give the title compound as a colorless solid (1.6 mg, 21%). ¹H NMR (CDCl₃) δ 8.97-8.93 (m, 1H), 8.70 (bs, 1H), 8.36-8.31 (m, 1H), 7.92 (bs, 1H), 7.29 (t, J=9.9 Hz, 1H), 6.76 (s, 1H), 4.80 (s, 1H), 4.18 (s, 1H), 3.53-3.47 (m, 2H), 2.28 (t, J=7.2 Hz, 2H), 1.78-1.38 (m, 18H), 0.98-0.86 (m, 6H). MS (ESI): 426.2 (100, M+H−Boc).

### EXAMPLE 19

### Synthesis of 2-[(2-{[(N-{5-[N-((2R)-2-Amino-4-methylpentanoylamino)-carbamoyl]pentyl}carbamoyl)methyl]{2-[bis(carboxymethyl)amino]-ethyl}amino}ethyl)(carboxymethyl)amino]acetic Acid, Trifluoroacetic Acid Salt

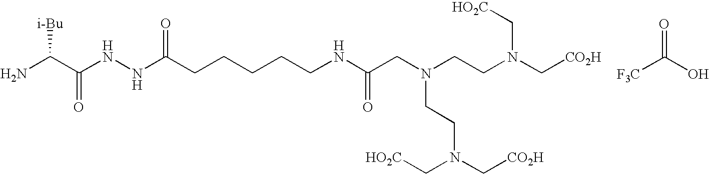

The product of Example 3B (116.1 mg, 0.200 mmol) was massed into a 5 mL round bottom flask and treated with a solution of piperidine in DMF (1:4 v/v, 4.00 mL) at 22° C. After stirring 0.5 hours, all volatiles were removed in vacuo. The resulting solid material was taken up in DMF (2.00 mL) and transferred to a previously prepared solution of 2-{bis[2-(bis {[(tert-butyl)oxycarbonyl]methyl}amino)ethyl]amino}acetic acid (136 mg, 0.220 mmol) in DMF (2.00 mmol) containing HBTU (83.4 mg, 0.220 mmol), HOBt (33.7 mg, 0.220 mmol) and i-Pr₂NEt (105 μL, 0.600 mmol). The resulting solution was maintained at 22° C. for 1 hour, then concentrated in vacuo and the residue purified by HPLC on a Phenomenex Luna C18 column (21.2×250 mm) using a 1.0%/min gradient of 50-80% acetonitrile containing 0.1% TFA at a flow rate of 20 mL/min. The main product peak eluting at 16 minutes was lyophilized to a white solid. The entire mass was taken up in CH₂Cl₂ (3.00 mL) and treated with TFA (750 μL, 9.74 mmol). After stirring 7 hours at 22° C., the resulting solution was concentrated in vacuo and purified by HPLC on a Phenomenex Luna C18 column (21.1×250 mm) using a 2.0%/min gradient of 0-40% acetonitrile containing 0.1% TFA at a flow rate of 20 mL/min. The main product peak eluting at 7 minutes was lyophilized to a white solid (101 mg, 0.0928 mmol; 46.4%). ¹H NMR (DMSO-d₆, 600 MHz): δ 10.39 (1H, s), 9.99 (1H, s), 8.42 (1H, t, J=5.4 Hz), 8.19 (3H, br s), 4.14 (2H, s), 3.77 (1H, br s), 3.50 (8H, s), 3.36 (4H, brt, J=5.4 Hz), 3.11 (2H, td, J=6.8, 6.1 Hz), 3.04 (3H, brt, J=5.6 Hz), 2.16 (2H, t, J=7.4 Hz), 1.76-1.71 (1H, m), 1.63 (4H, m), 1.47-1.42 (2H, m), 1.32-1.27 (2H, m), 0.92 (3H, d, J=6.5 Hz), 0.90 (3H, d, J=6.5 Hz). ¹³C NMR (DMSO-d₆, 150 MHz) δ 172.7, 170.8, 167.6, 164.4, 54.3, 53.9, 52.1, 49.6, 48.6, 40.4, 38.7, 32.9, 28.5, 25.9, 24.6, 23.4, 22.5, 21.9. MS (ESI): 634.4 (61.2, M+H), 317.9 (100).

### EXAMPLE 20

### Synthesis of N-{(2R)-2-[(tert-Butoxy)carbonylamino]-3-phenylpropanoylamino}-6-aminohexanamide, Trifluoroacetic Acid Salt

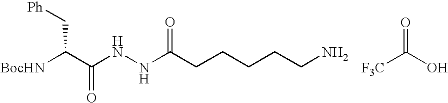

A solution of Boc-D-Phe-OH (10 mg, 0.038 mmol) and HOAt (5.2 mg, 0.038 mmol) in dry DMF (1.00 mL) was successively treated with collidine (35 μL, 0.26 mmol) and DIC (5.9 μL, 0.038 mmol) then stirred 5 minutes at 22° C. The product of Example 3A (18.0 mg, 37.4 μmol) was added in one portion and the resulting solution stirred 1 hour at 22° C. All volatiles were then removed in vacuo and the resulting oil treated with a solution of piperidine in DMF (1:4 v/v, 1.00 mL). The solution was stirred 0.5 hours, then concentrated in vacuo and the crude residue purified by HPLC on a Phenomenex Luna C18 column (21.2×250 mm) using a 1.0%/min gradient of 5-35% acetonitrile containing 0.1% TFA at a flow rate of 20 mL/min. The main product peak eluting at 23 minutes was lyophilized to a white solid (7.7 mg, 0.015 mmol; 40%). ¹H NMR (DMSO-d₆, 600 MHz) δ 9.97 (1H, s), 9.83 (1H, s), 7.64 (3H, br s), 7.31-7.26 (4H, m), 7.19 (1H, t, J=7.0 Hz), 6.91 (1H, d, J=8.7 Hz), 4.23 (1H, ddd, J=10.8, 8.9, 3.8 Hz), 2.99 (1H, dd, J=13.9, 3.5 Hz), 2.80-2.73 (3H, m), 2.14 (2H, t, J=7.3 Hz), 1.54 (4H, tt, J=7.6, 7.5 Hz), 1.33 (2H, tt, J=8.0, 7.3 Hz), 1.29 (9H, s). MS (ESI): 393.4 (100, M+H). HRMS: Calcd for C₂₀H₃₃N₄O₄: 393.2496; Found: 393.2500.

### EXAMPLE 21

### Synthesis of 6-Amino-N-{2-[(tert-Butoxy)carbonylamino]-2-methylpropanoylamino}hexanamide, Trifluoroacetic Acid Salt

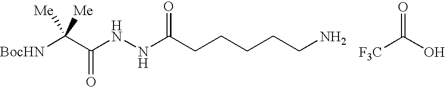

A solution of Boc-Aib-OH (25.0 mg, 0.123 mmol) and HOAt (14.0 mg, 0.103 mmol) in dry DMF (1.00 mL) was successively treated with collidine (75.5 μL, 0.571 mmol) and DIC (16 μL, 0.102 mmol) then stirred 5 minutes at 22° C. The product of Example 3A (30.0 mg, 62.3 μmol) was added in one portion and the resulting solution stirred 1.5 hours at 22° C. All volatiles were then removed in vacuo, and the crude residue purified by HPLC on a Phenomenex Luna C18 column (21.2×250 mm) using a 1.0%/min gradient of 45-75% acetonitrile containing 0.1% TFA and 10% H₂O at a flow rate of 20 mL/min. The main product peak eluting at 28 minutes was lyophilized to a white solid then treated with a solution of piperidine in DMF (1:4 v/v, 1.00 mL). The solution was stirred 0.5 hours, then concentrated in vacuo and the crude residue purified by HPLC on a Phenomenex Luna C18 column (21.2×250 mm) using a 1.0%/min gradient of 0-30% acetonitrile containing 0.1% TFA and 10% H₂O at a flow rate of 20 mL/min. The main product peak eluting at 17 minutes was lyophilized to a white solid (11.4 mg, 25.6 μmol; 41.2%). ¹H NMR (DMSO-d₆, 600 MHz): δ 9.68 (1H, br s), 9.38 (1H, d, J=1.7 Hz), 7.65 (3H, br s), 2.77 (2H, tq, J=7.4, 5.7 Hz), 2.10 (2H, t, J=7.4 Hz), 1.55-1.49 (4H, m), 1.37 (9H, s), 1.35 (6H, s), 1.31 (2H, m). MS (ESI): 331.4 (100, M+H). HRMS: Calcd for C₁₅H₃₁N₄O₄ (M+H): 331.2340; Found: 331.2339.

### EXAMPLE 22

### Synthesis of N-{(2R)-2-[(tert-Butoxy)carbonylamino]-5-[(imino{[(2,2,5,7,8-pentamethylchroman-6-yl)sulfonyl]amino}methyl)amino]pentanoylamino}-6-aminohexanamide, Trifluoroacetic Acid Salt

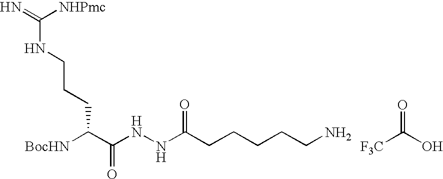

A solution of Boc-D-Arg(Pmc)-OH (66.2 mg, 0.122 mmol) and HOAt (14.0 mg, 0.103 mmol) in dry DMF (1.00 mL) was successively treated with collidine (75.5 μL, 0.571 mmol) and DIC (16.0 μL, 0.102 mmol) then stirred 5 minutes at 22° C. The product of Example 3A (30.0 mg, 62.3 μmol) was added in one portion and the resulting solution stirred 1.5 hours at 22° C. All volatiles were then removed in vacuo, and the resulting oil treated with solution of piperidine in DMF (1:4 v/v, 4.00 mL). The solution was stirred 0.5 hours, then concentrated in vacuo and the crude residue purified by HPLC on a Phenomenex Luna C18 column (21.2×250 mm) using a 1.0%/min gradient of 25-55% acetonitrile containing 0.1% TFA and 10% H₂O at a flow rate of 20 mL/min. The main product peak eluting at 22 minutes was lyophilized to a white solid (25.7 mg, 32.9 μmol; 52.7%). ¹H NMR (DMSO-d₆, 600 MHz): δ 9.80 (1H, s), 9.79 (1H, s), 7.62 (3H, br s), 6.87 (1H, d, J=8.2 Hz), 3.94 (1H, dt, J=8.0, 7.8 Hz), 3.03 (2H, m), 2.77 (2H, tq, J=6.1, 5.9 Hz), 2.59 (2H, t, J=7.5 Hz), 2.03 (3H, s), 1.78 (2H, t, J=6.8 Hz), 1.61-1.44 (7H, m), 1.37 (9H, s), 1.34-1.30 (2H, m), 1.26 (3H, s). MS (ESI): 1335.6 (18.5, 2M+H), 668.4 (100, M+H). HRMS: Calcd for C₃₁H₅₄N₇O₇S (M+H): 668.3800; Found: 668.3799.

### EXAMPLE 23

### Synthesis of tert-Butyl (4R)-4-[N-(6-Aminohexanoylamino)carbamoyl]-4-[(tert-butoxy)carbonylamino]butanoate, Trifluoroacetic Acid Salt

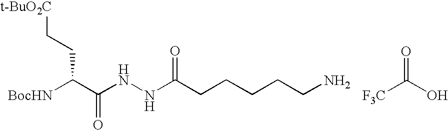

A solution of Boc-D-Glu(But)-OH (37.2 mg, 0.123 mmol) and HOAt (14.0 mg, 0.103 mmol) in dry DMF (1.00 mL) was successively treated with collidine (75.5 μL, 0.571 mmol) and DIC (16.0 μL, 0.102 mmol) then stirred 5 minutes at 22° C. The product of Example 3A (30.0 mg, 62.3 μmol) was added in one portion and the resulting solution stirred 1.5 hours at 22° C. All volatiles were then removed in vacuo, and the resulting oil treated with a solution of tris(2-aminoethyl)amine in DMF (1:4 v/v, 4.00 mL). The solution was stirred 0.5 hours, then concentrated in vacuo and the crude residue purified by HPLC on a Phenomenex Luna C18 column (21.2×250 mm) using a 1.0%/min gradient of 15-45% acetonitrile containing 0.1% TFA at a flow rate of 20 mL/min. The main product peak eluting at 17 minutes was lyophilized to a white solid (24.3 mg, 44.6 μmol; 71.6%). ¹H NMR (DMSO-d₆, 600 MHz): δ 8.77 (1H, s), 8.68 (1H, s), 7.32 (3H, br s), 5.76 (1H, br s), 4.11 (1H, br s), 2.96 (2H, t, J=6.8 Hz), 2.22 (2H, t J=6.8 Hz), 2.02-1.97 (1H, m), 1.85-1.80 (1H, m), 1.70 (2H, tt, J=7.6, 7.5 Hz), 1.65 (2H, tt, J=7.0, 6.9 Hz), 1.54 (2H, m), 1.44 (9H, s), 1.42 (9H, s). MS (ESI): 431.3 (100, M+H).

### EXAMPLE 24

### Synthesis of 6-Amino-N-({[(tert-Butoxy)carbonylamino]cyclopentyl}carbonylamino)hexanamide, Trifluoroacetic Acid Salt

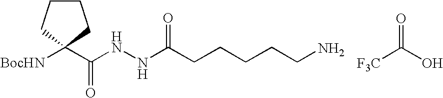

### Part A—Preparation of N-({[(tert-butoxy)carbonylamino]cyclopentyl}carbonylamino)-6-[(fluoren-9-ylmethoxy)carbonylamino]hexanamide

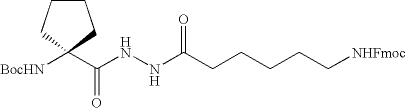

A solution of 1-tert-butoxycarbonylamino cyclopentanecarboxylic acid (72.0 mg, 0.314 mmol) and HOAt (35.6 mg, 0.262 mmol) in dry DMF (3.50 mL) was successively treated with collidine (193 μL, 1.46 mmol) and DIC (40.2 μL, 0.257 mmol) then stirred 5 minutes at 22° C. The product of Example 3A (100 mg, 0.208 mmol) was added in one portion and the resulting solution stirred 3 hours at 22° C. All volatiles were then removed in vacuo, and the crude residue purified by HPLC on a Phenomenex Luna C18 column (21.2×250 mm) using a 1.0%/min gradient of 40-90% acetonitrile containing 0.1% TFA and 10% H₂O at a flow rate of 20 mL/min. The main product peak eluting at 16 minutes was lyophilized to a white solid (50.0 mg, 86.4 μmol; 41.6%). ¹H NMR (C₆D₆, 600 MHz): δ 9.50 (1H, br s), 8.62 (1H, br s), 7.59 (2H, d, J=7.4 Hz), 7.50 (2H, br d, J=7.1 Hz), 7.23 (2H, t, J=7.3 Hz), 7.19 (2H, t, J=7.1 Hz), 4.47 (2H, br d, J=5.8 Hz), 4.40 (1H, br s), 4.05 (1H, br s), 2.88 (2H, m), 2.45-2.25 (2H, m), 2.02-1.69 (4H, m), 1.62-1.33 (4H, m), 1.43 (9H, s), 1.14-1.03 (4H, m). MS (ESI): 601.3 (58.5, M+Na), 479.4(100, M−Boc). HRMS: Calcd for C₃₂H₄₃N₄O₆ (M+H): 579.3177; Found: 579.3180.

### Part B—Preparation of 6-Amino-N-({[(tert-Butoxy)carbonylamino]cyclopentyl}carbonylamino)hexanamide, Trifluoroacetic Acid Salt

The product of Part A (40.0 mg, 69.1 μmol) was treated with a solution of piperidine in DMF (1:4 v/v, 1.00 mL). The solution was maintained for 0.5 hours, then concentrated in vacuo and the crude residue purified by HPLC on a Phenomenex Luna C18 column (21.2×250 mm) using a 1.0%/min gradient of 5-35% acetonitrile containing 0.1% TFA at a flow rate of 20 mL/min. The main product peak eluting at 12 minutes was lyophilized to a white solid (24.0 mg, 51.0 mol; 73.8%). ¹H NMR (C₆D₆, 600 MHz): δ 10.29 (1H, br s), 9.66 (1H, br s), 8.63 (3H, br s), 6.69 (1H, br s), 2.71 (2H, t, J=7.2 Hz), 2.49 (2H, dt, J=13.4, 7.5 Hz), 2.27 (2H, t, J=7.1 Hz), 2.17 (2H, br s), 1.67-1.57 (8H, m), 1.47 (9H, s), 1.29 (2H, tt, J=7.6, 7.4 Hz). MS (ESI): 357.4 (100, M+H). HRMS: Calcd for C₁₇H₃₃N₄O₄: 357.2502 (M+H); Found: 357.2491.

### EXAMPLE 25

### Synthesis of N-{(2R)-2-[(tert-Butoxy)carbonylamino]propanoylamino}-6-aminohexanamide, Trifluoroacetic Acid Salt

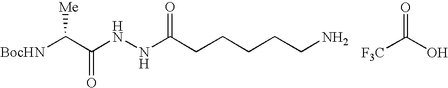

A solution of Boc-D-Ala-OH (23.2 mg, 0.123 mmol) and HOAt (14.0 mg, 0.103 mmol) in dry DMF (1.00 mL) was successively treated with collidine (75.5 μL, 0.571 mmol) and DIC (16.0 μL, 0.102 mmol) then stirred 5 minutes at 22° C. The product of Example 3A (30.0 mg, 62.3 μmol) was added in one portion and the resulting solution stirred 2 hours at 22° C. All volatiles were then removed in vacuo, and the resulting oil treated with a solution of tris(2-aminoethyl)amine in DMF (1:4 v/v, 4.00 mL). The solution was stirred 0.5 hours, then concentrated in vacuo and the crude residue purified by HPLC on a Phenomenex Luna C18 column (21.2×250 mm) using a 1.5%/min gradient of 0-30% acetonitrile containing 0.1% TFA at a flow rate of 20 mL/min. The main product peak eluting at 12 minutes was lyophilized to a white solid (21.3 mg, 49.5 μmol; 79.4%). ¹H NMR (DMSO-d₆, 600 MHz): δ 9.76 (1H, s), 9.73 (1H, s), 7.62 (3H, br s), 6.90 (1H, d, J=7.4 Hz), 4.02 (1H, dq, J=7.2, 7.1 Hz), 2.77 (2H, tq, J=5.9, 5.8 Hz), 2.11 (2H, t, J=7.4 Hz), 1.55-1.50 (4H, m), 1.34-1.29 (2H, m), 1.37 (9H, s), 1.20 (3H, d,J=7.1 Hz). MS (ESI): 317.4 (100, M+H), 261.3 (9.5). HRMS: Calcd for C₁₄H₂₉N₄O₄: 317.2183; Found: 317.2186.

### EXAMPLE 26

### Synthesis of (3S)-N-(6-Aminohexanoylamino)-3-[(tert-butoxy)carbonylamino]-5-methylhexanamide, Trifluoroacetic Acid Salt

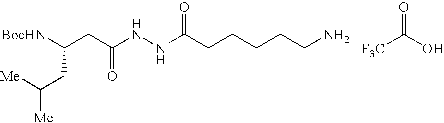

A solution of Boc-β-homoLeu-OH (66.5 mg, 0.271 mmol) and HOAt (36.9 mg, 0.271 mmol) in dry DMF (3.60 mL) was successively treated with collidine (119 μL, 0.900 mmol) and DIC (42.0 μL, 0.268 mmol) then stirred 5 minutes at 22° C. The product of Example 3A (87.0 mg, 0.181 mmol) was added in one portion and the resulting solution stirred 2 hours at 22° C. All volatiles were then removed in vacuo, and the resulting oil treated with a solution of piperidine in DMF (1:4 v/v, 3.60 mL). The solution was maintained for 0.5 hours, then concentrated in vacuo and the crude residue purified by HPLC on a Phenomenex Luna C18 column (21.2×250 mm) using a 1.0%/min gradient of 5-35% acetonitrile containing 0.1% TFA at a flow rate of 20 mL/min. The main product peak eluting at 27 minutes was lyophilized to a white solid (31.4 mg, 64.5 μmol; 35.7%). ¹H NMR (DMSO-d₆, 600 MHz): δ 9.68 (1H, s), 7.68 (2H, br s), 6.59 (1H, d, J=8.9 Hz), 3.85-3.80 (1H, m), 2.77 (2H, tq, J=6.0, 5.8 Hz), 2.21 (2H, ABXX′, J_{AB}=14.3 Hz, J_{AX}=J_{BX}=8.3 Hz, J_{AX′}=J_{BX′}=5.3 Hz), 2.11 (2H, t, J=7.4 Hz), 1.59-1.49 (5H, m), 1.37 (9H, s), 1.34-1.29 (3H, m), 1.20 (1H, ddd, J=13.3, 9.2, 3.9 Hz), 0.84 (3H, d, J=6.9 Hz), 0.83 (3H, d, J=7.0 Hz). ¹³C NMR (DMSO-d₆, 150 MHz) δ 170.6, 168.8, 154.8, 77.3, 45.5, 44.1, 42.9, 38.5, 32.7, 28.1(3), 26.6, 25.2, 24.3, 24.2, 23.2, 21.5. MS (ESI): 373.4 (100, M+H). HRMS: Calcd for C₁₈H₃₇N₄O₄: 373.2809; Found: 373.2808.

### EXAMPLE 27

### Synthesis of (2R)-6-{(2S)-2-[(tert-Butoxy)carbonylamino]-4-methylpentanoylamino}-N-(6-aminohexanoylamino)-2-[(tert-butoxy)carbonylamino]hexanamide, Trifluoroacetic Acid Salt

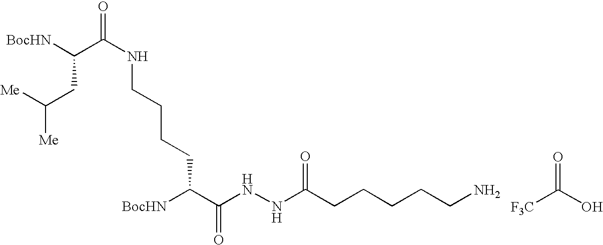

### Part A—Preparation of (2R)-6-{(2S)-2-[(tert-Butoxy)carbonylamino]-4-methylpentanoylamino}-2-[(tert-butoxy)carbonylamino]hexanoic acid, Trifluoroacetic Acid Salt

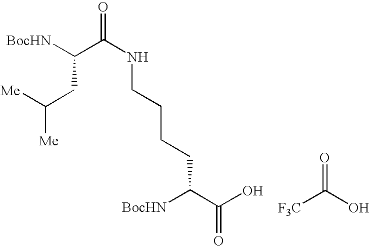

Boc-D-Lys(Fmoc)-OH (260 mg, 0.555 mmol) was treated with a solution of piperidine in DMF (1:4 v/v, 4.00 mL). The solution was maintained for 0.5 hours, then concentrated in vacuo and dried on the vacuum manifold for 18 hours to insure complete removal of excess piperidine. The resulting solid material was taken up in DMF (2.00 mL) and transferred to a previously prepared solution of Boc-Leu-OH (193 mg, 0.830 mmol) in DMF (2.00 mL) containing HBTU (263 mg, 0.694 mmol), HOBt (106 mg, 0.692 mmol) and i-Pr₂NEt (483 μL, 2.77 mmol). The resulting solution was maintained at 22° C. for 1 hour, then concentrated in vacuo. The residue was purified by HPLC on a Phenomenex Luna C18 column (21.2×250 mm) using a 2.0%/min gradient of 35-75% acetonitrile containing 0.1% TFA at a flow rate of 20 mL/min. The main product peak eluting at 15 minutes was lyophilized to a white solid (203 mg, 0.442 μmol; 79.6%). MS (ESI): 482.4 (30, M+Na), 460.4 (14, M+H), 360 (100, M−Boc). This material was used without further purification in the subsequent step.

### Part B—Preparation of (2R)-6-{(2S)-2-[(tert-Butoxy)carbonylamino]-4-methylpentanoylamino}-N-(6-aminohexanoylamino)-2-[(tert-butoxy)carbonylamino]hexanamide, Trifluoroacetic Acid Salt

A solution of the product of Part A (45.0 mg, 97.9 μmol) and HOAt (12.3 mg, 90.4 μmol) in dry DMF (2.00 mL) was successively treated with collidine (53.9 μL, 0.408 mmol) and DIC (14.1 μL, 90.1 μmol) then stirred 5 minutes at 22° C. The product of Example 3A (30.0 mg, 62.3 μmol) was added in one portion and the resulting solution stirred 3 hours at 22° C. All volatiles were then removed in vacuo, and the resulting oil treated with a solution of tris(2-aminoethyl)amine in DMF (1:4 v/v, 2.00 mL). The solution was maintained for 0.5 hours, then concentrated in vacuo and the crude residue purified by HPLC on a Phenomenex Luna C18 column (21.2×250 mm) using a 2.0%/min gradient of 0-40% acetonitrile containing 0.1% TFA at a flow rate of 20 mL/min. The main product peak eluting at 20 minutes was lyophilized to a white solid (25.3 mg, 36.1 μmol; 57.9%). ¹H NMR (DMSO-d₆, 600 MHz): δ 9.76 (1H, br s), 7.72 (1H, t, J=5.5 Hz), 7.61 (2H, br s), 6.79 (1H, d, J=8.2 Hz), 6.71 (1H, d, J=8.0 Hz), 3.94-3.89 (1H, m), 3.06-2.97 (2H, m), 2.77 (2H, td, J=5.9, 5.8 Hz), 2.11 (2H, t, J=7.3 Hz), 1.55-1.50 (2H, m), 1.37 (18H, s), 1.43-1.23 (13H, m), 0.87 (3H, d, J=6.6 Hz), 0.85 (3H, d, J=6.7 Hz). MS (ESI): 587.4 (100, M+H). HRMS: Calcd for C₂₈H₅₅N₆O₇: 587.4127; Found: 587.4122.

### EXAMPLE 28

### Synthesis of 6-{(2R)-2-[(Fluoren-9-ylmethoxy)carbonylamino]-4-methylpentanoylamino}-N-aminohexanamide, Formic Acid Salt

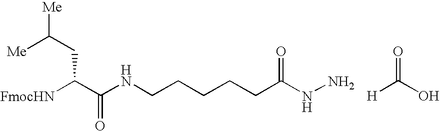

### Part A—Preparation of 6-{(2R)-2-[(Fluoren-9-ylmethoxy)carbonylamino]-4-methylpentanoylamino}-N-[(tert-butoxy)carbonylamino]hexanamide, Trifluoroacetic Acid Salt

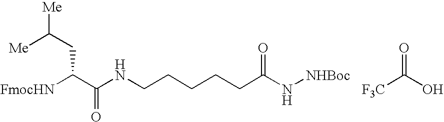

To a solution of the product of Example 1B (123 mg, 0.342 mmol) in dry DMF (5.00 mL) was added Fmoc-D-Leu-OH (145.0 mg, 0.410 mmol), HBTU (143 mg, 0.377 mmol) and HOBt (52.0 mg, 0.340 mmol) followed by i-Pr₂NEt (179 μL, 1.03 mmol) at 22° C. After stirring 1.5 hours, the solution was diluted with ethyl acetate and H₂O (50 mL each) with transfer to a separatory funnel. The layers were separated and the aqueous layer washed with ethyl acetate (2×20 mL). The combined ethyl acetate layers were consecutively washed with 0.1 N HCl and saturated solutions of NaHCO₃ and NaCl (30 mL each), then dried over MgSO₄, filtered and concentrated in vacuo. The resulting pale yellow oil (˜200 mg) was used without further purification in the subsequent step. MS (ESI): 603.3 (90, M+Na), 581.4 (100, M+H), 481.4 (94, M−Boc).

### Part B—Preparation of 6-{(2R)-2-[(Fluoren-9-ylmethoxy)carbonylamino]-4-methylpentanoylamino}-N-aminohexanamide, Formic Acid Salt

A solution of the product of Part A (200 mg, 0.342 μmol) in CH₂Cl₂ (3.00 mL) at 22° C. was treated with TFA (3.00 mL). After stirring 0.5 hours, the solution was concentrated in vacuo and the crude residue purified by HPLC on a Phenomenex Luna C18 column (21.2×250 mm) using a 2.0%/min gradient of 25-75% acetonitrile containing 0.1% HCO₂H at a flow rate of 20 mL/min. The main product peak eluting at 11 minutes was lyophilized to a white solid (99.0 mg, 0.188 mmol; 54.9% over two steps). ¹H NMR (C₆D₆, 600 MHz): δ 7.76 (1H, br s), 7.65 (2H, d, J=7.2 Hz), 7.60 (2H, d, J=7.3 Hz), 7.25-7.17 (4H, m), 4.57 (1H, td, J=8.9, 5.5 Hz), 4.38 (2H, ABqd, J_{AB}=10.7 Hz, J_{d}=7.4 Hz), 4.14 (1H, t, J=7.3 Hz), 3.34-3.19 (2H, m), 2.30 (1H, t, J=6.9 Hz), 2.17 (2H, t, J=7.3 Hz), 1.86 (1H, qq, J=6.9, 6.7 Hz), 1.82-1.75 (2H, m), 1.64 (2H, tt, J=7.6, 7.5 Hz), 1.51-1.44 (2H, m), 1.29 (2H, tt, J=7.6, 7.4 Hz), 0.99 (3H, d, J=6.4 Hz), 0.97 (3H, d, J=6.5 Hz). ¹³C NMR (C₆D₆, 150 MHz) δ 172.9, 172.8, 156.8, 144.7, 144.6, 141.6, 127.4, 125.8, 120.2, 66.6, 54.1, 47.7, 42.4, 39.2, 34.0, 29.4, 26.6, 25.3, 25.1, 23.4, 22.2. MS (ESI): 503.4 (15.5, M+Na), 481.4 (100, M+H). HRMS: Calcd for C₂₇H₃₇N₄O₄ (M+H): 481.2809; Found: 481.2811.

### EXAMPLE 29

### Synthesis of (2R)—N-{[4-(Aminomethyl)phenyl]carbonylamino}-2-[(tert-butoxy)carbonylamino]-4-methylpentanamide, Trifluoroacetic Acid Salt

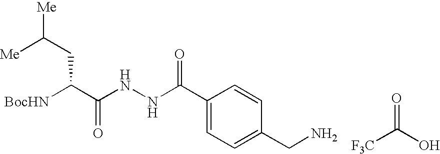

### Part A—Preparation of N-[(tert-Butoxy)carbonylamino](4-{[(fluoren-9-ylmethoxy)carbonylamino]methyl}phenyl)carboxamide

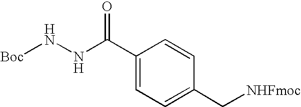

A solution of Fmoc-Amb-OH (1.00 g, 2.68 mmol) in dry DMF (10.0 mL) at 22° C. was treated with HBTU (1.22 g, 3.22 mmol) followed by i-Pr₂NEt (2.30 mL, 13.2 mmol) and tert-butyl carbazate (354 mg, 2.68 mmol). After 2 hours of reaction time, all volatiles were removed in vacuo. The resulting oil was taken up in ethyl acetate (50 mL), washed with saturated solutions of NaHCO₃ (2×15 mL) and NaCl (1×15 mL) then dried over MgSO₄, filtered through a plug of silica and concentrated in vacuo to a pale yellow crystalline solid (1.20 g, 2.46 mmol, 91.9%). This material was used directly in the subsequent step. ¹H NMR (CDCl₃, 600 MHz): a 9.50 (1H, br s), 7.71 (1H, br s), 7.40 (2H, br d, J=6.6 Hz), 7.31 (2H, d, J=7.5 Hz), 7.20 (2H, d, J=7.4 Hz), 6.94 (2H, t, J=7.4 Hz), 6.90 (1H, t, J=5.9 Hz), 6.86-6.84 (4H, m), 3.97 (2H, d, J=6.8 Hz), 3.87 (2H, d, J=6.0 Hz), 3.77 (1H, t, J=6.6 Hz), 1.02 (9H, s). MS (ESI): 875.3 (100, 2M−Boc). 388.2 (90, M−Boc).

### Part B—Preparation of (2R)—N-{[4-(Aminomethyl)phenyl]carbonylamino}-2-[(tert-butoxy)carbonylamino]-4-methylpentanamide, Trifluoroacetic Acid Salt

The product of Part A (100 mg, 0.205 mmol) was treated with a solution of TFA in CH₂Cl₂ (1:1 v/v, 2.00 mL) at 22° C. After 0.25 hours all volatiles were removed in vacuo, the residue taken up in dry DMF (2.00 mL) then treated with collidine (70.0 μL, 0.530 mmol). The resulting solution was transferred to a previously prepared solution of Boc-D-Leu-OH (71.0 mg, 0.307 mmol), HOAt (35.0 mg, 0.257 mmol), collidine (190 μL, 1.44 mmol) and DIC (40.1 μL, 0.256 mmol) in dry DMF (2.0 mL). After 0.5 hours at 22° C., an additional 1.50 equiv of activated amino acid solution were transferred to the reaction mixture; complete consumption of free hydrazide was then observed within 4 hours. After concentration in vacuo, the crude residue was purified by HPLC on a Phenomenex Luna C18 column (21.2×250 mm) using a 1.2%/min gradient of 50-80% acetonitrile containing 0.1% TFA at a flow rate of 20 mL/min. The main product peak eluting at 20 minutes was lyophilized to a white solid. The entire mass was subsequently treated with a solution of piperidine in DMF (1:4 v/v, 3.00 mL). After stirring 3 hours at 22° C., all volatiles were removed in vacuo and the crude residue purified by HPLC on a Phenomenex Luna C18 column (21.2×250 mm) using a 1.2%/min gradient of 10-40% acetonitrile containing 0.1% TFA at a flow rate of 20 mL/min. The main product peak eluting at 20 minutes was lyophilized to a white solid (50.7 mg, 0.103 mmol; 50.2%). ¹H NMR (DMSO-d₆, 600 MHz): δ 9.96 (1H, br s), 8.19 (2H, br s), 7.92 (2H, AB, J_{AB}=8.2 Hz), 7.55 (2H, AB, J_{AB}=8.2 Hz), 6.92 (1H, d, J=8.2 Hz), 4.13-4.09 (1H, m), 4.11 (2H, s), 1.74-1.70 (1H, m), 1.51-1.49 (2H, m), 1.39 (9H, s), 0.91 (3H, d, J=6.6 Hz), 0.89 (3H, d, J=6.5 Hz). ¹³C NMR (DMSO-d₆, 150 MHz): δ 172.1, 164.8, 156.7, 155.2, 137.7, 132.4, 128.7, 127.7, 77.9, 51.3, 44.2, 41.9, 41.6, 40.9. MS (ESI): 757.3 (100, 2M+H), 379.4 (38.8, M+H). HRMS: Calcd for C₁₉H₃₁N₄O₄: 379.2340; Found: 379.2338.

### EXAMPLE 30

### Synthesis of (2R)-2-[(tert-Butoxy)carbonylamino]-4-methyl-N-(4-piperidylcarbonylamino)pentanamide, Trifluoroacetic Acid Salt

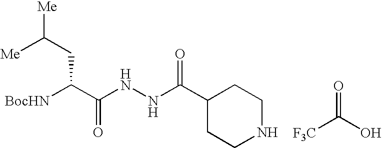

### Part A—Preparation of Fluoren-9-ylmethyl 4-{N-[(tert-butoxy)carbonylamino]-carbamoyl}piperidinecarboxylate

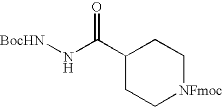

A solution of piperidine-1,4-dicarboxylic acid mono(9H-fluoren-9-ylmethyl) ester (500 mg, 1.42 mmol) and HOAt (179 mg, 1.32 mmol) in dry DMF (10.0 mL) was successively treated with collidine (1.10 mL, 8.32 mmol) and DIC (204 μL, 1.30 mmol) then stirred 5 minutes at 22° C. tert-Butyl carbazate (157 mg, 1.19 mmol) was added in one portion and the resulting solution stirred 16 hours at 22° C. All volatiles were then removed in vacuo, and the crude residue dissolved in ethyl acetate (70 mL) and washed with saturated solutions of NaHCO₃ (6×25 mL) and NaCl (2×25 mL), then dried over MgSO₄, filtered and concentrated in vacuo. Purification by HPLC on a Phenomenex Luna C18 column (21.2×250 mm) using a 1.0%/min gradient of 55-80% acetonitrile containing 0.1% TFA and 10% H₂O at a flow rate of 20 mL/min. The main product peak eluting at 12 minutes was lyophilized to a white solid (541 mg, 1.16 mmol; 97.9%). ¹H NMR (CDCl₃, 600 MHz): δ (2.6:1 mixture of rotamers; data for major rotamer) 7.77 (2H, d, J=7.5 Hz), 7.57 (2H, d, J=7.4 Hz), 7.40 (2H, dd, J=7.4, 7.4 Hz), 7.32 (2H, dd, J=7.6, 7.4 Hz), 4.45 (2H, br s), 4.40 (2H, d, J=6.8 Hz), 4.24 (1H, t, J=6.6 Hz), 2.86 (2H. br s), 2.35 (1H, m), 1.82 (3H, br s), 1.66 (3H, br s), 1.48 (9H, s). ¹³C NMR (CDCl₃, 150 MHz): δ (major rotamer) 174.2, 155.4, 144.2, 141.6, 127.9, 127.3, 125.1, 120.2, 82.6, 67.6, 47.6, 43.4, 41.0, 28.2. MS (ESI): 488.3 (100, M+Na), 301.4 (16.4). HRMS: Calcd for C₂₆H₃₂N₃O₅: 466.2336; Found: 466.2337.

### Part B—Preparation of (2R)-2-[(tert-Butoxy)carbonylamino]-4-methyl-N-(4-piperidylcarbonylamino)pentanamide, Trifluoroacetic Acid Salt

The product of Part A (50.0 mg, 0.107 mmol) was treated with a solution of TFA in CH₂Cl₂ (1:1 v/v, 1.5 mL) at 22° C. After 0.25 hours all volatiles were removed in vacuo, the residue taken up in dry DMF (1.00 mL) then treated with collidine (25.0 μL, 0.189 mmol). The resulting solution was transferred to a previously prepared solution of Boc-D-Leu-OH (37.3 mg, 0.161 mmol), HOAt (18.4 mg, 0.135 mmol), collidine (50.0 μL, 0.378 mmol) and DIC (21.0 μL, 0.134 mmol) in dry DMF (1.00 mL). After 1 hour at 22° C. all volatiles were removed in vacuo, and the crude residue treated with a solution of tris(2-aminoethyl)amine in DMF (1:4 v/v, 4.00 mL); complete deprotection was observed within 0.5 hours. The resulting solution was concentrated to a yellow oil that was purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 18 minutes was lyophilized to a white solid (15.3 mg, 32.5 μmol; 30.3%). ¹H NMR (DMSO-d₆, 600 MHz): δ 9.89 (1H, s), 9.82 (1H, s), 8.39 (2H, br s), 6.88 (1H, d, J=8.3 Hz), 4.02 (1H, ddd, J=9.9, 8.5, 5.1 Hz), 3.31-3.28 (2H, m), 2.91 (2H, brt, J=11.8 Hz), 1.87-1.83 (2H, m), 1.77-1.62 (3H, m), 1.48-1.39 (2H, m), 1.37 (9H, s), 0.88 (3H, d, J=6.6 Hz), 0.85 (3H, d, J=6.6 Hz). MS (ESI): 357.3 (100, M+H), 301.4 (16.4). HRMS: Calcd for C₁₇H₃₃N₄O₄(M+H): 357.2496; Found: 357.2496.

### EXAMPLE 31

### Synthesis of 2-({2-[(2-{4-[N-((2R)-2-Amino-4-methylpentanoylamino)-carbamoyl]piperidyl}-2-oxoethyl){2-[bis(carboxymethyl)amino]ethyl}-amino]ethyl}(carboxymethyl)amino)acetic Acid, Trifluoroacetic Acid Salt

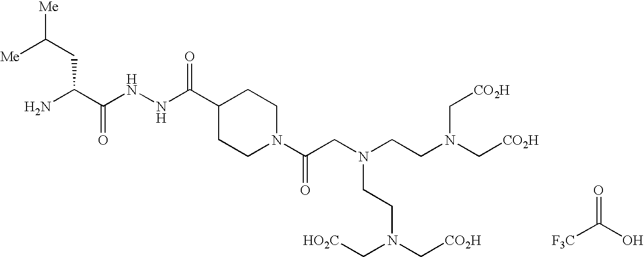

The product of Example 30B (12.0 mg, 25.5 μmol) was added in one portion to a previously prepared solution of 2-{bis[2-(bis {[(tert-butyl)oxycarbonyl]methyl}-amino)ethyl]amino}acetic acid (27.0 mg, 43.7 μmol) in DMF (1.50 mmol) containing HBTU (14.7 mg, 38.8 mmol), HOBt (5.9 mg, 38.5 mmol) and i-Pr₂NEt (29.3 μL, 0.168 mmol). The resulting solution was maintained at 22° C. for 1 hour, then concentrated in vacuo. The residue was then treated with a solution of TFA in CH₂Cl₂ (3:7 v/v, 1.00 mL) for 18 hours then concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 13 minutes was lyophilized to a white solid (10.3 mg, 9.5 μmol; 46.4%). ¹H NMR (DMSO-d₆, 600 MHz): δ 10.46 (1H, br s), 10.13 (1H, d, J=12.7 Hz), 8.20 (3H, br s), 6.49 (1H, s), 4.60 (2H, br s), 4.32 (1H, br d, J=13.5 Hz), 3.77 (1H, t, J=6.7 Hz), 3.67 (1H, br d, J=12.2 Hz), 3.67 (7H, s), 3.09-3.04 (5H, m), 2.77 (1H, brt, J=12.1 Hz), 2.54 (1H, m), 1.79-1.71 (3H, m), 1.66-1.53 (3H, m), 1.51-1.44 (1H, m), 0.92 (3H, d, J=6.5 Hz), 0.90 (3H, d, J=6.5 Hz). MS (ESI): 632.4 (78.3, M+H), 518.9 (48.4), 316.4 (100, M+2H). HRMS: Calcd for C₂₆H₄₆N₇O₁₁: 632.3250; Found: 632.3215. The optical purity of the product was established by chiral GLC analysis; 99.3% D-leucine.

### EXAMPLE 32

### Synthesis of N-((2R)-2-Amino-4-methylpentanoylamino)-6-((2R)-3-fluoro-2-hydroxypropoxy)hexanamide, Trifluoroacetic Acid Salt

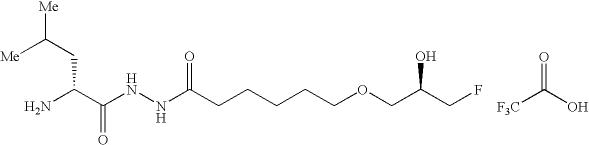

### Part A—Preparation of Methyl 6-{(2R)-2-[(2-Methoxyethoxy)methoxy]-3-[(4-methylphenyl)sulfonyloxy]propoxy}hexanoate

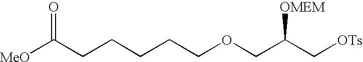

A solution of 6-[2-hydroxy-3-(toluene-4-sulfonyloxy)-propoxy]hexanoic acid methyl ester (1.00 g, 2.67 mmol) (Kazi, A. B.; Hajdu, J. Tetrahedron Lett. 1992, 33, 2291) in dry CH₂Cl₂ (9.00 mL) was treated with i-Pr₂NEt (1.00 mL, 5.74 mmol) and MEM chloride (460 μL, 4.03 mmol); complete protection was noted after 4 hours at 22° C. The reaction mixture was diluted with CH₂Cl₂ (30 mL) then washed with a saturated solution of NaHCO₃ (2×15 mL) followed by H₂O (2×15 mL), then the dried over MgSO₄, filtered and concentrated in vacuo. Purification by chromatography on silica (2:3 ethyl acetate:hexanes containing 0.5% Et₃N; Rf=0.6 in 1:1 ethyl acetate:hexanes containing 0.5% Et₃N) afforded a colorless oil (0.870 g, 1.88 mmol; 70.4%). ¹H NMR (CDCl₃, 600 MHz): δ 7.78 (2H, AB, J=8.3 Hz), 7.34 (2H, AB, J=8.4 Hz), 4.71 (2H, ABq, J_{AB}=7.1 Hz), 4.16 (1H, dd, J=10.3, 4.2 Hz), 4.09 (1H, dd, J=10.4, 5.8 Hz), 3.94-3.91 (1H, m), 3.66-3.65 (2H, m), 3.66 (3H, s), 3.50 (2H, td, J=4.0, 1.6 Hz), 3.45 (2H, ABqd, J_{AB}=10.3 Hz, J_{d}=5.0 Hz), 3.39-3.34 (2H, m), 3.36 (3H, s), 2.44 (3H, s), 2.29 (2H, t, J=7.5 Hz), 1.61 (2H, tt, J=7.7, 7.6 Hz), 1.50 (2H, tt, J=7.6, 6.6 Hz), 1.33-1.28 (2H, m). MS (ESI): 485.3 (67.3, M+Na) 480.3 (100, M+H₂O), 375.3 (23.3).

### Part B—Preparation of 6-{(2R)-2-[(2-Methoxyethoxy)methoxy]-3-[(4-methylphenyl)sulfonyloxy]propoxy}hexanoic Acid

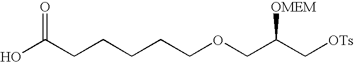

A solution of the product of Part A (250 mg, 0.540 mmol) in THF/H₂O (4:1 v/v 4.00 mL) was treated with LiOH.H₂O (68.0 mg, 1.63 mmol) in one portion and left to stir 18 hours at 22° C. The pH of the reaction mixture was then adjusted to 4-5 with 0.1 N HCl and the resulting biphase diluted with ethyl acetate (50 mL). The layers were separated and the ethyl acetate layer washed with a saturated solution of NaCl (50 mL), dried over MgSO₄, filtered and concentrated in vacuo to a colorless oil (223 mg, 0.497 mmol; 92.0%). This material was used without further purification in the subsequent step. ¹H NMR (DMSO-d₆, 600 MHz): δ 12.0 (1H, br s), 7.78 (2H, AB, J=8.3 Hz), 7.49 (2H, AB, J=8.0 Hz), 4.62 (2H, ABq, J_{AB}=6.9 Hz), 4.09 (1H, dd, J=10.5, 3.5 Hz), 4.01 (1H, dd, J=10.4, 5.5 Hz), 3.82 (1H, qd, J=5.5, 3.5 Hz), 3.53-3.51 (2H, m), 3.40 (2H, t, J=4.5 Hz), 3.38 (1H, dd, J=10.0, 5.4 Hz), 3.33 (1H, dd, J=10.2, 5.8 Hz), 3.28 (2H, t, J=6.5 Hz), 3.22 (3H, s), 2.42 (3H, s), 2.17 (2H, t, J=7.4 Hz), 1.46 (2H, tt, J=7.6, 7.5 Hz), 1.39 (2H, tt, J=7.5, 6.6 Hz), 1.24-1.19 (2H, m). MS (ESI): 471.2 (85.4, M+Na), 466.2 (100, M+H₂O), 171.4 (80.1), 115.4 (68.7).

### Part C—Preparation of (2R)-3-[5-(N-{(2R)-2-[(tert-Butoxy)carbonylamino]-4-methylpentanoylamino}carbamoyl)pentyloxy]-2-[(2-methoxyethoxy)methoxy]propyl 4-Methylbenzenesulfonate

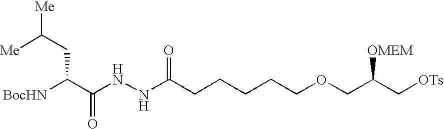

The product of Part B (50.0 mg, 0.111 mmol) and HOAt (15.3 mg, 0.112 mmol) were dissolved in dry DMF (1.00 mL) and treated with collidine (103 μL, 0.779 mmol) followed by DIC (17.5 μL, 0.112 mmol). After stirring 5 minutes at 22° C., Boc-D-Leu-NHNH₂ (27.3 mg, 0.111 mmol), prepared by treatment of Boc-D-Leu-OMe with 4 equiv of hydrazine hydrate in hot ethanol, was added in one portion and the resulting solution stirred overnight. Following 20 hours total reaction time, all volatiles were removed in vacuo and the crude residue purified by HPLC on a Phenomenex Luna C18 column (21.2×250 mm) using a 1.0%/min gradient of 45-75% acetonitrile containing 0.1% TFA and 10% H₂O at a flow rate of 20 mL/min. The main product peak eluting at 15 minutes was lyophilized to a white solid (37.9 mg, 56.1 μmol; 50.3%). ¹H NMR (CDCl₃, 600 MHz): δ 8.66 (1H, br s), 7.92 (1H, br s), 7.79 (2H, AB, J_{AB}=8.3 Hz), 7.34 (2H, AB, J_{AB}=8.0 Hz), 4.70 (2H, ABq, J_{AB}=7.1 Hz), 4.20 (1H, br s), 4.16 (1H, dd, J=10.4, 4.2 Hz), 4.09 (1H, dd, J=10.3, 5.8 Hz), 3.92 (1H, qd, J=5.4, 4.1 Hz), 3.64 (2H, t, J=4.6 Hz), 3.49 (2H, ddd, J=5.8, 3.5, 1.1 Hz), 3.45 (2H, dd, J=5.2, 4.8 Hz), 3.37 (2H, m), 3.36 (3H, s), 2.44 (3H, s), 2.24 (2H, t, J=7.4 Hz), 1.69 (2H, m), 1.66 (2H, tt, J=7.9, 7.5 Hz), 1.52 (2H, m), 1.43 (11H, s), 1.38-1.33 (2H, m). MS (ESI): 698.3 (50.4, M+Na), 576.3 (100, M−Boc), 500.3 (80.1). HRMS: Calcd for C₃₁H₅₄N₃O₁₁S: 676.3474; Found: 676.3494. The optical purity of the product was established by chiral GLC analysis; 99.2% D-leucine.

### Part D—Preparation of Methyl 6-((2R)-3-Fluoro-2-hydroxypropoxy)hexanoate

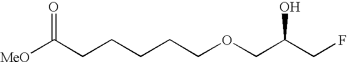

An oven-dried one dram vial was charged with KF (35.0 mg, 0.60 mmol), and Kryptofix-222 [4,7,13,16,21,24-hexaoxa-1,10-diazobicyclo-(8,8,8) hexacosane](226.0 mg, 0.600 mmol) and anhydrous acetonitrile (100 μL). The solution was then concentrated in vacuo; additional acetonitrile (3×100 μL) was used to azeotrope any remaining water. To this residue was then added a solution of 6-[2-hydroxy-3-(toluene-4-sulfonyloxy)-propoxy]hexanoic acid methyl ester³ (75.0 mg; 0.200 mmol) in dry acetonitrile (200 μL). The vial was then sealed with a Teflon screw cap, heated to 140° C. and stirred 1 hour. After cooling to 22° C., the solution was partitioned between ethyl acetate and water (1 mL each), the layers separated and the aqueous layer washed with ethyl acetate (2×1 mL). The combined organic layers were dried over MgSO₄, filtered, concentrated in vacuo and purified by chromatography on silica (R_{f}=0.5 in 1:1 Et₂O/pentane; KMnO₄) to afford a colorless oil (18.3 mg, 82.3 μmol; 41.1%). ¹H NMR (CDCl₃, 600 MHz): δ 4.44 (2H, dABX, J_{d}=47.2 Hz, J_{AB}=9.5 Hz, J_{AX}=4.5 Hz, J_{BX}=5.4 Hz), 4.00 (1H, dtt, J=18.2, 5.8, 4.5 Hz), 3.66 (3H, s), 3.52 (1H, ddd, J=9.7, 4.5, 1.3 Hz), 3.49-3.44 (3H, m), 2.31 (2H, t, J=7.4 Hz), 1.64 (2H, tt, J=7.8, 7.7 Hz), 1.59 (2H, tt, J=7.2, 6.5 Hz), 1.40-1.35 (2H, m). ¹⁹F NMR (CDCl₃, 565 MHz) δ −232.2 (1F, td, J=47.2, 18.1 Hz). MS (ESI): 223.3 (100, M+H).

### Part E—Preparation of 6-((2R)-3-Fluoro-2-hydroxypropoxy)-N-aminohexanamide

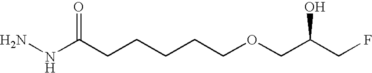

The product of Part D (40.0 mg, 0.180 mmol) was dissolved in absolute ethanol (500 μL), treated with hydrazine hydrate (37.2 μL, 0.719 mmol) then heated to 100° C. and maintained for 18 hours. After cooling to 22° C., the reaction solution was loaded directly onto a silica gel column and eluted with 9:1 CH₂Cl₂/methanol containing 1% Et₃N to afford, after concentration, a white solid (31.3 mg, 0.141 mmol; 78.2%). ¹H NMR (DMSO-d₆, 600 MHz): δ 8.91 (1H, br s), 5.11 (1H, d, J=5.3 Hz), 4.34 (2H, dABX, J_{d}=47.7 Hz, J_{AB}=9.5 Hz, J_{AX}=3.5 Hz, J_{BX}=5.5 Hz), 4.20 (1H, br s), 3.78 (1H, dttd, J=21.5, 5.7, 5.6, 3.5 Hz), 3.38-3.35 (2H, m), 3.33 (2H, dd, J=5.9, 1.5 Hz), 3.31 (2H, br s), 2.00 (2H, t, J=7.4 Hz), 1.51-1.45 (4H, m), 1.28-1.22 (2H, m). MS (ESI): 223.3 (52.0, M+H), 130.3 (100), 122.3 (60.0), 120.2 (67.0).

### Part F—Preparation of N-((2R)-2-Amino-4-methylpentanoylamino)-6-((2R)-3-fluoro-2-hydroxypropoxy)hexanamide, Formic Acid Salt

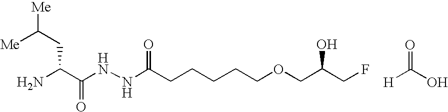

A solution of Boc-D-Leu-OH (31.2 mg, 0.135 mmol) and HOAt (15.3 mg, 0.112 mmol) in dry DMF (1.00 mL) was successively treated with collidine (59.5 μL, 0.450 mmol) and DIC (17.6 μL, 0.112 mmol) then stirred 5 minutes at 22° C. The product of Part E (20.0 mg, 90.0 μmol) was added in one portion and the resulting solution stirred 1 hour at 22° C. All volatiles were then removed in vacuo, and the resulting oil treated with a solution of TFA in CH₂Cl₂ (1:1 v/v, 2.00 mL). The solution was stirred 0.25 hours, then concentrated in vacuo and the crude residue purified by HPLC on a Phenomenex Luna C18 column (21.2×250 mm) using a 1.0%/min gradient of 0-20% acetonitrile containing 0.1% HCO₂H at a flow rate of 20 mL/min. The main product peak eluting at 21 minutes was lyophilized to a white solid (8.3 mg, 22 μmol; 24%). ¹H NMR (DMSO-d₆, 600 MHz): δ 9.99 (1H, s), 5.10 (1H, d, J=5.3 Hz), 4.35 (2H, DABX, J_{d}=47.7 Hz, J_{AB}=9.5 Hz, J_{AX}=3.6 Hz, J_{BX}=5.5 Hz), 3.81-3.75 (1H, m), 3.73 (1H, dd, J=7.5, 6.8 Hz), 3.38 (2H, ABqt, J_{AB}=9.5 Hz, Jₜ=6.5 Hz), 3.34 (2H, dd, J=5.7, 1.6 Hz), 2.15 (2H, t, J=7.3 Hz), 1.76-1.69 (1H, m), 1.61-1.47 (6H, m), 1.33-1.28 (2H, m), 0.92 (3H, d, J=6.5 Hz), 0.90 (3H, d, J=6.5 Hz). ¹⁹F NMR (DMSO-d₆, 565 MHz) δ −230.0 (IF, td, J=47.9, 21.6 Hz). MS (ESI): 336.4 (100, M+H). The optical purity of the product was established by chiral GLC analysis; 99.6% D-leucine.

### EXAMPLE 33

### Synthesis of 2-[5-(N-{(2R)-2-[(tert-Butoxy)carbonylamino]-4-methylpentanoylamino}carbamoyl)pentyloxy]ethyl 4-Methylbenzenesulfonate

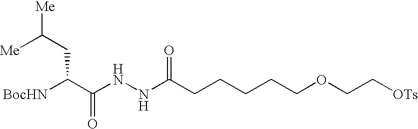

### Part A—Preparation of Methyl 6-(2-Hydroxyethoxy)hexanoate

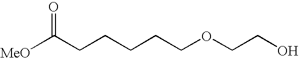

A 100 mL round bottom flask fitted with a dry ice condenser was charged with a solution methyl caproate (2.00 g, 13.7 mmol) (freshly prepared from δ-caprolactone, see: Padwa, A.; Danca, M., D. Org. Lett. 2002, 4, 715) in dry CH₂Cl₂ (30.0 mL). The vessel was cooled to −78° C. and approximately 1.5 equiv ethylene oxide were condensed into the solution. The reaction mixture was warmed to −10° C., then treated with BF₃.OEt₂ (520 μL, 4.10 mmol) and stirred 0.5 hours. The solution was then poured into cold saturated aqueous NaCl and the layers separated. The CH₂Cl₂ layer was washed with saturated NaCl (2×20 mL) then dried over MgSO₄, filtered and concentrated in vacuo. Purification by chromatography on silica (1:1 ethyl acetate:pentane; R_{f}=0.4) followed by distillation under reduced pressure afforded a colorless oil (530 mg, 2.79 mmol; 20.3%). ¹H NMR (CDCl₃, 600 MHz): δ 3.70 (2H, dd, J=4.7, 4.5 Hz), 3.65 (3H, s), 3.51 (2H, dd, J=4.7, 4.5 Hz), 3.46 (2H, t, J=6.5 Hz), 2.30 (2H, t, J=7.5 Hz), 1.64 (2H, tt, J=7.7, 7.5 Hz), 1.59 (2H, tt, J=7.5, 6.6 Hz), 1.40-1.35 (2H, m). ¹³C NMR (CDCl₃, 150 MHz): δ 174.3, 72.0, 71.2, 62.1, 51.7, 34.2, 29.5, 25.9, 24.9. MS (EST): 191.2 (8.5, M+H), 159.2 (100), 141.2 (20.1), 129.2 (58.0), 115.3 (65.9).

### Part B—Preparation of Methyl 6-{2-[(4-Methylphenyl)sulfonyloxy]ethoxy}hexanoate

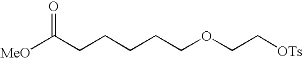

A solution of the product of Part A (150 mg, 0.788 mmol) in dry pyridine (957 μL, 11.8 mmol) was cooled to 0° C. and treated with TsCl (181 mg, 0.949 mmol) in one portion. After stirring 0.5 hours the solution was warmed to 22° C. and stirred 0.25 hours to ensure complete conversion. The reaction mixture was diluted with ethyl acetate (50 mL) and washed with 5% aqueous CuSO₄ (5×10 mL) followed by a saturated solution of NaCl (15 mL). The ethyl acetate layer was dried over MgSO₄, filtered and concentrated in vacuo. Purification by chromatography on silica (1:2 ethyl acetate:pentane; R_{f}=0.5) afforded a colorless oil (171 mg, 0.496 mmol; 63.0%). ¹H NMR (CDCl₃, 600 MHz): δ 7.79 (2H, AB, J_{AB}=8.3 Hz), 7.33 (2H, AB, J_{AB}=8.0 Hz), 4.14 (2H, dd, J=4.9, 4.8 Hz), 3.65 (3H, s), 3.59 (2H, dd, J=4.9, 4.8 Hz), 3.37 (2H, t, J=6.5 Hz), 2.44 (3H, s), 2.29 (2H, t, J=7.5 Hz), 1.60 (2H, tt, J=7.7, 7.6 Hz), 1.50 (2H, tt, J=7.6, 6.6 Hz), 1.34-1.29 (2H, m). MS (ESI): 345.2 (49.7, M+H), 313.2, (100), 199.2 (35.2).

### Part C—Preparation of (6-{2-[(4-Methylphenyl)sulfonyloxy]ethoxy}hexanoic Acid

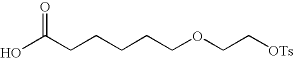

A solution of the product of Part B (150 mg, 0.436 mmol) in THF/H₂O (4:1 v/v 2.00 mL) was treated with LiOH.H₂O (54.8 mg, 1.31 mmol) in one portion and left to stir 18 hours at 22° C. The pH of the reaction mixture was then adjusted to 4-5 with 0.1 N HCl and the resulting biphase diluted with ethyl acetate (100 mL). The layers were separated and the ethyl acetate layer washed with a saturated solution of NaCl (50 mL), then dried over MgSO₄, filtered and concentrated in vacuo to a colorless oil (143 mg, 0.432 mmol; >98%). This material was used directly in the subsequent step. ¹H NMR (CDCl₃, 600 MHz): δ 7.80 (2H, AB, J_{AB}=8.3 Hz), 7.34 (2H, AB, J_{AB}=8.0 Hz), 4.15 (2H, dd, J=4.9, 4.8 Hz), 3.60 (2H, dd, J=4.9, 4.8 Hz), 3.39 (2H, t, J=6.5 Hz), 2.45 (3H, s), 2.35 (2H, t, J=7.5 Hz), 1.63 (2H, tt, J=7.7, 7.5 Hz), 1.53 (2H, tt, J=7.5, 6.6 Hz), 1.38-1.33 (2H, m). MS (ESD: 353.1 (12.9, M+Na), 348.2 (21.2, M+H₂O), 313.2 (100, M−H₂O), 199.2 (67.3), 141.1 (30.6).

### Part D—Preparation of 2-[5-(N-{(2R)-2-[(tert-Butoxy)carbonylamino]-4-methylpentanoylamino}carbamoyl)pentyloxy]ethyl 4-Methylbenzenesulfonate

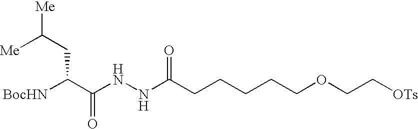

The product of Part C (40.0 mg, 0.121 mmol) and HOAt (16.6 mg, 0.121 mmol) were dissolved in dry DMF (1.00 mL) and treated with collidine (112 μL, 0.848 mmol) followed by DIC (19.0 μL, 0.121 mmol). After stirring 5 minutes at 22° C., Boc-D-Leu-NHNH₂ (29.7 mg, 0.121 mmol) was added in one portion and the resulting solution stirred overnight. Following 20 hours total reaction time, all volatiles were removed in vacuo and the crude residue purified by HPLC on a Phenomenex Luna C18 column (21.2×250 mm) using a 1.0%/min gradient of 45-75% acetonitrile containing 0.1% HCO₂H at a flow rate of 20 mL/min. The main product peak eluting at 16 minutes was lyophilized to a white solid (18.7 mg, 33.5 μmol; 27.7%). ¹H NMR (CDCl₃, 600 MHz): δ 8.68 (1H, br s), 7.90 (1H, br s), 7.81 (2H, AB, J_{AB}=8.2 Hz), 7.35 (2H, AB, J_{AB}=8.3 Hz), 4.85 (1H, br s), 4.22 (1H, br s), 4.16-4.14 (2H, m), 3.62-3.60 (2H, m), 3.41 (2H, t, J=6.4 Hz), 2.46 (3H, s), 2.26 (2H, t, J=7.4 Hz), 1.74-1.69 (1H, m), 1.68 (2H, tt, J=7.9, 7.6 Hz), 1.55-1.52 (4H, m), 1.45 (9H, s), 1.39 (2H, tt, J=7.9, 6.8 Hz), 0.96 (3H, d, J=6.4 Hz), 0.94 (3H, d, J=6.2 Hz). MS (ESI): 580.3 (26.8, M+Na), 502.2 (72.9, M−t−Bu), 458.3 (100, M−Boc). HRMS: Calcd for C₂₆H₄₄N₃O₈S (M+H): 558.2844; Found: 558.2842.

### EXAMPLE 34

### Synthesis of N-[(N-{1-[N-(1-{N-[(1R)-1-(N-{(1S)-3-Methyl-1-[N-(2-methylpropyl)carbamoyl]butyl}carbamoyl)-3-methylbutyl]carbamoyl}(1S)-4-(amidinoamino)butyl)carbamoyl](1S)-3-phenylpropyl}carbamoyl)methyl](2S)-2-[((2S)-1-acetylpyrrolidin-2-yl)carbonylamino]-4-methylpentanamide, Trifluoroacetic Acid Salt

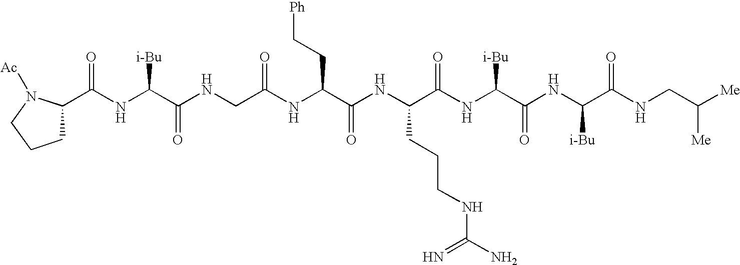

### Part A—Preparation of N-{(1R)-3-Methyl-1-[N-(2-methylpropyl)carbamoyl]butyl}(2S)-2-[(tert-butoxy)carbonylamino]-4-methylpentanamide

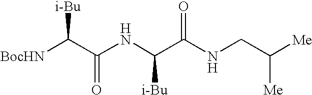

Boc-D-Leu-NHi-Bu (220 mg, 0.768 mmol) (prepared from Boc-D-Leu-OH and i-BuNH₂, see: Okuyama, A.; Naito, K. Leucine derivatives, gelatinase inhibitors, and pharmaceuticals containing them. Japanese Patent 10045699 A2, 1998) was treated with a solution of TFA in CH₂Cl₂ (1:1 v/v, 6.00 mL) at 22° C. After stirring 0.5 hours, all volatiles were removed in vacuo and the residue taken up in dry DMF (1.00 mL) then transferred to a previously prepared solution of Boc-Leu-OH (211 mg, 0.846 mmol), HBTU (306 mg, 0.806 mmol), HOBt (118 mg, 0.770 mmol) and i-Pr₂NEt (535 μL, 3.07 mmol) in dry DMF (3.00 mL). An additional amount of DMF (2×0.5 mL) was used to quantitate the transfer. After 0.75 hours, the reaction mixture was diluted with H₂O (100 mL) then washed with ethyl acetate (3×30 mL). The combined organic layers were washed with 5% aqueous citric acid (2×25 mL) followed by saturated solutions of NaHCO₃ and NaCl (25 mL each) then dried over MgSO₄, filtered and concentrated in vacuo to a white solid (297 mg, 0.743 mmol; 96.5%). This material was used directly in the subsequent step. ¹H NMR (DMSO-d₆, 600 MHz): δ 7.36 (1H, br s), 7.12 (1H, br s), 5.57 (1H, br s), 4.76 (1H, br s), 4.43 (1H, br s), 3.15 (1H, dt, J=6.6, 6.5 Hz), 3.10-3.05 (1H, m), 1.89-1.67 (6H, m), 1.53-1.44 (1H, m), 1.43 (9H, s), 0.95 (6H, br d, J=3.8 Hz), 0.91 (3H, br d, J=5.7 Hz), 0.88 (9H, br d, J=6.6 Hz). ¹³C NMR (DMSO-d₆, 150 MHz) δ 173.6, 172.2, 160.0, 79.4, 53.9, 52.3, 47.1, 41.8, 41.3, 28.9, 28.4, 25.2, 25.1, 23.1, 22.3, 22.2, 20.3. MS (ESI): 422.4 (17.4, M+Na), 400.4 (100, M+H), 344.4 (63.2). HRMS: Calcd for C₂₁H₄₂N₃O₄(M+H): 400.3170; Found: 400.3175.

### Part B—Preparation of Fmoc-R(Pmc)-HMPB BHA Resin

HMPB-BHA resin (10.0 g, substitution level=0.61 mmol/g) was placed in a 200 mL Advanced ChemTech reaction vessel and swollen by washing with DMF (2×45 mL). A solution of Fmoc-Arg(Pmc)-OH (12.1 g, 18.3 mmol) in DMF (45.0 mL) was added to the vessel and the mixture shaken for 0.25 hours. Pyridine (2.22 mL, 27.5 mmol) followed by 2,6-dichlorobenzoyl chloride (2.62 mL, 18.3 mmol) in DMF (45.0 mL) were added and the mixture shaken for 5 hours at 22° C. The resin was washed with DMF, CH₂Cl₂, methanol, CH₂Cl₂ and DMF (3×90 mL each) then treated with DMF (90.0 mL), pyridine (2.47 mL, 30.5 mmol) and benzoyl chloride (2.12 mL, 18.3 mmol) and the vessel shaken for 3 hours. Final washing was then performed with DMF, CH₂Cl₂, methanol and CH₂Cl₂ (3×90 mL each) and the loading (0.44 mmol/g) determined by fulvene-piperidine assay.

### Part C—Preparation of Fmoc-PLG˜Hphe-R(Pmc)-HMPB BHA Resin

The Fmoc-Arg(Pmc)-HMPB BHA resin of Part B (2.00 g, substitution level=0.45 mmol/g) was placed in a 50 ml Advanced ChemTech reaction vessel. The resin was swollen by washing with DMF (2×20 mL), and the following steps were performed: (Step 1) The Fmoc group was removed using 20% piperidine in DMF (20 mL) for 30 minutes. (Step 2) The resin was washed thoroughly (20 mL volumes) with DMF (3×), dichloromethane (3×), methanol (3×), dichloromethane (3×), DMF (3×). (Step 3) Fmoc-Arg(Pmc)-OH (1.44 g, 3.6 mmol), HOBt (0.551 g, 3.6 mmol), HBTU (1.36 g, 3.6 mmol) in 10 mL of DMF and 1.5 mL of DIEA were added to the resin and the reaction was allowed to proceed for 4 hours. (Step 4) The resin was washed thoroughly (20 ml volumes) with DMF (3×), dichloromethane (3×), methanol (3×), dichloromethane (3×), DMF (3×). (Step 5) The coupling reaction was found to be more than 95% complete as assessed by the semi-quantitative ninhydrin assay and quantitative picric assay or fulvene-piperidine assay. Steps 1-5 were repeated until the sequence PLG˜-Hphe-R had been attained.

### Part D—Preparation of Ac-PLG˜Hphe-R(Pmc)-OH

The peptide-resin prepared in Part C was treated with 20% piperidine in DMF (20 mL) for 30 minutes, and washed thoroughly (20 mL volumes) with DMF (3×), dichloromethane (3×), methanol (3×), dichloromethane (3×), DMF (3×). The resin was treated with a solution of acetic anhydride (0.666 mL, 6.6 mmol) and DIEA (1.4 mL, 7.92 mmol) in DMF (20 mL) for 2.0 hours, washed thoroughly (20 mL volumes) with DMF (3×), dichloromethane (3×), methanol (3×), and dichloromethane (3×), and dried under vacuum.

The peptide-resin was placed in a 60 mL fritted glass funnel and washed with dichloromethane (2×40 mL). The peptide-resin was treated with a solution of 5:1:94 trifluoroacetic acid:Et₃SiH:dichloromethane (20 mL) for 2 minutes. The solution was filtered, by the application of pressure, directly into a solution of 10:90 pyridine:methanol (4.0 mL). The cleavage step was repeated eight times. The combined filtrates were concentrated to remove dichloromethane and methanol, providing a colorless oily solid. Trituration with water (40 mL) gave a colorless dry solid, which was collected by filtration. This crude product was purified by HPLC on a Phenomenex Luna C18(2) column (41.4×250 mm) using a 0.9%/minute gradient of 36 to 63% acetonitrile containing 0.1% TFA at a flow rate of 80 mL/min. The main product peak was lyophilized to give the title compound as a colorless solid (280 mg, 34%; HPLC purity 100%). MS: 966.5 (100, M+H), 483.8 (65, M+2H); HRMS: Calcd for C₄₅H₆₇N₈O₁₀S (M+H): 911.4695; Found: 911.4680.

### Part E—Preparation of N-[(N-{1-[N-(1-{N-[(1R)-1-(N-{(1S)-3-Methyl-1-[N-(2-methylpropyl)carbamoyl]butyl}carbamoyl)-3-methylbutyl]carbamoyl}(1S)-4-(amidinoamino)butyl)carbamoyl](1S)-3-phenylpropyl}carbamoyl)methyl](2S)-2-[((2S)-1-acetylpyrrolidin-2-yl)carbonylamino]-4-methylpentanamide, Trifluoroacetic Acid Salt

The product of Part A (15.0 mg, 37.6 μmol) was treated with a solution of TFA in CH₂Cl₂ (1:1 v/v, 2.00 mL) at 22° C. After stirring 0.5 hours, all volatiles were removed in vacuo and the residue taken up in dry DMF (2.00 mL). The solution was successively treated with the product of Part D (34.2 mg, 37.5 μmol), HOBt (5.7 mg, 37 μmol), i-Pr₂NEt (26.1 μL, 0.150 mmol) and HBTU (14.2 mg, 37.4 μmol) then stirred 0.75 hours at 22° C. The resulting solution was concentrated in vacuo and the residue treated with a solution of TFA in CH₂Cl₂ (1:1 v/v, 4.00 mL) at 22° C. After stirring 1.5 hours, all volatiles were removed in vacuo and the residue purified by HPLC on a Phenomenex Luna C18 column (21.2×250 mm) using a 2.0%/min gradient of 10-50% acetonitrile containing 0.1% TFA at a flow rate of 20 mL/min. The main product peak eluting at 23 minutes was lyophilized to a white solid (23.0 mg, 22.1 μmol; 58.9%). ¹H NMR (CDCl₃, 600 MHz): δ 8.80 (1H, br d, J=5.4 Hz), 8.67 (1H, br s), 8.06 (1H, br d, J=5.0 Hz), 7.72 (1H, br s), 7.45 (1H, d, J=5.4 Hz), 7.38 (1H, d, J=7.5 Hz), 7.26 (1H, brt, J=5.8 Hz), 7.15-7.05 (7H, m), 6.85 (2H, br s), 4.27 (1H, ddd, J=11.8, 8.4, 3.8 Hz), 4.18 (1H, ddd, J=9.9, 7.5, 4.6 Hz), 4.13-4.09 (2H, m), 4.01 (1H, dt, J=6.5, 6.1 Hz), 3.93 (1H, dt, J=8.9, 5.6 Hz), 3.74 (1H, dd, J=15.9, 6.7 Hz), 3.62 (1H, dt, J=9.4, 6.2 Hz), 3.57 (1H, dd, J=15.9, 4.4 Hz), 3.35 (1H, dt, J=9.8, 6.8 Hz), 3.23-3.17 (1H, m), 3.00 (1H, ddd, J=16.0, 11.3, 5.8 Hz), 2.93 (1H, dt, J=6.6, 6.6 Hz), 2.72-2.67 (2H, m), 2.59 (1H, ddd, J=16.0, 9.6, 6.6 Hz), 2.11-1.89 (6H, m), 1.99 (3H, s), 1.86-1.79 (2H, m), 1.66-1.53 (12H, m), 0.84 (3H, d, J=6.3 Hz), 0.84 (3H, d, J=6.2 Hz), 0.79 (3H, d, J=6.2 Hz), 0.78 (3H, d, J=6.4 Hz), 0.76 (3H, d, J=6.0 Hz), 0.75 (3H, d, J=6.0 Hz), 0.73 (3H, d, J=6.7 Hz). MS (ESI): 926.7 (100, M+H), 464.0 (47.3, M+2H). HRMS: Calcd for C₄₇H₈₁N₁₁O₈: 463.8129 (M+2H); Found: 463.8131. The optical purity of the product was established by chiral GLC analysis; 68.0% L-leucine.

### EXAMPLE 35

### Synthesis of N-({N-[1-(N-{1-[N-((1R)-1-{N-[1-(N-{(1S)-3-Methyl-1-[N-(2-methylpropyl)carbamoyl]butyl}carbamoyl)(1S)-3-methylbutyl]carbamoyl}-3-methylbutyl)carbamoyl](1S)-4-(amidinoamino)butyl}carbamoyl)(1S)-3-phenylpropyl]carbamoyl}methyl)(2S)-2-[((2S)-1-acetylpyrrolidin-2-yl)carbonylamino]-N-(4-aminobutyl)-4-methylpentanamide, Trifluoroacetic Acid Salt

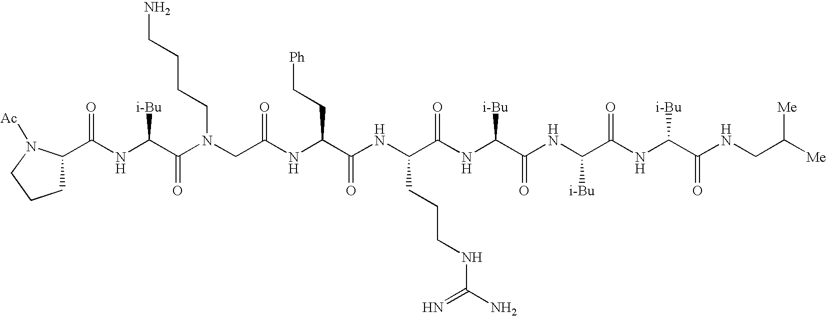

### Part A—Preparation of Ac-PL-NLys(Boc)˜Hphe-R(Pmc)-L-OH

The MacroKan reaction vessel containing the Fmoc-PL-NLys(Boc)˜Hphe-R(Pmc)-L-HMPB-BHA resin (prepared according to the procedure found in Example 14B; 0.318 g, substitution level=0.44 mmol/g), was placed in DMF (10.0 mL) and stirred 3 minutes. The DMF was decanted and the resin treated with a solution of piperidine in DMF (1:4 v/v, 25.0 mL) for 1.5 hours at 22° C. The solution was decanted, and the resin washed with CH₂Cl₂ (9×10 mL) and DMF (3×10 mL) then treated with DMF (20.0 mL), i-Pr₂NEt (123 μL, 0.706 mmol) and Ac₂O (66.0 μL, 0.700 mmol). After 15 hours at 22° C., the solution was decanted and the resin washed with DMF (3×10 mL) and CH₂Cl₂ (9×10 mL) then dried under reduced pressure. The resin was removed from the MacroKan and placed in a scintered glass funnel of medium porosity. The resin was washed with a solution of TFA in CH₂Cl₂ (1:99 v/v, 9×10 mL) and the filtrate collected, concentrated in vacuo and purified by HPLC on a Phenomenex Luna C18 column (21.2×250 mm) using a 1.8%/min gradient of 50-95% acetonitrile containing 0.1% TFA and 10% H₂O at a flow rate of 20 mL/min. The main product peak eluting at 17 minutes was lyophilized to a pale yellow powder (80.0 mg, 66.9 μmol; 47.8%). MS (ESI): 1195.7 (100, M+H), 548.4 (33.4, M+2H−Boc). HRMS: Calcd for C₆₀H₅₇N₁₀O₁₃S: 1195.6795; Found: 1195.6799.

### Part B—Preparation of N-({N-[1-(N-{1-[N-((1R)-1-{N-[1-(N-{(1S)-3-Methyl-1-[N-(2-methylpropyl)carbamoyl]butyl}carbamoyl)(1S)-3-methylbutyl]carbamoyl}-3-methylbutyl)carbamoyl](1S)-4-(amidinoamino)butyl}carbamoyl)(1S)-3-phenylpropyl]carbamoyl}methyl)(2S)-2-[((2S)-1-acetylpyrrolidin-2-yl)carbonylamino]-N-(4-aminobutyl)-4-methylpentanamide, Trifluoroacetic Acid Salt

The product of Example 34A (15.0 mg, 37.6 μmol) was treated with a solution of TFA in CH₂Cl₂ (1:1 v/v, 2.00 mL) at 22° C. After stirring 0.5 hours, all volatiles were removed in vacuo and the residue taken up in dry DMF (3.00 mL). The solution was successively treated with the product of Example 35A (44.8 mg, 37.5 μmol), HOBt (5.7 mg, 37 μmol), i-Pr₂NEt (26.1 μL, 0.150 mmol) and HBTU (14.2 mg, 37.4 μmol) then stirred 0.75 hours at 22° C. The resulting solution was concentrated in vacuo and the residue treated with a solution of TFA in CH₂Cl₂ (1:1 v/v, 3.00 mL) at 22° C. After stirring 2.5 hours, all volatiles were removed in vacuo and the residue purified by HPLC on a Phenomenex Luna C18 column (21.2×250 mm) using a 2.0%/min gradient of 30-70% acetonitrile containing 0.1% TFA at a flow rate of 20 mL/min. The main product peak eluting at 10 minutes was lyophilized to a white solid (38.0 mg, 28.4 μmol; 75.6%). ¹H NMR (CDCl₃, 600 MHz): δ 8.16-8.00 (3H, m), 7.74-7.60 (3H, m), 7.31-7.02 (9H, m), 4.34 (1H, dt, J=9.6, 5.7 Hz), 4.31-4.28 (1H, m), 4.25 (1H, ddd, J=10.1, 7.8, 4.8 Hz), 4.22-4.09 (3H, m), 4.08-4.03 (1H, m), 4.00 (1H, dt, J=10.4, 5.8 Hz), 3.51-3.23 (4H, m), 3.13-3.00 (2H, m), 2.90-2.49 (12H, m), 2.01-1.85 (3H, m), 1.89 (3H, s), 1.81-1.41 (17H, m), 1.30-1.14 (3H, m), 0.83-0.71 (30H, m). MS (ESI): 1110.8 (40, M+H), 556.0 (100, M+2H). HRMS: Calcd for C₅₇H₁₀₁N₁₃O₉(M+2H): 555.8917; Found: 555.8918. The optical purity of the product was established by chiral GLC analysis; 75.1% L-leucine.

### EXAMPLE 36

### Synthesis of N-[(2R)-2-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-5-[(imino{[(2,2,5,7,8-pentamethylchroman-6-yl)sulfonyl]amino}methyl)amino]pentanoylamino-}4-methylpentanoylamino)-4-methylpentanoylamino]-6-aminohexanamide, Trifluoroacetic Acid Salt

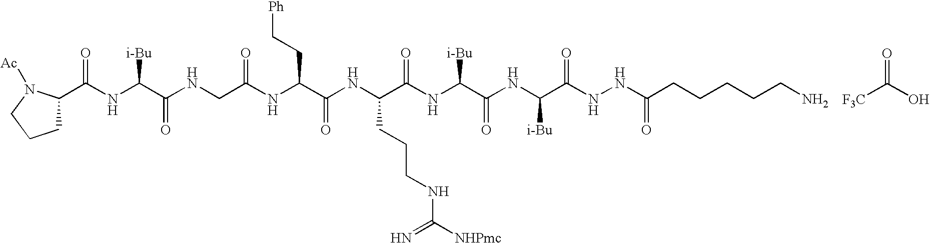

### Part A—Preparation of N-((2R)-2-Amino-4-methylpentanoylamino)-6-[(fluoren-9-ylmethoxy)carbonylamino]hexanamide, Trifluoroacetic Acid Salt

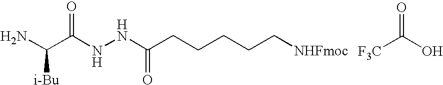

A solution of Boc-D-Leu-OH (77.8 mg, 0.312 mmol) and HOAt (35.6 mg, 0.262 mmol) in dry DMF (3.00 mL) was successively treated with collidine (193 μL, 1.46 mmol) and DIC (40.2 μL, 0.257 mmol) then stirred 5 minutes at 22° C. The product of Example 3A (100 mg, 0.208 mmol) was added in one portion and the resulting solution stirred 2 hours at 22° C. All volatiles were then removed in vacuo, and the resulting oil treated with a solution of TFA in CH₂Cl₂ (1:1 v/v, 6.00 mL). The solution was maintained for 0.5 hours, then concentrated in vacuo and the crude residue purified by HPLC on a Phenomenex Luna C18 column (21.2×250 mm) using a 1.4%/min gradient of 20-55% acetonitrile containing 0.1% TFA at a flow rate of 20 mL/min. The main product peak eluting at 17 minutes was lyophilized to a white solid (37.0 mg, 62.2 μmol; 30.0%). The material was used directly in the subsequent step.

### Part B—Preparation of N-[2-((2R)-2-Amino-4-methylpentanoylamino)(2S)-4-methylpentanoylamino]-6-[(fluoren-9-ylmethoxy)carbonylamino]hexanamide, Trifluoroacetic Acid Salt

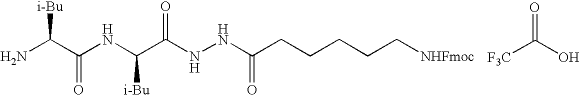

The product of Part A (35.0 mg, 58.9 μmol) was dissolved in dry DMF (3.00 mL) and transferred to a previously prepared solution of Boc-Leu-OH (22.0 mg, 88.2 μmol), HOBt (11.3 mg, 73.8 μmol), i-Pr₂NEt (41.0 μL, 0.235 mmol) and HBTU (27.9 mg, 73.6 μmol) in DMF (2.00 mL) then stirred 1 hour at 22° C. The resulting solution was concentrated in vacuo and the residue treated with a solution of TFA in CH₂Cl₂ (1:1 v/v, 6.00 mL) at 22° C. After stirring 0.5 hours, all volatiles were removed in vacuo and the residue purified by HPLC on a Phenomenex Luna C18 column (21.2×250 mm) using a 1.5%/min gradient of 30-60% acetonitrile containing 0.1% TFA and 10% H₂O at a flow rate of 20 mL/min. The main product peak eluting at 17 minutes was lyophilized to a white solid (34.0 mg, 48.0 μmol; 81.6%). ¹H NMR (DMSO-d₆, 600 MHz): δ 9.98 (1H, s), 9.73 (1H, s), 8.80 (1H, d, J=8.6 Hz), 8.09 (3H, br s), 7.89 (2H, d, J=7.5 Hz), 7.68 (2H, d, J=7.4 Hz), 7.41 (2H, t, J=7.4 Hz), 7.33 (2H, t, J=7.3 Hz), 7.24 (1H, t, J=5.5 Hz), 4.48 (1H, td, J=8.6, 5.7Hz), 4.29 (2H, d, J=6.9 Hz), 4.20 (1H, t, J=6.7 Hz), 3.81 (1H, br s), 2.96 (2H, td, J=6.4, 6.3 Hz), 2.11 (2H, t, J=7.3 Hz), 1.64-1.48 (8H, m), 1.39 (2H, tt, J=7.4, 6.8 Hz), 1.26 (2H, tt, J=8.0, 6.9 Hz), 0.90 (3H, d, J=6.4 Hz), 0.90 (3H, d, J=6.6 Hz), 0.90 (3H, d, J=6.4 Hz), 0.85 (3H, d, J=6.5 Hz). MS (ESI): 594.4 (100, M+H). HRMS: Calcd for C₃₃H₄₈N₅O₅ (M+H): 594.3650; Found: 594.3646.

### Part C—Preparation of N-[(2R)-2-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-5-[(iminoethyl)amino]pentanoylamino}-4-methylpentanoylamino)-4-methylpentanoylamino]-6-aminohexanamide, Trifluoroacetic Acid Salt

A solution of the product of Part B (21.0 mg, 29.7 μmol) was successively treated with the product of Example 34D (27.1 mg, 29.7 μmol), HOBt (4.6 mg, 0.030 mmol), i-Pr₂NEt (26.0 μL, 0.149 mmol) and HBTU (11.3 mg, 29.8 μmol) then stirred 2 hours at 22° C. The resulting solution was concentrated in vacuo and the residue treated with a previously prepared solution of piperidine in DMF (1:4 v/v, 10.0 mL) at 22° C. After stirring 0.5 hours, all volatiles were removed in vacuo and the residue purified by HPLC on a Phenomenex Luna C18 column (21.2×250 mm) using a 1.5%/min gradient of 30-60% acetonitrile containing 0.1% TFA and 10% H₂O at a flow rate of 20 mL/min. The main product peak eluting at 21 minutes was lyophilized to a white solid (30.0 mg, 21.8 μmol; 73.3%). ¹H NMR (DMSO-d₆, 600 MHz): δ 9.83 (1H, br s), 9.72 (1H, br s), 8.22 (0.5H, d, J=8.0 Hz), 8.18 (0.5H, t, J=5.7 Hz), 8.11 (0.5H, d, J=8.2 Hz), 8.07-8.01 (3H, m), 7.97 (0.5H, d, J=8.0 Hz), 7.89-7.83 (1H, m), 7.80 (0.5H, d, J=8.3 Hz), 7.25 (2H, t, J=7.4 Hz), 7.17-7.15 (3H, m), 4.38-4.16 (7H, m), 3.79 (0.5H, dd, J=16.5, 5.5 Hz), 3.72-3.69 (1.5H, m), 3.49-3.29 (2H, m), 3.07-2.99 (2H, m), 2.79-2.74 (2H, m), 2.61-2.54 (3H, m), 2.48 (6H, br s), 2.10 (2H, t, J=7.4 Hz), 2.03 (3H, s), 1.92-1.79 (5H, m), 1.77 (2H, t, J=6.9 Hz), 1.71-1.37 (19H, m), 1.31 (2H, tt, J=8.0, 7.4 Hz), 1.26 (6H, s), 0.87 (3H, d, J=6.5 Hz), 0.86 (3H, d, J=6.5 Hz), 0.81 (3H, d, J=6.6 Hz), 0.81 (3H, d, J=6.6 Hz), 0.79 (3H, d, J=6.6 Hz), 0.75 (3H, d, J=6.5 Hz). MS (ESI): 1264.7 (36.3, M+H), 633.3 (100, M+2H). HRMS: Calcd for C₆₃H₁₀₂N₁₃O₁₂S: 632.8779; Found: 632.8786.

### EXAMPLE 37

### N-[(N-{1-[N-(1-{N-[1-(N-{(1R)-3-Methyl-1-[N-(4-piperidylcarbonylamino)-carbamoyl]butyl}carbamoyl)(1S)-3-methylbutyl]carbamoyl}(1S)-4-[(imino{[(2,2,5,7,8-pentamethylchroman-6-yl)sulfonyl]amino}methyl)amino]butyl)carbamoyl]-(1S)-3-phenylpropyl}carbamoyl)methyl](2S)-2-[((2S)-1-acetylpyrrolidin-2-yl)carbonylamino]-4-methylpentanamide, Trifluoroacetic Acid Salt

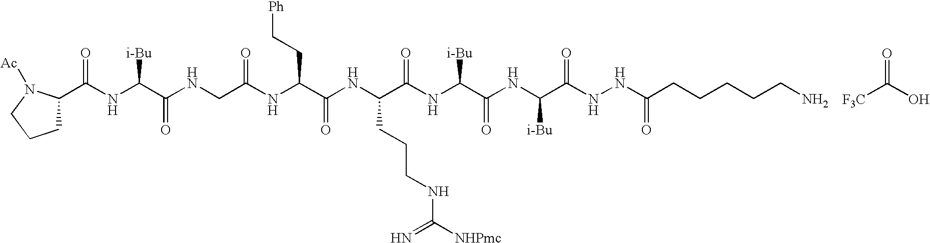

### Part A—Preparation of Fluoren-9-ylmethyl 4-{N-[(2R)-2-((2S)-2-Amino-4-methylpentanoylamino)-4-methylpentanoylamino]carbamoyl}piperidinecarboxylate, Trifluoroacetic Acid Salt

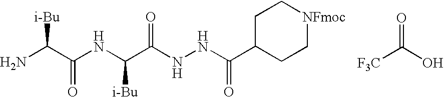

The product of Example 30B (70.0 mg, 0.149 mmol) was added in one portion to a previously prepared solution of Fmoc-OSu (55.3 mg, 0.164 mmol) and i-Pr₂NEt (78.0 μL, 0.448 mmol) in dry DMF (3.00 mL) at 22° C. After stirring 1 hour the solution was partitioned between ethyl acetate and H₂O (30 mL each), the layers separated and the aqueous layer washed with ethyl acetate (15 mL). The combined organic layers were washed with 5% aqueous citric acid (2×15 mL) followed by saturated solutions of NaHCO₃ and NaCl (15 mL each). The resulting solution was dried over MgSO₄, filtered and concentrated in vacuo and the residue treated with a solution of TFA in CH₂Cl₂ (1:1 v/v, 4.00 mL) at 22° C. After stirring 0.5 hours, all volatiles were removed in vacuo and the residue purified by HPLC on a Phenomenex Luna C18 column (21.2×250 mm) using a 1.5%/min gradient of 30-60% acetonitrile containing 0.1% TFA and 10% H₂O at a flow rate of 20 mL/min. The main product peak eluting at 12 minutes was lyophilized to a white solid (73.0 mg, 0.105 mmol; 70.8%). The entire mass was dissolved in dry DMF (3.00 mL) and transferred to a previously prepared solution of Boc-Leu-OH (37.6 mg, 0.151 mmol), HOBt (19.3 mg, 0.126 mmol), i-Pr₂NEt (70.0 μL, 0.402 mmol) and HBTU (47.8 mg, 0.126 mmol) in DMF (2.00 mL) then stirred 1 hour at 22° C. The resulting solution was concentrated in vacuo and the residue treated with a solution of TFA in CH₂Cl₂ (1:1 v/v, 6.00 mL) at 22° C. After stirring 0.5 hours, all volatiles were removed in vacuo and the residue purified by HPLC on a Phenomenex Luna C18 column (21.2×250 mm) using a 1.5%/min gradient of 30-60% acetonitrile containing 0.1% TFA and 10% H₂O at a flow rate of 20 mL/min. The main product peak eluting at 15 minutes was lyophilized to a white solid (58.0 mg, 82.2 μmol; 81.3%). ¹H NMR (DMSO-d₆, 600 MHz): δ 10.00 (1H, s), 9.81 (1H, s), 8.80 (1H, d, J=8.5 Hz), 8.11 (3H, br s), 7.89 (2H, d, J=7.5 Hz), 7.62 (2H, d, J=7.5 Hz), 7.42 (2H, t, J=7.4 Hz), 7.33 (2H, t, J=7.5 Hz), 4.47 (1H, td, J=8.7, 5.9 Hz), 4.36 (2H, br d, J=6.1 Hz), 4.27 (1H, t, J=6.5 Hz), 4.00-3.79 (3H, m), 2.82 (2H, br s), 2.41 (1H, tt, J=11.2, 3.8 Hz), 1.95 (3H, s), 1.65-1.50 (8H, m), 1.42-1.37 (2H, m), 0.91 (3H, d, J=6.5 Hz), 0.90 (3H, d, J=6.4 Hz), 0.85 (3H, d, J=6.5 Hz). ¹³C NMR (DMSO-d₆, 150 MHz): δ 172.8, 170.2, 168.8, 157.8 (q, J=151 Hz), 154.3, 143.9, 140.8, 127.6, 127.1, 124.9, 120.1, 116.9 (q, J=299 Hz), 66.5, 50.9, 49.5, 46.7,42.8, 41.4, 40.3, 27.9, 24.1, 23.6, 23.0, 22.6, 21.8, 21.2. MS (ESI): 592.3 (100, M+H). HRMS: Calcd for C₃₃H₄₆N₅O₅ (M+H): 592.3493; Found: 592.3479.

### Part B—Preparation of N-[(N-{1-[N-(1-{N-[1-(N-{(1R)-3-Methyl-1-[N-(4-piperidylcarbonylamino)carbamoyl]butyl}carbamoyl)(1S)-3-methylbutyl]-carbamoyl}-(1S)-4-[(imino{[(2,2,5,7,8-pentamethylchroman-6-yl)sulfonyl]amino}-methyl)-amino]butyl)carbamoyl](1S)-3-phenylpropyl}-carbamoyl)-methyl](2S)-2-[((2S)-1-acetylpyrrolidin-2-yl)carbonylamino]-4-methylpentanamide, Trifluoroacetic Acid Salt

A solution of the product of Part A (21.0 mg, 29.7 μmol) was successively treated with the product of Example 34D (27.1 mg, 29.7 μmol), HOBt (4.6 mg, 0.030 mmol), i-Pr₂NEt (26.0 μL, 0.149 mmol) and HBTU (11.3 mg, 29.8 μmol) then stirred 2 hours at 22° C. The resulting solution was concentrated in vacuo and the residue treated with a previously prepared solution of piperidine in DMF (1:4 v/v, 10.0 mL) at 22° C. After stirring 0.5 hours, all volatiles were removed in vacuo and the residue purified by HPLC on a Phenomenex Luna C18 column (21.2×250 mm) using a 1.5%/min gradient of 30-60% acetonitrile containing 0.1% TFA and 10% H₂O at a flow rate of 20 mL/min. The main product peak eluting at 21 minutes was lyophilized to a white solid (30.0 mg, 21.8 μmol; 73.3%). ¹H NMR (DMSO-d₆, 600 MHz): δ 9.88 (1H, s), 9.87 (1H, s), 8.56 (1H, br d, J=10.0 Hz), 8.29-8.24 (1H, m), 8.22 (0.5H, d, J=8.0 Hz), 8.18 (0.5H, t, J=5.6 Hz), 8.11 (0.5H, d, J=8.1 Hz), 8.08-8.01 (3H, m), 7.97 (0.5H, t, J=8.0 Hz)7.87-7.83 (1H, m), 7.80 (0.5H, d, J=8.2 Hz), 7.25 (2H, dd, J=7.5, 7.4 Hz), 7.17-7.13 (3H, m), 4.38-4.16 (8H, m), 3.79 (0.5H, dd, J=16.4, 5.4 Hz), 3.72-3.69 (2H, m), 3.51-3.28 (5H, m), 3.07-2.99 (2H, m), 2.94-2.89 (2H, m), 2.59-2.53 (4H, m), 2.47 (6H, br s), 2.03 (3H, br s), 1.95 (3H, s), 1.92-1.38 (30H, m), 1.26 (6H, s), 0.87 (3H, d, J=6.2 Hz), 0.86 (3H, d, J=6.2 Hz), 0.81 (3H, d, J=6.5 Hz), 0.81 (3H, d, J=6.6 Hz), 0.79 (3H, d, J=6.7 Hz), 0.75 (3H, d, J=6.6 Hz). MS (ESI): 1262.7 (23.9, M+H), 632.0 (100, M+2H). HRMS: Calcd for C₆₃H₁₀₁N₁₃O₁₂S (M+2H): 631.8701; Found: 631.8705.

### EXAMPLE 38

### Synthesis of 2-[(2-{[(N-{5-[N-((2R)-2-Amino-3-phenylpropanoylamino)-carbamoyl]pentyl}carbamoyl)methyl]{2-[bis(carboxymethyl)amino]ethyl}-amino}ethyl)(carboxymethyl)amino]acetic Acid, Trifluoroacetic Acid Salt

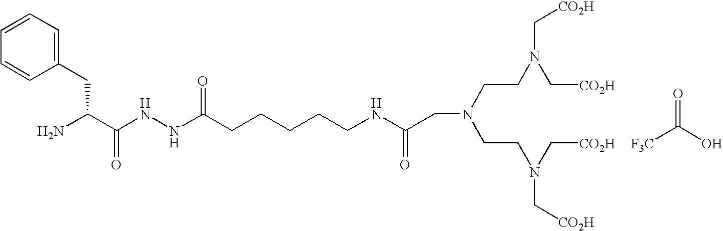

### Part A—Preparation of N-{(2R)-2-[(tert-Butoxy)carbonylamino]-3-phenylpropanoylamino}-6-[(fluoren-9-ylmethoxy)carbonylamino]hexanamide

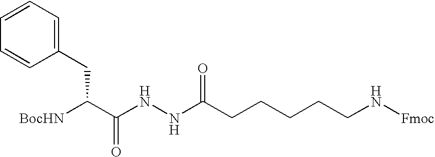

A solution of Boc-D-Phe-OH (242 mg, 0.90 mmol), HBTU (342 mg, 0.90 mmol), HOBt (140 mg, 0.90 mmol), and DIEA (525 μL, 3.0 mmol) in DMF (5.0 mL) was stir at room temperature under nitrogen for 20 minutes. The product of Example 3A (371 mg, 0.75 mmol) was added to the reaction in a single portion, followed by DIEA (525 μL, 3 mmol) (pH=10). The solution was stirred at ambient temperatures for 2.5 hours and added dropwise to water (500 mL). The resulting precipitate was collected by filtration, washed with water, and dried to give an off-white solid, (360 mg, 65%). MS (ESI): 515.3 (100, M+H−Boc), 637.3 (10, M+Na).

### Part B—Preparation of 2-[(2-{[(N-{5-[N-((2R)-2-Amino-3-phenylpropanoylamino)-carbamoyl]pentyl}carbamoyl)methyl]{2-[bis(carboxymethyl)amino]ethyl}-amino}ethyl)(carboxymethyl)amino]acetic acid, Trifluoroacetic Acid Salt

The product of Part A (307 mg, 0.5 mmol) was dissolved in 20:80 Piperdine:DMF (5.0 mL) and stirred under nitrogen at room temperature for 30 minutes. The solution was concentrated under reduced pressure, redissolved in DMF (1.0 mL), and added to a previously prepared solution of 2-{bis[2-(bis {[(tert-butyl)oxycarbonyl]methyl}-amino)ethyl]amino}acetic acid (370 mg, 0.50 mmol), HBTU (228 mg, 0.60 mmol), HOBt (92 mg, 0.60 mmol), and DIEA (0.21 mL, 2.4 mmol) in DMF (4 mL). The solution was stirred under nitrogen at ambient temperatures for 30 minutes, concentrated, and the resulting residue was taken up in ethyl acetate (50 mL). The solution was washed consecutively with 10% citric acid (2×50 mL), 0.1 N NaOH (2×50 mL), and water (50 mL). The organic layer was dried (MgSO4), filtered, and concentrated to produce a yellow oil. MS (ESI): 992.7 (100, M+H).

The above oil was dissolved in 90:9:1 TFA:dichloromethane:TIS (5.0 mL) and stirred at room temperature under nitrogen for 3 hours. The solution was concentrated and the resulting oily solid was purified by HPLC on a Phenomenex Luna C18(2) column (41.4×250 mm) using a 0.9%/min gradient of 0 to 27% acetonitrile containing 0.1% TFA at a flow rate of 80 mL/min. The main product peak eluting at 22.6 minutes was lyophilized to give 40.2 mg (6% overall) of the title compound as a colorless solid. MS (ESI): 668.4 (60, M+H), 461.2 (100, M+H-Leu). HRMS: Calcd for C₂₉H₄₃FeN₇O₁₁ (M−2H+Fe): 721.23645; Found: 721.2374; Chiral analysis: 98.2% D-Phe.

### EXAMPLE 39

### Synthesis of (2R)—N—{[N-(4-Aminobutyl)carbamoyl]methyl}-2-[(tert-butoxy)carbonylamino]-4-methylpentanamide, Trifluoroacetic Acid Salt

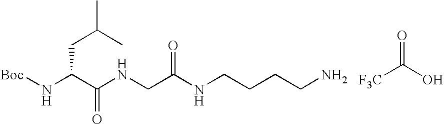

### Part A—Preparation of 2-Amino-N-{4-[(fluoren-9-ylmethoxy)carbonylamino]-butyl}acetamide, Trifluoroacetic Acid Salt

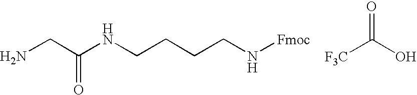

A solution of Boc-Gly-OH (175 mg, 1.0 mmol), HBTU (454 mg, 1.2 mmol), HOBt (183 mg, 1.2 mmol), and DIEA (0.63 mL, 3.6 mmol) in DMF (2.5 mL) was stir at room temperature under nitrogen for 15 minutes. N-(4-Aminobutyl)(fluoren-9-ylmethoxy)carboxamide (346 mg, 1.0 mmol) was added and the solution was stirred at ambient temperatures under nitrogen 18 hours. The solution was concentrated and the residue was taken up in ethyl acetate (50 mL). The solution was washed consecutively with 10% citric acid (2×50 mL), saturated NaHCO₃ (2×50 mL), and water (50 mL). The ethyl acetate layer was dried (MgSO₄), filtered, and concentrated to produce a white powder (475 mg, 95%). MS (ESD: 468.3 (100, M+H).

The product of Part A was dissolved in 1:1 TFA:dichloromethane(5 mL) and stirred at room temperature under nitrogen for 10 minutes. The solution was concentrated to give the title compound as a yellow/brown oil (373 mg, 100%). MS (ESI): 368.3 (100, M+H), 735.3 (10, 2M+H).

### Part B—Preparation of (2R)—N—{[N-(4-Aminobutyl)carbamoyl]methyl}-2-[(tert-butoxy)carbonylamino]-4-methylpentanamide, Trifluoroacetic Acid Salt

A solution of Boc-D-Leu-OH (299 mg, 1.2 mmol), HBTU (909 mg, 2.4 mmol), HOBt (367 mg, 2.4 mmol), and DIEA (418 μL, 2.4 mmol) in DMF (5 mL) was stir at room temperature under nitrogen for 20 minutes and treated with the product of Part A (364 mg, 1.0 mmol) in one portion. The solution was stirred for 30 minutes and concentrated. The resulting residue was dissolved in ethyl acetate (50 mL) and washed consecutively with 10% citric acid (2×50 mL), saturated NaHCO₃ (2×50 mL), and water (50 mL). The organic phase was dried (MgSO₄), filtered, and concentrated to give 725 mg of a colorless solid. A 100 mg portion of the solid was dissolved in 1:1 DMF:TAEA (2.0 mL) and stirred at room temperature under nitrogen for 30 minutes. The DMF was removed under reduced pressure and the oily solid was dissolved in water (0.5 mL). The solution was then purified by HPLC on a Phenomenex Luna C18(2) column (41.4×250 mm) using a 0.9%/min gradient of 9 to 36% acetonitrile containing 0.1% TFA at a flow rate of 80 mL/min. The main product peak centered at 21.5 minutes. was lyophilized to give the title compound as a colorless solid (40 mg, 61%, HPLC purity 100%) ¹H NMR (CD₃CN): δ 7.55(s, 1H), 7.35 (s, 3H), 7.13 (s, 1H), 5.87 (s, 1H), 3.96 (q, J=7.2 Hz, 1H), 3.79 (dd, J₁=6.6 Hz, J₂=16.8 Hz, 1H), 3.69 (dd, J₁=6.6 Hz, J₂=16.8 Hz, 1H), 3.30-3.21 (m, 1H), 3.19-3.11 (m, 1H), 3.00-2.91 (m, 2H), 1.70-1.62 (m, 3H), 1.59-1.48 (m, 4H), 1.42 (s, 9H), 0.96-0.88 (m, 6H). MS (ESI): 359.3 (60, M+H). HRMS: Calcd for C₁₇H₃₅N₄O₄ (M+H): 359.2653; Found: 359.2650; Chiral analysis: 95.9% D-Leu.

### EXAMPLE 40

### Synthesis of N-{(2R)-2-[(tert-Butoxy)carbonylamino]-4,N-dimethylpentanoylamino}-6-aminohexanamide, Trifluoroacetic Acid Salt

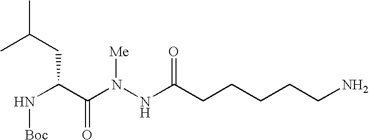

### Part A—Preparation of N-{(2R)-2-[(tert-Butoxy)carbonylamino]-4,N-dimethylpentanoylamino}-6-[(fluoren-9-ylmethoxy)carbonylamino]hexanamide

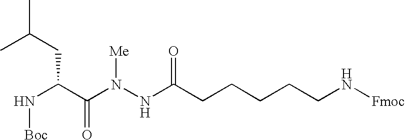

A solution of the product of Example 9A (194.5 mg, 0.403 mmol) in 50:50 TFA:dichloromethane (1.5 mL) was stirred at room temperature for 0.5 hours and concentrated under vacuum. The resulting viscous oil was dissolved in DMF (2.0 mL) and treated with Boc-D-Leu-OH (100 mg, 0.403 mmol), HBTU (184 mg, 0.484 mmol), and DIEA (0.141 mL, 0.806 mmol). This solution was stirred at room temperature under nitrogen for 3 hours, and the volatiles were removed under vacuum. The resulting residue was dissolved in ethyl acetate (20 mL) and washed consecutively with 10% citric acid (20 mL), saturated NaHCO₃ (20 mL), water (20 mL), and saturated NaCl (20 mL), dried (MgSO₄), filtered, and concentrated. The crude product was purified by flash chromatography on silica gel eluting with 1:2 pentane:ethyl acetate to give the title compound as a viscous colorless oil (118 mg, 49%, HPLC purity 100%). ¹H NMR (CDCl₃): δ 8.78 (bs, 1H), 7.77 (d, J=7.8 Hz, 2H), 7.60 (d, J=7.2 Hz, 2H), 7.41 (t, J=7.5 Hz, 2H), 7.23 (t, J=7.5 Hz, 2H), 4.95 (d, J=7.6 Hz, 1H), 4.86 (bs, 1H), 4.60-4.54 (m, 1H), 4.41 (d, J=7.2 Hz, 2H), 4.26-4.19 (m, 1H). MS (ESI): 495.3 (70, M−Boc+H), 617.4 (100, M+Na).

### Part B—Preparation of N-{(2R)-2-[(tert-Butoxy)carbonylamino]-4,N-dimethylpentanoylamino}-6-aminohexanamide, Trifluoroacetic Acid Salt

A solution of the product of Part A (117.7 mg, 0.198 mmol) in TAEA (0.35 mL, 2.341 mmol) and DMF (1.0 mL) was stirred at room temperature under nitrogen for 20 minutes and concentrated under reduced vacuum. The resulting crude product was purified by HPLC on a Phenomenex Luna C18(2) column (21.2×250 mm) using a 0.9%/min gradient of 18 to 45% acetonitrile containing 0.1% TFA at a flow rate of 20 mL/min. The main product peak eluting at 17.7 minutes was lyophilized to give the title compound as a colorless solid (53.6 mg, 73%, HPLC purity 100%). ¹H NMR (1:1 CD₃CN:D₂O): δ 4.51-4.30 (m, 1H), 2.99 (s, 3H), 2.87 (t, J=7.5 Hz, 2H), 2.39-2.27 (m, 2H), 1.68-1.10 (m, 18H), 0.94-0.70 (m, 6H). MS (ESI): 373.4 (100, M+H); HRMS: Calcd for C₁₈H₃₇N₄O₄ (M+H): 373.2809; Found: 373.2815.

### EXAMPLE 41

### Synthesis of 2-{[2-({[N-({4-[N-((2R)-2-Amino-4-methylpentanoylamino)-carbamoyl]phenyl}methyl)carbamoyl]methyl}{2-[bis(carboxymethyl)amino]-ethyl}amino)ethyl](carboxymethyl)amino}acetic Acid, Trifluoroacetic Acid Salt

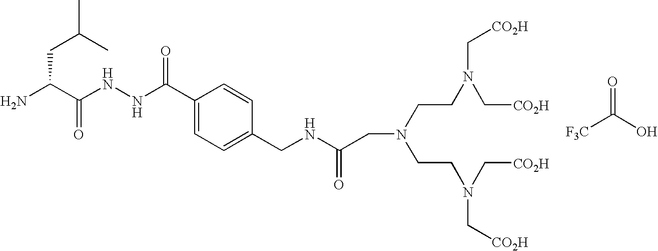

### Part A—Preparation of tert-Butyl 2-[(2-{[(N-{[4-(N-{(2R)-2-[(tert-Butoxy)-carbonylamino]-4-methylpentanoylamino}carbamoyl)phenyl]methyl}carbamoyl)-methyl][2-(bis{[(tert-butyl)oxycarbonyl]methyl}amino)ethyl]amino}ethyl){[(tert-butyl)oxycarbonyl]methyl}amino]acetate

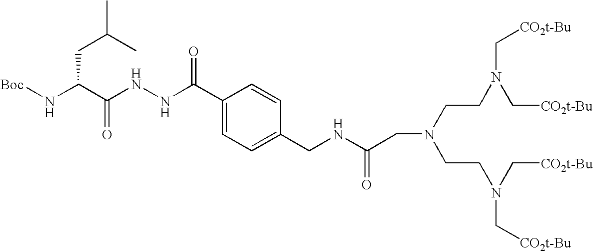

A solution of 2-{bis[2-(bis{[(tert-butyl)oxycarbonyl]methyl}amino)-ethyl]amino}acetic acid (230 mg, 0.37 mmol), HBTU (156 mg, 0.40 mmol), and DIEA (140 μL, 0.80 mmol) in DMF (1 mL) was stirred for 10 minutes at room temperature under nitrogen, and treated with the product of Example 29 (126 mg, 0.33 mmol) and DIEA (70 μL, 0.33 mmol) (pH=10). The solution was stirred 30 minutes. and concentrated under reduced pressure. Then resulting crude product was purified by HPLC on a Phenomenex Luna C18(2) column (41.4×250 mm) using a 0.9%/min gradient of 45 to 72% acetonitrile containing 0.1% TFA at a flow rate of 80 mL/min. The main product peak centered at 21 minutes. was lyophilized to give the title compound as a colorless solid (130 mg, 40%) ¹H NMR (DMSO-d₆): δ 10.32 (s, 1H), 9.92 (s, 1H), 9.01 (t, J=6.0 Hz, 1H), 7.85 (d, J=8.1 Hz, 2H), 7.40 (d, J=8.1 Hz, 2H), 6.89 (d, J=8.4 Hz, 1H), 4.43 (d, J=6.0 Hz, 2H), 4.28 (s, 2H), 4.15-4.08 (m, 1H), 3.51-3.32 (m, 12H), 3.09-3.00 (m, 4H), 1.77-1.68 (m, 1H), 1.56-1.30 (m, 38H), 0.95-0.86 (m, 6H). MS (ESI): 978.7 (100, M+H).

### Part B—Preparation of 2-{[2-({[N-({4-[N-((2R)-2-Amino-4-methylpentanoylamino)-carbamoyl]phenyl}methyl)carbamoyl]methyl}{2-[bis(carboxymethyl)amino]-ethyl}amino)ethyl](carboxymethyl)amino}acetic acid, Trifluoroacetic Acid Salt

A solution of the product of Part A in 92:8 TFA:TIS (8.0 mL) was stirred at room temperature under nitrogen for 2 hours and concentrated under reduced pressure. The residue was purified by HPLC on a Phenomenex Luna C18(2) column (41.4×250 mm) using a method consisting of isocratic conditions for 10 minutes at 1.8% acetonitrile containing 0.1% TFA and a flow rate of 80 mL/min, followed by a 0.9%/min gradient of 1.8 to 28.8% acetonitrile containing 0.1% TFA at a flow rate of 80 mL/min. The main product peak was lyophilized to give the title compound as a colorless solid (75 mg, 88%, HPLC purity 100%). MS (ESI): 654.3 (100, M+H), 327.6 (75, M+2H). HRMS: Calcd for C₂₈H₄₁FeN₇O₁₁ (M−2H+Fe): 707.2208; Found: 707.2202; Chiral analysis: 99.8% D-Leu.

### EXAMPLE 42

### Synthesis of 2-({2-[({N-[(4-{[N-((2R)-2-Amino-4-methylpentanoylamino)-carbamoyl]methyl}phenyl)methyl]carbamoyl}methyl){2-[bis(carboxymethyl)amino]ethyl}amino]ethyl}(carboxymethyl)amino)acetic acid, Trifluoroacetic Acid Salt

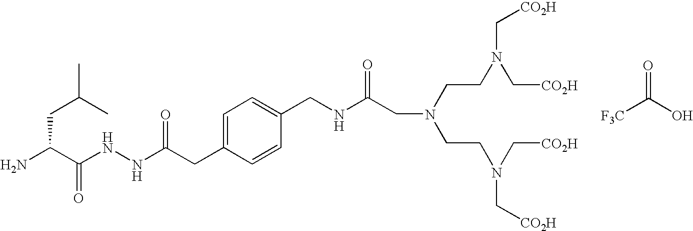

### Part A—Preparation of tert-Butyl 2-{[2-({[N-({4-[(N-{(2R)-2-[(tert-Butoxy)-carbonylamino]-4-methylpentanoylamino}carbamoyl)methyl]phenyl}methyl)-carbamoyl]methyl}[2-(bis{[(tert-butyl)oxycarbonyl]methyl}amino)ethyl]-amino)ethyl]{[(tert-butyl)oxycarbonyl]methyl}amino}acetate

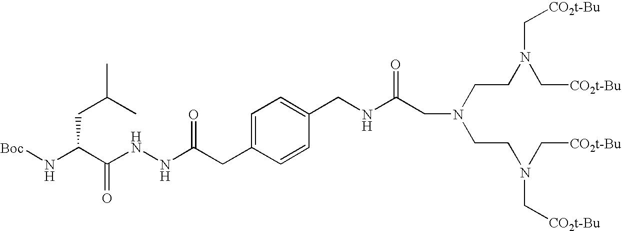

A solution of 2-{bis[2-(bis{[(tert-butyl)oxycarbonyl]methyl}-amino)-ethyl]amino}acetic acid (53mg, 92 μmol), HBTU (31 mg, 83 μmol), and DIEA (26 μL, 152 μmol) in DMF (1.0 mL) was stirred at room temperature under nitrogen 15 minutes. The product of Example 4 (30 mg, 76 μmol) was added to the solution, stirring was continued for 30 minutes, and the volatiles were removed under reduced pressure. The crude product was purified by HPLC on a Phenomenex Luna C18(2) column (41.4×250 mm) using a 0.9%/min gradient of 45% to 72% acetonitrile containing 0.1% TFA at a flow rate of 80 mL/min. The main product peak eluting at 24.6 minutes was lyophilized to give the title compound as a colorless solid (33 mg, 43%, HPLC purity 100%). MS (ESI): 992.7 (100, M+H)

### Part B—Preparation of 2-({2-[({N-[(4-{[N-((2R)-2-Amino-4-methylpentanoylamino)carbamoyl]methyl}phenyl)methyl]carbamoyl}methyl){2-[bis(carboxymethyl)amino]ethyl}amino]ethyl}(carboxymethyl)amino)acetic Acid, Trifluoroacetic Acid Salt

A solution of the product of Part B in 92:8 TFA:TIS (2.0 mL) was stirred at room temperature under nitrogen for 2.5 hours and concentrated under reduced pressure. The crude product was purified by HPLC on a Phenomenex Luna C18(2) column (41.4×250 mm) using a 0.9%/min gradient of 1.8 to 28.8% acetonitrile containing 0.1% TFA at a flow rate of 80 mL/min. The main product peak eluting at 14.8 minutes was lyophilized to give the title compound as a colorless solid (19 mg, 95%, HPLC purity 100%). ¹HNMR(1:1 Pyridine-d₅:DMSO-d₆): δ 10.90(s, 1H), 9.00 (s, 1H), 7.54 (bs, 8H), 7.45-7.33 (m, 4H), 4.50 (d, J=5.4 Hz, 2H), 4.20 (s, 1H), 3.80-3.62 (m, 12H), 3.16-2.97 (m, 8H), 2.00-1.76 (m, 3H), 0.96-0.83 (m, 6H). MS (ESI): 668.7 (60, M+H), 334.4 (100, M+2H). HRMS: Calcd for C₂₉H₄₆N₇O₁₁ (M+H): 668.324982; Found: 668.3253; Chiral analysis: 99.0% D-Leu.

### EXAMPLE 43

### Synthesis of 2-[10-({N-[(4-{[N-((2R)-2-Amino-4-methylpentanoylamino)-carbamoyl]methyl}phenyl)methyl]carbamoyl}methyl)-1,4,7,10-tetraaza-4,7-bis(carboxymethyl)cyclododecyl]acetic Acid, Trifluoroacetic Acid Salt

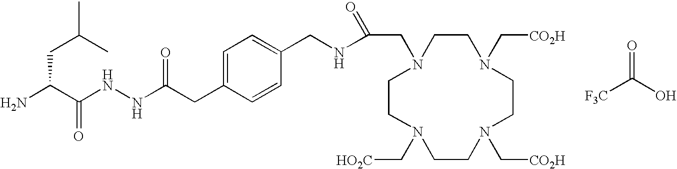

### Part A—Preparation of tert-Butyl 2-(7-{[N-({4-[(N-{(2R)-2-[(tert-Butoxy)-carbonylamino]-4-methylpentanoylamino}carbamoyl)methyl]phenyl}-methyl)-carbamoyl]methyl}-1,4,7,10-tetraaza-4,10-bis{[(tert-butyl)oxycarbonyl]methyl}-cyclododecyl)acetate

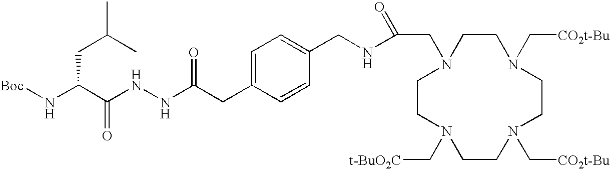

A solution of DOTA(Ot-Bu)₃—OH (66 mg, 115 μmol), HBTU (37 mg, 100 μmol), and DIEA (60 μL, 342 μmol) in DMF (2.0 mL) was stirred at room temperature under nitrogen for 15 minutes. The product of Example 4 (30 mg, 76 μmol) was added in a single portion and the solution was stirred for an additional 15 minutes. The volatiles were removed under reduced pressure and the crude material was purified by HPLC on a Phenomenex Luna C18(2) column (41.4×250 mm) using a 0.9%/min gradient of 27 to 54% acetonitrile containing 0.1% TFA at a flow rate of 80 mL/min. The main product peak eluting at approximately 20.8 minutes was lyophilized to give the title compound as a colorless solid (44 mg, 62%, HPLC purity 100%). MS (ESI): 947.7 (95, M+H), 446.4( 100, M+2H); Chiral analysis: 98.7% D-Leu.

### Part B—Preparation of 2-[10-({N-[(4-{[N-((2R)-2-Amino-4-methylpentanoylamino)-carbamoyl]-methyl}phenyl)methyl]carbamoyl}methyl)-1,4,7,10-tetraaza-4,7-bis(carboxymethyl)cyclododecyl]acetic Acid, Trifluoroacetic Acid Salt

A solution of the product of Part A in 92:8 TFA:TIS (2.0 mL) was stirred at room temperature under nitrogen for 3 hours and concentrated under vacuum. The crude product was purified by HPLC on a Phenomenex Luna C18(2) column (41.4×250 mm) using a 0.9%/min gradient of 1.8 to 28.8% acetonitrile containing 0.1% TFA at a flow rate of 80 mL/min. The product fraction eluting at 14.8 minutes was lyophilized to give the title compound as a colorless solid (19 mg, 95%, HPLC purity 100%). ¹H NMR (1:1 Pyridine-d₅:DMSO-d₆): δ 10.89 (s, 1H), 8.98 (s, 1H), 7.42-7.34 (m, 4H), 5.73 (bs, 7H), 4.50-4.42 (m, 2H), 4.22-4.16 (m, 1H), 3.84-3.72 (m, 6H), 3.72-3.61 (m, 2H), 3.59-3.50 (m, 2H), 3.25-2.80 (m, 16H), 2.00-1.76 (m, 3H), 0.95-0.86 (m, 6H). MS (ESI): 679.5 (70, M+H), 340.4 (100, M+2H). HRMS: Calcd for C₃₁H₅₁N₈O₉ (M+H): 679.3774; Found: 679.378; Chiral analysis: 99.0% D-Leu.

### EXAMPLE 44

### Synthesis of 3-({N-[(4-{[N-((2R)-2-Amino-4-methylpentanoylamino)-carbamoyl]methyl}phenyl)methyl]carbamoyl}methyl)-7-amino-4-methyl-2-oxo-2H-chromene-6-sulfonic Acid, Trifluoroacetic Acid Salt

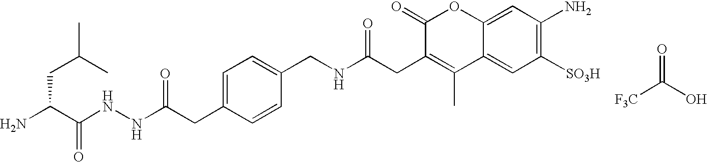

### Part A—Preparation of 3-{[N-({4-[(N-{(2R)-2-[(tert-Butoxy)carbonylamino]-4-methylpentanoylamino}carbamoyl)methyl]phenyl}methyl)carbamoyl]methyl}-7-amino-4-methyl-2-oxo-2H-chromene-6-sulfonic Acid

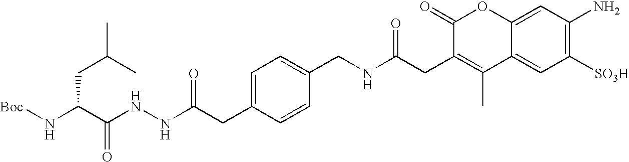

The product of Example 4 (4.1 mg, 0.0081 mmol) was dissolved in DMF (2.0 mL) and treated with sufficient DIEA (10 μL) to give a solution of pH=10. The solution was treated with Alexa Fluor 350™ succinimidyl ester (5.1 mg, 0.010 mmol) and stirred at ambient temperature under nitrogen for 30 minutes. Concentration gave a pale yellow solid, which was purified by HPLC on a Phenomenex Luna C18(2) column (21.2×250 mm) using a 0.9%/min gradient of 18 to 45% acetonitrile containing 0.1% TFA at a flow rate of 20 mL/min. The product fraction was lyophilized to give the title compound as a colorless solid (3.1 mg, 56%, HPLC purity 100%). MS (ESI): 1275.3 (5, 2M−Boc+H), 710.2 (15, M+Na), 588.2 (100, M−Boc+H).

### Part B—Preparation of 3-({N-[(4-{[N-((2R)-2-Amino-4-methylpentanoylamino)-carbamoyl]methyl}phenyl)methyl]carbamoyl}methyl)-7-amino-4-methyl-2-oxo-2H-chromene-6-sulfonic Acid, Trifluoroacetic Acid Salt

The product of Part A was dissolved in 50:50 TFA:dichloromethane (3.0 mL) and allowed to stand under nitrogen at ambient temperature for 10 minutes. The volatiles were removed and the resulting oil was lyophilized from 50:50 acetonitrile:H₂O (6.0 mL) to give the title compound as a colorless solid (3.1 mg, 98%, HPLC purity 99%). ¹H NMR (1:1 CD₃CN: D₂O): δ 7.96 (s, 1H), 7.21 (a portion of AA′BB′ system, J=7.9 Hz, 2H), 7.17 (b portion of AA′BB′ system, J=7.9 Hz, 2H), 6.62 (s, 1H), 4.26 (s, 2H), 3.62 (bs, 1H), 3.53 (s, 4H), 2.30 (s, 3H), 1.65-1.45 (m, 3H), 0.90-0.82 (m, 6H). MS (ESI): 1175.3 (11, 2M+H), 588.1 (100, M+H); HRMS: Calcd for C₂₇H₃₄N₅O₈S (M+H): 588.2128; Found: 588.2125.

### EXAMPLE 45

### Synthesis of N—[(N-{1-[N-(1-{N-[4-({N-[(1R)-1-(N-Aminocarbamoyl)-3-methylbutyl]carbamoyloxy}methyl)phenyl]carbamoyl}(1S)-3-methylbutyl)carbamoyl](1S)-3-phenylpropyl}carbamoyl)methyl](2S)-2-[((2S)-1-acetylpyrrolidin-2-yl)carbonylamino]-N-{4-[(tert-butoxy)carbonylamino]butyl}-4-methylpentanamide, Trifluoroacetic Acid Salt

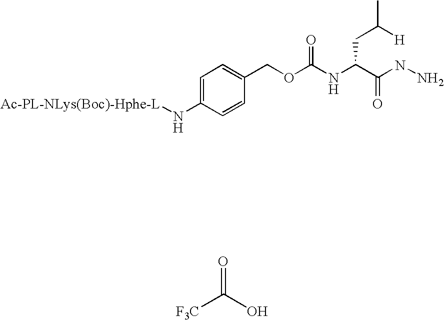

### Part A—Preparation of Fmoc-Hphe-HMPB BHA Resin

HMPB-BHA resin (5.00 g, substitution level=0.80 mmol/g) was placed in a 200 mL Advanced ChemTech reaction vessel and swollen by washing with DMF (3×50 mL). A solution of Fmoc-Hphe-OH (4.85 g, 12.1 mmol) in DMF (50.0 mL) was added to the vessel and the mixture shaken for 0.25 hours. Pyridine (1.62 mL, 20.0 mmol) followed by 2,6-dichlorobenzoyl chloride (1.72 mL, 12.0 mmol) in DMF (50.0 mL) were added and the mixture shaken for 8 hours at 22° C. The resin was washed with DMF, CH₂Cl₂, methanol, CH₂Cl₂ and DMF (3×90 mL each) then treated with DMF (50.0 mL), pyridine (1.62 mL, 20.0 mmol) and benzoyl chloride (1.40 mL, 12.1 mmol) and the vessel shaken for 3 hours. Final washing was then performed with DMF, CH₂Cl₂, methanol and CH₂Cl₂ (3×50 mL each) and the loading (0.60 mmol/g) determined by fulvene-piperidine assay.

### Part B—Preparation of Fmoc-PL-NLys(Boc)˜Hphe-HMPB-BHA Resin

The Fmoc-Hphe-HMPB BHA resin of Part A (2.00 g, substitution level=0.60 mmol/g) was placed in a 100 ml Advanced ChemTech reaction vessel. The resin was swollen by washing with DMF (2×30 mL), and the following steps were performed: (Step 1) The Fmoc group was removed upon exposure to a solution of piperidine in DMF (1:4 v/v, 30 mL) for 0.5 hours. (Step 2) The resin washed with DMF, CH₂Cl₂, methanol, CH₂Cl₂ and DMF (3×30 mL each). (Step 3) A solution of Fmoc-NLys(Boc)-OH (2.25 g, 4.80 mmol), HOBt (735 mg, 4.80 mmol), HBTU (1.82 g, 4.80 mmol) and i-Pr₂NEt (2.09 mL, 12.0 mmol) in DMF (30.0 mL) was added to the resin and the reaction vessel shaken 4 hours. (Step 4) The resin washed with DMF, CH₂Cl₂, methanol, CH₂Cl₂ and DMF (3×30 mL each). (Step 5) The coupling reaction was found to be more than 95% complete as assessed by the fulvene-piperidine assay. Steps 1-5 were repeated with Fmoc-Leu-OH and Fmoc-Pro-OH respectively, to complete the sequence PL-NLys(Boc)-Hphe.

### Part C—Preparation of Ac-PL-NLys(Boc)˜Hphe-OH

The peptide-resin of Part A (2.12 g) was placed in a 100 mL Advanced ChemTech reaction vessel and swollen by washing with DMF (2×30 mL). The resin was treated with 20% piperidine in DMF (30 mL) for 30 minutes to remove the Fmoc protecting group, followed by washing (30 ml volumes) with DMF (3×), dichloromethane (3×), methanol (3×), dichloromethane (3×), and DMF (3×). Acetic anhydride (0.40 mL, 4.2 mmol), DIEA (0.74 mL, 4.2 mmol), and DMF (30 mL) were added and the mixture was gently agitated for 2 hours. The peptide-resin was washed (30 mL volumes) with DMF (3×), dichloromethane (3×), methanol (3×), and dichloromethane (3×), and dried under vacuum. The peptide-resin was placed in a sintered glass funnel and treated with 1% TFA in dichloromethane (12 mL) for 2 minutes. The solution was filtered, by application of nitrogen pressure, directly into a flask containing 1:9 pyridine:methanol (2.0 mL). The cleavage procedure was repeated ten (10) times. The combined filtrates were concentrated to give a colorless oily solid. This crude product was triturated with water (2×25 mL) and dried under reduced pressure to give a dry solid. This solid was purified by HPLC on a Phenomenex Luna C18(2) column (41.4×250 mm) using a 0.9%/min gradient of 27 to 54% acetonitrile containing 0.1% TFA at a flow rate of 80 mL/min. The main product peak eluting at 22.6 minutes was lyophilized to give 239.1 mg (43%) of the title compound as a colorless solid with 100% purity by HPLC. ¹H NMR (1:1 CD₃CN:D₂O): δ 8.54 (d, J=7.2 Hz, 3H), 8.36 (br, 3H), 8.22 (br, 3H), 8.14 (t, J=7.2 Hz, 2H), 8.06 (t, 6H), 5.72 (br, 3H), 5.08 (s, 1H), 4.97 (s, 1H), 4.85 (s, 2H), 4.74 (t, J=7.2 Hz, 2H), 2.53-2.31 (m, 6H), 2.24 (t, 3H), 1.66 (t, J=5.4 Hz, 9H), 1.60 (d, J=6.0 Hz, 3H), 1.57 (d, J=6.0 Hz, 3H), 1.58 (m, 3H); ¹³C NMR (1:1 CD₃CN+D₂O): δ 173.7, 168.2, 161.8, 161.6, 157.0, 143.9, 141.2, 137.1, 133.6,128.8, 128.1, 127.5, 125.4, 120.8, 120.3, 118.7, 117.9, 115.9, 67.6, 66.4, 52.7, 52.3, 46.8, 40.6, 40.1, 24.5, 24.2, 22.4, 22.1, 21.3, 20.9, 0.97, 0.83, 0.69, 0.56, 0.42, 0.28, 0.14. MS (ESI): 660.5 (30, M+H), 560.4 (100, M+H−Boc); HRMS: Calcd for C₃₄H₅₄N₅O₈ (M+H): 660.3967; Found: 660.3964.

### Part D—Preparation of (2R)-2-[(tert-Butoxy)carbonylamino]-N-[(fluoren-9-ylmethoxy)carbonylamino]-4-methylpentanamide

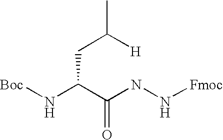

A solution of Boc-D-Leu-OH (150 mg, 0.649 mmol), 9-fluorenylmethyl carbazate (164.9 mg, 0.649 mmol), HOAt (88.3 mg, 0.649 mmol), and collidine (428.5 μL, 3.243 mmol) in DMF (1 mL) was treated with DIC (200.8 μL, 1.297 mmol), and stirred at room temperature under nitrogen for 3 hours. The reaction mixture was diluted with ethyl acetate (25 mL), washed consecutively with 10% citric acid (3×25 mL), 10% NaHCO₃ (3×25 mL), water (25 mL), and saturated NaCl (25 mL), dried ( MgSO₄), filtered, and concentrated under reduced pressure to give the title compound as a colorless solid. (302 mg, 100%). ¹H NMR (CDCl₃): δ 8.21 (s, 1H), 7.74 (d, J=7.8 Hz, 2H), 7.57 (d, J=7.8 Hz, 2H), 7.38 (t, J=7.5 Hz, 2H), 7.29 (t, J=7.5 Hz, 2H), 6.79 (s, 1H), 4.85 (s, 1H), 4.43 (d, J=7.2 Hz, 2H), 4.18 (s, 1H), 3.96 (s, 1H), 1.72 (m, 2H), 1.43 (s, 9H), 1.25 (m, 1H), 0.94 (d, J=6.6 Hz, 3H), 0.92 (d, J=6.6 Hz, 3H). MS: m/e 368.3 [M+H−Boc](35%), 490.2 [M+Na] (100%).

### Part E—Preparation of N-(4-{[N-((1R)-1-{N-[(Fluoren-9-ylmethoxy)-carbonylamino]carbamoyl}-3-methylbutyl)carbamoyloxy]methyl}phenyl)(2S)-2-amino-4-methylpentanamide, Trifluoroacetic Acid Salt

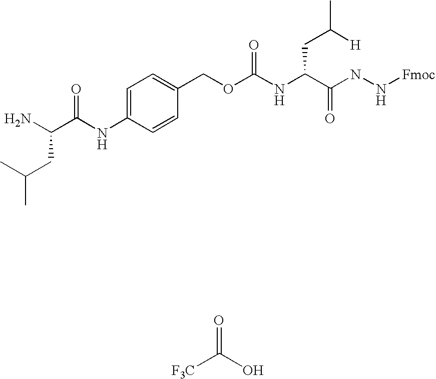

The product of Part C (65.0 mg, 0.139 mmol) was dissolved in 50:50 TFA:dichloromethane (1.0 mL) and stirred at room temperature under nitrogen for 30 minutes and concentrated under reduced pressure. The resulting residue was dissolved in DMF (0.5 mL) along with DIEA (60 μL, 0.344 mmol), HOBt (21.3 mg, 0.139 mmol), and the product of Example 13B (69.7 mg, 0.139 mmol). The reaction was stirred at room temperature under nitrogen for 18 hours, and the solvent was removed under reduced pressure. The resulting residue was dissolved in 50:50 TFA:dichloromethane (1.0 mL), stirred at room temperature under nitrogen for 20 minutes, and concentrated under vacuum. The resulting residue was purified by HPLC on a Phenomenex Luna C18(2) column (21.2×250 mm) using a 0.9%/min gradient of 18 to 45% acetonitrile containing 0.1% TFA at a flow rate of 20 mL/min. The main product peak eluting at 19.5 minutes was lyophilized to give the title compound as a colorless solid (56.1 mg, 64%, HPLC purity 100%). MS (ESI): 1259.4 (35, 2M+H), 630.3 (100, M+H).

### Part F—Preparation of N—[(N-{1-[N-(1-{N-[4-({N-[(1R)-1-(N-Aminocarbamoyl)-3-methylbutyl]carbamoyloxy}methyl)phenyl]carbamoyl}(1S)-3-methylbutyl)-carbamoyl](1S)-3-phenylpropyl}carbamoyl)methyl](2S)-2-[((2S)-1-acetylpyrrolidin-2-yl)carbonylamino]-N-{4-[(tert-butoxy)carbonylamino]butyl}-4-methylpentanamide, Trifluoroacetic Acid Salt

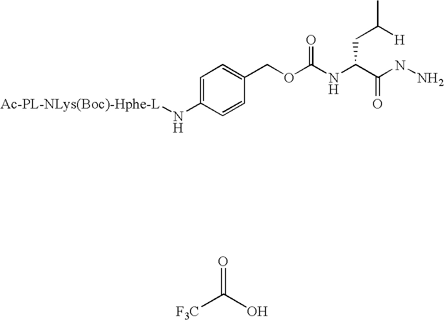

The product of Part D (19.1 mg, 0.030 mmol), the product of Part B (20.0 mg, 0.030 mmol), and HOAt (4.1 mg, 0.030 mmol) were dissolved in DMF (0.6 mL) and treated with collidine (20.0 μL, 0.152 mmol) and DIC (4.5 μL, 0.061 mmol). The solution was stirred at room temperature under nitrogen for 18 hours. TAEA (0.125 mL, 0.8 mmol) was added and the reaction solution was stirred an additional 20 minutes. The volatiles were removed under reduced pressure, and the resulting residue was purified by HPLC on a Phenomenex Luna C 18(2) column (21.2×250 mm) using a 0.9%/min gradient of 18 to 54% acetonitrile containing 0.1% TFA at a flow rate of 20 mL/min. The main product peak eluting at 33.4 minutes was lyophilized to give the title compound as a colorless solid (12.1 mg, 38%, HPLC purity 100%). ¹H NMR (1:1 CD₃CN+D₂O): δ 8.25-8.21 (m, 2H), 8.05-7.97 (m, 4H), 7.94-7.87 (m, 3H), 5.84-5.63 (m, 2H), 5.19-4.89 (m, 3H), 4.80-4.59 (m, 2H), 4.28-3.97 (m, 4H), 3.78-3.66 (m, 2H), 3.41-3.27 (m, 2H), 2.88-2.03 (m, 33H), 1.68-1.46 (m, 18H). MS (ESI): 1049.7 (100, M+H); HRMS: Calcd for C₅₄H₈₅N₁₀O₁₁ (M+H): 1049.6394; Found: 1049.6366. Chiral analysis: 66.6% L-Leu, 99.4% L-Hphe.

### EXAMPLE 46

### Synthesis of N—[(N-{1-[N-(1-{N-[4-({N-[(1R)-1-(N-Aminocarbamoyl)-3-methylbutyl]carbamoyloxy}methyl)phenyl]carbamoyl}(1S)-4-[(imino{[(2,2,5,7,8-pentamethylchroman-6-yl)sulfonyl]amino}methyl)amino]butyl)carbamoyl](1S)-3-phenylpropyl}carbamoyl)methyl](2S)-2-[((2S)-1-acetylpyrrolidin-2-yl)carbonylamino]-4-methylpentanamide, 2,2,2-Trifluoroacetic Acid Salt

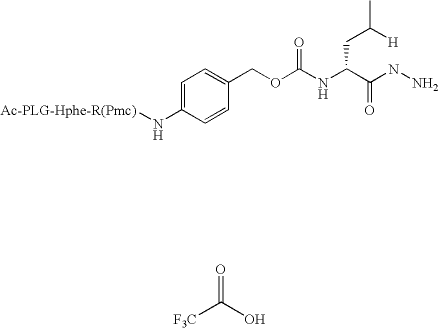

### Part A—Preparation of Fmoc-PLG˜Hphe-HMPB-BHA Resin

The Fmoc-Hphe-HMPB BHA resin of Example 45A (2.00 g, substitution level=0.60 mmol/g) was placed in a 100 ml Advanced ChemTech reaction vessel. The resin was swollen by washing with DMF (2×30 mL), and the following steps were performed: (Step 1) The Fmoc group was removed upon exposure to a solution of piperidine in DMF (1:4 v/v, 30 mL) for 0.5 hours. (Step 2) The resin washed with DMF, CH₂Cl₂, methanol, CH₂Cl₂ and DMF (3×30 mL each). (Step 3) A solution of Fmoc-Gly-OH (1.43 g, 4.80 mmol), HOBt (735 mg, 4.80 mmol), HBTU (1.82 g, 4.80 mmol) and i-Pr₂NEt (2.09 mL, 12.0 mmol) in DMF (30.0 mL) was added to the resin and the reaction vessel shaken 4 hours. (Step 4) The resin washed with DMF, CH₂Cl₂, methanol, CH₂Cl₂ and DMF (3×30 mL each). (Step 5) The coupling reaction was found to be more than 95% complete as assessed by the fulvene-piperidine assay. Steps 1-5 were repeated with Fmoc-Leu-OH and Fmoc-Pro-OH respectively, to complete the sequence PLG˜Hphe.

### Part B—Preparation of Ac-PLG˜Hphe-OH

The peptide-resin of Part A (0.918 g) was placed in a 100 mL Advanced ChemTech reaction vessel and swollen by washing with DMF (2×30 mL). The resin was treated with 20% piperidine in DMF (30 mL) for 30 minutes to remove the Fmoc protecting group, followed by washing (30 ml volumes) with DMF (3×), dichloromethane (3×), methanol (3×), dichloromethane (3×), and DMF (3×). Acetic anhydride (0.40 mL, 4.2 mmol), DIEA (0.74 mL, 4.2 mmol), and DMF (30 mL) were added and the mixture was gently agitated for 2 hours. The peptide-resin was washed (30 mL volumes) with DMF (3×), dichloromethane (3×), methanol (3×), and dichloromethane (3×), and dried under vacuum. The peptide-resin was placed in a sintered glass funnel and treated with 1% TFA in dichloromethane (12 mL) for 2 minutes. The solution was filtered, by application of nitrogen pressure, directly into a flask containing 1:9 pyridine:methanol (2.0 mL). The cleavage procedure was repeated ten (10) times. The combined filtrates were concentrated to give a colorless oily solid. This crude product was triturated with water (2×25 mL) and dried under reduced pressure to give a dry solid. This solid was purified by HPLC on a Phenomenex Luna C18(2) column (41.4×250 mm) using a 0.9%/min gradient of 18 to 45% acetonitrile containing 0.1% TFA at a flow rate of 80 mL/min. The main product peak eluting at 23.8 minutes was lyophilized to give 192.0 mg (71%) of the title compound as a colorless solid with 100% purity by HPLC. ¹H NMR (1:1 CD₃CN+D₂O): δ 7.99 (t, J=7.5 Hz, 2H), 7.94-7.88 (m, 3H), 5.11-4.90 (m, 3H), 4.63-4.51 (m, 2H), 4.26-4.07 (m, 2H), 3.42-3.26 (m, 2H), 3.00-2.77 (m, 2H), 2.73-2.63 (m, 4H), 2.61-2.53 (m, 3H), 2.40-2.22 (m, 3H), 1.64-1.53 (m, 6H); ¹³C NMR (1:1 CD₃CN+D₂O): δ 173.93, 173.59, 173.55, 172.19, 170.18, 140.52, 140.42, 128.03, 125.67, 59.88, 51.86, 51.49, 47.96, 41.84, 38.76, 32.10, 30.84, 29.08, 24.01, 23.89, 21.84, 21.10, 20.08. MS: m/e 489.4 (100, M+H).

### Part C—Preparation of (2S)-2-[(tert-Butoxy)carbonylamino]-N-[4-(hydroxymethyl)-phenyl]-5-[(imino{[(2,2,5,7,8-pentamethylchroman-6-yl)sulfonyl]amino}methyl)-amino]pentanamide

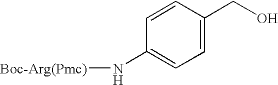

A solution of Boc-Arg(Pmc)-OH (500 mg, 0.925 mmol), p-aminobenzyl alcohol (170.9 mg, 1.388 mmol), and EEDQ (377.4 mg, 1.388 mmol) in 1:1 toluene:ethanol (10 mL) was stirred at room temperature under nitrogen for 48 hours. The volatiles were removed under reduced pressure. The resulting residue was dissolved in ethyl acetate (10 mL), washed consecutively with 5% NaHCO₃ (3×20 mL), 10% citric acid (2×20 mL), water (20 mL), and saturated NaCl (20 mL), dried (MgSO₄), filtered, and concentrated under reduced pressure to give the title compound as a colorless solid (550 mg, 92%). MS (ESI): 1291.7 (20, 2M+H), 646.3 (100, M+H); HRMS: Calcd for C₃₂H₄₈N₅O₇S (M+H): 646.3269; Found: 646.3272.

### Part D—Preparation of (4-{(2S)-2-[(tert-Butoxy)carbonylamino]-5-[(imino{[(2,2,5,7,8-pentamethylchroman-6-yl)sulfonyl]amino}methyl)amino]pentanoylamino}phenyl)methyl (4-nitrophenoxy)formate

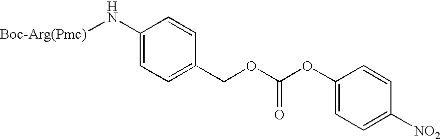

A solution of the product from Part C (535 mg, 0.828 mmol), 4-nitrophenyl chloroformate (251 mg, 1.243 mmol), and pyridine (105 mg, 1.325 mmol) in dichloromethane (5.0 mL) was stirred at room temperature under nitrogen for 18 hours. Additional 4-nitrophenyl chloroformate (188 mg, 0.932 mmol) and pyridine (84 mg, 1.06 mmol) were added and stirring was continued for an additional 1.5. The solvents were evaporated and the resulting crude product was purified by flash chromatography on silica gel, eluting with 4:1 dichloromethane:acetonitrile followed by 1:1 dichloromethane:acetonitrile. The title compound was obtained as a colorless solid (424 mg, 63%, HPLC purity 100%). MS: 1621.7 (15, 2M+H), 811.3 (100, M+H).

### Part E—Preparation of (2R)-2-{[(4-{(2S)-2-Amino-5-[(imino {[(2,2,5,7,8-pentamethylchroman-6-yl)sulfonyl]amino}methyl)amino]pentanoylamino}phenyl)-methoxy]carbonylamino}-N-amino-4-methylpentanamide, 2,2,2-Trifluoroacetic Acid Salt

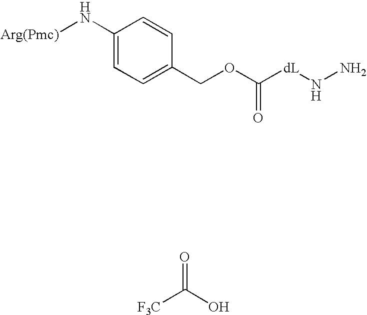

A solution of the product of Example 45C (83 mg, 0.176 mmol) in 50:50 TFA:dichloromethane (5.0 mL) was stirred for 20 minutes at ambient temperature under nitrogen and concentrated to dryness under reduced pressure. The resulting amber oil was dissolved in DMF (1.0 mL) and adjusted to pH 9 with DIEA (70 μL, 0.402 μmol). The product of Example 46D (95 mg, 0.117 mmol) and HOBt (18 mg, 0.117 mmol) were added and the solution was stirred at room temperature under nitrogen for 4 hours and concentrated under reduced pressure. The resulting residue was taken up in 30:70 TFA:dichloromethane (5.0 mL), stirred at room temperature under nitrogen for 1 hour, and concentrated to give an oily solid. This oily residue was purified by HPLC on a Phenomenex Luna C18(2) column (21.2×250 mm) using a 0.9%/min gradient of 18 to 63% acetonitrile containing 0.1% TFA at a flow rate of 20 mL/min. The main product peak eluting at 48.8 minutes was lyophilized to give the title compound as a colorless solid (65 mg, 82%, HPLC purity 100%). MS (ESI): 1878.9 (10, 2M+H), 939.5 (100, M+H).

### Part F—Preparation of N—[(N-{1-[N-(1-{N-[4-({N-[(1R)-1-(N-Aminocarbamoyl)-3-methylbutyl]carbamoyloxy}methyl)phenyl]carbamoyl}(1S)-4-[(imino{[(2,2,5,7,8-pentamethylchroman-6-yl)sulfonyl]amino}methyl)amino]butyl)carbamoyl](1S)-3-phenylpropyl}carbamoyl)methyl](2S)-2-[((2S)-1-acetylpyrrolidin-2-yl)carbonylamino]-4-methylpentanamide, 2,2,2-Trifluoroacetic Acid Salt

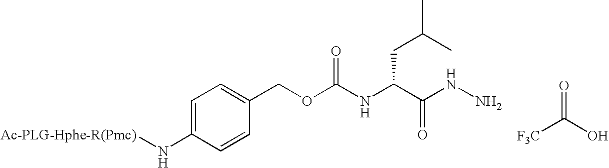

A solution of the product of Part E (25 mg, 0.027 mmol), the product of Part B (13 mg, 0.027 mmol), HOAt (4 mg, 0.027 mmol), and collidine (18 μL, 0.133 mmol) in DMF (0.5 mL) was treated with DIC (8.2 μL, 0.053 mmol) and stirred at room temperature under nitrogen for 18 hours. Additional product of Part E (13 mg, 0.014 mmol) was added and stirring was continued for an additional 18 hours. TAEA (0.1 mL, 0.8 mmol) was added to the reaction solution and stirring was continued at room temperature under nitrogen for 20 minutes. The volatiles were removed under reduced pressure, and the resulting residue was purified by HPLC on a Phenomenex Luna C18(2) column (21.2×250 mm) using a 0.45%/min gradient of 36 to 49.5% acetonitrile containing 0.1% TFA at a flow rate of 20 mL/min. The main product peak eluting at 29.3 minutes was lyophilized to give the title compound as a colorless solid (7.2 mg, 23%, HPLC purity 100%). ¹H NMR (1:1 CD₃CN+D₂O): δ 8.21-8.15 (m, 2H), 8.01-7.94 (m, 2H), 7.94-7.89 (m, 2H), 7.86-7.80 (m, 3H), 5.75 (d, J=12 Hz, 1H), 5.60 (d, J=12 Hz, 1H), 5.05-4.87 (m, 2H), 4.77-4.69 (m, 1H), 4.59-4.44 (m, 2H), 4.18-4.00 (m, 2H), 3.79 (bs, 2H), 3.35-3.19 (m, 4H), 3.13 (s, 3H), 3.11 (s, 3H), 2.83-2.55 (m, 11H), 2.54-2.32 (m, 7H), 2.30-2.07 (m, 8H), 1.91 (s, 6H), 1.56-1.42 (m, 12H). MS (ESI): 1187.7 (100, M+H). Chiral analysis: 97.0% L-Hphe.

### EXAMPLE 47

### Synthesis of 2-{[2-({[N-({4-[N-((2R)-2-Amino-3-phenylpropanoylamino)-carbamoyl]phenyl}methyl)carbamoyl]methyl}{2-[bis(carboxymethyl)amino]-ethyl}amino)ethyl](carboxymethyl)amino}acetic Acid, Trifluoroacetic Acid Salt

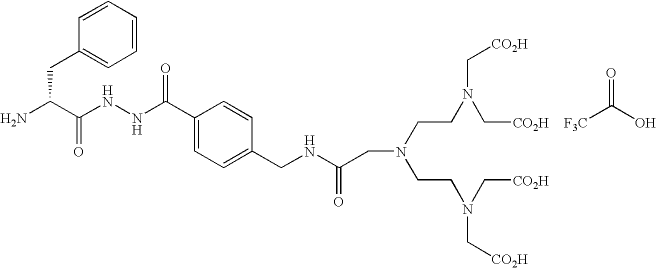

### Part A—Preparation of (2R)—N-{[4-(Aminomethyl)phenyl]carbonylamino}-2-[(tert-butoxy)carbonylamino]-3-phenylpropanamide, Trifluoroacetic Acid Salt

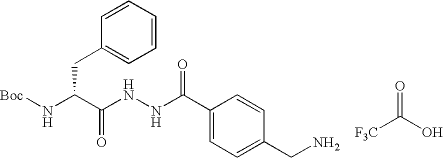

The product of Example 29A (279 mg, 0.573 mmol) was dissolved in 50:50 TFA:dichloromethane (2.0 mL), stirred for 20 minutes under nitrogen at ambient temperature and concentrated to dryness. The resulting oily residue was dissolved in DMF (1.0 mL) and adjusted to pH 9 by addition of DIEA (0.4 mL, 0.230 mmol). The solution was treated with Boc-D-Phe-OH (160 mg, 0.573 mmol), HOBt (105 mg, 0.687 mmol), HBTU (261 mg, 0.687 mmol), and DIEA (0.15 mL, 0.086 mmol) and stirred at room temperature under nitrogen for 2 hours. The solution was treated with TAEA (0.4 mL) and stirring was continued for 2 hours. The volatiles were removed under vacuum and the resulting residue was and purified by HPLC on a Phenomenex Luna C18(2) column (41.4×250 mm) using a 0.9%/min gradient of 13.5 to 40.5% acetonitrile containing 0.1% TFA at a flow rate of 80 mL/min. The main product peak eluting at 21.4 minutes was lyophilized to give the title compound as a colorless solid (140 mg, 57%, HPLC purity 100%). ¹H NMR (1:1 CD₃CN:D₂O): δ 7.83 (a portion of AA′BB′ quartet, J=8.4 Hz, 2H), 7.51 (b portion of AA′BB′ quartet, J=8.4 Hz, 2H), 7.33-7.20 (m, 5H), 4.43-4.37 (m, 1H), 4.14 (s, 2H), 3.22-3.14 (m, 1H), 2.88-2.76 (m, 1H), 1.27(s, 9H); ¹³C NMR (1:1 CD₃CN:D₂O): δ 173.47, 168.53, 162.70 (q, J=28 Hz), 157.38, 138.11, 137.97, 133.14, 130.34, 130.28, 129.55, 129.22, 127.86, 117.69 (q, J=291 Hz), 81.45, 55.60, 43.53, 38.55, 28.51. MS (ESI): 825.5 (100, 2M+H), 413.4 (65, M+H), 313.4 (70, M+H); HRMS: Calcd for C₂₂H₂₉N₄O₄ (M+H): 413.2183; Found: 413.2186.

### Part B—Preparation of 2-{[2-({[N-({4-[N-((2R)-2-Amino-3-phenylpropanoylamino)-carbamoyl]phenyl}methyl)carbamoyl]methyl}{2-[bis(carboxymethyl)amino]ethyl}-amino)ethyl](carboxymethyl)amino}acetic Acid, Trifluoroacetic Acid Salt

A solution of the product of Part A (120 mg, 0.291 mmol), DTPA (180 mg, 0.291 mmol), HBTU (132 mg, 0.349 mmol), and DIEA (101.4 μL, 0.582 mmol) in anhydrous DMF (2 mL) was stirred at room temperature under nitrogen for 20 minutes. The volatiles were removed under reduced pressure, and the resulting residue was dissolved in 90:9:1 TFA:dichloromethane:TIS (2.0 mL) and stirred at room temperature under nitrogen for 5 hours. The solution was concentrated and the crude product was purified by HPLC on a Phenomenex Luna C18(2) column (41.4×250 mm) using a method which was isocratic for 5 minutes at 1.8% acetonitrile with a flow rate of 80 mL/min, followed by a 0.9%/min gradient of 1.8 to 28.8% acetonitrile containing 0.1% TFA at a flow rate of 80 mL/min. The main product peak eluting at 20.1 minutes was lyophilized to give the title compound as a colorless solid (90 mg, 45%, HPLC purity 87%). MS (ESI): 344.6 (100, M+2H)), 688.3 (95, M+H); HRMS: Calcd for C₃₁H₄₂N₇O₁₁ (M+H): 688.2937; Found: 688.2936. Chiral analysis: 99.2% D-Hphe.

### EXAMPLE 48

### Synthesis of 2-({2-[({N-[(2R)-2-Amino-3-(4-phenylphenyl)propanoylamino]-carbamoyl}methyl){2-[bis(carboxymethyl)amino]ethyl}amino]ethyl}-(carboxymethyl)amino)acetic Acid, Trifluoroacetic Acid Salt

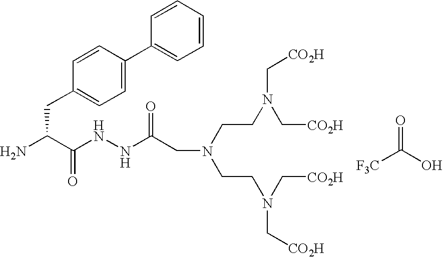

### Part A—Preparation of (2R)—N-Amino-2-[(tert-butoxy)carbonylamino]-3-(4-phenylphenyl)propanamide, 2,2,2-trifluoroacetic Acid

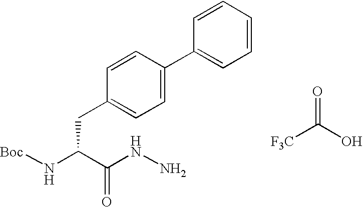

A solution of 9-fluorenylmethyl carbazate (223 mg, 0.879 mmol), BOC-D-Bip-OH (300 mg, 0.879 mmol), HBTU (400 mg, 1.054 mmol), and DIEA (0.31 mL, 1.757 mmol) in DMF (3.0 mL) was stirred at room temperature under nitrogen for 18 hours. The solution was treated with TAEA (0.5 mL) and stirring was continued for 2 hours. The volatiles were removed under vacuum and the resulting residue was purified by HPLC on a Phenomenex Luna C18(2) column (41.2×250 mm) using a 0.9%/min gradient of 27 to 54% acetonitrile containing 0.1% TFA) at a flow rate of 80 mL/min. The main product peak eluting at 18.1 minutes was lyophilized to give the title compound as a colorless solid (170 mg, 92%, HPLC purity 100%). MS (ESI): 256.4 (40, M−Boc+H), 300.3 (100, M−t−Bu+H), 378.3 (10, M+Na); HRMS: Calcd for C₂₀H₂₆N₃O₃ (M+H): 356.1969; Found: 356.1968.

### Part B—Preparation of 2-({2-[({N-[(2R)-2-Amino-3-(4-phenylphenyl)-propanoylamino]carbamoyl}methyl){2-[bis(carboxymethyl)amino]ethyl}amino]ethyl}(carboxymethyl)amino)acetic Acid, Trifluoroacetic Acid Salt

A solution of the product of Part A (160 mg, 0.450 mmol), DTPA (278 mg, 0.450 mmol), and HBTU (205 mg, 0.540 mmol) and DIEA (157 μL, 0.900 mmol) in anhydrous DMF (2 mL) was stirred at room temperature under nitrogen for 45 minutes. The solvents were removed under reduced vacuum and the resulting residue was dissolved in 90:10:2 TFA:dichloromethane:TIS (5.0 mL). The solution was stirred at room temperature under nitrogen for 4 hours and concentrated to an oily solid. The crude product was purified by HPLC on a Phenomenex Luna C18(2) column (41.2×250 mm) using a 0.9%/min gradient of 9 to 36% acetonitrile containing 0.1% TFA at a flow rate of 80 mL/min. The main product peak eluting at 17.6 minutes was lyophilized to give the title compound as a colorless solid (208 mg, 73%, HPLC purity 100%). MS (ESD): 316.3 (45, M+2H), 631.3 (100, M+H); HRMS: Calcd for C₂₉H₃₉N₆O₁₀ (M+H): 631.2722; Found: 631.2729.

### EXAMPLE 49

### Synthesis of 2-{[2-({[N-(5-{N-[((2S)Pyrrolidin-2-yl)carbonylamino]carbamoyl}-pentyl)carbamoyl]methyl}{2-[bis(carboxymethyl)amino]ethyl}amino)-ethyl](carboxymethyl)amino}acetic Acid, Trifluoroacetic Acid Salt

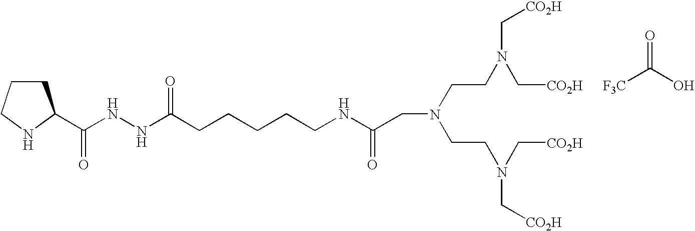

### Part A—Preparation of tert-Butyl (2S)-2-(N-{6-[(fluoren-9-ylmethoxy)carbonylamino]hexanoylamino}carbamoyl)pyrrolidinecarboxylate

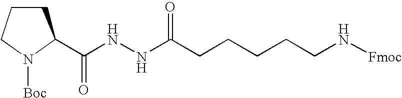

A solution of Boc-Pro-OH (193 mg, 0.9 mmol), HBTU (342 mg, 0.9 mmol), HOBt (140 mg, 0.9 mmol), and DIEA (525 μL, 3 mmol) in DMF (5.0 mL) was stir at room temperature under nitrogen for 20 minutes. The product of Example 3A (360 mg, 0.75 mmol) was added in one portion, followed by DIEA (525 μL, 3 mmol) to give a solution with pH=10. The solution was stirred at room temperature for two hours and added dropwise to water (500 mL). The resulting precipitate was collected by filtration on a medium fritted funnel, washed with water (75 mL), and dried to give the title compound as an off-white solid (156 mg, 31%, HPLC purity 100%). MS (ESI): 465.2 (100, M+H−Boc), 587.3 (10, M+Na).

### Part B—Preparation of 2-{[2-({[N-(5-{N-[((2S)Pyrrolidin-2-yl)carbonylamino]-carbamoyl}pentyl)carbamoyl]methyl}{2-[bis(carboxymethyl)amino]ethyl}amino)-ethyl](carboxymethyl)amino}acetic Acid, Trifluoroacetic Acid Salt

The product of Part A (156 mg, 275 μmol) was dissolved in 20:80 piperdine:DMF (5.0 mL), stirred under nitrogen at room temperature for 30 minutes, and concentrated to dryness under vacuum. The residue was dissolved in DMF (1.0 mL) and treated with DIEA (400 μL, 1.80 mmol). In a separate flask a solution of 2-{bis[2-(bis{[(tert-butyl)oxycarbonyl]methyl}amino)ethyl]amino}acetic acid (207 mg, 330 μmol), HBTU (125 mg, 330 μmol), and DIEA (800 μL, 3.60 mmol) in DMF (4.0 mL) was stirred at room temperature under nitrogen for 15 minutes. The two DMF solutions were combined, stirred an additional 30 minutes, and concentrated under reduced pressure to give a yellow oil. MS (ESI): 942.7 (100, M+H).

The above oil was dissolved in a 90:9:1 TFA:dichloromethane:TIS (5.0 mL), stirred at room temperature under nitrogen for 3 hours, and concentrated under reduced pressure to yield a yellow oil. This crude product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27.9% acetonitrile containing 0.1% TFA at a flow rate of 80 mL/min. The product fraction eluting at 16.8 minutes was lyophilized to give the title compound as a colorless solid (75 mg, 97%, HPLC purity 100%) ¹H NMR (DMSO-d₆): δ 12.02 (bs, 4H), 10.35 (s, 1H), 9.96 (s, 1H), 9.48 (bs, 1H), 8.70 (bs, 1H), 8.42 (t, J=5.7 Hz, 1H), 4.25-4.19 (m, 1H), 4.19-4.06 (m, 2H), 3.68-3.40 (m, 6H), 3.40-3.30 (m, 4H), 3.30-3.19 (m, 2H), 3.19-3.09 (m, 2H), 3.09-2.97 (m, 4H), 2.38-2.29 (m, 1H), 2.15 (t, J=7.5 Hz, 2H), 1.98-1.85 (m, 3H), 1.53 (quin, J=7.5 Hz, 2H), 1.44 quin, J=7.5 Hz, 2H), 1.34-1.23 (m, 4H). MS (ESI): 618.3 (100, M+H), 309.7 (60, M+2H). HRMS: Calcd for C₂₅H₄₁FeN₇O₁₁: (M−2H+Fe): 671.2208; Found: 671.2202.

### EXAMPLE 50

### Synthesis of 2-({2-[({N-[(4-{[N-((2R)-2-Amino-3-phenylpropanoylamino)-carbamoyl]methyl}phenyl)methyl]carbamoyl}methyl){2-[bis(carboxymethyl)amino]ethyl}amino]ethyl}(carboxymethyl)amino)acetic Acid, Trifluoroacetic Acid Salt

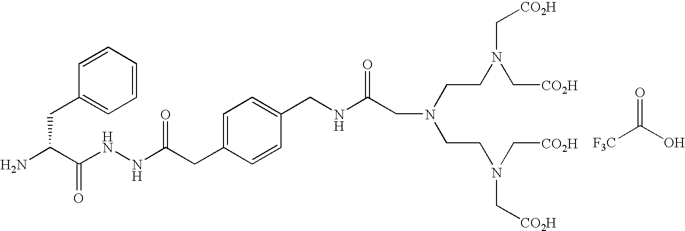

### Part A—Preparation of (2R)—N-{2-[4-(Aminomethyl)phenyl]acetylamino}-2-[(tert-butoxy)carbonylamino]-3-phenylpropanamide, Trifluoroacetic Acid Salt

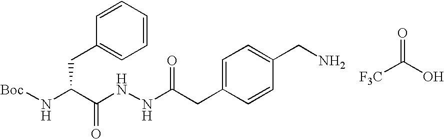

A solution of the product of Example 4A (303 mg, 0.754 mmol), Boc-D-Phe-OH (200 mg, 0.754 mmol), HBTU (343 mg, 0.905 mmol), and DIEA (0.263 mL, 1.508 mmol) in DMF (2.0 mL) was stirred at room temperature under nitrogen for 2 hours. The solution was treated with TAEA (0.4 mL) and stirring was continued for an additional 2 hours. The solvents were removed under vacuum and the resulting residue was purified by HPLC on a Phenomenex Luna C18(2) column (41.2×250 mm) using a 0.9%/min gradient of 18 to 45% acetonitrile containing 0.1% TFA at a flow rate of 80 mL/min. The main product peak eluting at 16.9 minutes was lyophilized to give the title compound as a colorless solid (242 mg, 75%, HPLC purity 93%). MS (ESI): 427.4 (100, M+H), 853.5 (60, 2M+H); HRMS: Calcd for C₂₃H₃₁N₄O₄ (M+H): 427.2340; Found: 427.2344.

### Part B—Preparation of 2-({2-[({N-[(4-{[N-((2R)-2-Amino-3-phenylpropanoylamino)carbamoyl]methyl}phenyl)methyl]carbamoyl}methyl){2-[bis(carboxymethyl)amino]ethyl}amino]ethyl}(carboxymethyl)amino)acetic Acid, Trifluoroacetic Acid Salt

The product of Part A (202 mg, 0.473 mmol) was dissolved in DMF (2.0 mL) and treated with 2-{bis[2-(bis{[(tert-butyl)oxycarbonyl]methyl}amino)-ethyl]amino}acetic acid (293 mg, 0.473 mmol), HBTU (215 mg, 0.568 mmol), and DIEA (165 μL, 0.947 mmol). The solution was stirred at room temperature under nitrogen for 45 minutes and diluted with ethyl acetate (40 mL). The ethyl acetate solution was washed consecutively with 1 N NaOH (2×40 mL) and saturated NaCl (40 mL), and concentrated under reduced pressure. The resulting residue was dissolved in 90:10:3 TFA:dichloromethane:TIS (5 mL) and stirred at room temperature under nitrogen for 2 hours. The volatiles were removed under reduced pressure and the crude product was purified by HPLC on a Phenomenex Luna C18(2) column (41.4×250 mm) using a 0.9%/min gradient of 1.8 to 28.8% acetonitrile containing 0.1% TFA at a flow rate of 80 mL/min. The main product peak eluting at 14.5 minutes was lyophilized to give the title compound as a colorless solid (135 mg, 41%, HPLC purity 95%). MS (ESI): 351.9 (100, M+2H), 702.4 (70, M+H); HRMS: Calcd for C₃₂H₄₄N₇O₁₁ (M+H): 702.3093; Found: 702.3092. Chiral analysis: 100.0% D-Hphe.

### EXAMPLE 51

### Synthesis of 2-[(2-{[(N-{5-[N-({(2R)-1-[(tert-Butyl)oxycarbonyl]pyrrolidin-2-yl}carbonylamino)carbamoyl]pentyl}carbamoyl)methyl]{2-[bis(carboxymethyl)amino]ethyl}amino}ethyl)(carboxymethyl)amino]acetic Acid, Trifluoroacetic Acid Salt

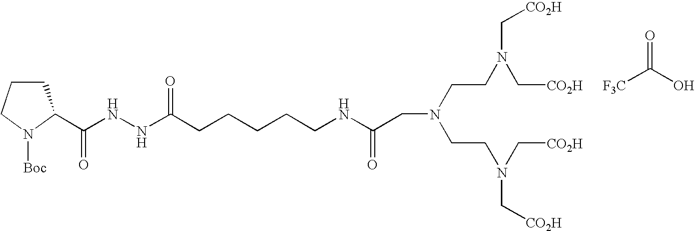

### Part A—Preparation of tert-Butyl (2R)-2-(N-{6-[(fluoren-9-ylmethoxy)carbonylamino]hexanoylamino}carbamoyl)pyrrolidinecarboxylate

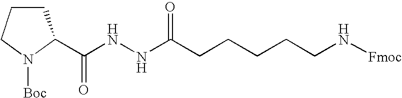

A solution of Boc-D-Pro-OH (378 mg, 1.8 mmol), HBTU (682 mg, 1.8 mmol), HOBt (276 mg, 1.5 mmol), and DIEA (700 μL, 4.0 mmol) in DMF (5 mL) was stirred at room temperature under nitrogen for 20 minutes. The product of Example 3A (360 mg, 0.75 mmol) was added in one portion, followed by DIEA (700 μL, 4.0 mmol) to raise the pH to 10. The solution was stirred for 2 hours at ambient temperatures and concentrated under reduced pressure. The residue was dissolved in ethyl acetate (500 mL) and washed consecutively with 10% citric acid (500 mL), saturated NaHCO₃ (500 mL), and saturated NaCl (500 mL). The ethyl acetate layer was dried (MgSO₄), filtered, and concentrated to give the title compound as an off-white solid (760 mg, 84%). MS (ESI): 465.2 (100, M+H−Boc), 587.3 (10, M+Na).

### Part B—Preparation of 2-[(2-{[(N-{5-[N-({(2R)-1-[(tert-Butyl)oxycarbonyl]pyrrolidin-2-yl}carbonylamino)carbamoyl]pentyl}carbamoyl)methyl]{2-[bis(carboxymethyl)-amino]ethyl}amino}ethyl)(carboxymethyl)amino]acetic Acid, Trifluoroacetic Acid Salt

The product of Part A (282 mg, 0.5 mmol) in 20:80 piperidine:DMF (5.0 mL) was stirred under nitrogen at room temperature for 30 minutes and concentrated under reduced pressure. The residue was dissolved in DMF (1.0 mL) and treated with DIEA (100 μL, 0.6 mmol) to give a solution of pH 10. In a separate flask a solution of 2-{bis[2-(bis{[(tert-butyl)oxycarbonyl]methyl}amino)ethyl]amino}acetic acid (370 mg, 0.5 mmol), HBTU (189 mg, 0.5 mmol), and DIEA (210 μL, 2.4 mmol) in DMF (4.0 mL) was stirred at room temperature under nitrogen for 15 minutes. The two DMF solutions were combined, allowed to stand at ambient temperature under nitrogen for 1 hour, and concentrated under reduced pressure. The crude product was dissolved in ethyl acetate (500 mL) and washed consecutively with 10% citric acid (2×50 mL), 1 N NaOH (2×50 mL), and saturated NaCl (50 mL). The ethyl acetate layer was dried (MgSO₄), filtered, and concentrated to give a yellow oil. MS (ESI): 942.7 (100, M+H).

The above oil was dissolved in 90:9:1 TFA:dichloromethane:TIS (5 mL), stirred at room temperature under nitrogen for 3 hours, and concentrated under reduced pressure. The product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27.9% acetonitrile containing 0.1% TFA at a flow rate of 80 mL/min. The product fraction eluting at 16.8 minutes was lyophilized to give the title compound as a colorless solid (111 mg, 36%, HPLC purity 100%). ¹H NMR (DMSO-d₆): δ 12.05 (bs, 4H), 10.36 (s, 1H), 9.96 (s, 1H), 9.50 (bs, 1H), 8.70 (bs, 1H), 8.43 (t, J=5.7 Hz, 1H), 4.26-4.19 (m, 1H), 4.19-4.12 (m, 2H), 3.68-3.43 (m, 6H), 3.40-3.30 (m, 4H), 3.30-3.19 (m, 2H), 3.19-3.09 (m, 2H), 3.09-2.98 (m, 4H), 2.38-2.30 (m, 1H), 2.15 (t, J=7.5 Hz, 2H), 1.98-1.87 (m, 3H), 1.53 quin, J=7.5 Hz, 2H), 1.44 quin, J=7.5 Hz, 2H), 1.35-1.23 (m, 4H). MS (ESI): 618.5 (100, M+H); 309.8 (60, M+2H). HRMS: Calcd for C₂₅H₄₁FeN₇O₁₁ (M−2H+Fe): 671.2208; Found: 671.2204.

### EXAMPLE 52

### Synthesis of 2-{[2-({[N-(5-{N-[(2R)-2-Amino-3-(phenylmethoxy)propanoylamino]-carbamoyl}pentyl)carbamoyl]methyl}{2-[bis(carboxymethyl)amino]ethyl}-amino)ethyl](carboxymethyl)amino}acetic Acid, Trifluoroacetic Acid Salt

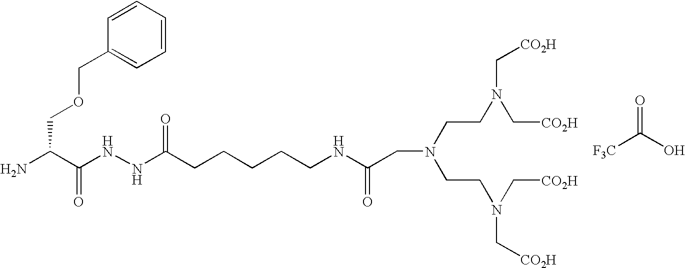

### Part A—Preparation of N-{(2R)-2-[(tert-Butoxy)carbonylamino]-3-(phenylmethoxy)propanoylamino}-6-aminohexanamide, Trifluoroacetic Acid Salt

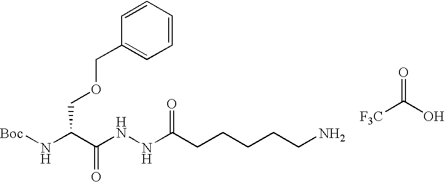

A solution of the product of Example 3A (311 mg, 0.847 mmol), BOC-D-Ser(Bzl)-OH (250 mg, 0.847 mmol), HBTU (385 mg, 1.016 mmol), and DIEA (0.30 mL, 1.693 mmol) in DMF (2.0 mL) was stirred at room temperature under nitrogen for 45 minutes. The solution was treated with TAEA (0.5 mL) and stirring was continued for an additional 2 hours. The solution was concentrated under vacuum and the resulting residue was purified by HPLC on a Phenomenex Luna C18(2) column (41.4×250 mm) using a 0.9%/min gradient of 18 to 45% acetonitrile containing 0.1% TFA at a flow rate of 80 mL/min. The main product peak eluting at 18.5 minutes was lyophilized to give the title compound as a colorless solid (75 mg, 21%, HPLC purity 95%). MS (ESI): 423.3(100, M+H); HRMS: Calcd for C₂₁H₃₅N₄O₅ (M+H): 423.2602; Found: 423.2602.

### Part B—Preparation of 2-{[2-({[N-(5-{N-[(2R)-2-Amino-3-(phenylmethoxy)-propanoylamino]carbamoyl}pentyl)carbamoyl]methyl}{2-[bis(carboxymethyl)-amino]ethyl}amino)ethyl](carboxymethyl)amino}acetic Acid, Trifluoroacetic Acid Salt

The product of Part A (70 mg, 0.166 mmol) was dissolved in DMF (2.0 mL) and treated with 2-{bis[2-(bis{[(tert-butyl)oxycarbonyl]methyl}amino)-ethyl]amino}acetic acid (102 mg, 0.166 mmol), HBTU (75 mg, 0.199 mmol) and TEA (46 μL, 0.331 mmol). The solution was stirred at room temperature under nitrogen for 45 minutes and diluted with ethyl acetate (40 mL). The resulting solution was washed consecutively with 1 N NaOH (2×40 mL) and saturated NaCl (40 mL), dried (MgSO₄), filtered, and concentrated. The resulting residue was dissolved in 90:10:3 TFA:dichloromethane:TIS (5.0 mL) and stirred at room temperature under nitrogen for 3 hours. The volatiles were removed under reduced pressure and the crude product was purified by HPLC on a Phenomenex Luna C18(2) column (41.4×250 mm) using a 0.9%/min gradient of 1.8 to 19.8% acetonitrile containing 0.1% TFA at a flow rate of 80 mL/min. The main product peak eluting at 17.7 minutes was lyophilized to give the title compound as a colorless solid (78 mg, 67%, HPLC purity 100%). ¹H NMR (1:1 CD₃CN:D₂O): δ 7.92-7.85 (m, 5H), 5.10 (q, J=10.8 Hz, 2H), 4.77 (t, J=4.8 Hz, 1H), 4.43-4.33 (m, 4H), 4.20 (s, 8H), 3.80-3.65 (m, 10H), 2.78 (t, J=7.5 Hz, 2H), 2.10 quin, J=7.5 Hz, 2H), 2.01 quin, J=7.5 Hz, 2H), 1.83 quin, J=7.5 Hz, 2H); ¹³C NMR (1:1 CD₃CN:D₂O): δ 176.24, 173.92, 168.41, 168.00, 162.10 (q, J=34.5 Hz), 138.65, 130.21, 129.76, 129.68, 118.21 (q, J=291 Hz), 74.78, 68.99, 56.82, 56.45, 53.65, 53.61, 51.95, 40.78, 34.74, 29.62, 27.24, 26.06. MS (ESI): 349.8 (199, M+2H), 698.4 (80, M+H); HRMS: Calcd for C₃₀H₄₈N₇O₁₂ (M+H): 698.3355; Found: 698.3358.

### EXAMPLE 53

### Synthesis of 2-{[2-({[N-(5-{N-[(2S)-2-Amino-3-(phenylmethylthio)-propanoylamino]carbamoyl}pentyl)carbamoyl]methyl}{2-[bis(carboxymethyl)amino]ethyl}amino)ethyl](carboxymethyl)amino}acetic Acid, Trifluoroacetic Acid Salt

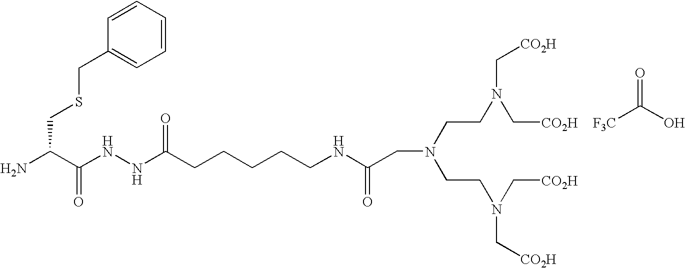

### Part A—Preparation of N-{(2S)-2-[(tert-Butoxy)carbonylamino]-3-(phenylmethylthio)propanoylamino}-6-aminohexanamide, Trifluoroacetic Acid Salt

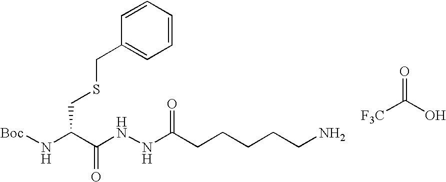

A solution of the product of Example 3A (295 mg, 0.803 mmol), Boc-D-Cys(Bzl)-OH (250 mg, 0.803 mmol), HBTU (365 mg, 0.963 mmol), and DIEA (0.28 mL, 1.606 mmol) in DMF (2 mL) was stirred at room temperature under nitrogen for 4 hours. The solution was treated with TAEA (0.5 mL) and stirring was continued for an additional 40 minutes. The solution was concentrated under vacuum and the resulting residue was purified by HPLC on a Phenomenex Luna C18(2) column (41.4×250 mm) using a 0.9%/min gradient of 18 to 45% acetonitrile containing 0.1% TFA at a flow rate of 80 mL/min. The main product peak eluting at 24.4 minutes was lyophilized to give the title compound as a colorless solid (224 mg, 64%, HPLC purity 95%). MS (ESI): 439.2 (100, M+H); HRMS: Calcd for C₂₁H₃₅N₄O₄S (M+H): 439.2374; Found: 439.2375.

### Part B—Preparation of 2-{[2-({[N-(5-{N-[(2S)-2-Amino-3-(phenylmethylthio)-propanoylamino]carbamoyl}pentyl)carbamoyl]methyl}{2-[bis(carboxymethyl)-amino]ethyl}amino)ethyl](carboxymethyl)amino}acetic Acid, Trifluoroacetic Acid Salt

A solution of the product of Part A (214 mg, 0.456 mmol) in DMF (2.0 mL) was treated with 2-{bis[2-(bis{[(tert-butyl)oxycarbonyl]methyl}amino)-ethyl]amino}acetic acid (301 mg, 0.456 mmol), HBTU (222 mg, 0.547 mmol), and DIEA (127 μL, 0.912 mmol), and stirred at room temperature under nitrogen for 45 minutes. The reaction was diluted with ethyl acetate (40 mL), washed consecutively with 1 N NaOH (2×40 mL) and saturated NaCl (40 mL), dried (MgSO₄), filtered, and concentrated. The resulting residue was dissolved in 90:10:3 TFA:dichloromethane:TIS (5.0 mL) and stirred at room temperature under nitrogen for 3 hours. The volatiles were removed under reduced pressure and the crude product was purified by HPLC on a Phenomenex Luna C18(2) column (41.4×250 mm) using a 0.9%/min gradient of 1.8 to 28.8% acetonitrile containing 0.1% TFA at a flow rate of 80 mL/min. The main product peak eluting at 19.6 minutes was lyophilized to give the title compound as a colorless solid (204 mg, 59%, HPLC purity 100%). MS (ESI): 357.9 (100, M+2H), 714.4 (70, M+H); HRMS: Calcd for C₃₀H₄₈N₇O₁₁S (M+H): 714.3127; Found: 714.3126. Chiral analysis: 97.0% D-Cys(Bzl).

### EXAMPLE 54

### Synthesis of 2-[(2-{[(N-{5-[N-((2R)-2-Amino-4-phenylbutanoylamino)-carbamoyl]pentyl}carbamoyl)methyl]{2-[bis(carboxymethyl)amino]ethyl}-amino}ethyl)(carboxymethyl)amino]acetic Acid, Trifluoroacetic Acid Salt

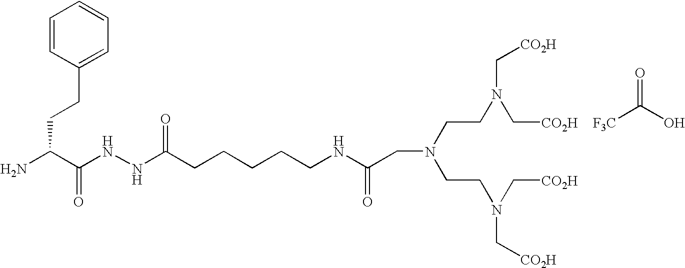

A solution of Boc-D-Hphe-OH (502 mg, 1.8 mmol), HBTU (568 mg, 1.5 mmol), HOBt (230 mg, 1.5 mmol), and DIEA (750 μL, 4.28 mmol) in DMF (5.0 mL) was stirred at room temperature under nitrogen for 20 minutes. The product of Example 3A (720 mg, 1.5 mmol) was added, followed by DIEA (750 μL, 4.28 mmol) to raise the pH to 10. The solution was stirred 18 hours at ambient temperature under nitrogen and concentrated under reduced pressure. The resulting residue was dissolved in ethyl acetate (50 mL) and washed consecutively with 10% citric acid (2×50 mL), saturated NaHCO₃ (2×50 mL), and saturated NaCl (50 mL). The ethyl acetate layer was dried (MgSO₄), filtered, and concentrated to give a yellow oil (715 mg). MS (ESI): 529.3 (100, M+H−Boc).

The above oil (315 mg) was dissolved in 50:50 DEA:acetonitrile (2.0 mL), stirred at ambient temperature for 30 minutes, and concentrated under reduced pressure. The residue was dissolved in DMF (1.0 mL) and treated with DIEA (200 μL, 1.2 mmol) to give a solution of pH 10. In a separate flask a solution of 2-{bis[2-(bis{[(tert-butyl)oxycarbonyl]methyl}amino)ethyl]amino}acetic acid (370 mg, 0.5 mmol), HBTU (208 mg, 0.55 mmol), and DIEA (200 μL, 1.2 mmol) in DMF (5.0 mL) was stirred at room temperature under nitrogen for 15 minutes. The two DMF solutions were combined, stirred at ambient temperature for 2 hours, and concentrated under reduced pressure. The resulting crude product was dissolved in ethyl acetate (50 mL), and the solution was washed consecutively with 10% citric acid (2×50 mL), saturated NaHCO₃ (2×50 mL), and saturated NaCl (50 mL). The ethyl acetate layer was dried (MgSO₄), filtered, and concentrated to yield a yellow oil. MS (ESI): 1006.5 (100, M+H).

The above oil was dissolved in a 90:9:1 TFA:dichloromethane:TIS (5.0 mL) and stirred at room temperature under nitrogen for 4 hours. The volatiles were removed under reduced pressure. The crude product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27.9% acetonitrile containing 0.1% TFA at a flow rate of 80 mL/min. The main product peak eluting at 29.5 minutes was lyophilized to give the title compound as a colorless solid (129 mg, 38%, HPLC purity 100%) ¹H NMR (DMSO-d₆): δ 12.05 (bs, 4H), 10.35 (s, 1H), 9.98 (s, 1H), 8.47-23 (m, 4H), 7.32 (t, J=7.5 Hz, 2H), 7.24-7.18 (m, 3H), 4.14 (s, 2H), 3.92 (s, 1H), 3.70-3.40 (m, 8H), 3.40-3.23 (m, 4H), 3.11 (q, J=6.6 Hz, 2H), 3.04 (t, J=5.7 Hz, 4H), 2.77-2.64 (m, 2H), 2.17 (t, J=7.5 Hz, 2H), 2.08-1.97 (m, 2H), 1.55 quin, J=7.5 Hz, 2H), 1.45 quin, J=7.5 Hz, 2H), 1.31 quin, J=7/5 Hz, 2H). MS (ESI): 682.3 (95, M+H); 341.9 (100, M+2H). HRMS: Calcd for C₃₀H₄₅FeN₇O₁₁ (M−2H+Fe): 735.2521; Found: 735.2519.

### EXAMPLE 55

### Synthesis of 2-[(2-{[(N-{[4-({N-[(2R)-2-Amino-3-(4-ethoxyphenyl)-propanoylamino]carbamoyl}methyl)phenyl]methyl}carbamoyl)methyl]{2-[bis(carboxymethyl)amino]ethyl}amino}ethyl)(carboxymethyl)amino]acetic Acid, Trifluoroacetic Acid Salt

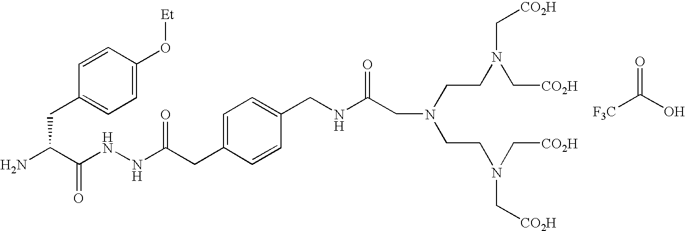

A solution of Boc-D-Tyr(OEt)-OH (557 mg, 1.8 mmol), HBTU (568 mg, 1.5 mmol), HOBt (230 mg, 1.5 mmol), and DIEA (750 μL, 4.28 mmol) in DMF (5.0 mL) was stirred at room temperature under nitrogen for 20 minutes. The solution was treated with the product of Example 4A (602 mg, 1.5 mmol), followed by DIEA (750 μL, 4.28 mmol) and stirring was continued for 18 hours. The solution was concentrated under reduced pressure and the residue was dissolved in ethyl acetate (50 mL). The ethyl acetate solution was washed with 10% citric acid (2×50 mL), saturated NaHCO₃ (2×50 mL), and saturated NaCl (50 mL). The ethyl acetate layer was dried (MgSO₄), filtered, and concentrated to give a yellow oil (285 mg). MS (ESI): 593.4 (100, M+H−Boc).

A solution of the above oil (275 mg) in 50:50 DEA:acetonitrile (5.0 mL) was allowed to stand under nitrogen at room temperature for 30 minutes and concentrated under vacuum. The resulting residue was taken up in DMF (1.0 mL) and treated with DIEA (150 μL, 1.0 mmol). In a separate flask a solution of 2-{bis[2-(bis{[(tert-butyl)oxycarbonyl]methyl}amino)ethyl]amino}acetic acid (295 mg, 0.48 mmol), HBTU (168 mg, 0.44 mmol), HOBt (67 mg, 044 mmol), and DIEA (150 μL, 1.0 mmol) in DMF (5.0 mL) was stirred at room temperature under nitrogen for 15 minutes. The two DMF solutions were combined and stirred an additional 2 hours. The solution was concentrated and the residue was redissolved in ethyl acetate (50 mL). The ethyl acetate solution was washed with 1 N NaOH (50 mL), dried (MgSO₄), filtered, and concentrated to yield a yellow-orange oil. MS (ESI): 1070.5 (100, M+H).

The above oil was dissolved in 90:8:2 TFA:dichloromethane:TIS (10 mL), stirred at room temperature under nitrogen for 4 hours, and concentrated under vacuum. The resulting crude product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27.9% acetonitrile containing 0.1% TFA at a flow rate of 80 mL/min after 10 minutes at 0.9% acetonitrile. The product fraction eluting at 35 minutes was lyophilized to give the title compound as a colorless solid (52 mg, 14%, HPLC purity 100%). ¹H NMR (DMSO-d₆): δ 12.05 (bs, 4H), 10.58 (s, 1H), 10.41 (s, 1H), 8.91 (s, 1H), 8.18 (bs, 3H), 7.30-7.15 (m, 6H), 6.87 (d, J=8.4 Hz, 2H), 4.33 (d, J=5.4 Hz, 2H), 4.23 (s, 1H), 4.00 (q, J=6.9 Hz, 2H), 3.74-3.43 (m, 12H), 3.43-3.26 (m, 4H), 3.10-2.97 (m, 5H), 2.94-2.87 (m, 1H), 1.31 (t, J=6.9 Hz, 3H). MS (ESI): 746.4 (100, M+H); 373.8 (100, M+H). HRMS: Calcd for C₃₄H₄₅FeN₇O₁₂ (M+2H+Fe): 799.2470; Found: 799.2462.

### EXAMPLE 56

### Synthesis of 2-({2-[({N-[(4-{[N-((2R)-2-Amino-3-cyclohexylpropanoylamino)-carbamoyl]methyl}phenyl)methyl]carbamoyl}methyl){2-[bis(carboxymethyl)amino]ethyl}amino]ethyl}(carboxymethyl)amino)acetic Acid, Trifluoroacetic Acid Salt

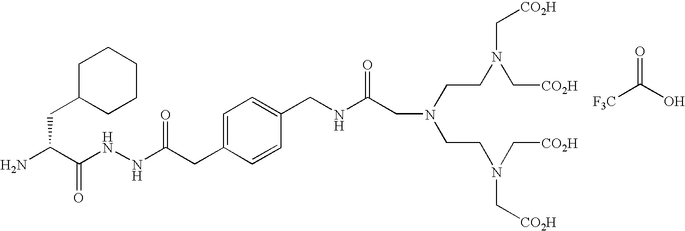

### Part A—Preparation of (2R)—N-{2-[4-(Aminomethyl)phenyl]acetylamino}-2-[(tert-butoxy)carbonylamino]-3-cyclohexylpropanamide, Trifluoroacetic Acid Salt

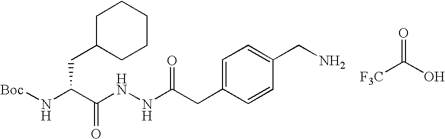

The DCHA salt of Boc-D-Cha-OH (337 mg, 0.744 mmol) was suspended in ethyl acetate (20 mL) in a separating funnel and washed with ice-cold 2 M H₂SO₄ (1.0 mL). The ethyl acetate layer was removed and set aside. The aqueous layer was diluted with cold water (10 mL) and extracted with ethyl acetate (2×20 mL). The combined ethyl acetate layers were washed with water (2×20 mL), dried (MgSO₄), filtered, and concentrated under reduced pressure at not more than 40° C. to give a colorless viscous solid (179 mg, 90% yield). This solid was taken up in DMF (2.0 mL) and treated with the product of Example 4A (265 mg, 0.660 mmol), HBTU (300 mg, 0.792 mmol), and sufficient DIEA to give a solution of pH=8. The solution was stirred at room temperature under nitrogen for 2 hours, treated with TAEA (0.5 mL), and stirred for an additional hour. The volatiles were removed under vacuum and the resulting residue was and purified by HPLC on a Phenomenex Luna C18(2) column (41.4×250 mm) using a 0.9%/min gradient of 18 to 45% acetonitrile containing 0.1% TFA at a flow rate of 80 mL/min. The main product peak eluting at 22.0 minutes was lyophilized to give the title compound as a colorless solid (155 mg, 54%, HPLC purity 93%). MS (ESI): 433.5 (100, M+H), 865.7 (60, 2M+H); HRMS: Calcd for C₂₃H₃₇N₄O₄ (M+H): 433.2809; Found: 433.2806.

### Part B—Preparation of 2-({2-[({N-[(4-{[N-((2R)-2-Amino-3-cyclohexylpropanoylamino)carbamoyl]methyl}phenyl)methyl]carbamoyl}methyl){2-[bis(carboxymethyl)amino]ethyl}amino]ethyl}(carboxymethyl)amino)acetic Acid, Trifluoroacetic Acid Salt

The product of Part A (140 mg, 0.324 mmol) was dissolved in DMF (2.0 mL) and treated with 2-{bis[2-(bis{[(tert-butyl)oxycarbonyl]methyl}amino)-ethyl]amino}acetic acid (200 mg, 0.324 mmol), HBTU (147 mg, 0.388 mmol), and DIEA (113 μL, 0.647 mmol). The resulting solution was stirred at room temperature under nitrogen for 4 hand diluted with ethyl acetate (40 mL). The solution was washed consecutively with 0.5 N NaOH (2×40 mL) and saturated NaCl (40 mL), and concentrated. The resulting oily solid was dissolved in 90:10:3 TFA:dichloromethane:TIS (5.0 mL), stirred at room temperature under nitrogen for 2 hours and concentrated under vacuum. The resulting crude product was purified by HPLC on a Phenomenex Luna C18(2) column (41.4×250 mm) using a 0.9%/min gradient of 6.3 to 24.3% acetonitrile containing 0.1% TFA at a flow rate of 80 mL/min. The main product peak eluting at 16.1 minutes was lyophilized to give the title compound as a colorless solid (151 mg, 66%, HPLC purity 100%). MS (ESI): 354.9 (100, M+2H), 708.5 (60, M+H); HRMS: Calcd for C₃₂H₄₇FeN₇O₁₁ (M+Fe-2H): 761.2677; Found: 761.2679. Chiral analysis: 99.8% D-Cha.

### EXAMPLE 57

### Synthesis of 2-({2-[({N-[(4-{2-[N-((2R)-2-Amino-4-methylpentanoylamino)-carbamoyl]ethyl}phenyl)methyl]carbamoyl}methyl){2-[bis(carboxymethyl)amino]-ethyl}amino]ethyl}(carboxymethyl)amino)acetic Acid, Trifluoroacetic Acid Salt

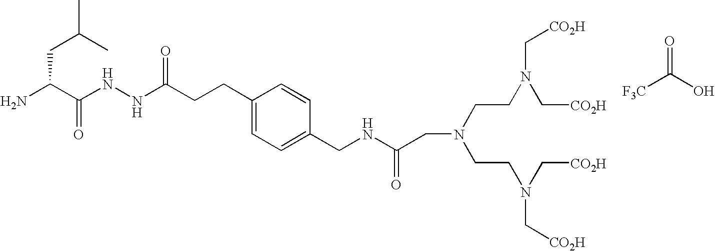

### Part A—Preparation of 3-(4-{[(Fluoren-9-ylmethoxy)carbonylamino]-methyl}phenyl)propanoic Acid

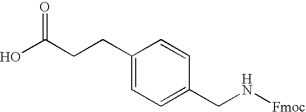

3-[4-(Aminomethyl)phenyl]propanoic acid (12.1 g, 0.0302 mol), prepared according to the procedure of Loeffler and Mar (J. Med. Chem. 1975, 18, 287-292) was dissolved in a solution of 5% Na₂CO₃ (125 mL), water (125 mL) and acetone (300 mL), and treated with Fmoc-OSu (12.2 g 0.0362 mol). The resulting solution was stirred at ambient temperatures for 2 hours and the pH was adjusted to 4-5 using 5 N HCl. The mixture was reduced approximately by half resulting in the formation of a large amount of solid colorless precipitate. This solid was collected by filtration, washed with water, and dried. Recrystallization from water gave the title compound as a colorless solid, MP 164.5-166° C. (4.927 g, 41%, HPLC purity=98%). ¹H NMR (DMSO-d₆): δ 7.89 (d, J=7.2 Hz, 2H), 7.78 (t, J=6.0 Hz, 1H), 7.70 (d, J=7.2 Hz, 2H), 7.42 (t, J=7.5 Hz, 2H), 7.34 (t, J=7.5 Hz, 2H), 7.16 (a portion of AA′BB′ quartet, J=7.8 Hz, 2H), 7.13 (b portion of AA′BB′ quartet, J=7.8 Hz, 2H), 4.34 (d, J=6.7 Hz, 2H), 4.22 (t, J=6.7 Hz, 1H), 4.14 (d, J=6.0 Hz, 2H), 2.80 (t, J=7.5 Hz, 2H), 2.52 (t, J=7.5 Hz, 2H); ¹³C NMR (DMSO-d₆): δ 173.64, 156.26, 143.84, 140.70, 139.31, 137.33, 128.04, 127.35, 126.98, 125.19, 120.03, 65.25, 46.78, 43.46, 35.23, 29.94. MS (ESI): 402.2 (100, M+H). HRMS: Calcd for C₂₅H₂₄NO₄ (M+H): 402.1700; Found: 402.1696.

### Part B—Preparation of N-Amino-3-(4-{[(fluoren-9-ylmethoxy)carbonylamino]-methyl}phenyl)propanamide, Trifluoroacetic Acid Salt

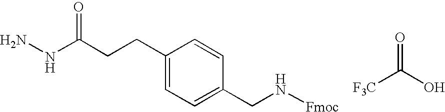

A solution of the product of Part A (3.0 g, 0.007 mol), t-butyl carbazate (1.0 g, 0.007 mol), HBTU (3.4 g, 0.009 mmol), and DIEA (2.6 mL, 0.015 mmol) was stirred at room temperature under nitrogen for 2 hours. The reaction was diluted with ethyl acetate (100 mL), washed consecutively with 10% citric acid (3×100 mL), 0.5 N NaOH (3×100 mL), and saturated NaCl (100 mL), dried (MgSO₄), filtered, and concentrated. The resulting solid was dissolved in 20 mL of TFA:dichloromethane (50:50) and stirred at room temperature for 30 minutes. The volatiles were removed under reduced vacuum and the resulting crude product was taken up in 60:40 acetonitrile:water (100 mL) and lyophilized to give the title compound as a yellow solid (4.238 g, 107%, HPLC purity 95%). ¹H NMR (DMF-d₇): δ 7.94 (d, J=7.2 Hz, 2H), 7.80 (t, J=6.0 Hz, 1H), 7.75 (t, J=7.2 Hz, 2H), 7.45 (t, J=7.5 Hz, 2H), 7.35 (t, J=7.5 Hz, 2H), 7.25 (a portion of AA′BB′ quartet, J=8.4 Hz, 2H), 7.20 (b portion of AA′BB′ quartet, J=8.4 Hz, 2H), 4.36 (d, J=7.2 Hz, 2H), 4.31 (d, J=6.0 Hz, 2H), 4.28 (t, J=7.2 Hz, 1H), 2.56 (t, J=7.8 Hz, 2H), remaining methylene in under solvent peak at 2.92; ¹³C NMR (DMF-d₇): δ 171.62, 159.58 (d, J=34.3 Hz), 157.08, 144.67, 141.53, 140.14, 138.17, 128.59, 128.02, 127.65, 127.43, 125.66, 120.41, 119.29 (q, J=252.7 Hz), 66.38, 47.57, 44.34, 30.96. MS (ESI): 416.2 (100, M+H). HRMS: Calcd for C₂₅H₂₆N₃O₃ (M+H): 416.1969; Found: 416.1969.

### Part C—Preparation of (2R)—N-{3-[4-(Aminomethyl)phenyl]propanoylamino}-2-[(tert-butoxy)carbonylamino]-4-methylpentanamide, Trifluoroacetic Acid Salt

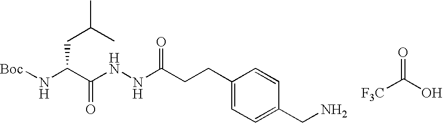

A solution of Boc-D-Leu-OH (200 mg, 0.865 mmol), the product of Part B (359 mg, 0.865 mmol), HBTU (394 mg, 1.038 mmol), and DIEA (0.301 mL, 1.729 mmol) in DMF (2.0 mL) was stirred at room temperature under nitrogen for 2 hours. The solution was treated with TAEA (0.50 mL) and stirring was continued for 1 hour. The volatiles were removed under vacuum and the resulting residue was and purified by HPLC on a Phenomenex Luna C18(2) column (41.2×250 mm) using a 0.9%/min gradient of 18 to 45% acetonitrile containing 0.1% TFA at a flow rate of 80 mL/min. The main product peak eluting at 15.6 minutes was lyophilized to give the title compound as a colorless solid (246 mg, 70%, HPLC purity 100%). MS (ESI): 407.4 (100, M+H); HRMS: Calcd for C₂₁H₃₅N₄O₄ [M+H]: 407.2653, Found: 407.2647.

### Part D—Preparation of 2-({2-[({N-[(4-{2-[N-((2R)-2-Amino-4-methylpentanoylamino)carbamoyl]ethyl}phenyl)methyl]carbamoyl}methyl){2-[bis(carboxymethyl)amino]ethyl}amino]ethyl}(carboxymethyl)amino)acetic Acid, Trifluoroacetic Acid Salt

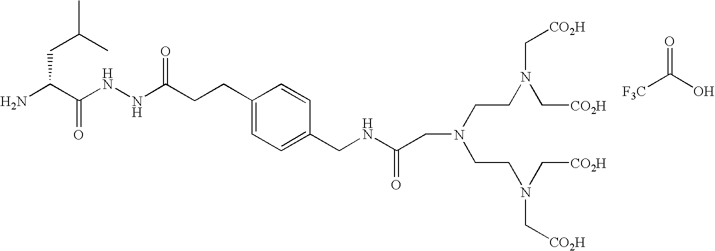

A solution of DTPA (304 mg, 0.492 mmol), the product of Part C (200 mg, 0.492 mmol), HBTU (224 mg, 0.590 mmol) and DIEA (sufficient to bring the pH to 10) in DMF (2.0 mL) was stirred at room temperature under nitrogen for 4 hours. The reaction was diluted with ethyl acetate (40 mL), washed consecutively with 0.5 N NaOH (2×40 mL) and saturated NaCl (40 mL), dried (MgSO₄), filtered, and concentrated under vacuum. The resulting residue was dissolved in 90:10:3 TFA:dichloromethane:TIS (5.0 mL) and stirred at room temperature under nitrogen for 2 hours. The volatiles were removed under vacuum and the crude product was purified by HPLC on a Phenomenex Luna C18(2) column (41.2×250 mm) using a 0.9%/min gradient of 1.8 to 19.8% acetonitrile containing 0.1% TFA at a flow rate of 80 mL/min. The main product fraction eluting at 16.4 minutes was lyophilized to give the title compound as a colorless solid (185 mg, 55%, HPLC purity 100%). MS (ESI): 682.3 (50, M+H), 341.9 (100, M+2H). HRMS: Calcd for C₃₀H₄₅FeN₇O₁₁ (M+Fe-2H): 735.2521; Found: 735.2519; Chiral analysis: 99.6% D-Leu.

### EXAMPLE 58

### Synthesis of 2-[(2-{[(N-{5-[N-((2R)-2-Amino-3-imidazol-4-ylpropanoylamino)-carbamoyl]pentyl}carbamoyl)methyl]{2-[bis(carboxymethyl)amino]ethyl}-amino}ethyl)(carboxymethyl)amino]acetic Acid, Trifluoroacetic Acid Salt

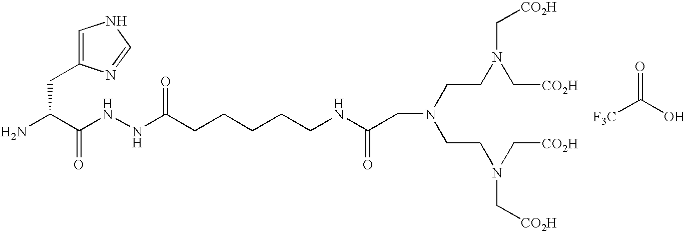

### Part A—Preparation of N-{(2R)-2-[(tert-butoxy)carbonylamino]-3-imidazol-4-ylpropanoylamino}-6-aminohexanamide, Acetic Acid Salt

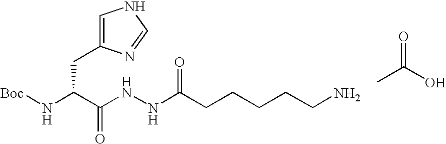

A solution of Boc-D-His-OH (220 mg, 0.862 mmol), the product of Example 3A (316.7 mg, 0.862 mmol), HBTU (392 mg, 1.034 mmol), and DIEA (0.300 mL, 1.724 mmol) in DMF (10 mL) was stirred at room temperature under nitrogen for 2 hours. The solution was treated with TAEA (0.5 mL) was stirred for an additional 2 hours. The solution was concentrated under vacuum and the resulting residue was and purified by HPLC on a Phenomenex Luna C18(2) column (41.4×250 mm) using a method which was isocratic for 10 minutes at 1.8% acetonitrile with a flow rate of 80 mL/min, followed by a 0.9%/min gradient of 1.8 to 28.8% acetonitrile containing 15 mM NH₄OAc (pH 7) at a flow rate of 80 mL/min. The main product peak eluting at 22.4 minutes was lyophilized to give the title compound as a colorless solid (39 mg, 10%, HPLC purity 95%). ¹H NMR (1:1 CD₃CN:D₂O): δ 7.84 (s, 1H), 6.98 (s, 1H), 4.39-4.23 (m, 1H), 3.10-3.02 (m, 1H), 2.92-2.82 (m, 3H), 2.23 (t, J=7.2 Hz, 2H), 1.83 (s, 3H), 1.62-1.51 (m, 4H), 1.37-1.25 (m, 11H); ¹³C NMR (1:1 CD₃CN:D₂O): δ 179.84, 175.52, 172.88, 157.41, 136.01, 132.57, 118.42, 81.70, 54.00, 40.23, 34.03, 29.72, 28.53, 27.35, 26.09, 25.27, 23.62. MS (ESI): 383.4 (100, M+H), 283.4 (25, M-Boc+H).

### Part B—Preparation of tert-Butyl 2-({2-[({N-[5-(N-{(2R)-2-[(tert-Butoxy)-carbonylamino]-3-imidazol-4-ylpropanoylamino}carbamoyl)pentyl]carbamoyl}-methyl)[2-(bis{[(tert-butyl)oxycarbonyl]methyl}amino)ethyl]amino]ethyl}{[(tert-butyl)oxycarbonyl]methyl}amino)acetate

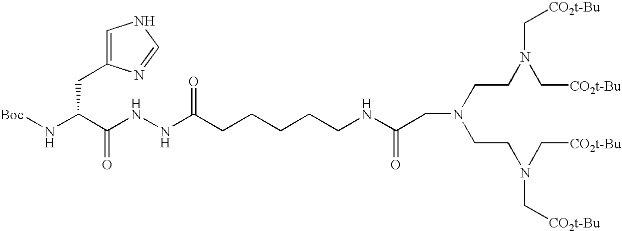

A solution of 2-{bis[2-(bis{[(tert-butyl)oxycarbonyl]methyl}amino)ethyl]-amino}acetic acid (prepared according to the procedure described in Journal of Organic Chemistry (1993), 58(5), 1151-8, 71 mg, 0.115 mmol), the product of Part A (44 mg, 0.115 mmol), and HBTU (52 mg, 0.137 mmol), and collidine (30 μL, 0.229 mmol) in anhydrous DMF (2 mL) was stirred at room temperature under nitrogen for 2 hours. The solvents were removed under reduced pressure and the crude product was purified by HPLC on a Phenomenex Luna C18(2) column (21.2×250 mm) using a 0.9%/min gradient of 27 to 54% acetonitrile containing 0.1% TFA at a flow rate of 20 mL/min. The main product peak eluting at 26.4 minutes was lyophilized to give the title compound as a colorless solid (30 mg, 27%, HPLC purity 100%). MS (ESI): 982.7 (40, M+H), 492.0 (100, M+2H); HRMS: Calcd for C₄₇H₈₄N₉O₁₃ (M+H): 982.6183; Found: 982.6188.

### Part C—Preparation of 2-[(2-{[(N-{5-[N-((2R)-2-Amino-3-imidazol-4-ylpropanoylamino)carbamoyl]pentyl}carbamoyl)methyl]{2-[bis(carboxymethyl)amino]ethyl}amino}ethyl)(carboxymethyl)amino]acetic Acid, Trifluoroacetic Acid Salt

A solution of the product of Part B (28 mg, 0.029 mmol) in 90:10:3 TFA:dichloromethane:TIS (5.0 mL) was stirred at room temperature under nitrogen for 3 hours and concentrated to give an oily solid. The solid was lyophilized from 50:50 acetonitrile:H₂O (5.0 mL) to give the title compound as a colorless solid (14 mg, 75%, HPLC purity 90%). MS (ESI): 658.3 (70, M+H), 329.7 (100, M+2H); HRMS: Calcd for C₂₆H₄₄N₉O₁₁ (M+H): 658.3155; Found: 658.3154.

### EXAMPLE 59

### Synthesis of 2-[(2-{[(N-{[4-({N-[(2S)-2-Amino-3-(phenylmethylthio)-propanoylamino]carbamoyl}methyl)phenyl]methyl}carbamoyl)methyl]{2-[bis(carboxymethyl)amino]ethyl}amino}ethyl)(carboxymethyl)amino]acetic Acid, Trifluoroacetic Acid Salt

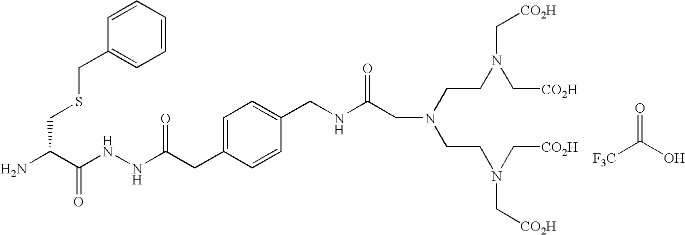

### Part A—Preparation of (2S)—N-{2-[4-(Aminomethyl)phenyl]acetylamino}-2-[(tert-butoxy)carbonylamino]-3-(phenylmethylthio)propanamide, Trifluoroacetic Acid Salt

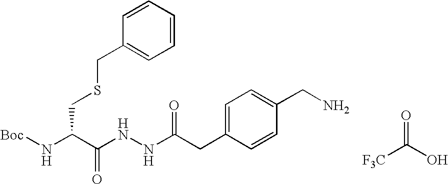

A solution of Boc-D-Cys(Bzl)-OH (250 mg, 0.803 mmol), the product of Example 4A (322 mg, 0.803 mmol), HBTU (365 mg, 0.963 mmol), and DIEA (0.280 mL, 1.606 mmol) in DMF (2.0 mL) was stirred at room temperature under nitrogen for 20 hours. The solution was treated with TAEA (0.5 mL) and stirring was continued for an additional 40 minutes. The volatiles were removed under vacuum and the resulting residue was purified by HPLC on a Phenomenex Luna C18(2) column (41.4×250 mm) using a 0.9%/min gradient of 18 to 45% acetonitrile containing 0.1% TFA at a flow rate of 80 mL/min. The main product peak eluting at 22.8 minutes was lyophilized to give the title compound as a colorless solid (151 mg). ¹H NMR (1:1 CD₃CN:D₂O): δ 7.37-7.27 (m, 8H), 7.25-7.20 (m, 1H), 4.25 (bs, 1H), 4.05 (s, 2H), 3.72 (s, 2H), 3.58 (s, 2H), 2.90-2.81 (m, 1H), 2.78-2.61 (m, 1H), 1.37 (s, 9H). MS (ESI): 473.4 (100, M+H); HRMS Calcd for C₂₄H₃₃N₄O₄S (M+H): 473.2217; Found: 473.2219.

### Part B—Preparation of 2-[(2-{[(N-{[4-({N-[(2S)-2-Amino-3-(phenylmethylthio)-propanoylamino]carbamoyl}methyl)phenyl]methyl}carbamoyl)methyl]{2-[bis(carboxymethyl)amino]ethyl}amino}ethyl)(carboxymethyl)amino]acetic Acid, Trifluoroacetic Acid Salt

The product of Part A (128 mg, 0.292 mmol) was dissolved in DMF (2 mL) along with 2-{bis[2-(bis{[(tert-butyl)oxycarbonyl]methyl}amino)ethyl]amino}acetic acid (180 mg, 0.292 mmol), HBTU (133 mg, 0.350 mmol), and DIEA (102 μL, 0.584 mmol). The resulting solution was stirred at room temperature under nitrogen for 45 minutes and diluted with ethyl acetate (40 mL). The ethyl acetate solution was washed consecutively with 1 N NaOH (2×40 mL) and saturated NaCl (40 mL), and concentrated under reduced pressure. The resulting residue was dissolved in 90:10:3 TFA:dichloromethane:TIS (5.0 mL) and stirred at room temperature under nitrogen for 5 hours. The volatiles were removed under reduced pressure and the crude product was purified by HPLC on a Phenomenex Luna C18(2) column (41.4×250 mm) using a 0.9%/min gradient of 6.3 to 24.3% acetonitrile containing 0.1% TFA at a flow rate of 80 mL/min. The main product peak eluting at 18.0 minutes was lyophilized to give the title compound as a colorless solid (142 mg, 65%, HPLC purity 96%). ¹H NMR (1:1 CD₃CN:D₂O): δ 7.32-7.21 (m, 9H), 4.33 (s, 2H), 4.09 (t, J=6.6 Hz, 1H), 3.93 (s, 2H), 3.78 (d, J=7.2 Hz, 1H), 3.74 (d, J=7.2 Hz, 1H), 3.62 (s, 8H), 3.56 (s, 2H), 3.22 (t, J=5.4 Hz, 4H), 3.15 (t, J=5.4 Hz, 4H), 2.95 (dd, H_{b} of abc system, J_{ab}=J_{ac}=6.6 Hz, J_{bc}=8.1 Hz, 1H), 2.88 (dd, H_{c} of abc system, J_{ab}=J_{ac}=6.6 Hz, J_{bc}=8.1 Hz, 1H); ¹³C NMR (1:1 CD₃CN:D₂O): δ 173.39, 173.14, 168.10, 167.90, 162.51 (q, J=34.8 Hz), 138.50, 137.91, 134.45, 130.63, 130.04, 129.83, 128.95, 128.52, 117.64 (q, J=290 Hz), 56.18, 55.92, 53.30, 52.18, 51.39, 43.76, 40.69, 36.79, 32.72. MS (ESI): 748.3 (88, M+H), 374.9 (100, M+2H); HRMS: Calcd for C₃₃H₄₃FeN₇O₁₁S (M+Fe-2H): 801.2085; Found: 801.2079; Chiral analysis: 97.9% D-Cys(Bzl).

### EXAMPLE 60

### Synthesis of 2-({2-[({N-[(4-{2-[N-((2R)-2-Amino-3-phenylpropanoylamino)-carbamoyl]ethyl}phenyl)methyl]carbamoyl}methyl){2-[bis(carboxymethyl)amino]-ethyl}amino]ethyl}(carboxymethyl)amino)acetic Acid, Trifluoroacetic Acid Salt

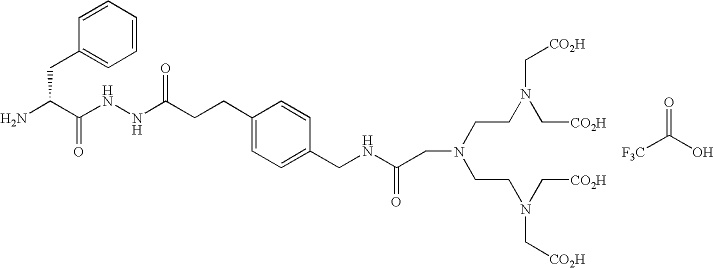

### Part A—Preparation of (2R)—N-{3-[4-(Aminomethyl)phenyl]propanoylamino}-2-[(tert-butoxy)carbonylamino]-3-phenylpropanamide, Trifluoroacetic Acid Salt

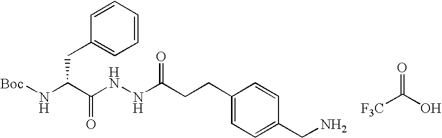

A solution of Boc-D-Phe-OH (250 mg, 0.942 mmol), the product of Example 57B (392 mg, 0.942 mmol), and DIEA (0.328 mL, 1.885 mmol) in DMF (2.0 mL) was treated with HBTU (429 mg, 1.131 mmol) and stirred at room temperature under nitrogen for 20 hours. The solution was treated with TAEA (0.5 mL) and stirring was continued for an additional 40 minutes. The solvents were removed under vacuum and the resulting residue was purified by HPLC on a Phenomenex Luna C18(2) column (41.4×250 mm) using a 0.9%/min gradient of 18 to 40.5% acetonitrile containing 0.1% TFA at a flow rate of 80 mL/min. The main product peak eluting at 18.1 minutes was lyophilized to give the title compound as a colorless solid (250 mg, 60%, HPLC purity 95%). MS (ESI): 441.4 (100, M+H); HRMS Calcd for C₂₄H₃₃N₄O₄ (M+H): 441.2496; Found: 441.2497.

### Part B—Preparation of 2-({2-[({N-[(4-{2-[N-((2R)-2-Amino-3-phenylpropanoylamino)carbamoyl]ethyl}phenyl)methyl]carbamoyl}methyl){2-[bis(carboxymethyl)amino]ethyl}amino]ethyl}(carboxymethyl)amino)acetic Acid, Trifluoroacetic Acid Salt

A solution of 2-{bis[2-(bis{[(tert-butyl)oxycarbonyl]methyl}amino)ethyl]-amino}acetic acid (292 mg, 0.472 mmol), the product of Part A (208 mg, 0.472 mmol), and DIEA (164 μL, 0.944 mmol) in DMF (2.0 mL) was treated with HBTU (215 mg, 0.567 mmol) and stirred at room temperature under nitrogen for 45 minutes. The reaction mixture was diluted with ethyl acetate (40 mL), washed consecutively with 0.5 N NaOH (2×40 mL) and saturated NaCl, dried (MgSO₄), filtered, and concentrated. The solvents were removed under vacuum and the resulting residue was dissolved in 90:10:3 TFA:dichloromethane:TIS (5.0 mL) and stirred at room temperature under nitrogen for 5 hours. The volatiles were removed under vacuum and the crude product was purified by HPLC on a Phenomenex Luna C18(2) column (41.4×250 mm) using a 0.9%/min gradient of 1.8 to 19.8% acetonitrile containing 0.1% TFA at a flow rate of 80 mL/min. The main product peak eluting at 16.1 minutes was lyophilized to give the title compound as a colorless solid (232 mg, 69%, HPLC purity 96%). ¹H NMR (1:1 CD₃CN:D₂O): δ 7.36-7.27 (m, 3H), 7.26-7.23 (m, 2H), 7.21-7.15 (m, 4H), 4.32 (s, 2H), 3.97 (s, 2H), 3.61 (s, 8H), 3.25 (t, J=6.0 Hz, 4H), 3.19-3.08 (m 6H), 2.84 (t, J=7.8 Hz, 2H), 2.53-2.46 (m, 2H), (missing methine was under HOD peak); ¹³C NMR (1:1 CD₃CN:D₂O): δ 174.58, 173.65, 168.82, 167.47, 162.42 (q, J=34.8 Hz), 140.79, 136.82, 134.64, 130.59, 130.09, 129.65, 128.92, 128.80, 117.60 (q, J=291 Hz), 56.05, 55.80, 54.22, 53.54, 51.18, 43.79, 37.64, 35.82, 31.33. MS (ESI): 716.4 (82, M+H), 358.8 (100, M+2H); HRMS: Calcd for C₃₃H₄₃FeN₇O₁₁ (M+Fe-2H): 769.2364; Found: 769.2367. Chiral analysis: 100.0% D-Phe.

### EXAMPLE 61

### Synthesis of 2-{[2-({[N-(5-{N-[(2R)-2-Amino-3-(4-phenylphenyl)propanoylamino]-carbamoyl}pentyl)carbamoyl]methyl}{2-[bis(carboxymethyl)amino]ethyl}-amino)ethyl](carboxymethyl)amino}acetic Acid, Trifluoroacetic Acid Salt

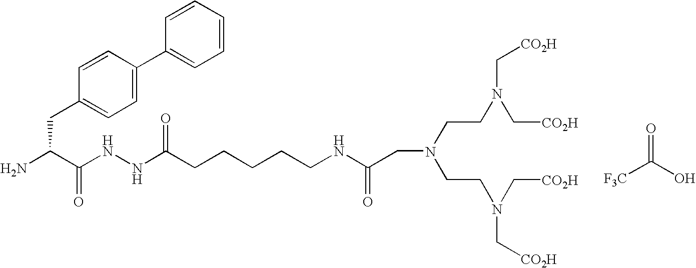

### Part A—Preparation of N-{(2R)-2-[(tert-butoxy)carbonylamino]-3-(4-phenylphenyl)propanoylamino}-6-[(fluoren-9-ylmethoxy)carbonylamino]hexanamide

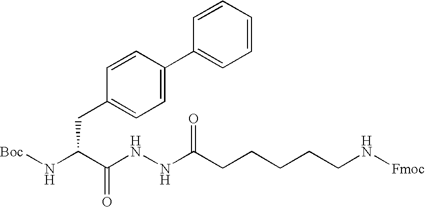

A solution of Boc-D-Bip-OH (409 mg, 1.2 mmol), HBTU (417 mg, 1.1 mmol), HOBt (168 mg, 1.1 mmol), and DIEA (350 μL, 2.0 mmol) in DMF (5.0 mL) was stirred at room temperature under nitrogen for 20 minutes. The product of Example 3A (481 mg, 1.0 mmol) was added in a single portion, followed by DIEA (350 μL, 2.0 mmol) to raise the pH to 10. The solution was stirred at ambient temperatures for 18 hours, and the volatiles were removed under vacuum. The resulting residue was triturated with ethyl acetate (50 mL) yielding a colorless solid that was collected by filtration and dried to give the title compound (814 mg, 98%, HPLC purity 100%). ¹H NMR (DMSO-d₆): δ 9.99 (s, 1H), 9.84 (s, 1H), 7.89 (d, J=7.2 Hz, 2H), 7.69 (d, J=7.8 Hz, 2H), 7.64 (d, J=7.2 Hz, 2H), 7.57 (d, J=7.8 Hz, 2H), 7.48-7.36 (m, 6H), 7.36-7.29 (m, 3H), 7.24 (t, J=5.4 Hz, 1H), 6.95 (d, J=8.4 Hz, 1H), 4.32-4.24 (m, 3H), 4.21 (t, J=6.9 Hz, 1H), 3.05 (d, J=10.2 Hz, 1H), 2.98 (q, J=6.4 Hz, 2H), 2.82 (t, J=12.3 Hz, 1H), 2.14 (t, J=7.2 Hz, 2H), 1.53 (t, J=7.2 Hz 2H), 1.41 (t, J=7.2 Hz, 2H), 1.36-1.12 (m, 11H). MS (ESI): 591.3 (100, M+H−Boc), 713.3 (20, M+H). HRMS: Calcd for C₄₁H₄₇FeN₄O₆ (M+H): 691.3496; Found: 691.3493.

### Part B—Preparation of 2-{[2-({[N-(5-{N-[(2R)-2-Amino-3-(4-phenylphenyl)-propanoylamino]carbamoyl}pentyl)carbamoyl]methyl}{2-[bis(carboxymethyl)-amino]ethyl}amino)ethyl](carboxymethyl)amino}acetic Acid, Trifluoroacetic Acid Salt

The product of Part A (203 mg, 0.30 mmol) was dissolved in 50:50 DEA:acetonitrile (5.0 mL) and stirred nitrogen at room temperature for 30 minutes. The solution was concentrated and the residue was dissolved in DMF (1.0 mL). In a separate flask a solution of 2-{bis[2-(bis{[(tert-butyl)oxycarbonyl]methyl}amino)-ethyl]amino}acetic acid (222 mg, 0.36 mmol), HBTU (125 mg, 0.33 mmol), and DIEA (100 μL, 0.60 mmol) in DMF (2.0 mL) was stirred at room temperature under nitrogen for 15 minutes. The two DMF solutions were combined and stirring was continued an additional 18 hours. The volatiles were removed by the use of reduced pressure and the resulting oily solid was taken up in ethyl acetate (50 mL). The ethyl acetate solution was washed consecutively with 10% citric acid (2×50 mL), saturated NaHCO₃ (2×50 mL), and saturated NaCl (50 mL), dried (MgSO₄), filtered, and concentrated to give a yellow oil. MS (ESI): 1086.6 (100, M+H).

The above oil was dissolved in 90:8:2 TFA:dichloromethane:TIS (10 mL), stirred at room temperature under nitrogen for 3 hours, and concentrated to dryness under vacuum. The crude product was purified by HPLC on a Phenomenex Luna C18(2) column (41.4×250 mm) using a 0.9%/min gradient of 9 to 36% acetonitrile containing 0.1% TFA at a flow rate of 80 mL/min. The main product fraction eluting at approximately 21 minutes was lyophilized to give the title compound as a colorless solid (108 mg, 49%, HPLC purity 100%). MS (ESI): 744.5 (95, M+H), 372.9 (100, 2M+H). HRMS: Calcd for C₃₅H₄₇FeN₇O₁₁ (M−2H+Fe): 797.2678; Found: 797.2687.

### EXAMPLE 62

### Synthesis of 2-{[2-({[N-(5-{N-[(4-{[N-((2R)-2-Amino-4-methylpentanoylamino-carbamoyl]methyl}phenyl)methyl]carbamoyl}pentyl)carbamoyl]methyl)}{2-[bis(carboxymethyl)amino]ethyl}amino)ethyl](carboxymethyl)amino}acetic Acid, Trifluoroacetic Acid Salt

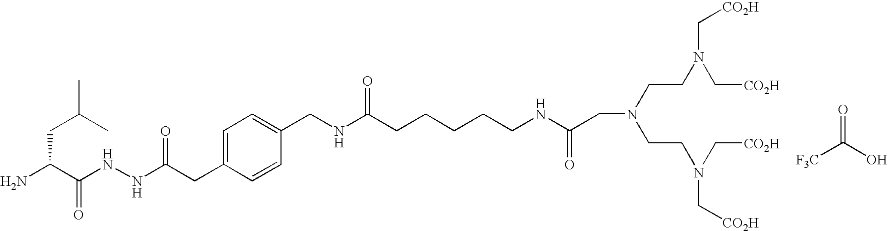

### Part A—Preparation of N-({4-[(N-{(2R)-2-[(tert-Butoxy)carbonylamino]-4-methylpentanoylamino}carbamoyl)methyl]phenyl}methyl)-6-[(fluoren-9-ylmethoxy)carbonylamino]hexanamide

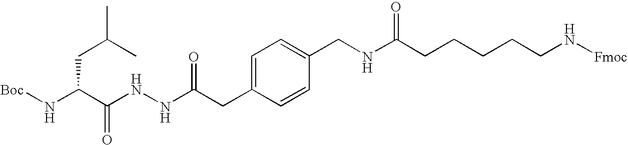

A solution of Boc-D-Leu-OH (1.197 g, 4.8 mmol), HBTU (1.669 g, 4.4 mmol), HOBt (0.679 g, 4.4 mmol), and DIEA (750 μL, 4.4 mmol) in DMF (15 mL) was stirred at room temperature under nitrogen for 30 minutes. The product of Example 4A (1.057 g, 2.0 mmol) was added to the reaction in a single portion, followed by DIEA (750 μL, 4.4 mmol) to give a solution of pH 10. The solution was stirred an additional 5 hours and added dropwise to water (500 mL). The resulting precipitate was collected by filtration and dried to give an off-white solid (940 mg, 76%, HPLC purity 85%). MS (ESI): 515.2 (100, M+H/-Boc), 632.3 (45, M+H); Chiral analysis: 99.5% D-Leu.

The above solid (203 mg, 0.30 mmol) was dissolved in 50:50 DEA:acetonitrile (2.0 mL) and stirred under nitrogen at room temperature for 30 minutes. The solution was concentrated under vacuum and the residue was dissolved in DMF (1.0 mL) and DIEA (150 μL, 0.9 mmol). In a separate flask a solution of Fmoc-6-Ahx-OH (212 mg, 0.60 mmol), HBTU (208 mg, 0.55 mmol), and DIEA (200 μL, 1.20 mmol) in DMF (5.0 mL) was stirred at room temperature under nitrogen for 15 minutes. The two DMF solutions were combined and stirred for an additional 18 hours. The solution was concentrated and the residue was triturated with ethyl acetate (50 mL) to give an off-white solid (100 mg, 46%, HPLC purity 95%). MS (ESI): 628.3 (100, M+H−Boc), 750.3 (35, M+Na). HRMS: Calcd for C₄₁H₅₄N₅O₇ (M+H−Boc): 628.3493; Found: 628.3497; Chiral Analysis: 99.7% D-Leu.

### Part B—Preparation of 2-{[2-({[N-(5-{N-[(4-{[N-((2R)-2-Amino-4-methylpentanoyl-amino)carbamoyl]methyl}phenyl)methyl]carbamoyl}pentyl)carbamoyl]methyl}{2-[bis(carboxymethyl)amino]ethyl}amino)ethyl](carboxymethyl)amino}acetic Acid, Trifluoroacetic Acid Salt

The product of Part A (73 mg, 0.10 mmol) was dissolved in 50:50 DEA:acetonitrile (3.0 mL) and stirred for 30 minutes under nitrogen at ambient temperature. The solution was concentrated under vacuum and the residue was redissolved in DMF (1.0 mL). In a separate flask a solution of2-{bis[2-(bis{[(tert-butyl)oxycarbonyl]methyl}amino)ethyl]amino}acetic acid (74 mg, 0.12 mmol), HBTU (42 mg, 0.11 mmol), and DIEA (50 μL, 0.30 mmol) in DMF (1.0 mL) was stirred at room temperature under nitrogen for 15 minutes. The two DMF solutions were combined and stirred an additional 18 hours. The solution was concentrated and the crude product was dissolved in ethyl acetate (50 mL). The ethyl acetate solution was washed consecutively with 10% citric acid (2×50 mL), saturated NaHCO₃ (2×50 mL), and saturated NaCl (50 mL). The ethyl acetate layer was dried (MgSO₄), filtered, and concentrated to produce a yellow oil. MS (ESI): 1105.6 (50, M+H).

The above oil was dissolved in 90:8:2 TFA:dichloromethane:TIS (10 mL) and stirred at room temperature under nitrogen for 3 hours. The volatiles were removed using reduced pressure. The crude product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27.9% acetonitrile containing 0.1% TFA at a flow rate of 80 mL/min. The product fraction eluting at approximately 29 minutes was lyophilized to give the title compound as a colorless solid (26 mg, 33%, HPLC purity 85%). MS (ESI): 628.3 (100, M+H−Boc), 750.3 (35, M+Na). HRMS: Calcd for C₃₅H₅₄FeN₈O₁₂ (M−2H+Fe): 834.3205; Found: 834.3214; Chiral Analysis: 95.9% D-Leu.

### EXAMPLE 63

### Synthesis of 2-{[2-({[N-({4-[(N-{(2R)-2-Amino-3-[4-(trifluoromethyl)phenyl]-propanoylamino}carbamoyl)methyl]phenyl}methyl)carbamoyl]methyl}{2-[bis(carboxymethyl)amino]ethyl}amino)ethyl](carboxymethyl)amino}acetic Acid, Trifluoroacetic Acid Salt

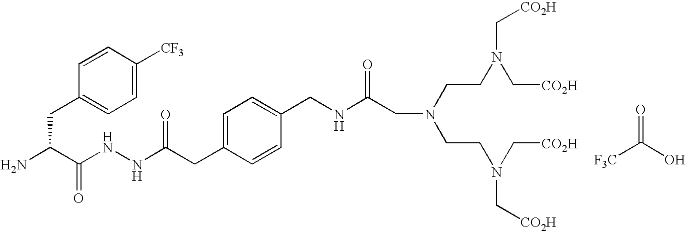

A solution of Boc-D-Phe(CF₃)—OH (466 mg, 1.4 mmol), HBTU (492 mg, 1.3 mmol), HOBt (203 mg, 1.3 mmol), and DIEA (435 μL, 2.5 mmol) in DMF (6.0 mL) was stirred at room temperature under nitrogen for 30 minutes. The product of Example 4A (515 mg, 1.0 mmol) was added to the reaction in a single portion, followed by DIEA (400 μL, 2.3 mmol) to give a pH 10 solution. The solution was stirred an additional 5 hours and added dropwise with stirring to water (500 mL). The resulting precipitate was collected by filtration and dried to give an pale yellow solid (293 mg). MS (ESI): 617.3 (35, M+H−Boc), 739.2 (20, M+Na).

The above solid (179 mg, 0.25 mmol) was dissolved in 50:50 DEA:acetonitrile (5.0 mL) and allowed to stand 30 minutes under nitrogen at ambient temperature. The solution was concentrated and the resulting oily residue was triturated with cyclohexane (3×30 mL). The resulting solid was collected by vacuum filtration and dried to give a flowable yellow powder. This powder was added in a single portion to a previously prepared solution of 2-{bis[2-(bis{[(tert-butyl)oxycarbonyl]methyl}amino)ethyl]amino}acetic acid(185 mg, 0.30 mmol), HBTU (104 mg, 0.28 mmol), and DIEA (175 μL, 1.0 mmol) in DMF (5.0 mL). The solution was stirred at room temperature under nitrogen for 1 hour, and the volatiles were removed under reduced pressure. The resulting oily solid was dissolved in ethyl acetate (50 mL) and washed with 1 N NaOH (50 mL). The ethyl acetate layer was dried (MgSO₄), filtered, and concentrated to give a orange oil. MS (ESI): 1094.4 (100, M+H).

The above oil was dissolved in 90:8:2 TFA:dichloromethane:TIS (10 mL), stirred at room temperature under nitrogen for 6 hours, and concentrated under reduced pressure. The crude product was purified by HPLC on a Phenomenex Luna C18(2) column (41.4×250 mm) using a 0.9%/min gradient of 9 to 36% acetonitrile containing 0.1% TFA at a flow rate of 80 mL/min. The product fraction eluting ati 6.8 minutes was lyophilized to give the title compound as a colorless solid (48 mg, 25%, HPLC purity 100%). MS (ESI): 770.4 (75, M+H), 385.8 (100, M+2H). HRMS: Calcd for C₃₃H₄₀F₃FeN₇O₁₁ (M−2H+Fe): 823.2082; Found: 823.2088.

### EXAMPLE 64

### Synthesis of 2-[(2-{[(N-{[4-({N-[((3R)(3-1,2,3,4-Tetrahydroisoquinolyl))-carbonylamino]carbamoyl}methyl)phenyl]methyl}carbamoyl)methyl]{2-[bis(carboxymethyl)amino]ethyl}amino}ethyl)(carboxymethyl)amino]acetic Acid, Trifluoroacetic Acid Salt

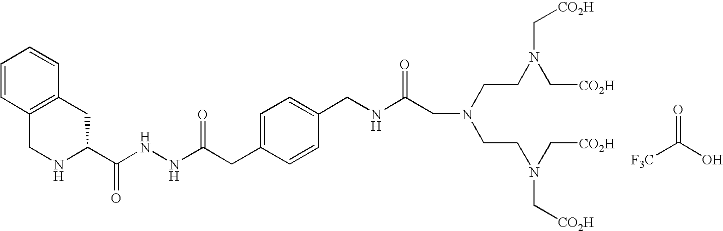

### Part A—Preparation of tert-Butyl (3R)-3-{N-[2-(4-{[(fluoren-9-ylmethoxy)-carbonylamino]methyl}phenyl)acetylamino]carbamoyl}-1,2,3,4-tetrahydroisoquinoline-2-carboxylate

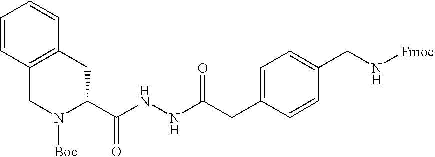

A solution of Boc-D-Tic-OH (333 mg, 1.2 mmol), HBTU (417 mg, 1.1 mmol), HOBt (168 mg, 1.1 mmol), and DIEA (300 μL, 1.7 mmol) in DMF (5.0 mL) was stirred at room temperature under nitrogen for 30 minutes. The product of Example 4A (515 mg, 1.0 mmol) was added to the reaction, followed by DIEA (300 μL, 1.7 mmol) to give a pH 10 solution. The solution was stirred an additional 2 hours, and added dropwise with stirring to water (500 mL). The resulting precipitate was collected by filtration and dried to give the title compound as an off-white solid (571 mg, 86%, HPLC purity 95%). MS (ESI): 561.3 (70, M+H−Boc), 683.3 (30, M+Na).

### Part B—Preparation of 2-[(2-{[(N-{[4-({N-[((3R)(3-1,2,3,4-Tetrahydroisoquinolyl))-carbonylamino]carbamoyl}methyl)phenyl]methyl}carbamoyl)methyl]{2-[bis(carboxymethyl)amino]ethyl}amino}ethyl)(carboxymethyl)amino]acetic Acid, Trifluoroacetic Acid Salt

The product of Part A (330 mg, 0.50 mmol) was dissolved in 50:50 DEA:acetonitrile (4.0 mL), stirred under nitrogen at room temperature for 30 minutes, and concentrated. The resulting solid was triturated with cyclohexane (3×30 mL), and the resulting solid was collected by filtration and dried to give a fine yellow powder. This solid was added to a previously prepared solution of 2-{bis[2-(bis{[(tert-butyl)oxycarbonyl]methyl}amino)ethyl]amino}acetic acid (370 mg, 0.60 mmol), HBTU (208 mg, 0.55 mmol), HOBt (84 mg, 0.55 mmol), and DIEA (350 μL, 2.0 mmol) in DMF (5.0 mL) and the resulting solution was stirred at room temperature under nitrogen for 1 hour. The volatiles were removed under reduced pressure, and the residue was dissolved in ethyl acetate (50 mL). The ethyl acetate solution was washed consecutively with 10% citric acid (2×50 mL), 0.1 N NaOH (2×50 mL), and saturated NaCl (50 mL). The ethyl acetate layer was dried (MgSO₄), filtered, and concentrated to yield a yellow oil. MS (ESI): 1038.5 (100, M+H).

The above oil was dissolved in 90:8:2 TFA:dichloromethane:TIS (10 mL), stirred at room temperature under nitrogen for 6 hours, and concentrated under reduced pressure. The crude product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27% acetonitrile containing 0.1% TFA at a flow rate of 80 mL/min. The main product fraction was lyophilized to give the title compound as a colorless solid (39 mg, 11%, HPLC purity 90%). ¹H NMR (1:1 CD₃CN:D₂O): δ 7.31-7.15 (m, 8H), 4.45-4.23 (m, 5H), 3.86 (s, 2H), 3.68-3.60 (m, 8H), 3.58 (s, 2H), 3.37-3.12 (m, 10H); ¹³C NMR (1:1 CD₃CN:D₂O): δ 173.37, 173.07, 169.09, 168.53, 138.04, 134.52, 131.07, 130.72, 129.96, 129.38, 129.06, 128.62, 128.32, 127.72, 56.47, 56.22, 54.85, 53.07, 51.79, 45.22, 43.82, 40.83, 30.15. MS (ESI): 714.3 (20, M+H), 357.9 (100, M+2H). HRMS: Calcd for C₃₃H₄₁FeN₇O₁₁ (M−2H+Fe): 767.2208: Found: 767.2203.

### EXAMPLE 65

### Synthesis of 2-({2-[({N-[(4-{N-[(2S)-2-Amino-3-(phenylmethylthio)propanoylamino]-carbamoyl}phenyl)methyl]carbamoyl}methyl){2-[bis(carboxymethyl)amino]-ethyl}amino]ethyl}(carboxymethyl)amino)acetic Acid, Trifluoroacetic Acid Salt

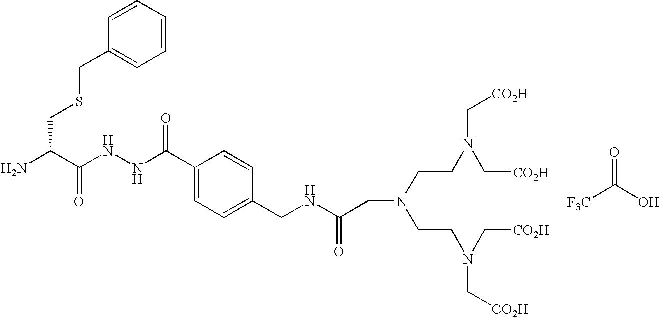

### Part A—Preparation of (2S)—N-{[4-(Aminomethyl)phenyl]carbonylamino}-2-[(tert-butoxy)carbonylamino]-3-(phenylmethylthio)propanamide, Trifluoroacetic Acid Salt

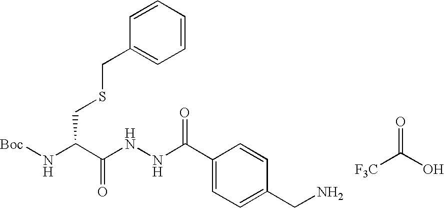

A solution of the intermediate product of Example 29B (445 mg, 1.148 mmol), Boc-D-Cys(Bzl)-OH (250 mg, 0.803 mmol), and DIEA (0.30 mL, 1.693 mmol) in DMF (2.0 mL) was treated with HBTU (365 mg, 0.963 mmol) and stirred at room temperature under nitrogen for 18 hours. The solution was treated with TAEA (0.4 mL) and stirring was continued for an additional 45 minutes. The volatiles were removed under vacuum and the resulting residue was purified by HPLC on a Phenomenex Luna C18(2) column (41.4×250 mm) using a 0.9%/min gradient of 18 to 45% acetonitrile containing 0.1% TFA at a flow rate of 80 mL/min. The main product peak eluting at 21.1 minutes was lyophilized to give the title compound as a colorless solid (148 mg, 40%, HPLC purity 90%). MS (ESI): 917.5 (100, 2M+H), 459.3 (50, M+H), 359.2 (70, M+H−Boc); HRMS Calcd for C₂₃H₃₁N₄O₄S: 481.1880; Found: 481.1886.

### Part B—Preparation of 2-({2-[({N-[(4-{N-[(2S)-2-Amino-3-(phenylmethylthio)-propanoylamino]carbamoyl}phenyl)methyl]carbamoyl}methyl){2-[bis(carboxymethyl)amino]ethyl}amino]ethyl}(carboxymethyl)amino)acetic Acid, Trifluoroacetic Acid Salt

A solution of 2-{bis[2-(bis{[(tert-butyl)oxycarbonyl]methyl}amino)-ethyl]amino}acetic acid (175 mg, 0.283 mmol), the product of Part A (130 mg, 0.283 mmol), and DIEA (99 μL, 0.331 mmol) in DMF (2.0 mL) wag treated with HBTU (129 mg, 0.340 mmol) and stirred at room temperature under nitrogen for 45 minutes. The reaction was diluted with ethyl acetate (100 mL), washed consecutively with 1 N NaOH (3×100 mL) and saturated NaCl (100 mL), dried (MgSO₄), filtered, and concentrated. The resulting residue was dissolved in 90:10:3 TFA:dichloromethane:TIS (15 mL)and the solution was stirred at room temperature under nitrogen for 5 hours. The solution was concentrated under reduced pressure and the crude product was purified by HPLC on a Phenomenex Luna C18(2) column (41.4×250 mm) using a 0.9%/min gradient of 1.8 to 28.8% acetonitrile containing 0.1% TFA at a flow rate of 80 mL/min. The main product peak eluting at 17.7 minutes was lyophilized to give the title compound as a colorless solid (140 mg, 67%, HPLC purity 100%). ¹H NMR (1:1 CD₃CN:D₂O): δ 7.77 (d, J=7.8 Hz, 2H), 7.40 (d, J=7.8 Hz, 2H), 7.38-7.31 (m, 4H), 7.29-7.25 (m, 1H), 4.42 (s, 2H), 3.91 (s, 2H), 3.84 (d, J=13.8 Hz, 1H), 3.81 (d, J=13.8 Hz, 1H), 3.64 (s, 8H), 3.23-3.13 (s, 8H), 3.03 (dd, H_{b} of abc system, J_{ab}=J_{ac}=6.0 Hz, J_{bc}=14 Hz, 1H), 2.94 (dd, H_{c} of abc system, J_{ab}=J_{ac}=6.0 Hz, J_{bc}=14 Hz, 1H), (missing methine was under HOD peak); ¹³C NMR (1:1 CD₃CN:D₂O): δ 173.07, 168.90, 168.58, 168.42, 162.50 (q, J=34.4 Hz), 144.09, 138.55, 131.16, 130.07, 129.84, 128.97, 128.85, 128.52, 117.66 (q, J=291 Hz), 56.26, 55.97, 52.99, 52.25, 51.59, 43.70, 36.80, 32.82. MS (ESI): 734.3 (100, M+H), 367.1 (80, M+2H); HRMS: Calcd for C₃₂H₄₁FeN₇O₁₁S (M+Fe-2H): 787.1929; Found: 787.1938.

### EXAMPLE 66

### Synthesis of 2-({2-[({N-[(4-{2-[N-((2R)-2-amino-4-phenylbutanoylamino)-carbamoyl]ethyl}phenyl)methyl]carbamoyl}methyl){2-[bis(carboxymethyl)amino]-ethyl}amino]ethyl}(carboxymethyl)amino)acetic acid, Trifluoroacetic Acid Salt

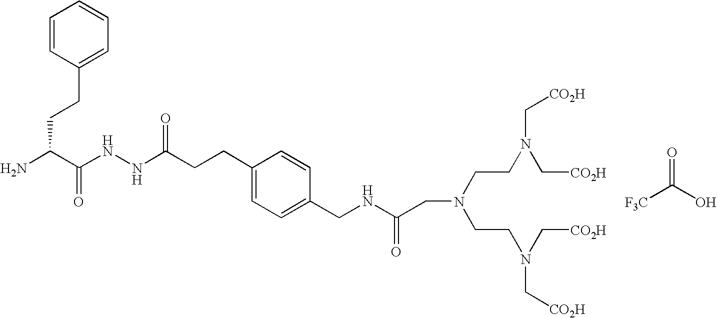

A solution of Boc-D-Hphe-OH (334 mg, 1.2 mmol), HBTU (417 mg, 1.1 mmol), HOBt (168 mg, 1.1 mmol), and DIEA (700 μL, 4.0 mmol) in DMF (5 mL) was stirred at room temperature under nitrogen for 20 minutes. The solution was treated with the product of Example 57B (529 mg, 1.0 mmol), followed by DIEA (650 μL, 3.7 mmol) to give a pH 10 solution. Stirring was continued 1 hour and the volatiles were removed under reduced pressure. The resulting residue was dissolved in ethyl acetate (50 mL) and washed consecutively with 10% citric acid (2×50 mL), 0.1 N NaOH (2×50 mL), and saturated NaCl (50 mL). The ethyl acetate layer was dried (MgSO₄), filtered, and concentrated to give an off-white solid (628 mg). MS (ESI): 577.2 (100, M+H−Boc), 699.2 (10, M+Na).

The above solid was dissolved in 50:50 DEA:acetonitrile (4.0 mL) and stirred under nitrogen at room temperature for 30 minutes. The solution was concentrated and the resulting residue was triturated with cyclohexane (3×30 mL) resulting in the formation of a yellow solid. This solid was added to a previously prepared solution of 2-{bis[2-(bis{[(tert-butyl)oxycarbonyl]methyl}amino)ethyl]amino}acetic acid (370 mg, 0.60 mmol), HBTU (208 mg, 0.55 mmol), HOBt (84 mg, 0.55 mmol), and DIEA (350 μL, 2.0 mmol) in DMF (5.0 mL), and stirring was continued an additional 30 minutes. The solution was concentrated under reduced pressure and the residue was dissolved in ethyl acetate (50 mL). The solution was washed consecutively with 10% citric acid (2×50 mL), 0.1 N NaOH (2×50 mL), and saturated NaCl (50 mL). The ethyl acetate layer was dried (MgSO₄) and concentrated to yield a yellow oil. MS (ESI): 1054.6 (100, M+H).

The above oil was dissolved in 90:9:1 TFA:dichloromethane:TIS (5.0 mL) and stirred at room temperature under nitrogen for 8 hours, and for an additional 2 hours at 40° C. The volatiles were removed under reduced pressure and the crude product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27.9% acetonitrile containing 0.1% TFA at a flow rate of 80 mL/min. The product peak eluting at 29.5 minutes was lyophilized to give the title compound as a colorless solid (46 mg, 12%, HPLC purity 100%). ¹H NMR (9:1 CD₃CN:D₂O): δ 7.33-7.28 (m, 2H), 7.26-7.16 (m, 7H), 4.36 (s, 2H), 4.18 (s, 2H), 3.52 (s, 8H), 3.34 (t, J=5.7 Hz, 4H), 3.09 (t, J=5.7 Hz, 4H), 2.90 (t, J=7.8 Hz, 2H), 2.77-2.66 (m, 2H), 2.55 (t, J=7.8 Hz, 2H), 2.11 (q, J=7.8 Hz, 2H); ¹³C NMR (9:1 CD₃CN:D₂O): δ 174.34, 173.77, 169.11, 166.08, 161.37 (q, J=34.6 Hz), 141.46, 141.05, 137.04, 129.73, 129.47, 128.81, 127.48, 117.48 (q, J=240.0 Hz), 55.97, 55.48, 54.42, 53.06, 50.48, 43.83, 35.98, 33.78, 31.46. MS (ESI): 730.3 (60, M+H); 365.7 (100, M+2H). HRMS: Calcd for C₃₄H₄₅FeN₇O₁₁ (M−2H+Fe): 783.2521; Found: 783.2517; Chiral Analysis: 99.5% D-Hphe.

### EXAMPLE 67

### Synthesis of 2-[(2-{[(N-{[4-(2-{N-[(2S)-2-Amino-3-(phenylmethylthio)-propanoylamino]carbamoyl}ethyl)phenyl]methyl}carbamoyl)methyl]{2-[bis(carboxymethyl)amino]ethyl}amino}ethyl)(carboxymethyl)amino]acetic Acid, Trifluoroacetic Acid Salt

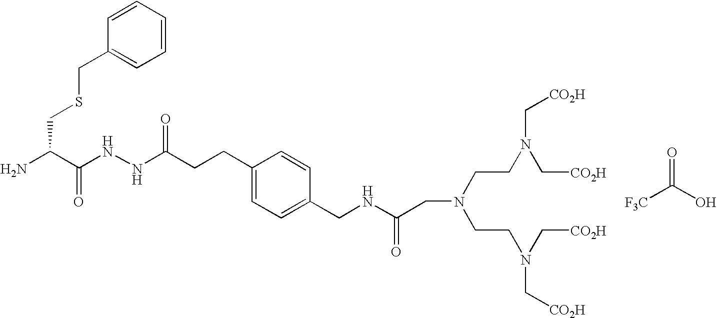

### Part A—Preparation of (2S)-2-[(tert-Butoxy)carbonylamino]-N-[3-(4-{[(fluoren-9-ylmethoxy)carbonylamino]methyl}phenyl)propanoylamino]-3-(phenylmethylthio)-propanamide

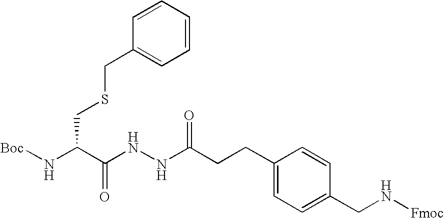

A solution of Boc-D-Cys(Bzl)-OH (374 mg, 1.2 mmol), HBTU (417 mg, 1.1 mmol), HOBt (168 mg, 1.1 mmol), and DIEA (700 μL, 4.0 mmol) in DMF (5.0 mL) was stirred at room temperature under nitrogen for 20 minutes. The solution was treated with the product of Example 57B (529 mg, 1.0 mmol) and DIEA (600 μL, 3.4 mmol) to give a pH 10 solution. The solution was stirred for an additional 18 hours and concentrated under reduced pressure. The resulting residue was dissolved in ethyl acetate (50 mL) and washed consecutively with 10% citric acid (2×50 mL), 0.1 N NaOH (2×50 mL), and saturated NaCl (50 mL). The ethyl acetate layer was dried (MgSO₄) and concentrated to give an off-white solid (607 mg). MS (ESI): 609.2 (100, M+H−Boc), 726.2 (5, M+NH₄).

### Part B—Preparation of 2-[(2-{[(N-{[4-(2-{N-[(2S)-2-Amino-3-(phenylmethylthio)-propanoylamino]carbamoyl}ethyl)phenyl]methyl}carbamoyl)methyl]{2-[bis(carboxymethyl)amino]ethyl}amino}ethyl)(carboxymethyl)amino]acetic Acid, Trifluoroacetic Acid Salt

The above solid was dissolved in 50:50 DEA:acetonitrile (4.0 mL), stirred under nitrogen at room temperature for 30 minutes and concentrated to give a yellow oily solid. The solid was triturated with cyclohexane (3×30 mL) and the resulting solid was collected by filtration and dried to give a fine yellow powder. This powder was added to a previously prepared solution of 2-{bis[2-(bis{[(tert-butyl)oxycarbonyl]methyl}amino)ethyl]amino}acetic acid (370 mg, 0.60 mmol), HBTU (208 mg, 0.55 mmol), HOBt (84 mg, 0.55 mmol), and DIEA (350 μL, 2.0 mmol) in DMF (5.0 mL), and stirred at room temperature under nitrogen for 30 minutes. The volatiles were removed under vacuum and the resulting oily solid was dissolved in ethyl acetate (50 mL). The ethyl acetate solution was washed consecutively with 10% citric acid (2×50 mL), 0.1 N NaOH (2×50 mL), and saturated NaCl (50 mL). The ethyl acetate layer was dried (MgSO₄) and concentrated to give a yellow oil. MS (ESI): 1086.5 (100, M+H).

A solution of the above oil in 90:9:1 TFA:dichloromethane:TIS (5.0 mL) was stirred at room temperature under nitrogen for 8 hours and then for 2 hours at 40° C. The solution was concentrated under reduced pressure and the crude product was purified by HPLC on a Phenomenex Luna C18(2) column (41.4×250 mm) using a method which was isocratic for 10 minutes at 0.9% acetonitrile with a flow rate of 80 mL/min, followed by a 0.9%/min gradient of 0.9 to 27.9% acetonitrile containing 0.1% TFA at a flow rate of 80 mL/min. The product peak eluting at 29.5 minutes was lyophilized to give the title compound as a colorless solid (46 mg, 12%, HPLC purity 100%). ¹H NMR (1:1 CD₃CN:D₂O): δ 7.34-7.30 (m, 4H), 7.29-7.23 (m, 1H), 7.20-7.15 (m, 4H), 4.31 (s, 2H), 4.09 (t, J=6.9 Hz, 1H), 3.95 (s, 2H), 3.76 (q, J=11.6 Hz, 2H), 3.62 (s, 8H), 3.24 (t, J=6.0 Hz, 4H), 3.15 (t, J=6.0 Hz, 4H), 3.00-2.83 (m, 4H), 2.53 (t, J=7.8 Hz, 2H); ¹³C NMR (1:1 CD₃CN:D₂O): δ 174.98, 174.08, 168.63, 168.18, 163.06 (q, J=34.6 Hz), 141.34, 139.08, 137.39, 130.60, 130.38, 130.21, 129.35, 129.07, 118.19 (q, J=290.9 Hz), 56.66, 56.40, 53.99, 52.74, 51.83, 44.34, 37.35, 36.39, 33.28, 31.89. MS (ESI): 762.2 (100, M+H), 381.7 (90, M+2H). HRMS: Calcd for C₃₄H₄₅FeN₇O₁₁S (M−2H+Fe): 815.2242; Found: 815.2243.

### EXAMPLE 68

### Synthesis of 2-{[2-({[N-(5-{N-[(4-{2-[N-((2R)-2-Amino-3-phenylpropanoylamino)-carbamoyl]ethyl}phenyl)methyl]carbamoyl}pentyl)carbamoyl]methyl}{2-[bis(carboxymethyl)amino]ethyl}amino)ethyl](carboxymethyl)amino}acetic Acid, Trifluoroacetic Acid Salt

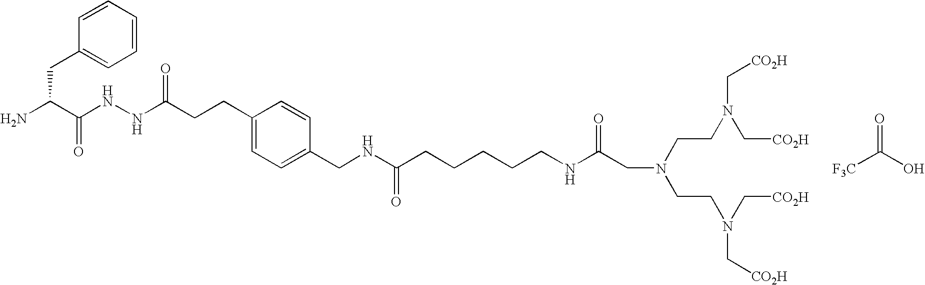

### Part A—Preparation of N-({4-[2-(N-{(2R)-2-[(tert-butoxy)carbonylamino]-3-phenylpropanoylamino}carbamoyl)ethyl]phenyl}methyl)-6-[(fluoren-9-ylmethoxy)carbonylamino]hexanamide

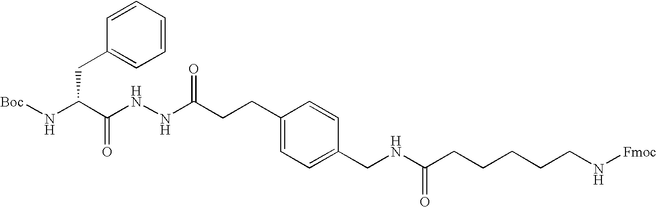

A solution of Boc-d-Phe-OH (477 mg, 1.80 mmol), the product of Example 57B (622 mg, 1.5 mmol), and DIEA (0.80 mL, 4.60 mmol) in DMF (4.0 mL) was treated with HBTU (682 mg, 1.8 mmol) and stirred at ambient temperature under nitrogen for 2 hours. The solution was concentrated under reduced pressure and the resulting viscous amber oil was dissolved in ethyl acetate (400 mL) and washed consecutively with water (100 mL), 10% citric acid (100 mL), 0.5 N NaOH (2×100 mL), water (100 mL), and saturated NaCl (100 mL), dried (MgSO₄), and concentrated to give an off-white solid (1.45 g). MS (ESI): 685.2 (6, M+Na), 563.3 (100, M+H−Boc).

The above solid was dissolved in 1:4 piperidine:DMF (5.0 mL) and stirred at ambient temperature under nitrogen for 30 minutes. The solution was concentrated under reduced pressure and the resulting residue was triturated with cyclohexane (2×50 mL) and dried to give a tan solid (799 mg). MS (ESI): 441.2 (100, M+H).

The above solid (790 mg) and DIEA (0.291 mL, 1.70 mmol) were dissolved in DMF (5.0 mL). In a separate flask a solution of the product of Example 3A (635 mg, 1.80 mmol) and DIEA (0.873 mL, 5.10 mmol) in DMF (4.0 mL) was treated with HBTU (644 mg, 1.70 mmol) and stirred at ambient temperature under nitrogen for 20 minutes. The two DMF solutions were combined and stirring was continued for an additional 2 hours. The solution was concentrated to dryness to yield an oily tan solid. Recrystallization from hot acetonitrile gave an off-white solid (493 mg, 42%, HPLC purity 85%). ¹H NMR (DMSO-d₆): δ 10.02 ((s, 1H), 9.91 (s, 1 H), 8.23 (t, J=6.0 Hz, 1H), 7.88 (d, J=7.8 Hz, 2H), 7.68 (d, J=7.2 Hz, 2H), 7.41 (t, J=7.5 Hz, 2H), 7.36-7.21 (m, 7H), 7.21-7.08 (m, 5H), 6.93 (d, J=8.4 Hz, 1H), 4.29 (d, J=7.2 Hz, 2H), 4.26-4.14 (m, 4H), 3.02-2.91 (m, 3H), 2.81 (t, J=7.8 Hz, 2H), 2.79-2.68 (m, 1 H), 2.43 (t, J=7.8 Hz, 2H), 2.12 (t, J=7.2 Hz, 2H), 1.51 (t, J=7.2 Hz, 2H), 1.40 (t, J=7.2 Hz, 2H), 1.34-1.10 (m, 11H). MS (ESI): 798.3 (7, M+Na), 776.3 (10, M+H), 676.3 (100, M+H−Boc).

### Part B—Preparation of 2-{[2-({[N-(5-{N-[(4-{2-[N-((2R)-2-Amino-3-phenylpropanoylamino)carbamoyl]ethyl}phenyl)methyl]carbamoyl}pentyl)carbamoyl]methyl}{2-[bis(carboxymethyl)amino]ethyl}amino)ethyl](carboxymethyl)amino}acetic Acid, Trifluoroacetic Acid Salt

The product of Part A (484 mg, 0.624 mmol) was dissolved in 1:4 piperidine:DMF (15 mL) and stirred at ambient temperature under nitrogen for 45 minutes. The volatiles were removed under reduced pressure and the resulting residue was triturated with cyclohexane (3×25 mL) to give an off-white solid (324 mg). MS (ESI): 554.2 (100, M+H).

The above solid (111 mg, 0.20 mmol) and DIEA (35 μL, 0.20 mmol) were dissolved in DMF (3.0 mL). In a separate flask a solution of 2-{bis[2-(bis{[(tert-butyl)oxycarbonyl]methyl}amino)ethyl]amino}acetic acid (148 mg, 0.24 mmol) and DIEA (70 μL, 0.40 mmol) in DMF (2.0 mL) was treated with HBTU (83 mg, 0.22 mmol) and stirred at ambient temperature under nitrogen for 10 minutes. The two DMF solutions were combined and stirring was continued for 3 hours. The volatiles were removed under reduced pressure to give a viscous amber oil. This oil was taken up in CHCl₃ (75 mL), washed consecutively with 0.5 N NaOH (25 mL), water (25 mL), and saturated NaCl (25 mL), dried (MgSO₄), and concentrated to give a viscous oil. This oil was dissolved in 90:7:3 TFA:dichloromethane:TIS (10 mL) and heated at 50° C. under nitrogen for 2 hours. The solution was concentrated under reduced pressure and the resulting residue was purified by HPLC on a Phenomenex Luna C18(2) column (41.4×250 mm) using a 0.9%/min gradient of 4.5 to 31.5% acetonitrile containing 0.1% TFA at a flow rate of 80 mL/min. The main product peak eluting at 24 minutes was lyophilized to give the title compound as a colorless solid (145 mg, 77%, HPLC purity 99%). ¹H NMR (1:1 CD₃CN:D₂O): δ 7.35-7.27 (m, 3H), 7.23 (d, J=7.2 Hz, 2H), 7.20-7.13 (m. 4H), 4.23 (s, 2H), 4.18-4.12 (m, 1H), 3.92 (s, 2H), 3.65 (s, 8H), 3.26 (t, J=6.0 Hz, 2H), 3.20-3.07 (m, 4H), 2.84 (t, J=7.8 Hz, 2H), 2.54-2.47 (m, 2H), 2.18 (t, J=7.5 Hz, 2H), 1.53 quin, J=7.8 Hz, 2H), 1.45 quin, J=7.5 Hz, 2H), 1.25 quin, J=7.5 Hz, 2H); ¹³C NMR (1:1 CD₃CN:D₂O): δ 176.47, 174.54, 173.68, 168.78, 167.15, 162.29 (q, J=35.0 Hz), 140.48, 137.56, 134.65, 130.59, 130.08, 129.54, 128.90, 128.56, 117.51 (q, J=290 Hz), 55.96, 55.70, 54.22, 53.50, 51.09, 43.45, 40.26, 37.64, 36.66, 35.88, 31.34, 29.11, 26.84, 26.11. MS (ESI): 829.5 (50, M+H), 415.4 (100, M+2H); HRMS: Calcd for C₃₉H₅₄FeN₈O₁₂ (M+Fe-2H): 882.3205; Found: 882.3199.

### EXAMPLE 69

### Synthesis of 2-{[2-({[N-({4-[N-((2R)-2-Amino-3-naphthylpropanoylamino)-carbamoyl]phenyl}methyl)carbamoyl]methyl}{2-[bis(carboxymethyl)amino]ethyl}-amino)ethyl](carboxymethyl)amino}acetic Acid, Trifluoroacetic Acid Salt

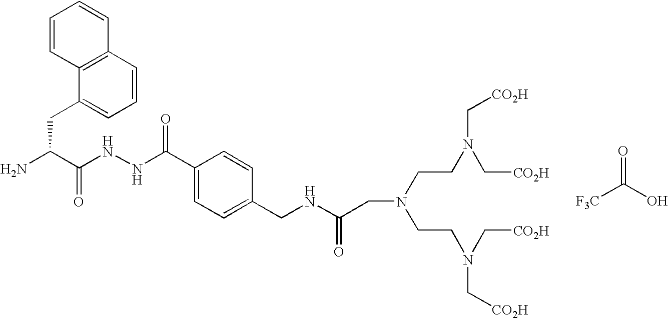

### Part A—Preparation of (2R)—N-{[4-(aminomethyl)phenyl]carbonylamino}-2-[(tert-butoxy)carbonylamino]-3-naphthylpropanamide, 2,2,2-trifluoroacetic acid

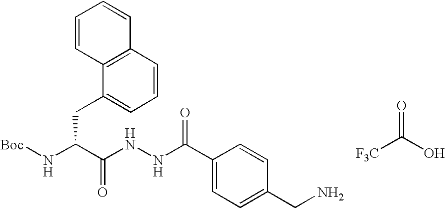

A solution of the intermediate product of Example 29B (439 mg, 1.134 mmol), Boc-D-1-Nal-OH (250 mg, 0.793 mmol), and DIEA (0.276 mL, 1.585 mmol) in DMF (2.0 mL) was treated with HBTU (361 mg, 0.951 mmol) and stirred at room temperature under nitrogen for 18 hours. The solution was treated with TAEA (0.5 mL) and stirring was continued for an additional 45 minutes. The volatiles were removed under vacuum and the resulting residue was purified by HPLC on a Phenomenex Luna C18(2) column (41.4×250 mm) using a 0.9%/min gradient of 18 to 45% acetonitrile containing 0.1% TFA at a flow rate of 80 mL/min. The main product peak eluting at 22.4 minutes was lyophilized to give the title compound as a colorless solid (193 mg, 53%, HPLC purity 90%). MS (ESI): 925.5 (95, 2M+H), 463.4 (100, M+H); HRMS Calcd for C₂₆H₃₁N₄O₄ (M+H): 463.2340; Found: 463.2337.

### Part B—Preparation of 2-{[2-({[N-({4-[N-((2R)-2-Amino-3-naphthylpropanoylamino)carbamoyl]phenyl}methyl)carbamoyl]methyl}{2-[bis(carboxymethyl)amino]ethyl}amino)ethyl](carboxymethyl)amino}acetic Acid, Trifluoroacetic Acid Salt

A solution of 2-{bis[2-(bis{[(tert-butyl)oxycarbonyl]methyl}amino)ethyl]-amino}acetic acid (191 mg, 0.309 mmol), the product of Part A (143 mg, 0.309 mmol), and DIEA (107 μL, 0.608 mmol) in DMF (2.0 mL) was treated with HBTU (141 mg, 0.547 mmol) and stirred at room temperature under nitrogen for 45 minutes. The reaction was diluted with ethyl acetate (80 mL), washed consecutively with 1 N NaOH (2×80 mL) and saturated NaCl (80 mL), dried (MgSO₄), and concentrated. The resulting residue was dissolved in 90:10:3 TFA:dichloromethane:TIS (10 mL) and the solution was stirred at room temperature under nitrogen for 5 hours. The solution was concentrated under reduced pressure and the crude product was purified by HPLC on a Phenomenex Luna C18(2) column (21.2×250 mm) using a 0.9%/min gradient of 6.3 to 24.3% acetonitrile containing 0.1% TFA at a flow rate of 20 mL/min. The main product peak eluting at 18.5 minutes was lyophilized to give the title compound as a colorless solid (180 mg, 79%, HPLC purity 93%). ¹H NMR (1:1 CD₃CN:D₂O): δ 8.72 (d, J=8.4 Hz, 1H), 8.56 (d, J=7.8 Hz, 1H), 8.50 (d, J=7.8 Hz, 1H), 8.32 (d, AA′ portion of AA′BB′ system, J=8.4 Hzm 2H), 8.25-8.20 (m, 1H), 8.19-8.15 (m, 1H), 8.11-8.04 (m, 1H), 7.99, (d, BB′ portion of AA′BB′ system, J=8.4 Hz, 2H), 5.02 (s, 2H), 4.97 (t, J=7.5 Hz, 1H), 4.48 (s, 2H), 4.33-4.21 (m, 10H), 3.80 (s, 8H); ¹³C NMR (1:1 CD₃CN:D₂O): δ 172.86, 169.08, 168.99, 168.89, 162.70 (q, J=34.6 Hz), 144.09, 134.96, 132.49, 131.10, 130.70, 130.05, 129.82, 129.77, 128.96, 128.82, 127.98, 127.31, 126.88, 124.23, 117.52 (q, J=290 Hz), 56.38, 56.08, 53.49, 52.83, 51.78, 43.70, 34.78. MS (ESI): 738.3 (50, M+H), 369.8 (100, M+2H); HRMS: Calcd for C₃₅H₄₁FeN₇O₁₁ (M+Fe-2H): 791.2208; Found: 791.2210.

### EXAMPLE 70

### Synthesis of 2-({2-[({N-[(4-{2-[N-((2R)-2-Amino-3-indol-3-ylpropanoylamino)-carbamoyl]ethyl}phenyl)methyl]carbamoyl}methyl){2-[bis(carboxymethyl)amino]-ethyl}amino]ethyl}(carboxymethyl)amino)acetic Acid, Trifluoroacetic Acid Salt

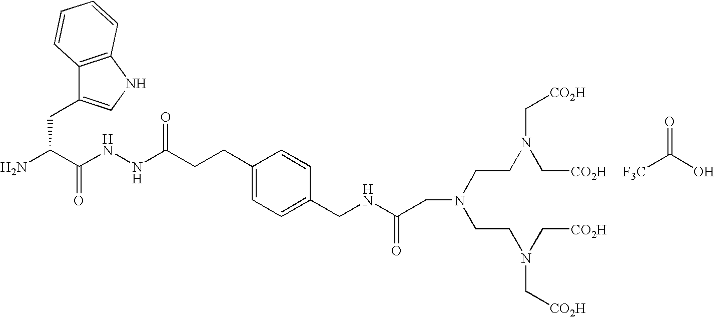

### Part A—Preparation of tert-Butyl 2-{[2-({[N-({4-[2-(N-{(2R)-2-[(tert-Butoxy)-carbonylamino]-3-indol-3-ylpropanoylamino}carbamoyl)ethyl]phenyl}methyl)-carbamoyl]methyl}[2-(bis{[(tert-butyl)oxycarbonyl]methyl}amino)ethyl]amino)-ethyl]{[(tert-butyl)oxycarbonyl]methyl}amino}acetate

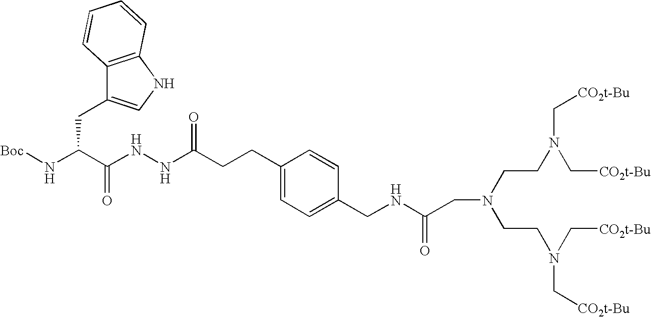

A solution of Boc-D-Trp-OH (120 mg, 0.394 mmol), the product of Example 57B (164 mg, 0.394 mmol), and DIEA (0.137 mL, 0.789 mmol) in DMF (2.0 mL) was treated with HBTU (179 mg, 0.473 mmol) and stirred at room temperature under nitrogen for 20 hours. The reaction solution was diluted with ethyl acetate (100 mL), washed consecutively with 10% citric acid (3×100 mL), 1 N NaOH (3×100), and saturated NaCl (100 mL), dried (MgSO₄), filtered, and concentrated under reduced pressure. The resulting residue was dissolved in 50:50 DEA:acetonitrile (50 mL) and the solution was stirred at room temperature under nitrogen for 45 minutes. The volatiles were removed under vacuum and the resulting residue was triturated by cyclohexane (3×25 mL) to give a colorless solid (158 mg). MS (ESI): 480.4 (100, M+H).

A solution of the above solid (158 mg, 0.329 mmol), 2-{bis[2-(bis{[(tert-butyl)oxycarbonyl]methyl}amino)ethyl]amino}acetic acid (244 mg, 0.395 mmol), and DIEA (115 μL, 0.659 mmol) in DMF (2.0 mL) was treated with HBTU (150 mg, 0.395 mmol) and the solution was stirred at room temperature under nitrogen for 18 hours. The reaction was diluted with ethyl acetate (100 mL), washed consecutively with 1 N NaOH (3×100 mL) and saturated NaCl (100 mL), dried (MgSO₄), and concentrated under reduced vacuum. The volatiles were removed under reduced pressure and the crude product was purified by HPLC on a Phenomenex Luna C18(2) column (41.4×250 mm) using a 0.9%/min gradient of 45 to 72% acetonitrile containing 0.1% TFA at a flow rate of 80 mL/min. The main product peak eluting at 21.9 minutes was lyophilized to give the title compound as a colorless solid (140 mg, 39%, HPLC purity 92%). MS (ESI): 1079.7 (100, M+H), 490.5 (30, M-Boc+2H); HRMS Calcd for C₅₆H₈₇N₈O₁₃ (M+H): 1079.6387; Found: 1079.6372.

### Part B—Preparation of 2-({2-[({N-[(4-{2-[N-((2R)-2-Amino-3-indol-3-ylpropanoylamino)carbamoyl]ethyl}phenyl)methyl]carbamoyl}methyl){2-[bis(carboxymethyl)amino]ethyl}amino]ethyl}(carboxymethyl)amino)acetic Acid, Trifluoroacetic Acid Salt

A solution of the product of Part A (110 mg, 0.102 mmol) in 80:10:10 TFA:dichloromethane:TIS (20 mL) was stirred at room temperature under nitrogen for 4 hours. After removal of the solvents under vacuum, the crude was purified by HPLC on a Phenomenex Luna C18(2) column (21.2×250 mm) using a 0.9%/min gradient of 1.8 to 28.8% acetonitrile containing 0.1% TFA at a flow rate of 20 mL/min. The main product peak eluting at 25.8 minutes was lyophilized to give the title compound as a colorless solid (44 mg, 57%, HPLC purity 94%). ¹H NMR (1:1 CD₃CN:D₂O): δ 7.57 (d, J=7.8 Hz, 1H), 7.42 (d, J=8.4 Hz, 1H), 7.22 (s, 1H), 7.21-7.12 (m, 5H), 7.07 (t, J=7.8 Hz, 1H), 4.31 (s, 2H), the methine signal was under the HOD peak, 3.85 (s, 2H), 3.63 (s, 8H), 3.35 (dd, X portion of AXY system Jₐₓ=7.5 Hz, J_{xy}=14 Hz, 1H), 3.26 (dd, Y portion of AXY system J_{ay}=7.5 Hz, J_{xy}=14 Hz, 1H), 3.22-3.12 (m, 8H), 2.84 (t, J=7.5 Hz, 2H), 2.50 (t, J=7.5 Hz, 2H); ¹³C NMR (1:1 CD₃CN:D₂O): δ 174.56, 173.00, 169.27, 168.36, 162.59 (q, J=34.5 Hz), 140.74, 137.39, 136.91, 129.65, 128.80, 127.79, 126.30, 122.99, 120.45, 119.22, 117.67 (q, J=291 Hz), 112.86, 107.13, 56.35, 56.07, 53.44, 52.99, 51.66, 43.74, 35.82, 31.34, 27.81. MS (ESI): 755.4 (70, M+H), 378.3 (100, M+2H); HRMS: Calcd for C₃₅H₄₄FeN₈O₁₁ (M+Fe-2H): 808.2473; Found: 808.2479.

### EXAMPLE 71

### Synthesis of 2-{[2-({[N-({4-[N-((2R)-2-Amino-5-phenylpentanoylamino)-carbamoyl]phenyl}methyl)carbamoyl]methyl}{2-[bis(carboxymethyl)amino]ethyl}-amino)ethyl](carboxymethyl)amino}acetic Acid, Trifluoroacetic Acid Salt

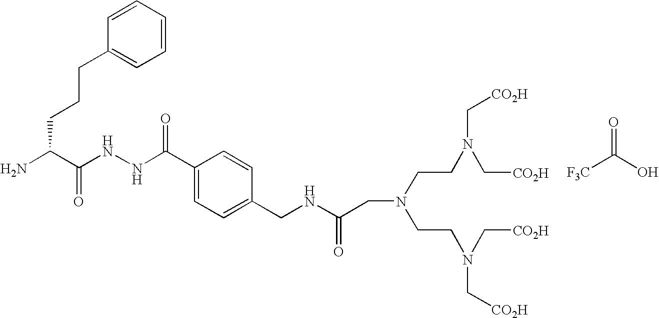

### Part A—Preparation of tert-Butyl 2-[(2-{[(N-{[4-(N-{(2R)-2-[(tert-Butoxy)-carbonylamino]-5-phenylpentanoylamino}carbamoyl)phenyl]methyl}carbamoyl)-methyl][2-(bis{[(tert-butyl)oxycarbonyl]methyl}amino)ethyl]amino}ethyl){[(tert-butyl)oxycarbonyl]methyl}amino]acetate

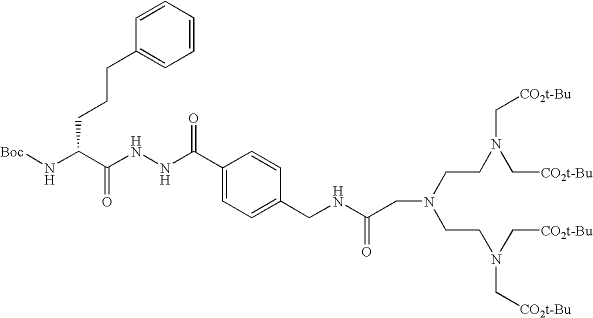

The DCHA salt of (2R)-2-[(tert-butoxy)carbonylamino]-5-phenylpentanoic acid (668 mg, 1.4 mmol) was suspended in ethyl acetate (50 mL), treated with ice-cold 2 M H₂SO₄ (1.2 equiv) and ice-cold water (20 mL), and shaken until the solids were dissolved. The aqueous layer was extracted with additional ethyl acetate (2×20 mL). The combined ethyl acetate layers were washed with water (2×20 mL), dried (MgSO₄), and concentrated under vacuum at a temperature not more than 40° C. to give an oily colorless solid. A solution of this solid, HBTU (492 mg, 1.3 mmol), HOBt (195 mg, 1.3 mmol), and DIEA (1.4 mL, 5.9 mmol) in DMF (5.0 mL) was stir at room temperature under nitrogen for 20 minutes and treated in a single portion with the product of Example 57B (644 mg). Stirring was continued for an additional 20 hours and the volatiles were removed under reduced pressure. The resulting residue was dissolved in ethyl acetate (100 mL) and washed consecutively with 10% citric acid (2×100 mL), 0.3 N NaOH (2×100 mL), and saturated NaCl (50 mL), dried (MgSO₄), and concentrated to yield a yellow oil (240 mg). MS (ESI): 685.2 (10, M+Na) 563.3 (100, M+H−Boc).

The above solid was dissolved in 50:50 DEA:acetonitrile (4.0 mL), stirred under nitrogen at room temperature for 45 minutes and concentrated to give a yellow oily solid. The oily solid was triturated with cyclohexane (3×30 mL) and the resulting solid was collected by filtration and dried to give a fine yellow powder (160 mg). A solution of the above solid (125 mg, 0.284 mmol), 2-{bis[2-(bis{[(tert-butyl)oxycarbonyl]methyl}amino)ethyl]amino}acetic acid (175 mg, 0.284 mmol), and DIEA (99 μL, 0.567 mmol) in DMF (2.0 mL) was treated with HBTU (129 mg, 0.340 mmol), and stirred at room temperature under nitrogen for 18 hours. The reaction solution was diluted with ethyl acetate (100 mL), washed consecutively with 1 N NaOH (3×100 mL), and saturated NaCl (100 mL), dried (MgSO₄), and concentrated under reduced vacuum. The solution was concentrated and the crude was purified by HPLC on a Phenomenex Luna C18(2) column (41.4×250 mm) using a 0.9%/min gradient of 49.5 to 76.5% acetonitrile containing 0.1% TFA at a flow rate of 80 mL/min. The main product peak eluting at 23.0 minutes was lyophilized to give the title compound as a colorless solid (67 mg, 23%, HPLC purity 90%). MS (ESI): 1040.4 (100, M+H).

### Part B—Preparation of 2-{[2-({[N-({4-[N-((2R)-2-Amino-5-phenylpentanoylamino)-carbamoyl]phenyl}methyl)carbamoyl]methyl}{2-[bis(carboxymethyl)amino]ethyl}-amino)ethyl](carboxymethyl)amino}acetic Acid, Trifluoroacetic Acid Salt

The product of Part A (67 mg, 0.064 mmol) was dissolved in 80:10:10 TFA:dichloromethane:TIS (20 mL) and stirred at room temperature under nitrogen for 4 hours. The volatiles were removed under vacuum and the crude was purified by HPLC on a Phenomenex Luna C18(2) column (21.2×250 mm) using a 0.9%/min gradient of 1.8 to 28.8% acetonitrile containing 0.1% TFA at a flow rate of 20 mL/min. The main product peak eluting at 23.3 minutes was lyophilized to give the title compound as a colorless solid (26 mg, 56%, HPLC purity 97%). ¹H NMR (1:1 CD₃CN:D₂O): δ 8.26 (d AA′ portion of AA′BB′ system, J=8.4 Hz, 2H), 7.91 (d BB′ portion of AA′BB′ system, J=8.4 Hz, 2H), 7.79 (t, J=7.2 Hz, 2H), 7.73 (d, J=6.6 Hz, 2H), 7.69 (t, J=6.6 Hz, 1H), 4.92 (s, 2H), 4.51 (t, J=6.6 Hz, 1H), 4.40 (s, 2H), 4.16 (s, 8H), 3.70 (s, 8H), 3.19-3.11 (m, 2H), 2.39 (q, J=6.6 Hz, 2H); ¹³C NMR (1:1 CD₃CN:D₂O): δ 172.97, 169.86, 168.97, 168.75, 162.57 (q, J=34.0 Hz), 144.05, 142.67, 131.24, 129.56, 129.50, 128.94, 128.86, 127.10, 117.67 (q, J=291 Hz), 56.34, 56.04, 53.07, 52.92, 51.71, 43.72, 35.62, 31.48, 26.94. MS (ESI): 716.4 (65, M+H), 358.8 (100, M+2H); HRMS: Calcd for C₃₃H₄₃FeN₇O₁₁ (M+Fe-2H): 769.2364; Found: 769.2368.

### EXAMPLE 72

### Synthesis of [H-D-Cys(Bzl)-Apph]₂EDTA[aminopropoxyethoxyethoxypropylamine-DTPA]₂, Trifluoroacetic Acid Salt

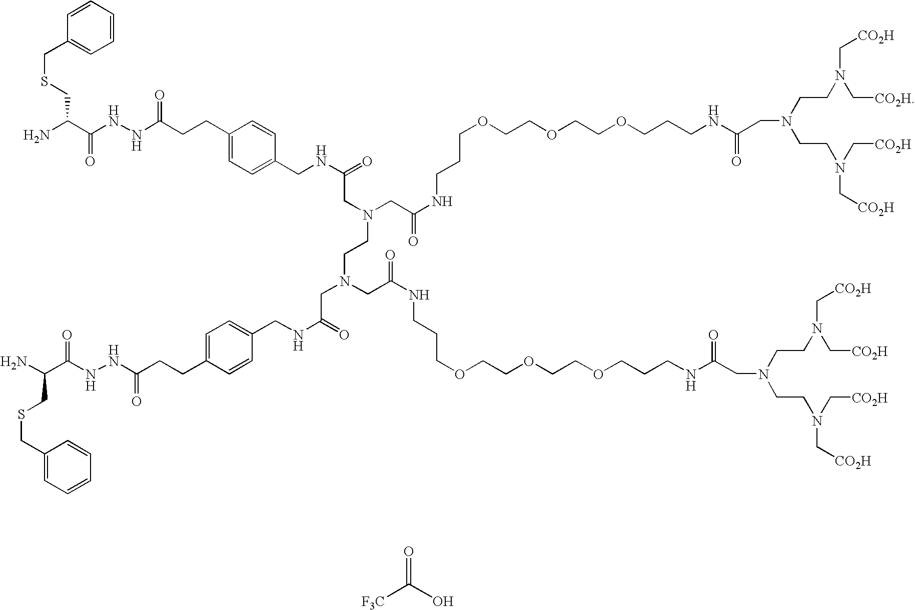

### Part A—Preparation of 2-{{[N-({4-[2-(N-{(2S)-2-[(tert-Butoxy)carbonylamino]-3-(phenylmethylthio)propanoylamino}carbamoyl)ethyl]phenyl}methyl)carbamoyl]methyl}[2-({[N-({4-[2-(N-{(2S)-2-[(tert-butoxy)carbonylamino]-3-(phenylmethylthio)-propanoylamino}carbamoyl)ethyl]phenyl}methyl)carbamoyl]methyl}(carboxymethyl)amino)ethyl]amino}acetic Acid

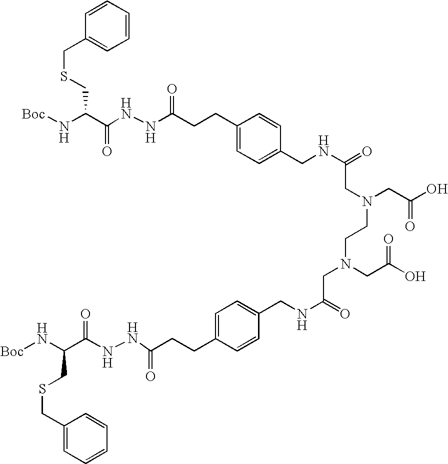

The product of Example 67A (160 mg, 0.263 mmol) was dissolved in 1:4 piperidine:DMF (25 mL) and stirred at ambient temperature under nitrogen for 30 minutes. The volatiles were removed under vacuum and the oily residue was triturated with ether (2×25 mL) to give a slightly oily yellow solid (151 mg). A solution of this solid and DIEA (46 μL, 0.263 mmol) in DMF (5.0 mL) was treated with EDTA dianhydride (35 mg, 0.136 mmol) and stirred at ambient temperature under nitrogen for 4 hours. The solution was concentrated and the resulting residue was triturated with ether (2×2.0 mL) to give an off-white solid (129 mg). This solid was recrystallized from 60:40 acetonitrile:H₂O (6.0 mL) to give the title compound as a colorless solid (64 mg, 39.6%, HPLC purity 94%). MS (ESI): 1229.4 (100, M+H), 515.3 (38, M+2H).

### Part B—Preparation of tert-Butyl 2-{[2-({[N-(3-{2-[2-(3-aminopropoxy)ethoxy]-ethoxy}propyl)carbamoyl]methyl}[2-(bis{[(tert-butyl)oxycarbonyl]methyl}-amino)ethyl]amino)ethyl]{[(tert-butyl)oxycarbonyl]methyl}amino}acetate

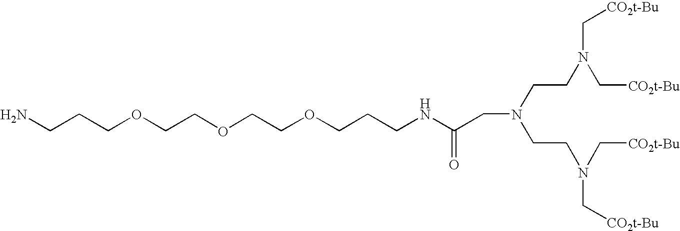

A solution of 2-{bis[2-(bis{[(tert-butyl)oxycarbonyl]methyl}amino)-ethyl]amino}acetic acid (494 mg, 0.800 mmol) and DIEA (0.42 mL, 2.40 mmol) in DMF (5.0 mL) was stirred at ambient temperature under nitrogen for 15 minutes, and treated with a solution of N-(3-{2-[2-(3-aminopropoxy)ethoxy]ethoxy}propyl)-(phenylmethoxy)carboxamide (283 mg, 0.800 mmol) in DMF (4.0 mL). The resulting solution was stirred for 8 hours and concentrated under reduced pressure to a viscous amber oil. This oil was taken up in CHCl₃ (75 mL) and washed consecutively with 10% citric acid (35 mL), 0.5 N NaOH (25 mL), and water (25 mL), dried (MgSO₄), and concentrated to give a viscous amber oil (776 mg). MS (ESI): 954.5 (100, M+H).

The above oil was dissolved in absolute ethanol (75 mL) and hydrogenated over 10% Pd/C (200 mg) on a Parr apparatus at a pressure of 60 psi. After 6 hours the reaction mixture was filtered through Celite®, and the filtrate was concentrated to give the title compound as an amber oil (680 mg). MS (ESI): 820.5 (100, M+H); HRMS: Calcd for C₄₀H₇₈N₅O₁₂ (M+H): 820.5642; Found: 820.5636.

### Part C—Preparation of [H-D-Cys(Bzl)-Apph]₂EDTA-[aminopropoxyethoxyethoxypropylamine-DTPA]₂, Trifluoroacetic Acid Salt

A solution of the product of Part A (63 mg, 0.0513 mmol), the product of Part B (131 mg, 0.160 mmol), and DIEA (66 μL, 0.372 mmol) in DMF (12 mL) was treated with HBTU (57 mg, 0.150 mmol) and stirred at ambient temperature under nitrogen for 6 h. The solution was concentrated under reduced pressure and the resulting residue was purified by HPLC on a Phenomenex Luna C18(2) column (21.2×250 mm) using a 0.9%/min gradient of 54 to 81% acetonitrile containing 0.1% TFA at a flow rate of 20 mL/min. The main product peak eluting as a broad peak from 19 to 26 minutes was lyophilized to give a colorless solid (47 mg).

The above solid was dissolved in 95:3:2 TFA:dichloromethane:TIS (4.0 mL) and heated at 50° C. under nitrogen for 1 hour. The solution was concentrated under reduced pressure and the resulting crude product was purified by HPLC on a Phenomenex Luna C18(2) column (21.2×250 mm) using a 0.6%/min gradient of 9 to 27% acetonitrile containing 0.1% TFA at a flow rate of 20 mL/min. The main product peak eluting at 22.8 minutes was lyophilized to give the title compound as a colorless solid (24 mg, 19%, HPLC purity 100%). MS (ESI): 1092.9 (35, M+2H), 729.0 (100, M+3H), 546.8 (10, M+4H); HRMS calcd for C₉₈H₁₄₇Fe₂N₂₀O₃₂S₂ (M+2Fe-3H): 763.9538; Found: 763.9545.

### EXAMPLE 73

### Synthesis of 2-{[2-({[N-({4-[N-((4E)(2R)-2-amino-5-phenylpent-4-enoylamino)-carbamoyl]phenyl}methyl)carbamoyl]methyl}{2-[bis(carboxymethyl)amino]-ethyl}amino)ethyl](carboxymethyl)amino}acetic acid, Trifluoroacetic Acid Salt

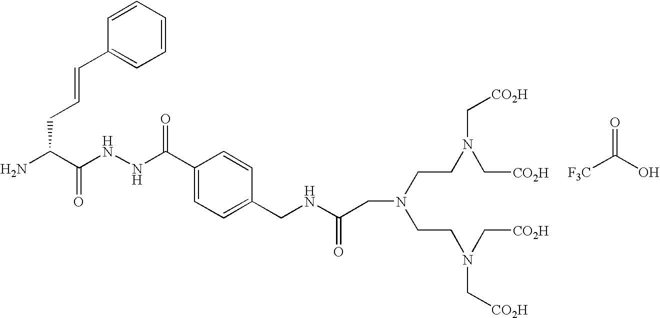

The DCHA salt of Boc-D-Stya-OH (406 mg, 0.859 mmol) was suspended in ethyl acetate (20 mL) in a separating funnel, treated with ice-cold 2 M H₂SO₄ (1.0 mL) and ice-cold water (10 mL), and shaken until the solids were dissolved. The aqueous layer was extracted with additional ethyl acetate (2×20 mL). The combined ethyl acetate layers were washed with water (2×20 mL), dried (MgSO₄), and concentrated under vacuum at a temperature not more than 40° C. to give an oily colorless solid (233 mg). A solution of the above solid, the intermediate product of Example 29B (443 mg, 1.14 mmol), and DIEA (0.279 mL, 1.60 mmol) in DMF (2.0 mL) was treated with HBTU (364 mg, 0.960 mmol) and stirred at room temperature under nitrogen 1 hour. The reaction solution was diluted with ethyl acetate (75 mL), washed consecutively with 10% citric acid (3×50 mL), 1 N NaOH (3×50 mL), and saturated NaCl (50 mL), dried (MgSO₄), and concentrated. The resulting residue was dissolved in 50:50 DEA:acetonitrile (50 mL) and stirred at room temperature under nitrogen for 45 minutes. The volatiles were removed under vacuum and the resulting residue was triturated with cyclohexane (3×50 mL) to give a colorless solid (251 mg). MS (ESI): 877.5 (100, 2M+H), 439.4 (90, M+H).

A solution of the above solid (200 mg, 0.456 mmol), 2-{bis[2-(bis{[(tert-butyl)oxycarbonyl]methyl}amino)ethyl]amino}acetic acid (338 mg, 0.547 mmol), and DIEA (159 μL, 0.912 mmol) in DMF (2.0 mL) was treated with HBTU (208 mg, 0.547 mmol) and stirred at room temperature under nitrogen for 1 hour. The reaction mixture was diluted with ethyl acetate (40 mL), washed consecutively with 10% citric acid (3×50 mL), 0.5 N NaOH (3×40 mL), and saturated NaCl (50 mL), dried (MgSO₄), and concentrated under reduced vacuum. The resulting residue was dissolved in 90:10:3 TFA:dichloromethane:TIS (5 mL) and stirred at room temperature under nitrogen for 5 hours. Volatiles were removed under reduced pressure and the crude product was purified by HPLC on a Phenomenex Luna C18(2) column (41.4×250 mm) using a 0.9%/min gradient of 1.8 to 28.8% acetonitrile containing 0.1% TFA at a flow rate of 80 mL/min. The main product peak eluting at 21.1 minutes was lyophilized to give the title compound as a colorless solid (86 mg, 26%, HPLC purity 96%). ¹H NMR (1:1 CD₃CN:D₂O): δ 7.76 (d, AA′ portion of AA′BB′ system, J=8.4 Hz, 2H), 7.42 (d, AA′ portion of AA′BB′ system, J=7.8 Hz, 2H), 7.40 (d, BB′ portion of AA′BB′ system, J=8.4 Hz, 2H), 7.35-7.27 (m, 2H), 7.25 (t, J=7.2 Hz, 1H), 6.63 (d, J=15.6 Hz, 1H), 6.20 (overlapping d and t, M portion of AMX system, Jₐₘ=15.6 Hz, Jₘₓ=7.8 Hz, 1H), 4.41 (s, 2H), missing methine was under HOD peak, 3.80 (s, 2H), 3.67 (s, 8H), 3.24-3.10 (m, 8H), 2.85-2.76 (m, 2H); ¹³C NMR (1:1 CD₃CN:D₂O): δ 172.50, 169.39, 169.28, 169.03, 162.65 (q, J=35.0 Hz), 144.13, 137.62, 136.66, 131.25, 129.80, 129.04, 128.60, 128.88, 127.54, 122.33, 117.75 (q, J=291 Hz), 56.57, 56.26, 52.83, 52.53, 52.05, 43.68, 35.35. MS (ESI): 714.3 (90, M+H), 357.8 (100, M+2H); HRMS: Calcd for C₃₃H₄₁FeN₇O₁₁ (M+Fe-2H): 767.2208; Found: 767.2206.

### EXAMPLE 74

### Synthesis of N-((2R)-2-{(2S)-2-[(2S)-2-(2-{(2S)-2-[((2S)-1-Acetylpyrrolidin-2-yl)carbonylamino]-N-(4-aminobutyl)-4-methylpentanoylamino}acetylamino)-4-methylpentanoylamino]-4-methylpentanoylamino}-4-methylpentanoylamino)-6-(acetylamino)hexanamide, Trifluoroacetic Acid Salt

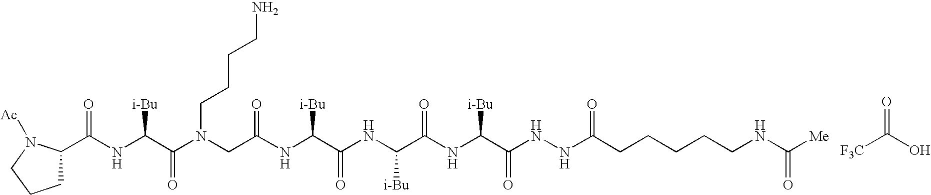

### Part A—Preparation of Ac-PL-NLys(Boc)-LL-OH

The procedure of Example 14, Parts B and C was used to prepare the title compound. Fmoc-Leu-HMPB BHA resin (0.260 g, substitution level=0.54 mmol/g) gave the title compound as a colorless solid (63.6 mg, 63%, HPLC purity 100%). MS (ESI): 725.4 (70, M+H), 625.3 (100, M+H−Boc); HRMS: Calcd for C₃₆H₆₅N₆O₉ (M+H): 725.4808; Found: 725.4809.

### Part B—Preparation of N-[(2S)-2-((2S)-2-{(2S)-2-[2-((2S)-2-[((2S)-1-Acetylpyrrolidin-2-yl)carbonylamino]-N-{4-[(tert-butoxy)carbonylamino]butyl}-4-methylpentanoylamino)acetyl-amino]-4-methylpentanoylamino}-4-methylpentanoylamino)-4-methylpentanoylamino]-6-aminohexanamide, Trifluoroacetic Acid Salt

A solution of the product of Example 34D (10.0 mg, 13.8 μmol) in DMF (1.00 mL) containing HOBt (2.1 mg, 13.7 μmol), i-Pr₂NEt (15.4 μL, 88.4 μmol) and HBTU (5.1 mg, 13.4 μmol) was transferred to a solution of the product of Part 36A (11.0 mg, 18.4 μmol) and i-Pr₂NEt (15.4 μL, 88.4 μmol) in DMF (1.00 mL). After 1 hour at 22° C., the solution was concentrated in vacuo and treated with a solution of piperidine in DMF (1:4 v/v, 2.00 mL) and stirred 0.5 hours. All volatiles were removed in vacuo and the residue purified by HPLC on a Phenomenex Luna C18 column (21.2×250 mm) using a 1.5%/min gradient of 30-60% acetonitrile containing 0.1% TFA at a flow rate of 20 mL/min. The main product peak eluting at 21 minutes was lyophilized to a white solid (8.8 mg, 8.2 μmol, 59%).

### EXAMPLE 75

### Synthesis of N-[(N-{1-[N-(1-{N-[(1R)-1-(N-{(1S)-1-[N-(2-{4-[(Acetylamino)methyl]phenyl}acetylamino)carbamoyl]-3-methylbutyl}carbamoyl)-3-methylbutyl]carbamoyl}(1S)-3-methylbutyl)carbamoyl](1S)-3-phenylpropyl}carbamoyl)methyl](2S)-2-[((2S)-1-acetylpyrrolidin-2-yl)carbonylamino]-N-(4-aminobutyl)-4-methylpentanamide, Trifluoroacetic Acid Salt

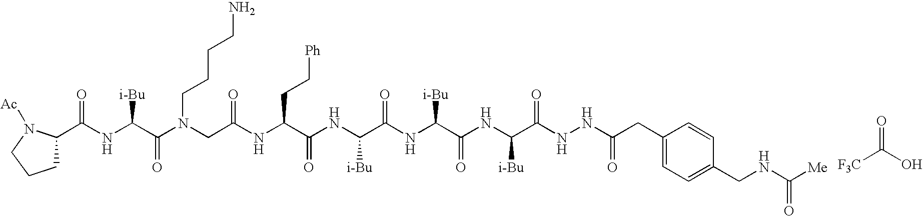

### Part A—Preparation of Fmoc-PL-NLys(Boc)˜Hphe-L-HMPB-BHA Resin

Fmoc-Leu-HMPB BHA resin (4.37 g, substitution level=0.51 mmol/g) was placed in a 200 ml Advanced ChemTech reaction vessel. The resin was swollen by washing with DMF (2×50 mL), and the following steps were performed: (Step 1) The Fmoc group was removed upon exposure to a solution of piperidine in DMF (1:4 v/v, 50 mL) for 0.5 hours. (Step 2) The resin washed with DMF, CH₂Cl₂, methanol, CH₂Cl₂ and DMF (3×50 mL each). (Step 3) A solution of Fmoc-Hphe-OH (2.68 g, 6.69 mmol), HOBt (1.02 g, 6.69 mmol), HBTU (2.54 g, 6.69 mmol) and i-Pr₂NEt (3.88 mL, 22.3 mmol) in DMF (50.0 mL) was added to the resin and the reaction vessel shaken 4 hours. (Step 4) The resin washed with DMF, CH₂Cl₂, methanol, CH₂Cl₂ and DMF (3×50 mL each). (Step 5) The coupling reaction was found to be more than 95% complete as assessed by the fulvene-piperidine assay. Steps 1-5 were repeated with Fmoc-NLys(Boc)-OH, Fmoc-Leu-OH and Fmoc-Pro-OH respectively, to complete the sequence PL-NLys(Boc)˜Hphe-Leu.

### Part B—Preparation of Ac-PL-NLys(Boc)˜Hphe-L-OH

The peptide-resin prepared in Part A was treated with a solution of piperidine in DMF (1:4 v/v, 20 mL) for 0.5 hours, then washed with DMF, CH₂Cl₂, methanol, CH₂Cl₂ and DMF (3×20 mL each). The resin was treated with a solution of Ac₂O (632 μL, 6.69 mmol) and i-Pr₂NEt (1.55 mL, 8.91 mmol) in DMF (20 mL), shaken 2 hours, then washed with DMF, CH₂Cl₂, methanol, and CH₂Cl₂ (3×20 mL each). The resin was transferred to a 60 mL sintered glass funnel of medium porosity, washed with CH₂Cl₂ (20 mL), then treated with a solution of TFA in CH₂Cl₂ (1:99 v/v, 9×20 mL). The suspension was filtered, by the application of pressure, directly into a solution of pyridine in methanol (1:9 v/v, 10.0 mL). The filtrate was concentrated in vacuo and purified by HPLC on a Phenomenex Luna C18 column (41.2×250 mm) using a 1.2%/min gradient of 40-70% acetonitrile containing 0.1% TFA and 10% H₂O at a flow rate of 80 mL/min. The main product peak eluting at 16 minutes was lyophilized to a white solid (553 mg, 0.715 mmol; 32.1%). MS (ESI): 795.6 (11.1, M+Na), 773.6 (41.6, M+H), 673.5 (100, M-Boc). HRMS: Calcd for C₄₀H₆₅N₆O₉: 773.4808; found: 773.4815. The optical purity of the product was established by chiral GLC analysis; 99.5% L-leucine.

### Part C—Preparation of N-({N-[1-(N-{1-[N-(1-{N-[(1R)-1-(N-{2-[4-(Aminomethyl)phenyl]acetylamino}carbamoyl)-3-methylbutyl]carbamoyl}(1S)-3-methylbutyl)carbamoyl](1S)-3-methylbutyl}carbamoyl)(1S)-3-phenylpropyl]carbamoyl}-methyl)(2S)-2-[((2S)-1-acetylpyrrolidin-2-yl)carbonylamino]-N-{4-[(tert-butoxy)carbonylamino]butyl}-4-methylpentanamide, Trifluoroacetic Acid Salt

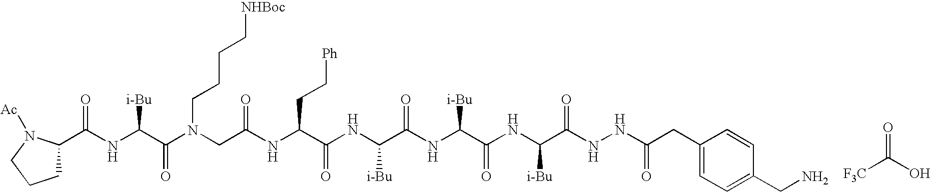

Boc-Leu-DLeu-OH (53.4 mg, 0.155 mmol) (Abdel-Magid, A. F.; Eggmann, U.; Maryanoff, C. A.; Thaler, A; Villani, F. J. Process for the Preparation of KL-4 Pulmonary Polypeptide Surfactant. U.S. Pat. No. 2,147,302, 2002) was treated with a solution of TFA in CH₂Cl₂ (1:1 v/v, 2.00 mL), stirred 0.5 hours, then concentrated in vacuo. The residue was redissolved in DMF (2.00 mL), then treated with i-Pr₂NEt (54.0 μL, 0.310 mmol) and transferred to a previously prepared solution of the product of Part B (120 mg, 0.155 mmol) in DMF (5.00 mL) containing HOBt (23.8 mg, 0.155 mmol), HBTU (58.8 mg, 0.155 mmol) and i-Pr₂NEt (108 μL, 0.620 mmol). After 2 hours at 22° C., the solution was concentrated and the crude oil purified by HPLC on a Phenomenex Luna C18 column (21.2×250 mm) using a 1.2%/min gradient of 40-75% acetonitrile containing 0.1% TFA and 10% H₂O at a flow rate of 20 mL/min. The main product peak eluting at 24 minutes was lyophilized to a white solid (50.0 mg, 50.0 μmol; 32.3%); a later eluting diastereomer was also isolated for a combined yield of 94.0 mg (94.1 μL, 60.7%). The optical purity of the desired material was established by chiral GLC analysis; 76.8% L-leucine.

A solution of the septapeptide (22.0 mg, 22.0 μmol) and HOAt (2.75 mg, 20.0 μmol) in dry DMF (2.00 mL) was successively treated with collidine (16.9 μL, 128 μmol) and DIC (3.1 μL, 20.0 μmol) then stirred 5 minutes at 22° C. The product of Part 4A (9.4 mg, 18 μmol) was added in one portion; additional DMF (2×0.50 mL) was used to wash the sides of the reaction vessel. After 22 hours at 22° C., a second portion of DIC (3.1 μL, 20.0 μmol) and Part 4A (9.4 mg, 18 μmol) were added and stirring continued for an additional 22 hours. With complete consumption of the peptide, all volatiles were then removed in vacuo and the resulting oil treated with a solution of piperidine in DMF (1:4 v/v, 2.00 mL). The solution was stirred 0.3 hours, then concentrated in vacuo and the crude residue purified by HPLC on a Phenomenex Luna C18 column (21.2×250 mm) using a 2.0%/min gradient of 20-60% acetonitrile containing 0. 1% TFA and 10% H₂O at a flow rate of 20 mL/min. The main product peak eluting at 22 minutes was lyophilized to a white solid (7.0 mg, 5.5 μmol; 24.9%). ¹H NMR (DMSO-d₆, 600 MHz): δ(3:2 mixture of rotamers) 10.08 (1H, s), 9.95 (0.6H, s), 9.94 (0.4H, s), 8.34-8.29 (1H, m), 8.18 (0.6H, d, J=8.0 Hz), 8.08 (3H, br s), 8.26-7.98 (1.6H, m), 7.95 (0.6H, d, J=9.5 Hz), 7.91-7.87 (2H, m), 7.84 (0.4H, d, J=7.8 Hz), 7.37 (2H, AB, J_{AB}=8.0 Hz), 7.33 (2H, AB, J_{AB}=8.1 Hz), 7.26 (2H, dd, J=7.6, 7.5 Hz), 7.19-7.15 (3H, m), 6.78 (0.6H, br s), 6.72 (0.4H, br s), 6.49 (1H, br s), 4.78 (0.6H, br s), 4.69 (0.6H, br s) 4.58 (0.6H, br s), 4.53 (0.6H, br s), 4.46-4.42 (1.4H, m), 4.37-4.27 (5.6H, m), 4.13 (1H, d, J=15.2 Hz), 4.02-3.99 (2.6H, m), 3.90 (0.4H, d, J=17.2 Hz), 3.86 (0.6H, d, J=17.2 Hz), 3.75 (0.4H, d, J=16.1 Hz), 3.71 (0.6H, d, J=15.9 Hz), 3.52-3.36 (4H, m), 3.47 (2H, s), 2.92 (1H, br s), 2.87 (1H, dd, J=6.5, 6.2 Hz), 2.58-2.53 (2H, m), 2.24-2.11 (1H, m), 1.98-1.73 (11H, m), 1.57 (7H, br s), 1.48-1.36 (11H, m), 1.36 (9H, s), 1.30-1.24 (1H, m), 0.89-0.71 (24 H, m). MS (ESI): 1160.8 (100, M+H), 531.0 (59.4). HRMS: Calcd for C₆₁H₉₈N₁₁O₁₁: 1160.7442; found: 1160.7458. The optical purity of the desired material was established by chiral GLC analysis; 77.1% L-leucine.

### EXAMPLE 76

### Synthesis of N-{[N-(1-{N-[1-(N-{(1R)-1-[N-((1S)-1-{N-[(1-acetyl(4-piperidyl))carbonylamino]carbamoyl}-3-methylbutyl)carbamoyl]-3-methylbutyl}carbamoyl)(1S)-3-methylbutyl]carbamoyl}(1S)-3-phenylpropyl)carbamoyl]methyl}(2S)-2-[((2S)-1-acetylpyrrolidin-2-yl)carbonylamino]-N-(4-aminobutyl)-4-methylpentanamide, Trifluoroacetic Acid Salt

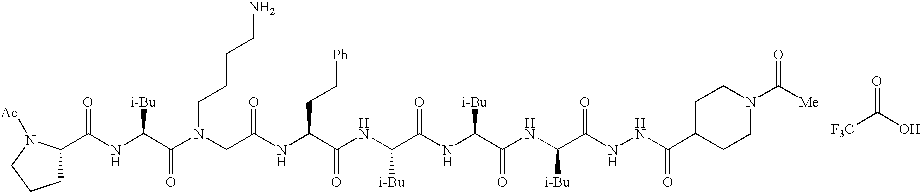

A solution of the septapeptide from Example 75C (22.0 mg, 22.0 μmol) and HOAt (2.75 mg, 20.0 μmol) in dry DMF (2.00 mL) was successively treated with collidine (16.9 μL, 128 μmol) and DIC (3.1 μL, 20.0 μmol) then stirred 5 minutes at 22° C. The product of Part 30A was deprotected with a solution of TFA in CH₂Cl₂ (1:1 v/v) and the resulting salt (8.8 mg, 18.4 μmol) added in one portion to the preactivated solution; additional DMF (2×0.50 mL) was used to wash the sides of the reaction vessel. After 2.5 hours at 22° C., a second portion of DIC (3.1 μL, 20.0 μmol) and the hydrazide (8.8 mg, 18.4 μmol) were added and stirring continued for an additional 24 h; a third addition was performed at the 26 hours time point. After an additional 18 hours, all volatiles were then removed in vacuo and the resulting oil treated with a solution of piperidine in DMF (1:4 v/v, 2.00 mL). The solution was stirred 0.3 hours, then concentrated in vacuo and the residue purified by HPLC on a Phenomenex Luna C18 column (21.2×250 mm) using a 2.0%/min gradient of 20-60% acetonitrile containing 0.1% TFA and 10% H₂O at a flow rate of 20 mL/min. The main product peak eluting at 22 minutes was lyophilized to a white solid (15.5 mg, 12.5 μmol; 56.8%). ¹H NMR (DMSO-d₆, 600 MHz): δ(2:1 mixture of rotamers) 9.90 (0.6H, s), 9.88 (1.4H, s), 8.50 (1H, br s), 8.34-8.29 (1H, m), 8.25-8.17 (1.5H, m), 8.03 (1.2H, m), 7.96 (0.6H, dd, J=8.5, 8.2 Hz), 7.92-7.83 (2H, m), 7.26 (2H, dd, J=7.6, 7.4 Hz), 7.18-7.15 (3H, m), 6.78 (0.5H, br s), 6.72 (0.5H, br s), 6.49 (0.5H, br s), 4.77 (0.4H, br s), 4.68 (0.4H, br s), 4.59 (0.4H, br s), 4.52 (0.4H, br s), 4.47-4.41 (1H, m), 4.37-4.27 (4.6H, m), 4.13 (0.7H, d, J=16.0 Hz), 3.90 (0.4H, d, J=17.3 Hz), 3.86 (0.4H, d, J=17.4 Hz), 3.75 (0.4H, d, J=15.8 Hz), 3.70 (0.4H, d, J=15.9 Hz), 3.51-3.28 (20H, m), 2.94-2.86 (4H, m), 2.59-2.53 (1.7H, m), 2.20-2.10 (0.7H, m), 1.97-1.70 (12.6H, m), 1.61-1.53 (5.7H, m), 1.50-1.36 (9H, s), 1.36 (9H, s), 1.30-1.24 (1.4H, m), 0.89-0.71 (24H, m). MS (ESI): 1124.8 (100, M+H), 513.0 (70.6). HRMS: Calcd for C₅₈H₉₈N₁₁O₁₁: 1124.7442; found: 1124.7440. The optical purity of the desired material was established by chiral GLC analysis; 74.3% L-leucine.

### EXAMPLE 77

### Synthesis of N-[(2R)-3-(4-{(2S)-2-[(2S)-2-(2-{(2S)-2-[((2S)-1-acetylpyrrolidin-2-yl)carbonylamino]-N-(4-aminobutyl)-4-methylpentanoylamino}acetylamino)-4-phenylbutanoylamino]-4-methylpentanoylamino}phenyl)-2-aminopropanoylamino]-6-(acetylamino)hexanamide, Trifluoroacetic Acid Salt

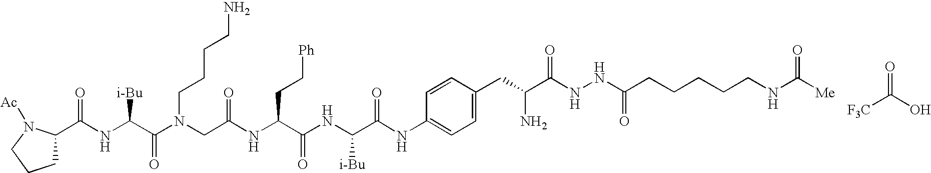

### Part A—Preparation of (2R)-3-[4-((2S)-2-{(2S)-2-[2-((2S)-2-[((2S)-1-acetylpyrrolidin-2-yl)carbonylamino]-N-{4-[(tert-butoxy)carbonylamino]butyl}-4-methylpentanoylamino)acetylamino]-4-phenylbutanoylamino}-4-methylpentanoylamino)phenyl]-2-[(tert-butoxy)carbonylamino]propanoic acid

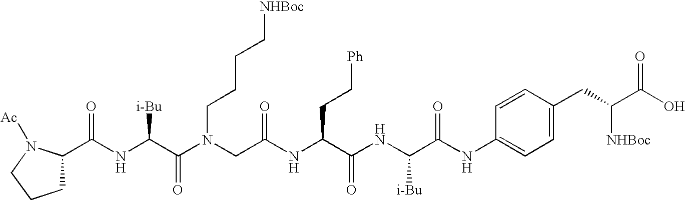

A solution of the product of Example 75B (76.3 mg, 98.7 μmol), HOBt (15.9 mg, 0.104 mmol), i-Pr₂NEt (69.0 μL, 0.396 mmol) and HBTU (39.4 mg, 0.104 mmol) in DMF (3.00 mL) was treated with a solution of Boc-DPhe(4-NH₂)—OH•TFA (35.0 mg, 88.8 μmol) and i-Pr₂NEt (17.0 μL, 97.6 μmol) in DMF (1.00 mL). After stirring 6 hours at 22° C., all volatiles were removed in vacuo and the residue purified by HPLC on a Phenomenex Luna C18 column (21.2×250 mm) using a 0.83%/min gradient of 45-70% acetonitrile containing 0.1% HCO₂H and 10% H₂O at a flow rate of 20 mL/min. The main product peak eluting at 25 minutes was lyophilized to a white solid (4.0 mg, 3.9 μmol; 3.9%); a later eluting diastereomer was also isolated for a combined yield of 8.0 mg (7.7 μL, 7.8%). MS (ESI): 1057.7 (26.9, M+Na), 1035.6 (33.7, M+H), 935.7 (100, M-Boc). The purified material was used directly in the subsequent step.

### Part B—Preparation of N-{(2R)-3-[4-((2S)-2-{(2S)-2-[2-((2S)-2-[((2S)-1-Acetylpyrrolidin-2-yl)carbonylamino]-N-{4-[(tert-butoxy)carbonylamino]butyl}-4-methylpentanoylamino)-acetylamino]-4-phenylbutanoylamino}-4-methylpentanoylamino)phenyl]-2-[(tert-butoxy)carbonylamino]propanoylamino}-6-aminohexanamide, Trifluoroacetic Acid Salt

A solution of the product of Part A (11.0 mg, 10.6 μmol) and HOAt (1.3 mg, 9.5 μmol) in DMF (1.00 mL) was successively treated with collidine (8.2 μL, 6.2 μmol) and DIC (1.5 μL, 9.7 μmol) then stirred 5 minutes at 22° C. The product of Example 3A (4.3 mg, 8.9 μmol) was added in one portion; additional DMF (2×0.50 mL) was used to wash down the sides of the reaction vessel. After 6 hours at 22° C., additional DIC (1.4 μL, 8.9 μmol) and the hydrazide (4.3 mg, 8.9 μmol) were added. After an additional 16 hours at 22° C., all volatiles were then removed in vacuo, and the resulting oil treated with a solution of piperidine in DMF (1:4 v/v, 2.50 mL). The solution was maintained for 0.5 hours, then concentrated in vacuo and the crude residue purified by HPLC on a Phenomenex Luna C18 column (21.2×250 mm) using a 2.0%/min gradient of 20-60% acetonitrile containing 0.1% TFA and 10% H₂O at a flow rate of 20 mL/min. The main product peak eluting at 22 minutes was lyophilized to a white solid (7.0 mg, 5.5 μmol; 51.6%). ¹H NMR (DMSO-d₆, 600 MHz): δ (1:1 mixture of rotamers) 9.93 (1H, s), 9.81 (1H, s), 8.33 (0.4H, dd, J=8.3, 8.1 Hz), 8.28 (0.4H, dd, J=8.0, 5.1 Hz), 8.18 (0.4H, d, J=7.5 Hz), 8.03-7.96 (1.5H, m), 7.61 (3H, br s), 7.49 (2H, br d, J=7.9 Hz), 7.27-7.24 (2H, m), 7.20 (2H, br d, J=8.0 Hz), 7.18-7.15 (3H, m), 6.84 (0.6H, d, J=8.6 Hz), 6.77 (0.4H, br s), 6.71 (0.4H, br s), 6.48 (1.4H, s), 4.79-4.75 (0.2H, m), 4.71-4.67 (0.3H, m), 4.62-4.58 (0.2H, br s), 4.56-4.52 (0.3H, br s), 4.47-4.42 (2H, m), 4.34 (0.5H, dt, J=8.4, 2.9 Hz), 4.21-4.17 (0.3H, m), 4.15 (0.3H, d, J=16.0 Hz), 4.10 (0.4H, d, J=15.8 Hz), 3.92 (0.2H, d, J=17.7 Hz), 3.88 (0.3 H, d, J=17.6 Hz), 3.77 (0.5H, d, J=15.5 Hz), 3.51-3.33 (3H, m), 3.09-2.98 (0.6H, m), 2.93-2.86 (3H, m), 2.80-2.75 (2H, m), 2.70 (1H, dd, J=12.4, 11.9 Hz), 2.59-2.55 (2H, m), 2.14 (2H, t, J=7.3 Hz), 1.98-1.89 (3.6H, m), 1.87-1.70 (5.6H, m), 1.67-1.46 (10.6H, m), 1.41-1.25 (15H, m), 1.28 (9H, s), 0.93-0.85 (9H, m), 0.83 (0.7H, d, J=6.2 Hz), 0.82 (0.7H, d, J=6.6 Hz), 0.77 (0.7H, d, J=6.2 Hz), 0.76 (0.7H, d, J=6.5 Hz). MS (ESI): 1162.8 (100, M+H), 532.0 (52.2). HRMS: Calcd for C₆₀H₉₆N₁₁O₁₂: 1162.7234; found: 1162.7245.

### EXAMPLE 78

### Synthesis of N-[(2R)-2-((2S)-2-{(2S)-2-[(2S)-2-(2-{(2S)-2-[((2S)-1-Acetylpyrrolidin-2-yl)carbonylamino]-N-(4-aminobutyl)-4-methylpentanoylamino}acetylamino)-4-methylpentanoylamino]-4-methylpentanoylamino}-4-methylpentanoylamino)-4-methylpentanoylamino]-6-(acetylamino)hexanamide, Trifluoroacetic Acid Salt

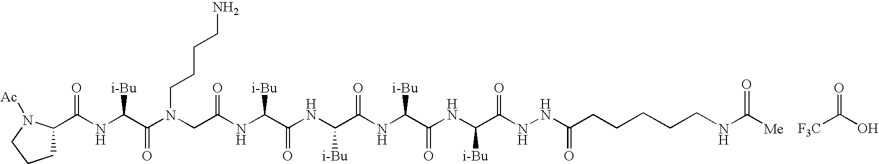

### Part A—Preparation of N-[2-((2R)-2-Amino-4-methylpentanoylamino)(2S)-4-methylpentanoylamino]-6-[(fluoren-9-ylmethoxy)carbonylamino]hexanamide, Trifluoroacetic Acid Salt

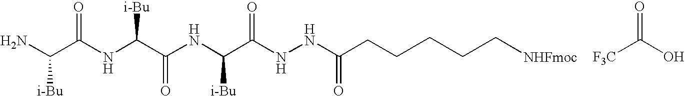

The product of Example 36B (103 mg, 0.146 mmol) was dissolved in dry DMF (1.00 mL) and transferred to a previously prepared solution of Boc-Leu-OH (40.4 mg, 0.175 mmol), HOBt (24.7 mg, 0.161 mmol), i-Pr₂NEt (178 μL, 1.02 mmol) and HBTU (60.8 mg, 0.160 mmol) in DMF (3.00 mL) then stirred 2 hours at 22° C. The resulting solution was concentrated in vacuo and the residue treated with a solution of TFA in CH₂Cl₂ (1:1 v/v, 2.00 mL) at 22° C. After stirring 0.5 hours, all volatiles were removed in vacuo and the residue purified by HPLC on a Phenomenex Luna C18 column (21.2×250 mm) using a 1.0%/min gradient of 15-45% acetonitrile containing 0.1% TFA at a flow rate of 20 mL/min. The main product peak eluting at 33 minutes was lyophilized to a white solid (23.0 mg, 28.0 μmol; 19.2%). MS (ESI): 707.6 (100, M+H).

### Part B—N-{(2R)-2-[(2S)-2-((2S)-2-{(2S)-2-[2-((2S)-2-[((2S)-1-Acetylpyrrolidin-2-yl)carbonylamino]-N-{4-[(tert-butoxy)carbonylamino]butyl}-4-methylpentanoylamino)acetyl-amino]-4-methylpentanoylamino}-4-methylpentanoylamino)-4-methylpentanoylamino]-4-methylpentanoylamino}-6-aminohexanamide, Trifluoroacetic Acid Salt

A solution of the product of Example 14C (20.8 mg, 34.0 μmol) in DMF (2.00 mL) containing HOBt (4.8 mg, 31 μmol), i-Pr₂NEt (34.5 μL, 0.198 mmol) and HBTU (11.8 mg, 31.1 μmol) was treated with the product of A (20.0 mg, 24.4 μmol) then stirred 2 hours at 22° C. The resulting solution was concentrated in vacuo and the residue treated with a previously prepared solution of piperidine in DMF (1:4 v/v, 2.00 mL) at 22° C. After stirring 0.5 hours, all volatiles were removed in vacuo and the residue purified by HPLC on a Phenomenex Luna C18 column (21.2×250 mm) using a 1.0%/min gradient of 22-52% acetonitrile containing 0.1% TFA at a flow rate of 20 mL/min. The main product peak eluting at 25 minutes was lyophilized to a white solid (16.8 mg, 14.1 μmol; 57.8%). MS (ESI): 1264.7 (36.3, M+H), 1078.8 (100, M+H), 490.0 (45.3).

### EXAMPLE 79

### Synthesis of N-[4-((2R)-2-{N-[6-(Acetylamino)hexanoylamino]carbamoyl}-2-aminoethyl)phenyl](2S)-2-[(2S)-2-(2-{(2S)-2-[((2S)-1-acetylpyrrolidin-2-yl)carbonylamino]-4-methylpentanoylamino}acetylamino)-4-phenylbutanoylamino]-6-(amidinoamino)hexanamide, Trifluoroacetic Acid Salt

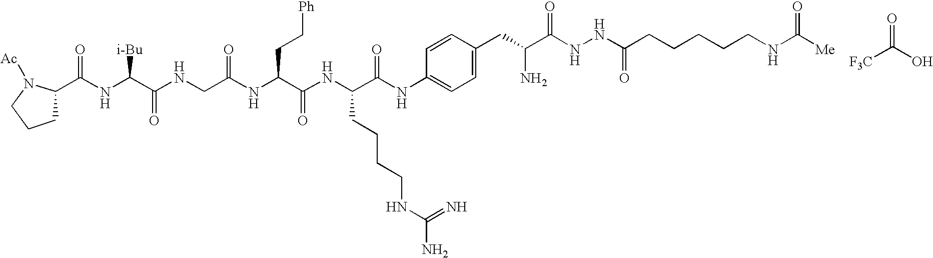

### Part A—Preparation of (2R)-3-(4-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-6-[(iminoethyl)amino]hexanoylamino}phenyl)-2-[(tert-butoxy)carbonylamino]propanoic Acid

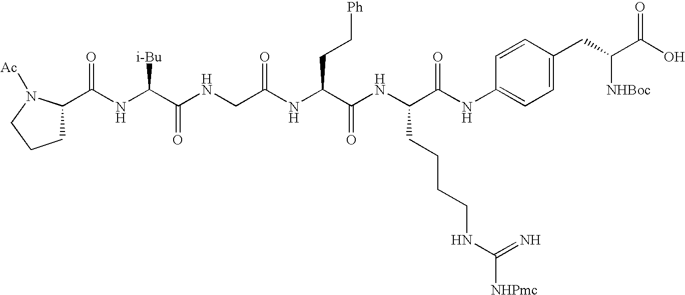

A solution of the product of Example 34D (1.00×10² mg, 0.110 mmol), HOBt (15.5 mg, 0.101 mmol), i-Pr₂NEt (128.0 μL, 0.734 mmol) and HBTU (38.3 mg, 0.101 mmol) in DMF (5.00 mL) was treated with Boc-DPhe(4-NH₂)—OH•TFA (36.2 mg, 91.8 μmol), followed by i-Pr₂NEt (48.0 μL, 0.275 mmol). After stirring 24 hours at 22° C., all volatiles were removed in vacuo and the residue purified by HPLC on a Phenomenex Luna C18 column (41.2×250 mm) using a 1.2%/min gradient of 40-75% acetonitrile containing 0.1% TFA and 10% H₂O at a flow rate of 80 mL/min. The main product peak eluting at 24 minutes was lyophilized to a white solid (17.0 mg, 14.5 □mol; 15.8%). MS (ESI): 1195.7 (12.1, M+Na), 1173.6 (100, M+H), 537.5 (45.2). The purified material was used directly in the subsequent step.

### Part B—Preparation of N-(4-{(2R)-2-Amino-2-[N-(6-aminohexanoylamino)carbamoyl]-ethyl}phenyl)(2S)-2-[(2S)-2-(2-{(2S)-2-[((2S)-1-acetylpyrrolidin-2-yl)carbonylamino]-4-methylpentanoylamino}acetylamino)-4-phenylbutanoylamino]-6-[(imino{[(2,2,5,7,8-pentamethylchroman-6-yl)sulfonyl]amino}methyl)amino]hexanamide, Trifluoroacetic Acid Salt

A solution of the product of Part A (17.0 mg, 14.5 μmol) and HOAt (1.8 mg, 13.1 μmol) in DMF (2.00 mL) was successively treated with collidine (12.0 μL, 90.8 μmol) and DIC (2.2 μL, 14.2 μmol) then stirred 5 minutes at 22° C. The product of Example 3A (6.1 mg, 12.7 μmol) was added in one portion; additional DMF (2×0.50 mL) was used to wash down the sides of the reaction vessel. After 6 hours at 22° C., additional DIC (2.2 μL, 14.2 μmol) and HOAt (1.8 mg, 13.1 μmol) were added. After an additional 16 hours at 22° C., all volatiles were removed in vacuo, and the resulting oil treated with a solution of piperidine in DMF (1:4 v/v, 2.00 mL). The solution was maintained for 0.5 hours, then concentrated in vacuo and the crude residue purified by HPLC on a Phenomenex Luna C18 column (21.2×250 mm) using a 1.3%/min gradient of 10-50% acetonitrile containing 0.1% TFA and 10% H₂O at a flow rate of 20 mL/min. The main product peak eluting at 27 minutes was lyophilized to a white solid (4.0 mg, 2.8 μmol; 22.3%). ¹H NMR (DMSO-d₆, 600 MHz): δ 9.95 (1H, s), 9.84 (1H, m), 8.25-8.20 (1H, m), 8.08-8.01 (2H, m), 7.92 (1H, dd, J=13.9, 7.7 Hz), 7.62 (3H, br s), 7.52-7.48 (2H, m), 7.27-7.20 (4H, m), 7.17-7.15 (3H, m), 6.88 (1H, t, J=8.9 Hz), 6.68 (1H, br s), 6.36 (2H, br s), 4.39-4.30 (3H, m), 4.24 (1H, dd, J=8.3, 3.0 Hz), 4.20-4.15 (2H, m), 3.49-3.28 (3H, m), 3.10-3.01 (2H, m), 2.95-2.91 (1H, m), 2.80-2.68 (3H, m), 2.62-2.52 (4H, m), 2.46-2.44 (6H, m), 2.13 (2H, dd, J=6.9, 6.7 Hz), 2.01-1.94 (6H, m), 1.85-1.70 (7H, m), 1.63-1.38 (9H, m), 1.34-1.28 (9H, m), 1.25 (3H, s), 1.24 (3H, s), 0.86-0.80 (6H, m). MS (ESI): 1300.7 (25.1, M+H), 651.0 (100, M+2H). HRMS: Calcd for C₆₅H₉₈N₁₃O₁₃S: 1300.7122; found: 1300.7099.

### Synthesis of N-((2R)-2-{(2S)-2-[(tert-Butoxy)carbonylamino]-4-methylpentanoylamino}-4-methylpentanoylamino)-6-aminohexanamide, Trifluoroacetic Acid Salt

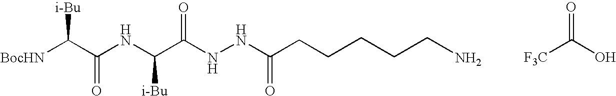

A solution of the product of Example 36B (25.0 mg, 42.1 μmol) in DMF (2.00 mL) was treated with i-Pr₂NEt (50.0 μL, 0.287 mmol) followed by Boc₂O (11.7 mg, 53.6 μL). After 1 hour at 22° C., all volatiles were removed in vacuo and the residue treated with a previously prepared solution of piperidine in DMF (1:4 v/v, 2.00 mL). The solution was maintained for 0.5 hours, then concentrated in vacuo and the crude residue purified by HPLC on a Phenomenex Luna C18 column (21.2×250 mm) using a 1.0%/min gradient of 15-45% acetonitrile containing 0.1% TFA and 10% H₂O at a flow rate of 20 mL/min. The main product peak eluting at 24 minutes was lyophilized to a white solid (4.0 mg, 6.8 μmol; 16.2%). MS (ESI): 472.4 (100, M+H).

### EXAMPLE 81

### Synthesis of 2-{[2-({[N-(5-{N-[(Aminocyclopentyl)carbonylamino]-carbamoyl}pentyl)carbamoyl]methyl}{2-[bis(carboxymethyl)amino]-ethyl}amino)ethyl](carboxymethyl)amino}acetic Acid, Trifluoroacetic Acid Salt

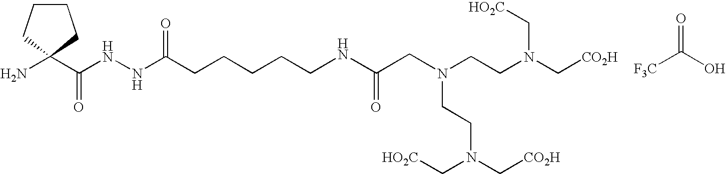

The product of Example 24B (194 mg, 0.412 mmol) was added in one portion to a previously prepared solution of 2-{bis[2-(bis{[(tert-butyl)oxycarbonyl]methyl}amino)-ethyl]amino}acetic acid (312 mg, 0.505 mmol) in DMF (10.0 mL) containing HBTU (172 mg, 0.453 mmol), HOBt (69.0 mg, 0.450 mmol) and i-Pr₂NEt (287 □L, 1.65 mmol). The resulting solution was maintained at 22° C. for 0.6 hours, then concentrated in vacuo and the residue treated with a solution of Et₃SiH in CH₂Cl₂ (9:1 v/v, 400 μL) followed by TFA (3.60 mL, 46.7 mmol). After stirring 3 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 2.5 minutes was lyophilized to a white solid (116 mg, 0.107 mmol; 26.0%). ¹H NMR (DMSO-d₆, 600 MHz): δ 9.94 (1H, br s), 9.82 (1H, s), 8.43 (1H, br t, J=4.5 Hz), 8.37 (2H, br s), 8.21 (2H, br s), 4.12 (2H, br s), 3.50 (8H, s), 3.34 (5H, br s), 3.11 (2H, td, J=6.9, 6.2 Hz); 3.03 (4H, br t, J=5.5 Hz), 2.23-2.18 (2H, m), 2.15 (2H, t, J=7.3 Hz), 1.91-1.80 (6H, m), 1.54 (2H, tt, J=7.5, 7.5 Hz), 1.44 (2H, tt, J=7.3, 7.3 Hz), 1.34-1.28 (2H, m). ¹³C NMR (DMSO-d₆, 151 MHz): δ 172.7, 171.4, 171.4, 157.7 (q, J=30.9 Hz), 117.2 (q, J=300 Hz), 65.2, 54.3, 52.2, 48.7, 40.1, 38.7, 36.2, 33.0, 28.5, 24.6, 24.2. MS (ESI): 632.4 (50.9, M+H), 316.9 (100, M+2H). HRMS: Calcd for C₂₆H₄₃FeN₇O₁₁: 685.2365; found: 685.2354.

### EXAMPLE 82

### Synthesis of 2-{[2-({[N-((2R)-2-Amino-4-methylpentanoylamino)carbamoyl]methyl}{2-[bis(carboxymethyl)amino]ethyl}amino)ethyl](carboxymethyl)amino}acetic Acid, Trifluoroacetic Acid Salt

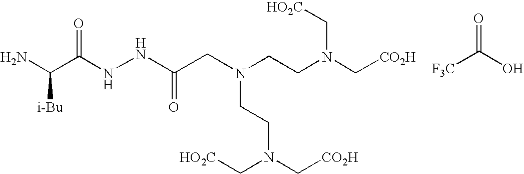

Freshly prepared Boc-D-Leu-NHNH₂ (200 mg, 0.557 mmol) was added in one portion to a previously prepared solution of 2-{bis[2-(bis{[(tert-butyl)oxycarbonyl]methyl}-amino)ethyl]amino}acetic acid (416 mg, 0.673 mmol) in DMF (10.0 mL) containing HBTU (233 mg, 0.614 mmol), HOBt (94.0 mg, 0.614 mmol) and i-Pr₂NEt (388 □L, 2.23 mmol). The resulting solution was maintained at 22° C. for 0.6 hours, then concentrated in vacuo and the residue treated with a solution of Et₃SiH in CH₂Cl₂ (9:1 v/v, 400 μL) followed by TFA (3.60 mL, 46.7 mmol). After stirring 3 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 4.0 minutes was lyophilized to a white solid (104 mg, 0.106 mmol; 19.1%). ¹H NMR (DMSO-d₆, 600 MHz): δ 10.82 (2H, br s), 8.29 (3H, br s), 4.28 (2H, ABq, J_{AB}=15.7 Hz), 3.84 (1H, br s), 3.53 (8H, s), 3.37 (4H, br t, J=5.4 Hz), 3.08 (4H, br t, J=5.9 Hz), 1.77-1.71 (1H, m), 1.60 (2H, ABqdd, J_{AB}=14.1 Hz, J_{dd}=7.3, 7.0 Hz), 0.93 (3H, d, J=6.5 Hz), 0.91 (3H, d, J=6.5 Hz). ¹³C NMR (DMSO-d₆, 151 MHz): δ 172.6, 167.6, 163.7, 157.8 (q, J=31.7 Hz), 116.9 (q, J=299 Hz), 54.3, 52.2, 52.1, 49.6, 48.7, 40.3, 23.4, 22.4, 21.9. MS (ESI): 521.3 (100, M+H), 261.4 (79.4, M+2H). HRMS: Calcd for C₂₀H₃₄FeN₆O₁₀: 574.1680; found: 574.1678. The optical purity of the product was established by chiral GLC analysis; 99.3% D-leucine.

### EXAMPLE 83

### Synthesis of N-[(2R)-3-(4-{(2S)-2-[(tert-Butoxy)carbonylamino]-4-methylpentanoylamino}phenyl)-2-[(tert-butoxy)carbonylamino]propanoylamino]-6-aminohexanamide, Formic Acid Salt

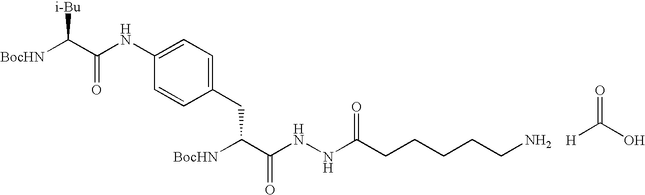

### Part A—Preparation of (2R)-3-(4-{(2S)-2-[(tert-Butoxy)carbonylamino]-4-methylpentanoylamino}phenyl)-2-[(tert-butoxy)carbonylamino]propanoic Acid

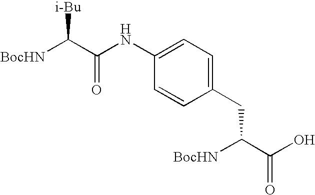

A solution of Boc-LLeu-OH (651 mg, 2.61 mmol) and HOBt (352 mg, 2.30 mmol) in dry DMF (10.0 mL) was successively treated with HBTU (872 mg, 2.30 mmol) and i-Pr₂NEt (1.46 mL, 15.2 mmol) then stirred 5 minutes at 22° C. A solution of Boc-DPhe(4-NH₂)—OH (587 mg, 2.09 mmol) in dry DMF (8.00 mL) was then added dropwise over 5 min; additional DMF (2×1.00 mL) was used to quantitate the transfer. After 1.5 hours at 22° C., the solution was concentrated in vacuo and the crude residue purified by HPLC on a Phenomenex Luna C18 column (41.2×250 mm) using a 1.8%/min gradient of 50-95% acetonitrile containing 0.1% TFA and 10% H₂O at a flow rate of 80 mL/min. The main product peak eluting at 12 minutes was lyophilized to a white solid (302 mg, 0.612 mmol; 29.2%). MS (ESI): 1009.6 (20.9, 2M+H), 887.5 (100), 516.4 (31.9, M+Na). HRMS: Calcd for C₂₅H₃₉N₃O₇Na: 516.2680; found: 516.2679.

### Part B—Preparation of N-[(2R)-3-(4-{(2S)-2-[(tert-Butoxy)carbonylamino]-4-methylpentanoylamino}phenyl)-2-[(tert-butoxy)carbonylamino]propanoylamino]-6-aminohexanamide, Formic Acid Salt

A solution of the product of Part A (77.0 mg, 0.156 mmol) and HOAt (17.8 mg, 0.130 mmol) in dry DMF (3.00 mL) was successively treated with collidine (126 μL, 0.955 mmol) and DIC (2.0×10¹ μL, 0.13 mmol) then stirred 5 minutes at 22° C. The product of Example 3A (50.1 mg, 0.104 mmol) was added in one portion and the resulting solution stirred 5 hours at 22° C.; additional DMF (2×1.00 mL) was used to wash the sides of the reaction vessel. All volatiles were then removed in vacuo and the resulting oil treated with a solution of piperidine in DMF (1:4 v/v, 4.00 mL). The solution was stirred 0.3 hours, then concentrated in vacuo and the crude residue purified by HPLC on a Phenomenex Luna C18 column (21.2×250 mm) using a 1.3%/min gradient of 20-60% acetonitrile containing 0.1% HCO₂H and 10% H₂O at a flow rate of 20 mL/min. The main product peak eluting at 14 minutes was lyophilized to a white solid (20.0 mg, 30.0 μmol; 28.8%). ¹H NMR (DMSO-d₆, 600 MHz): δ9.94 (1H, s), 9.82 (1H, d, J=6.0 Hz), 7.64 (3H, br s), 7.48 (2H, AB, J_{AB}=8.3 Hz), 7.21 (2H, AB, J_{AB}=8.4 Hz), 6.95 (1H, br d, J=7.8 Hz), 6.85 (1H, br d, J=8.7 Hz), 4.19 (1H, br s), 4.11 (1H, br s), 2.93 (1H, dd, J=13.8, 3.3 Hz), 2.77 (2H, br s), 2.70 (1H, dd, J=13.2, 11.1 Hz), 2.13 (2H, dd, J=7.4, 7.2 Hz), 1.63 (1H, br s), 1.56-1.48 (5H, m), 1.37 (9H, s), 1.35-1.25 (2H, m), 1.29 (9H, s), 0.89 (3H, d, J=6.6 Hz), 0.88 (3H, d, J=6.6 Hz). ¹³C NMR (DMSO-d₆, 151 MHz): δ171.5, 170.7(2), 155.4, 155.1, 137.3, 132.7, 129.4, 118.9, 78.0, 54.3, 53.5, 40.7, 38.7, 37.0, 32.8, 28.2, 28.1, 26.7, 25.3, 24.4, 24.3, 22.9, 21.6. MS (ESI): 621.5 (100, M+H). HRMS: Calcd for C₃₁H₅₃N₆O₇: 621.3970; found: 621.3900.

### EXAMPLE 84

### Synthesis of 2-[10-(2-{4-[N-((2R)-2-Amino-4-methylpentanoylamino)-carbamoyl]piperidyl}-2-oxoethyl)-1,4,7,10-tetraaza-4,7-bis(carboxymethyl)cyclododecyl]acetic Acid, Trifluoroacetic Acid Salt

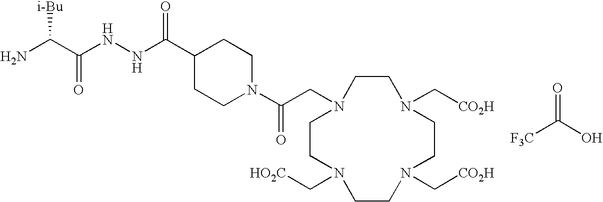

A solution of the product of Example 30B (30.0 mg, 63.8 μmol) and i-Pr₂NEt (11 μL, 63 μmol) in dry DMF (1.00 mL) was transferred to a previously prepared solution of (4,7,10-tris-tert-butoxycarbonylmethyl-1,4,7,10-tetraazacyclododec-1-yl)acetic acid (47.5 mg, 82.9 μmol) in DMF (3.00 mL) containing HBTU (26.6 mg, 70.1 μmol), HOBt (10.7 mg, 69.9 μmol) and i-Pr₂NEt (44 μL, 0.25 mmol); additional DMF (2×0.50 mL) was used to quantitate the transfer. The resulting solution was maintained at 22° C. for 3 hours, then concentrated in vacuo and the residue treated with a solution of Et₃SiH in TFA (9:1 v/v, 3.30 mL). After stirring 2.5 hours at 22° C., the resulting solution was concentrated in vacuo and purified by HPLC on a Phenomenex Luna C18 column (21.2×250 mm) using a 0.67%/min gradient of 0-20% acetonitrile containing 0.1% TFA at a flow rate of 20 mL/min. The main product peak eluting at 5.5 minutes was lyophilized to a white solid (65.0 mg, 53.6 μmol; 84.0%). MS (ESI): 643.3 (65.2, M+H), 530.3 (36.0), 322.3 (100, M+2H), 265.7 (49.7). HRMS: Calcd for C₂₈H₅₁N₈O₉: 643.3774; found: 643.3763. The optical purity of the product was established by chiral GLC analysis; 99.8% D-leucine.

### EXAMPLE 85

### Synthesis of N-{(1R)-3-Methyl-1-[N-(4-piperidylcarbonylamino)carbamoyl]-butyl}(2S)-2-[(tert-butoxy)carbonylamino]-4-methylpentanamide, Trifluoroacetic Acid Salt

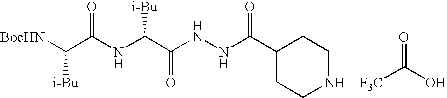

A solution of Boc-Leu-D-Leu-OH (51.7 mg, 0.150 mmol) and HOAt (17.1 mg, 0.125 mmol) in dry DMF (3.00 mL) was successively treated with collidine (92.5 μL, 0.700 mmol) and DIC (19.4 μL, 0.125 mmol) then stirred 5 minutes at 22° C. The product of Example 30A was deprotected with a solution of TFA in CH₂Cl₂ (1:1 v/v) and the resulting salt (48.1 mg, 0.100 mmol) added in one portion to the preactivated solution; additional DMF (0.50 mL) was used to wash the sides of the reaction vessel. After 2.5 hours at 22° C. all volatiles were removed in vacuo, and the crude residue treated with a solution of tris(2-aminoethyl)amine in DMF (1:4 v/v, 2.50 mL); complete deprotection was observed within 0.5 hours. The resulting solution was concentrated and the crude yellow oil purified by HPLC on a Phenomenex Luna C18 column (21.2×250 mm) using a 1.0%/min gradient of 10-40% acetonitrile containing 0.1% TFA and 10% H₂O at a flow rate of 20 mL/min. The main product peak eluting at 15 minutes was lyophilized to a white solid (23.0 mg, 39.4 μmol; 39.3%). ¹H NMR (DMSO-d₆, 600 MHz): δ 9.87 (1H, s), 9.78 (1H, s), 8.44 (1H, br s), 8.21 (1H, br s), 8.00 (1H, d, J=8.4 Hz), 7.70 (1H, br s), 6.91 (1H, d, J=7.5 Hz), 4.34 (1H, ddd, J=9.1, 5.8, 5.7 Hz), 3.96 (1H, ddd, J=8.0, 7.8, 7.0 Hz), 2.92 (3H, br s), 2.61 (1H, dd, J=6.2, 5.5 Hz), 1.85 (2H, dt, J=14.2, 3.4 Hz), 1.77-1.70 (2H, m), 1.64-1.45 (4H, m), 1.40-1.37 (2H, m), 1.36 (9H, s), 0.88 (3H, d, J=6.8 Hz), 0.87 (3H, d, J=6.7 Hz), 0.85 (3H, d, J=6.6 Hz), 0.82 (3H, d, J=6.5 Hz). MS (ESI): 470.4 (100, M+H). HRMS: Calcd for C₂₃H₄₄N₅O₅: 470.3337; found: 470.3341. The optical purity of the product was established by chiral GLC analysis; 51.2% L-leucine.

### EXAMPLE 86

### Synthesis of 2-[(2-{[(N-{5-[N-((2R)-2-Amino-3-methylbutanoylamino)-carbamoyl]pentyl}carbamoyl)methyl]{2-[bis(carboxymethyl)amino]ethyl}-amino}ethyl)(carboxymethyl)amino]acetic Acid, Trifluoroacetic Acid Salt

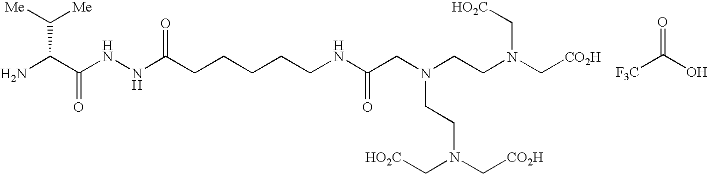

### Part A—Preparation of N-{(2R)-2-[(tert-Butoxy)carbonylamino]-3-methylbutanoylamino}-6-aminohexanamide, Trifluoroacetic Acid Salt

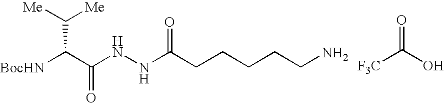

A solution of Boc-D-Val-OH (338 mg, 1.56 mmol) and HOAt (178 mg, 1.30 mmol) in dry DMF (5.00 mL) was successively treated with collidine (963 μL, 7.29 mmol) and DIC (5.00×10² μL, 1.04 mmol) then stirred 5 minutes at 22° C. The product of Example 3A (2.00×10² mg, 0.415 mmol) was added in one portion and the resulting solution stirred 1 hour at 22° C. All volatiles were then removed in vacuo and the resulting oil treated with a solution of piperidine in DMF (1:4 v/v, 5.00 mL). The solution was stirred 0.3 hours, then concentrated in vacuo and the crude residue resuspended in acetonitrile/H₂O (1:1 v/v; 10.0 mL), filtered and lyophilized to a white solid. The crude material thus obtained was purified by HPLC on a Phenomenex Luna C18 column (41.2×250 mm) using a 0.83%/min gradient of 10-35% acetonitrile containing 0.1% TFA and 10% H₂O at a flow rate of 80 mL/min. The main product peak eluting at 16 minutes was lyophilized to a white solid (233 mg, 0.508 mmol; 48.9%). ¹H NMR (DMSO-d₆, 600 MHz): δ 9.76 (1H, s), 9.72 (1H, s), 7.65 (3H, br s), 6.66 (1H, d, J=9.0 Hz), 3.81 (1H, dd, J=8.4, 7.9 Hz), 2.80-2.74 (2H, m), 2.11 (2H, t, J=7.3 Hz), 1.93-1.87 (1H, m), 1.56-1.50 (4H, m), 1.38 (9H, s), 1.34-1.29 (2H, m), 0.91 (3H, d, J=6.8 Hz), 0.86 (3H, d, J=6.7 Hz). ¹³C NMR (DMSO-d₆, 151 MHz): δ170.7, 170.2, 157.8 (q, J=32.2 Hz), 155.2, 116.8 (q, J=299 Hz), 77.9, 58.2, 38.7, 32.8, 30.4, 28.1, 26.7, 25.3, 24.3, 19.1, 18.3. MS (ESI): 345.4 (100, M+H). HRMS: Calcd for C₁₆H₃₃N₄O₄: 345.2496; found: 345.2493.

### Part B—Preparation of 2-[(2-{[(N-{5-[N-((2R)-2-Amino-3-methylbutanoylamino)-carbamoyl]pentyl}carbamoyl)methyl]{2-[bis(carboxymethyl)amino]ethyl}amino}ethyl)-(carboxymethyl)amino]acetic Acid, Trifluoroacetic Acid Salt

The product of Part A (2.00×10² mg, 0.436 mmol) was added in one portion to a previously prepared solution of 2-{bis[2-(bis{[(tert-butyl)oxycarbonyl]methyl}amino)-ethyl]amino}acetic acid (323 mg, 0.523 mmol) in DMF (7.00 mL) containing HBTU (182 mg, 0.480 mmol), HOBt (73.5 mg, 0.480 mmol) and i-Pr₂NEt (303 μL, 1.74 mmol); additional DMF (2×1.50 mL) was used to wash the sides of the reaction vessel. The resulting solution was maintained at 22° C. for 0.6 hours, then concentrated in vacuo and the residue treated with a solution of Et₃SiH in CH₂Cl₂ (9:1 v/v, 400 μL) followed by TFA (3.60 mL, 46.7 mmol). After stirring 2.5 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 8.0 minutes was lyophilized to a white solid (236 mg, 0.219 mmol; 50.3%). MS (ESI): 620.4 (87.4, M+H), 310.9 (100, M+2H). HRMS: Calcd for C₂₅H₄₃FeN₇O₁₁: 673.2365; found: 673.2370. The optical purity of the product was established by chiral GLC analysis; 100.0% D-valine.

### EXAMPLE 87

### Synthesis of 2-{[2-({[N-((2R)-2-Amino-3-phenylpropanoylamino)carbamoyl]methyl}{2-[bis(carboxymethyl)amino]ethyl}amino)ethyl](carboxymethyl)amino}acetic Acid, Trifluoroacetic Acid Salt

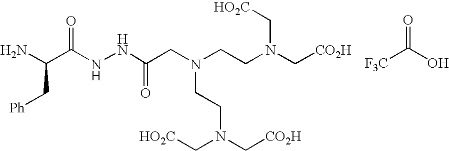

### Part A—Preparation of (2R)—N-Amino-2-[(tert-butoxy)carbonylamino]-3-phenylpropanamide

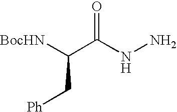

A suspension of Boc-DPhe-OMe (6.00 g, 21.5 mmol) in hydrazine hydrate (15.0 mL, 309 mmol) was warmed slowly to 70° C. over 15 minutes. Upon complete dissolution, the temperature was maintained for 18 hours during which time a white precipitate formed. The resulting suspension was cooled to 22° C., diluted with methanol (50 mL) and concentrated in vacuo. The white solid thus obtained was redissolved in hot ethyl acetate to consume excess hydrazine; upon cooling a heavy white precipitate of acetyl hydrazide formed that was subsequently removed by filtration. The filtrate was washed with saturated solutions of NaHCO₃ (3×50 mL) and NaCl (2×50 mL), then dried over MgSO₄, filtered and concentrated in vacuo to a white solid (4.20 g, 15.0 mmol; 70.0%). The product was used without further purification in the subsequent step. ¹H NMR (DMSO-d₆, 600 MHz): δ 9.09 (1H, s), 7.27-7.17 (5H, m), 6.86 (1H. br d, J=8.7 Hz), 4.20 (2H, br s), 4.11 (1H, ddd, J=9.8, 8.9, 4.8 Hz), 2.87 (1H, dd, J=13.7, 4.7 Hz), 2.74 (1H, dd, J=13.5, 10.2 Hz), 1.29 (9H, s). MS (ESI): 180.1 (86.8, M-Boc), 163.1 (100). The optical purity of the product was established by chiral GLC analysis; 98.5% D-phenylalanine.

### Part B—Preparation of 2-{[2-({[N-((2R)-2-Amino-3-phenylpropanoylamino)-carbamoyl]methyl}{2-[bis(carboxymethyl)amino]ethyl}amino)ethyl]-(carboxymethyl)amino}acetic Acid, Trifluoroacetic Acid Salt

The product of Part A (142 mg, 0.508 mmol) was added in one portion to a previously prepared solution of 2-{bis[2-(bis{[(tert-butyl)oxycarbonyl]methyl}amino)-ethyl]amino}acetic acid (383 mg, 0.620 mmol) in DMF (7.00 mL) containing HBTU (212 mg, 0.559 mmol), HOBt (86.0 mg, 0.562 mmol) and i-Pr₂NEt (353 μL, 2.03 mmol); additional DMF (2×1.50 mL) was used to wash the sides of the reaction vessel. The resulting solution was maintained at 22° C. for 0.6 hours, then concentrated in vacuo and the residue treated with a solution of Et₃SiH in CH₂Cl₂ (9:1 v/v, 400 μL) followed by TFA (3.60 mL, 46.7 mmol). After stirring 2.5 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 8.0 minutes was lyophilized to a white solid (277 mg, 0.274 mmol; 53.9%). MS (ESI): 555.3, (100, M+H), 278.3 (47.0, M+2H). HRMS: Calcd for C₂₃H₃₂FeN₆O₁₀: 608.1524; found: 608.1516. The optical purity of the product was established by chiral GLC analysis; 99.0% D-phenylalanine.

### EXAMPLE 88

### Synthesis of 2-{[2-({[N-(5-{N-[(2R)-2-Amino-3-(4-aminophenyl)propanoylamino]-carbamoyl}pentyl)carbamoyl]methyl}{2-[bis(carboxymethyl)amino]ethyl}-amino)ethyl](carboxymethyl)amino}acetic Acid, Trifluoroacetic Acid Salt

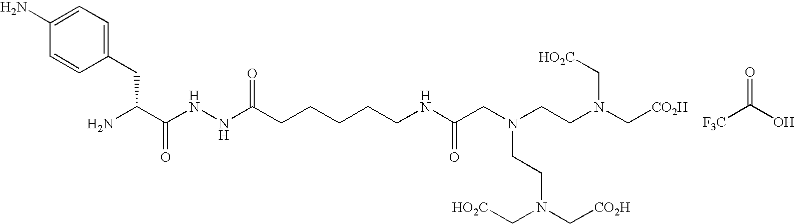

### Part A—Preparation of N-((2R)-2-[(tert-Butoxy)carbonylamino]-3-{4-[(tert-butoxy)carbonylamino]phenyl}propanoylamino)-6-aminohexanamide

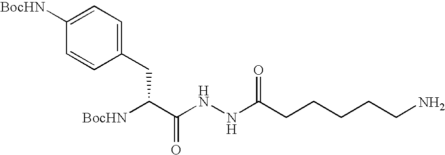

A solution of BOC-DPhe(4-NHBoc)-OH (96.6 mg, 0.254 mmol) and HOBt (35.0 mg, 0.229 mmol) in dry DMF (3.00 mL) was successively treated with HBTU (88.0 mg, 0.232 mmol) and i-Pr₂NEt (1.60×10² μL, 0.919 mmol) then stirred 5 minutes at 22° C. The product of Example 3A (111 mg, 0.231 mmol) was added in one portion and the resulting solution stirred 1 hour at 22° C. The crude reaction mixture thus obtained was partitioned between ethyl acetate (50 mL) and 10% aqueous citric acid (10 mL), the layers separated and the ethyl acetate layer washed with 10% aqueous citric acid (2×10 mL), saturated NaHCO₃ (3×10 mL) and saturated NaCl (10 mL). The resulting ethyl acetate solution was dried over MgSO₄, filtered and concentrated in vacuo to a white foam that was treated with a solution of Et₂NH in acetonitrile (1:1 v/v, 4.00 mL). The solution was stirred 1.5 hours, then concentrated in vacuo to a white foam that was used without further purification in the subsequent step.

### Part B—Preparation of 2-{[2-({[N-(5-{N-[(2R)-2-Amino-3-(4-aminophenyl)-propanoylamino]carbamoyl}pentyl)carbamoyl]methyl}{2-[bis(carboxymethyl)-amino]ethyl}amino)ethyl](carboxymethyl)amino}acetic Acid, Trifluoroacetic Acid Salt

A previously prepared solution of 2-{bis[2-(bis{[(tert-butyl)oxycarbonyl]-methyl}amino)-ethyl]amino}acetic acid (157 mg, 0.254 mmol) in acetonitrile (2.00 mL) containing HBTU (96.0 mg, 0.253 mmol), HOBt (39.0 mg, 0.255 mmol) and Et₃N (128 μL, 0.918 mmol) was transferred to a solution of the product of Part A (117 mg, 0.231 mmol) in acetonitrile (2.00 mL); additional acetonitrile was (2×1.50 mL) was used to quantitate the transfer. The resulting solution was maintained at 22° C. for 0.6 hours, then concentrated in vacuo and the residue treated with a solution of Et₃SiH in CH₂Cl₂ (4:1 v/v, 200 μL) followed by TFA (1.80 mL, 23.4 mmol). After stirring 2 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 7.0 minutes was lyophilized to a white solid (75.0 mg, 65.9 μmol; 28.6%). MS (ESI): 683.4 (81.8, M+H), 342 (100, M+2H). HRMS: Calcd for C₂₉H₄₄FeN₈O₁₁: 736.2474; found: 736.2462.

### EXAMPLE 89

### Synthesis of 2-[(2-{[(N-{5-[N-((2R)-2-Amino-3-cyclohexylpropanoylamino)-carbamoyl]pentyl}carbamoyl)methyl]{2-[bis(carboxymethyl)amino]-ethyl}amino}ethyl)(carboxymethyl)amino]acetic Acid, Trifluoroacetic Acid Salt

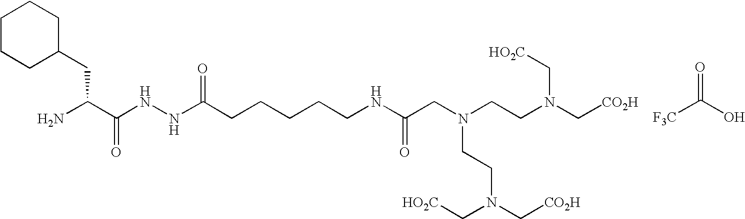

### Part A—Preparation of N-{(2R)-2-[(tert-Butoxy)carbonylamino]-3-cyclohexylpropanoylamino}-6-aminohexanamide

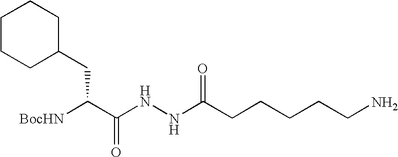

A solution of Boc-DCha-OH•DCHA (566 mg, 1.25 mmol) and HOBt (175 mg, 1.14 mmol) in dry DMF (8.00 mL) was successively treated with HBTU (432 mg, 1.14 mmol) and i-Pr₂NEt (725 μL, 4.16 mmol) then stirred 5 minutes at 22° C. The product of Example 3A (500 mg, 1.04 mmol) was added in one portion and the resulting solution stirred 0.5 hours at 22° C.; additional DMF (2×1.00 mL) was used to wash the sides of the reaction vessel. The crude reaction mixture thus obtained was partitioned between ethyl acetate (150 mL) and 10% aqueous citric acid (15 mL), the layers separated and the ethyl acetate layer washed with 10% aqueous citric acid (2×15 mL), saturated NaHCO₃ (3×15 mL) and saturated NaCl (15 mL). The resulting ethyl acetate solution was further washed with 1M KHSO₄ (2×15 mL) H₂O (15 mL) and saturated NaCl (15 mL), then dried over Na₂SO₄, filtered and concentrated in vacuo. The resulting oil was treated with a solution of Et₂NH in acetonitrile (1:1 v/v, 10.0 mL), stirred 0.3 hours, and then concentrated in vacuo. The resulting oil was used directly in the subsequent step.

### Part B—Preparation of 2-[(2-{[(N-{5-[N-((2R)-2-Amino-3-cyclohexylpropanoylamino)carbamoyl]pentyl}carbamoyl)methyl]{2-[bis(carboxymethyl)-amino]ethyl}amino}ethyl)(carboxymethyl)amino]acetic Acid, Trifluoroacetic Acid Salt

A previously prepared solution of 2-{bis[2-(bis{[(tert-butyl)oxycarbonyl]-methyl}amino)-ethyl]amino}acetic acid (704 mg, 1.14 mmol) in acetonitrile (5.00 mL) containing HBTU (432 mg, 1.04 mmol), HOBt (175 mg, 1.14 mmol) and Et₃N (741 μL, 5.32 mmol) was transferred to a solution of the product of Part A (414 mg, 1.04 mmol) in acetonitrile (2.00 mL); additional acetonitrile was (2×1.50 mL) was used to quantitate the transfer. The resulting solution was maintained at 22° C. for 0.6 hours, then concentrated in vacuo. The residue was redissolved in ethyl acetate (150 mL), washed with saturated solutions of NaHCO₃ (3×15 mL) and NaCl (15 mL), then dried over Na₂SO₄, filtered and concentrated in vacuo. The crude oil was treated with a solution of Et₃SiH in CH₂Cl₂ (4:1 v/v, 400 μL) followed by TFA (3.60 mL, 46.7 mmol). After stirring 2 hours at 22° C., the resulting solution was concentrated in vacuo and purified by HPLC on a Phenomenex Luna C18 column (21.2×250 mm) using a 1.1%/min gradient of 0-40% acetonitrile containing 0.1% TFA at a flow rate of 20 mL/min. The main product peak eluting at 17 minutes was lyophilized to a white solid (393 mg, 0.348 mmol; 33.5%). MS (ESI): 674.5 (100, M+H), 337.9 (42.4, M+2H). HRMS: Calcd for C₂₉H₄₉FeN₇O₁₁: 727.2834; found: 727.2836. The optical purity of the product was established by chiral GLC analysis; 99.8% D-cyclohexylalanine.

### EXAMPLE 90

### Synthesis of 2-({2-[({N-[(4-{N-[(Aminocyclopentyl)carbonylamino]-carbamoyl}phenyl)methyl]carbamoyl}-methyl){2-[bis(carboxymethyl)amino]-ethyl}amino]ethyl}(carboxymethyl)amino)acetic Acid, Trifluoroacetic Acid Salt

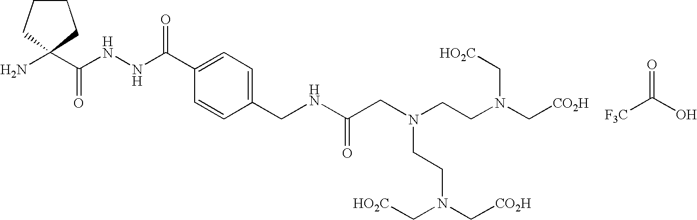

### Part A—Preparation of N-{[4-(Aminomethyl)phenyl]carbonylamino}{[(tert-butoxy)carbonylamino]cyclopentyl}carboxamide

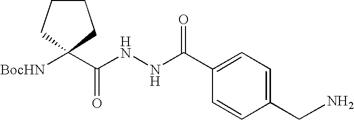

A solution of tert-butoxycarbonylaminocyclopentanecarboxylic acid (2.00×10² mg, 0.872 mmol) and HOBt (122 mg, 0.797 mmol) in dry DMF (5.00 mL) was successively treated with HBTU (303 mg, 0.799 mmol) and i-Pr₂NEt (507 μL, 2.91 mmol) then stirred 5 minutes at 22° C. The product of Example 29A was deprotected with a solution of TFA in CH₂Cl₂ (1:1 v/v) and the resulting salt (365 mg, 728 mmol) added in one portion to the preactivated solution; additional DMF (2×1.00 mL) was used to wash down the sides of the reaction vessel. After 0.5 hours at 22° C., the crude reaction mixture was partitioned between ethyl acetate (150 mL) and 10% aqueous citric acid (15 mL), the layers separated and the ethyl acetate layer washed with 10% aqueous citric acid (2×15 mL), saturated NaHCO₃ (3×15 mL), 1N NaOH (2×15 mL) and saturated NaCl (15 mL), then dried over Na₂SO₄, filtered and concentrated in vacuo. The resulting oil was treated with a solution of Et₂NH in acetonitrile (1:1 v/v, 6.00 mL), stirred 0.3 hours, and then concentrated in vacuo. The resulting oil was used directly in the subsequent step.

### Part B—Preparation of 2-({2-[({N-[(4-{N-[(Aminocyclopentyl)carbonylamino]-carbamoyl}phenyl)methyl]carbamoyl}methyl){2-[bis(carboxymethyl)amino]ethyl}-amino]ethyl}(carboxymethyl)amino)acetic Acid, Trifluoroacetic Acid Salt

A previously prepared solution of 2-{bis[2-(bis{[(tert-butyl)oxycarbonyl]-methyl}amino)-ethyl]amino}acetic acid (449 mg, 0.727 mmol) in acetonitrile (5.00 ML) containing HBTU (276 mg, 0.728 mmol), HOBt (111 mg, 0.725 mmol) and Et₃N (365 μL, 2.62 mmol) was transferred to a solution of the product of Part A (274 mg, 0.728 mmol) in acetonitrile (2.00 mL); additional acetonitrile was (2×1.50 mL) was used to quantitate the transfer. The resulting solution was maintained at 22° C. for 0.6 hours, then concentrated in vacuo and the residue treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 9.0 minutes was lyophilized to a white solid (213 mg, 0.192 mmol; 26.4%). MS (ESI): 652.3 (79.4, M+H), 326.3 (100, M+2H). HRMS: Calcd for C₂₈H₃₉FeN₇O₁₁: 705.2052; found: 705.2038.

### EXAMPLE 91

### Synthesis of 2-{[2-({[N-({4-[N-((2R)-2-Amino-3-cyclohexylpropanoylamino)carbamoyl]phenyl}methyl)carbamoyl]methyl}{2-[bis(carboxymethyl)amino]ethyl}amino)ethyl](carboxymethyl)amino}acetic Acid, Trifluoroacetic Acid Salt

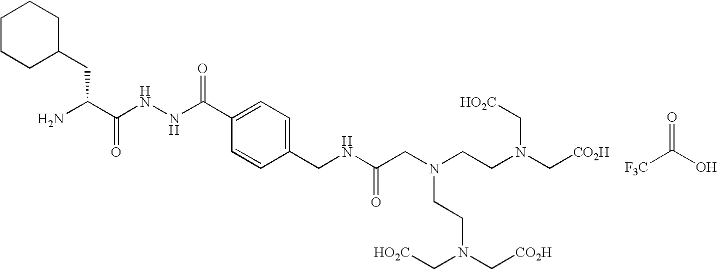

### Part A—Preparation of (2R)—N-{[4-(Aminomethyl)phenyl]carbonylamino}-2-[(tert-butoxy)carbonylamino]-3-cyclohexylpropanamide

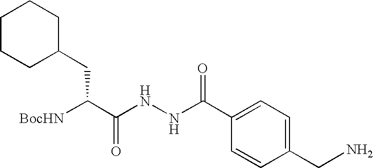

A solution of Boc-DCha-OH•DCHA (272 mg, 0.601 mmol) and HOBt (84.0 mg, 0.549 mmol) in dry acetonitrile (5.00 mL) was successively treated with HBTU (209 mg, 0.551 mmol) and Et₃N (278 μL, 1.99 mmol) then stirred 5 minutes at 22° C. The product of Example 29A was deprotected with a solution of TFA in CH₂Cl₂ (1:1 v/v) and the resulting salt (251 mg, 0.501 mmol) added in one portion to the preactivated solution; subsequent addition of DMF (4.00 mL) was necessary to maintain a homogeneous reaction mixture. After 0.5 hours at 22° C., the crude reaction mixture was diluted with ethyl acetate (100 mL), washed with 10% aqueous citric acid (2×10 mL), 1M KHSO₄ (2×10 mL), saturated NaHCO₃ (3×15 mL) and saturated NaCl (15 mL), then dried over Na₂SO₄, filtered and concentrated in vacuo. The resulting oil was treated with a solution of Et₂NH in acetonitrile (1:1 v/v, 5.00 mL), stirred 0.3 hours, and then concentrated in vacuo. The resulting oil was used directly in the subsequent step.

### Part B—Preparation of 2-{[2-({[N-({4-[N-((2R)-2-Amino-3-cyclohexylpropanoylamino)carbamoyl]phenyl}methyl)carbamoyl]methyl}{2-[bis(carboxymethyl)amino]ethyl}amino)ethyl](carboxymethyl)amino}acetic Acid, Trifluoroacetic Acid Salt

A previously prepared solution of 2-{bis[2-(bis{[(tert-butyl)oxycarbonyl]-methyl}amino)-ethyl]amino}acetic acid (408 mg, 0.660 mmol) in acetonitrile (4.00 mL) containing HBTU (251 mg, 0.662 mmol), HOBt (101 mg, 0.660 mmol) and Et₃N (278 μL, 1.99 mmol) was transferred to a solution of the product of Part A (209 mg, 0.501 mmol) in acetonitrile (3.00 mL); additional acetonitrile was (2×0.50 mL) was used to quantitate the transfer. The resulting solution was maintained at 22° C. for 0.5 hours, then concentrated in vacuo. The residue was redissolved in ethyl acetate (100 mL), washed with saturated solutions of NaHCO₃ (3×10 mL) and NaCl (10 mL), then dried over Na₂SO₄, filtered and concentrated in vacuo. The crude oil was treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 20 minutes was lyophilized to a white solid (187 mg, 0.162 mmol; 32.5%). ¹H NMR (DMSO-d₆, 600 MHz): δ 10.55 (1H, s), 10.52 (1H, s), 9.03 (1H, t, J=5.9 Hz), 8.27 (3H, br s), 7.87 (2H, AA′XX′, J_{AX}=8.3 Hz, J_{AA′}=1.9 Hz), 7.42 (2H, AX, J_{AX}=8.4 Hz), 4.44 (2H, d, J=5.8 Hz), 4.31 (2H, s), 3.91 (1H, br s), 3.52 (8H, s), 3.40 (4H, t, J=5.8 Hz), 3.07 (4H, t, J=5.9 Hz), 1.79-1.74 (2H, m), 1.71-1.59 (5H, m), 1.51 (1H, br s), 1.27-1.11 (3H, m), 0.91 (2H, ABqt, J_{AB}=12.5 Hz, Jₜ=3.4 Hz). ¹³C NMR (DMSO-d₆, 151 MHz): δ 172.6, 168.4, 165.1, 164.9, 157.9 (q, J=32.8 Hz), 142.5, 130.8, 127.6, 127.1, 116.6 (q, J=303 Hz), 54.3, 53.8, 52.2, 49.1, 48.7, 42.0, 32.7, 32.3, 32.0, 25.8, 25.4, 25.3. MS (ESI): 694.4 (94.0, M+H), 347.8 (M+2H). HRMS: Calcd for C₃₁H₄₈N₇O₁₁: 694.3406; found: 694.3407. The optical purity of the product was established by chiral GLC analysis; 94.2% D-cyclohexylalanine.

### EXAMPLE 92

### Synthesis of 2-({2-[(2-{4-[N-((2R)-2-Amino-3-cyclohexylpropanoylamino)-carbamoyl]piperidyl}-2-oxoethyl){2-[bis(carboxymethyl)amino]ethyl}-amino]ethyl}(carboxymethyl)amino)acetic Acid, Trifluoroacetic Acid Salt

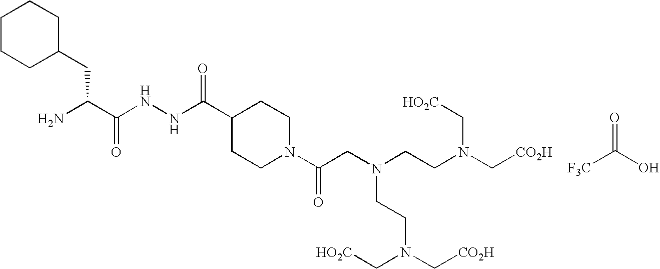

### Part A—Preparation of (2R)-2-[(tert-Butoxy)carbonylamino]-3-cyclohexyl-N-(4-piperidylcarbonylamino)propanamide

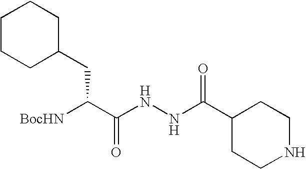

A solution of Boc-DCha-OH•DCHA (272 mg, 0.601 mmol) and HOBt (84.0 mg, 0.549 mmol) in dry acetonitrile (5.00 mL) was successively treated with HBTU (209 mg, 0.551 mmol) and Et₃N (278 μL, 1.99 mmol) then stirred 5 minutes at 22° C. The product of Example 30A was deprotected with a solution of TFA in CH₂Cl₂ (1:1 v/v) and the resulting salt (2.40×10² mg, 0.501 mmol) added in one portion to the preactivated solution. After 0.5 hours at 22° C., the crude reaction mixture was diluted with ethyl acetate (100 mL), washed with 10% aqueous citric acid (2×10 mL), 1M KHSO₄ (2×10 mL), saturated NaHCO₃ (3×15 mL) and saturated NaCl (15 mL), then dried over Na₂SO₄, filtered and concentrated in vacuo. The resulting oil was treated with a solution of Et₂NH in acetonitrile (1 :1 v/v, 5.00 mL), stirred 0.3 hours, and then concentrated in vacuo. The resulting oil was used directly in the subsequent step.

### Part B—Preparation of 2-({2-[(2-{4-[N-((2R)-2-Amino-3-cyclohexylpropanoylamino)carbamoyl]piperidyl}-2-oxoethyl){2-[bis(carboxymethyl)amino]ethyl}amino]ethyl}(carboxymethyl)amino)acetic Acid, Trifluoroacetic Acid Salt

A previously prepared solution of 2-{bis[2-(bis{[(tert-butyl)oxycarbonyl]-methyl}amino)-ethyl]amino}acetic acid (3.40×10² mg, 0.550 mmol) in acetonitrile (4.00 mL) containing HBTU (209 mg, 0.551 mmol), HOBt (84.0 mg, 0.549 mmol) and Et₃N (278 μL, 1.99 mmol) was transferred to a solution of the product of Part A (198 mg, 0.501 mmol) in acetonitrile (3.00 mL); additional acetonitrile was (2×0.50 mL) was used to quantitate the transfer. The resulting solution was maintained at 22° C. for 0.5 hours, then concentrated in vacuo. The residue was redissolved in ethyl acetate (100 mL), washed with saturated solutions of NaHCO₃ (3×10 mL) and NaCl (10 mL), then dried over Na₂SO₄, filtered and concentrated in vacuo. The crude oil was treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 18 minutes was lyophilized to a white solid (145 mg, 0.129 mmol; 25.7%). ¹H NMR (DMSO-d₆, 600 MHz): δ 10.44 (1H, d, J=9.1 Hz), 10.13 (1H, J=10.5 Hz), 8.22 (3H, br s), 4.67 (2H, AB, J_{AB}=16.1 Hz), 4.32 (1H, br d, J=12.6 Hz), 3.82 (1H, br s), 3.64 (1H, br d, J=12.5 Hz), 3.48 (8H, s), 3.36 (4H, br s), 3.06 (5H, br t, J=5.5 Hz), 2.77 (1H, dd, J=12.1, 11.4 Hz), 2.55 (1H, br s), 1.79-1.41 (13H, m), 1.23-1.08 (3H, m), 0.90-0.84 (2H, m). ¹³C NMR (DMSO-d₆, 151 MHz): δ 172.7, 172.5, 167.8, 163.2, 157.9 (q, J=33.2 Hz), 116.5 (q, J=297 Hz), 54.2, 52.4, 49.0, 48.8, 43.4, 40.9, 38.8, 32.6, 32.3, 32.0, 28.0, 27.7, 25.8, 25.5, 25.3. MS (ESI): 672.3 (88.9, M+H), 336.8 (100, M+2H). HRMS: Calcd for C₂₉H₅₀N₇O₁₁: 672.3563; found: 672.3565. The optical purity of the product was established by chiral GLC analysis; 91.2% D-cyclohexylalanine.

### EXAMPLE 93

### Synthesis of 2-{[2-({[N-({4-[N-((2R)-2-Amino-3-methylbutanoylamino)-carbamoyl]phenyl}methyl)carbamoyl]methyl}{2-[bis(carboxymethyl)amino]-ethyl}amino)ethyl](carboxymethyl)amino}acetic Acid, Trifluoroacetic Acid Salt

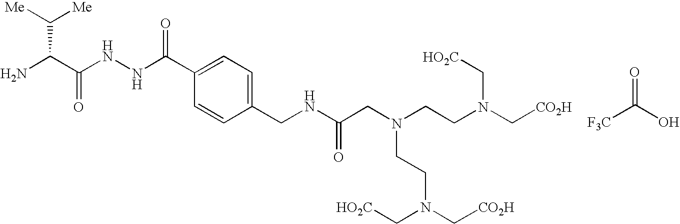

### Part A—Preparation of (2R)—N-{[4-(Aminomethyl)phenyl]carbonylamino}-2-[(tert-butoxy)carbonylamino]-3-methylbutanamide

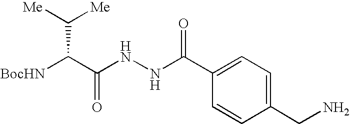

A solution of Boc-DVal-OH (1.30×10² mg, 0.598 mmol) and HOBt (84.0 mg, 0.549 mmol) in dry DMF (5.00 mL) was successively treated with HBTU (209 mg, 0.551 mmol) and Et₃N (278 μL, 1.99 mmol) then stirred 5 minutes at 22° C. The product of Example 29A was deprotected with a solution of TFA in CH₂Cl₂ (1:1 v/v) and the resulting salt (251 mg, 0.501 mmol) added in one portion to the preactivated solution. After 0.5 hours at 22° C., the crude reaction mixture was diluted with ethyl acetate (100 mL), washed with 10% aqueous citric acid (2×30 mL), saturated NaHCO₃ (3×30 mL), 1N NaOH (30 mL) and saturated NaCl (30 mL), then dried over Na₂SO₄, filtered and concentrated in vacuo. The resulting oil was treated with a solution of Et₂NH in acetonitrile (1:1 v/v, 5.00 mL), stirred 0.3 hours, and then concentrated in vacuo. The resulting oil was used directly in the subsequent step.

### Part B—Preparation of 2-{[2-({[N-({4-[N-((2R)-2-Amino-3-methylbutanoylamino)-carbamoyl]phenyl}methyl)carbamoyl]methyl}{2-[bis(carboxymethyl)amino]-ethyl}amino)ethyl](carboxymethyl)amino}acetic Acid, Trifluoroacetic Acid Salt

A previously prepared solution of 2-{bis[2-(bis{[(tert-butyl)oxycarbonyl]-methyl}amino)-ethyl]amino}acetic acid (3.40×10² mg, 0.550 mmol) in acetonitrile (4.00 mL) containing HBTU (209 mg, 0.551 mmol), HOBt (84.0 mg, 0.549 mmol) and Et₃N (278 □L, 1.99 mmol) was transferred to a solution of the product of Part A (182 mg, 0.501 mmol) in acetonitrile (3.00 mL); additional acetonitrile was (2×0.50 mL) was used to quantitate the transfer. The resulting solution was maintained at 22° C. for 0.5 hours, then concentrated in vacuo. The crude oil was treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 12 minutes was lyophilized to a white solid (1.0×101 mg, 9.1 μmol; 1.8%). ¹H NMR (DMSO-d₆, 600 MHz): δ 10.54 (1H, s), 10.41 (1H, s), 8.99 (1H, br t, J=5.8 Hz), 8.19 (3H, br s), 7.87 (2H, AB, J_{AB}=8.3 Hz), 7.42 (2H, AB, J_{AB}=8.3 Hz), 4.44 (2H, d, J=5.8 Hz), 4.29 (2H, br s), 3.70 (1H, br s), 3.51 (8H, s), 3.38 (4H, br s), 3.06 (4H, br t, J=4.9 Hz), 2.20-2.14 (1H, m), 1.05 (3H, d, J=6.9 Hz), 1.04 (3H, d, J=6.9 Hz). MS (ESI): 662.3 (9.7, M+Na), 640.4 (31.0, M+H), 320.9 (100, M+H). The optical purity of the product was established by chiral GLC analysis; 99.6% D-valine.

### EXAMPLE 94

### Synthesis of 2-({2-[(2-{4-[N-((2R)-2-Amino-3-phenylpropanoylamino)-carbamoyl]piperidyl}-2-oxoethyl){2-[bis(carboxymethyl)amino]-ethyl}amino]ethyl}(carboxymethyl)amino)acetic Acid, Trifluoroacetic Acid Salt

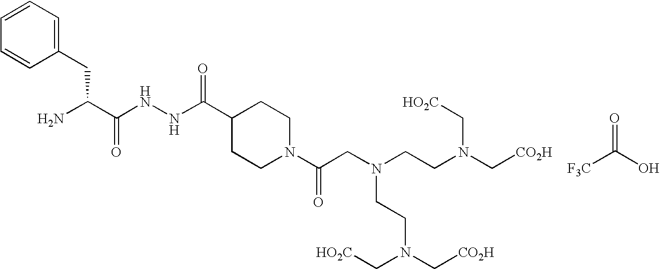

### Part A—Preparation of (2R)-2-[(tert-Butoxy)carbonylamino]-3-phenyl-N-(4-piperidylcarbonylamino)propanamide

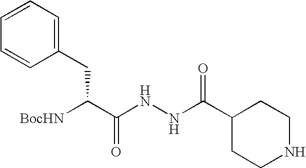

A solution of Boc-DPhe-OH (332 mg, 1.25 mmol) and HOBt (177 mg, 1.16 mmol) in dry DMF (5.00 mL) was successively treated with HBTU (435 mg, 1.15 mmol) and i-Pr₂NEt (727 μL, 4.17 mmol) then stirred 5 minutes at 22° C. The product of Example 30A was deprotected with a solution of TFA in CH₂Cl₂ (1:1 v/v) and the resulting salt (5.00×10² mg, 1.04 mmol) added in one portion to the preactivated solution. After 2 hours at 22° C., the crude reaction mixture was diluted with ethyl acetate (125 mL), washed with 10% aqueous citric acid (5×5 mL), saturated NaHCO₃ (5×5 mL) and saturated NaCl (2×5 mL), then dried over MgSO₄, filtered and concentrated in vacuo. The resulting solid was treated with a solution of Et₂NH in acetonitrile (1:1 v/v, 10.0 mL), stirred 0.3 hours, and then concentrated in vacuo. The resulting solid was used directly in the subsequent step.

### Part B—Preparation of 2-({2-[(2-{4-[N-((2R)-2-Amino-3-phenylpropanoylamino)-carbamoyl]piperidyl}-2-oxoethyl){2-[bis(carboxymethyl)amino]ethyl}-amino]-ethyl}(carboxymethyl)amino)acetic Acid, Trifluoroacetic Acid Salt

A previously prepared solution of 2-{bis[2-(bis{[(tert-butyl)oxycarbonyl]-methyl}amino)-ethyl]amino}acetic acid (348 mg, 0.563 mmol) in DMF (5.00 mL) containing HBTU (214 mg, 0.564 mmol) and Et₃N (285 □L, 2.04 mmol) was transferred to a flask containing the product of Part A (2.00×10² mg, 0.512 mmol). The resulting solution was maintained at 22° C. for 1 hour, then concentrated in vacuo and purified by HPLC on a Phenomenex Luna C18 column (21.2×250 mm) using a 1.8%/min gradient of 50-85% acetonitrile containing 0.1% TFA at a flow rate of 20 mL/min. The main product peak eluting at 19 minutes was lyophilized to a white solid. Global deprotection was then performed using TFA/CH₂Cl₂/Et₃SiH (90:8:2 v/v, 2.50 mL). After 3 hours at 22° C., the solution was concentrated in vacuo and purified by HPLC on a Phenomenex Luna C18 column (21.2×250 mm) using a 1.0%/min gradient of 0-20% acetonitrile containing 0.1% TFA at a flow rate of 20 mL/min. The main product peak eluting at 11 minutes was lyophilized to a white solid (212.5 mg, 0.189 mmol; 37.0%). ¹H NMR (DMSO-d₆, 600 MHz): δ10.10 (1H, br s), 7.31 (2H, dd, J=7.4, 7.0 Hz), 7.28 (2H, dd, J=6.6, 1.7 Hz), 7.25 (1H, tt, J=7.0, 1.6 Hz), 4.30 (1H, br d, J=12.4 Hz), 3.97 (1H, br d, J=12.5 Hz), 3.84 (1H, dd, J=8.0, 5.3 Hz), 3.73 (1H, s), 3.67 (2H, ABqd, J_{AB}=13.7 Hz, J_{d}=6.2 Hz), 3.37 (8H, s), 3.08 (1H, dd, J=13.9, 5.2 Hz), 3.01 (1H, br t, J=12.3 Hz), 2.88-2.77 (9H, m), 2.64 (1H, br t, J=12.5 Hz), 1.71 (2H, br s), 1.62-1.56 (1H, m), 1.49-1.41 (1H, m). MS (ESI): 666.4 (39.3, M+H), 333.8 (100, M+2H). HRMS: Calcd for C₂₉H₄₃N₇O₁₁Na: 688.2913; found: 688.2908. The optical purity of the product was established by chiral GLC analysis; 98.6% D-phenylalanine.

### EXAMPLE 95

### Synthesis of 2-[(2-{[(N-{5-[N-((2R)-2-Aminohexanoylamino)carbamoyl]-pentyl}carbamoyl)methyl]{2-[bis(carboxymethyl)amino]-ethyl}amino}ethyl)(carboxymethyl)amino]acetic Acid, Trifluoroacetic Acid Salt

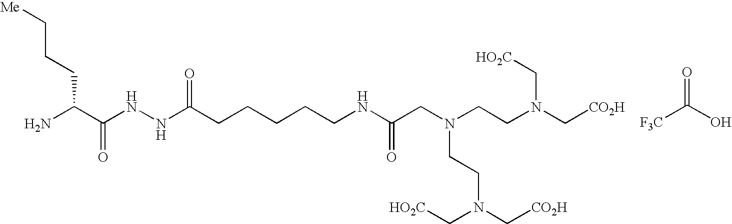

### Part A—Preparation of (2R)—N-(6-Aminohexanoylamino)-2-[(tert-butoxy)carbonylamino]hexanamide

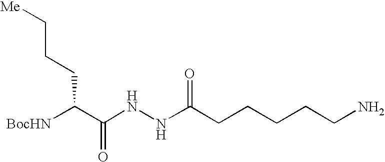

A solution of Boc-DNle-OH (139 mg, 0.601 mmol) and HOBt (84.0 mg, 0.549 mmol) in dry DMF (5.00 mL) was successively treated with HBTU (209 mg, 0.551 mmol) and Et₃N (278 μL, 1.99 mmol) then stirred 5 minutes at 22° C. The product of Example 3A (241 mg, 0.501 mmol) was added in one portion and the resulting solution stirred 0.5 hours at 22° C. The crude reaction mixture thus obtained was diluted with ethyl acetate (100 mL), washed with 10% aqueous citric acid (3×20 mL), saturated NaHCO₃ (3×20 mL), 1N NaOH (20 mL) and saturated NaCl (20 mL), dried over Na₂SO₄, filtered and concentrated in vacuo. The resulting crude material was treated with a solution of Et₂NH in acetonitrile (1:1 v/v, 10.0 mL), stirred 0.3 hours, and then concentrated in vacuo. The resulting oil was used directly in the subsequent step.

### Part B—Preparation of 2-[(2-{[(N-{5-[N-((2R)-2-Aminohexanoylamino)-carbamoyl]pentyl}carbamoyl)methyl]{2-[bis(carboxymethyl)amino]-ethyl}-amino}ethyl)(carboxymethyl)amino]acetic Acid, Trifluoroacetic Acid Salt

A previously prepared solution of 2-{bis[2-(bis{[(tert-butyl)oxycarbonyl]-methyl}amino)-ethyl]amino}acetic acid (3.40×10² mg, 0.550 mmol) in acetonitrile (4.00 mL) containing HBTU (209 mg, 0.551 mmol), HOBt (84.0 mg, 0.549 mmol) and Et₃N (278 μL, 1.99 mmol) was transferred to a solution of the product of Part A (179 mg, 0.501 mmol) in acetonitrile (3.00 mL); additional acetonitrile was (2×0.50 mL) was used to quantitate the transfer. The resulting solution was maintained at 22° C. for 0.5 hours, then concentrated in vacuo. The crude oil was treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 1.0%/min gradient of 0-20% acetonitrile containing 0.1% HCO₂H at a flow rate of 20 mL/min. The main product peak eluting at 5.0 minutes was lyophilized to a white solid (1.00×10² mg, 91.8 μmol; 18.3%); yield based on TFA salt following repeated lyophilization in acetonitrile/H₂O (1:1, v/v) containing 0.1% TFA. ¹H NMR (DMSO-d₆, 600 MHz): δ 10.07 (1H, br s), 8.02 (1H, t, J=5.7 Hz), 3.77 (1H, t, J=6.3 Hz), 3.40 (8H, br s), 3.17 (2H, br s), 3.07 (2H, td, J=6.6, 6.4 Hz), 2.82 (4H, br t, J=6.3 Hz), 2.70 (4H, br t, J=6.1 Hz), 2.16 (2H, t, J=7.3 Hz), 1.72 (2H, dt, J=7.9, 7.2 Hz), 1.53 (2H, tt, J=7.4, 7.4 Hz), 1.43 (2H, tt, J=7.2, 7.2 Hz), 1.38-1.25 (6H, m), 0.87 (3H, t, J=7.2 Hz). ¹³C NMR (DMSO-d₆, 151 MHz): δ 172.2, 171.0, 169.3, 167.5, 56.8, 55.7, 52.0, 51.1, 51.0, 38.2, 32.9, 30.9, 28.7, 26.0, 25.7, 24.7, 21.7, 13.6. MS (ESI): 634.4 (73.1, M+H), 317.9 (100, M+2H). HRMS: Calcd for C₂₆H₄₈N₇O₁₁: 634.3406; found: 634.3412. The optical purity of the product was established by chiral GLC analysis; 97.7% D-norleucine.

### EXAMPLE 96

### Synthesis of 2-[(2-{[2-(4-{[N-((2S)-2-Amino-4-methylpentanoylamino)-carbamoyl]methyl}piperidyl)-2-oxoethyl]{2-[bis(carboxymethyl)-amino]ethyl}amino}ethyl)(carboxymethyl)amino]acetic Acid, formic acid salt

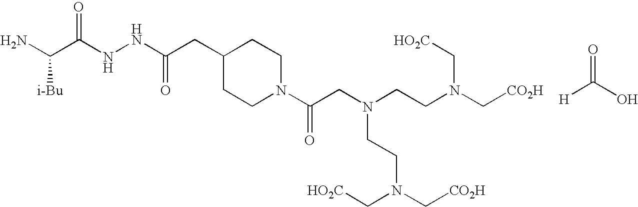

### Part A—Preparation of Fluoren-9-ylmethyl 4-[(N-aminocarbamoyl)methyl]-piperidinecarboxylate, Trifluoroacetic Acid Salt

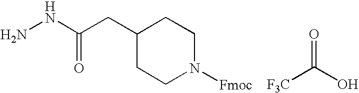

A solution of 2-{1-[(fluoren-9-ylmethyl)oxycarbonyl]-4-piperidyl}acetic acid (2.01 g, 5.50 mmol) in dry DMF (10.0 mL) was successively treated with HBTU (2.08 g, 5.48 mmol) and i-Pr₂NEt (1.74 mL, 9.99 mmol) then stirred 3 minutes at 22° C. tert-Butyl carbazate (0.660 g, 4.99 mmol) was added in one portion and the resulting solution stirred 1 hour at 22° C. The crude reaction mixture was diluted with ethyl acetate (250 mL), washed with 10% aqueous citric acid (3×20 mL), saturated NaHCO₃ (3×20 mL) and saturated NaCl (2×20 mL), then dried over MgSO₄, filtered and concentrated in vacuo. The resulting solid was dissolved in CH₂Cl₂ (5.00 mL) and treated with TFA (5.00 mL, 64.9 mmol). After 1 hour at 22° C., all volatiles were removed in vacuo and the residue redissolved in acetonitrile/H₂O (1:1 v/v) then lyophilized to an off white solid that was used without further purification in the subsequent step.

### Part B—Preparation of (2S)-2-[(tert-Butoxy)carbonylamino]-4-methyl-N-(2-(4-piperidyl)acetylamino)pentanamide

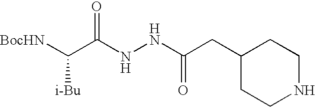

A solution of the product of Part A (3.00×10² mg, 0.608 mmol) in DMF (1.00 mL) containing i-Pr₂NEt (318 μL, 1.82 mmol) was transferred to a previously prepared solution of Boc-DLeu-OH (155 mg, 0.670 mmol) and HOBt (103 mg, 0.673 mmol) in dry DMF (2.00 mL) containing HBTU (254 mg, 0.670 mmol) and i-Pr₂NEt (212 μL, 1.22 mmol). After 0.5 hours at 22° C., the crude reaction mixture was diluted with ethyl acetate (80 mL), washed with 10% aqueous citric acid (5×5 mL), saturated NaHCO₃ (5×5 mL) and saturated NaCl (2×10 mL), then dried over MgSO₄, filtered and concentrated in vacuo. The resulting oil was treated with a solution of Et₂NH in acetonitrile (1:1 v/v, 3.00 mL), stirred 0.3 hours, and then concentrated in vacuo. The resulting solid was used directly in the subsequent step.

### Part C—Preparation of 2-[(2-{[2-(4-{[N-((2S)-2-Amino-4-methylpentanoylamino)-carbamoyl]methyl}piperidyl)-2-oxoethyl]{2-[bis(carboxymethyl)amino]-ethyl}-amino}ethyl)(carboxymethyl)amino]acetic Acid, formic acid salt

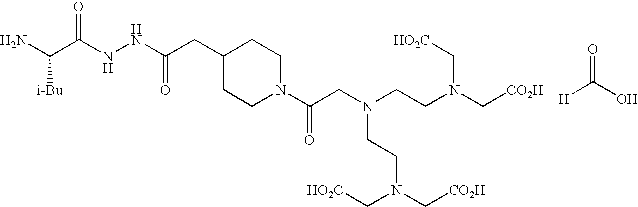

A previously prepared solution of 2-{bis[2-(bis{[(tert-butyl)oxycarbonyl]-methyl}amino)-ethyl]amino}acetic acid (352 mg, 0.570 mmol) in acetonitrile (3.00 mL) containing HBTU (216 mg, 0.569 mmol) and Et₃N (217 μL, 1.56 mmol) was transferred to a flask containing the product of Part B (192 mg, 0.518 mmol). The resulting solution was maintained at 22° C. for 0.25 hours, then concentrated in vacuo. Global deprotection was then performed using TFA/CH₂Cl₂/Et₃SiH (90:8:2 v/v, 3.00 mL). After 2.5 hours at 22° C., the solution was concentrated in vacuo and purified by HPLC on a Phenomenex Luna C18 column (21.2×250 mm) using a 1.0%/min gradient of 0-20% acetonitrile containing 0.1% HCO₂H at a flow rate of 20 mL/min. The main product peak eluting at 9.5 minutes was lyophilized to a white solid (48.0 mg, 54.8 μmol; 10.6%). ¹H NMR (DMSO-d₆, 600 MHz): δ 10.08 (1H, br s), 4.27 (1H, br d, J=12.5 Hz), 3.93 (1H, br s), 3.71 (1H, t, J=7.3 Hz), 3.65 (1H, d, J=15.1 Hz), 3.47 (1H, dd, J=15.1, 9.3 Hz), 3.36 (8H, s), 2.96-2.92 (1H, m), 2.82 (3H, br t, J=6.5 Hz), 2.80-2.66 (3H, m), 2.57-2.52 (1H, m), 2.13 (2H, br d, J=6.4 Hz), 1.90 (1H, br s), 1.76-1.65 (2H, m), 1.59 (1H, tt, J=6.9, 6.9 Hz), 1.52 (1H, tt, J=7.6, 6.6 Hz), 1.24-1.03 (2H, m), 0.92 (3H, d, J=6.5 Hz), 0.90 (3H, d, J=6.6 Hz). ¹³C NMR (DMSO-d₆, 151 MHz): δ 172.7, 169.6, 168.5, 167.1, 56.9, 51.3, 50.7, 49.9, 44.6, 41.1, 40.8, 40.1, 32.8, 30.9, 23.5, 22.5, 21.9. MS (ESI): 646.4 (100, M+H), 323.9 (952, M+2H). HRMS: Calcd for C₂₇H₄₅FeN₇O₁₁: 699.2521; found: 699.2522. The optical purity of the product was established by chiral GLC analysis; 99.1% D-leucine.

### EXAMPLE 97

### Synthesis of 2-[(2-{[2-(4-{[N-((2S)-2-Amino-3-phenylpropanoylamino)-carbamoyl]methyl}piperidyl)-2-oxoethyl]{2-[bis(carboxymethyl)amino]-ethyl}amino}ethyl)(carboxymethyl)amino]acetic Acid, Formic Acid Salt

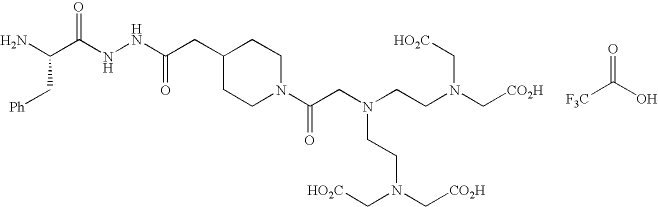

### Part A—Preparation of (2S)-2-[(tert-Butoxy)carbonylamino]-3-phenyl-N-(2-(4-piperidyl)acetylamino)propanamide

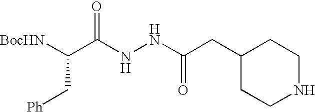

A solution of the product of Example 96A (3.00×10² mg, 0.608 mmol) in DMF (1.00 mL) containing i-Pr₂NEt (318 μL, 1.82 mmol) was transferred to a previously prepared solution of Boc-DPhe-OH (177 mg, 0.667 mmol) and HOBt (103 mg, 0.673 mmol) in dry DMF (2.00 mL) containing HBTU (254 mg, 0.670 mmol) and i-Pr₂NEt (212 μL, 1.22 mmol). After 0.5 hours at 22° C., the crude reaction mixture was diluted with ethyl acetate (80 mL), washed with 10% aqueous citric acid (5×5 mL), saturated NaHCO₃ (5×5 mL) and saturated NaCl (2×10 mL), then dried over MgSO₄, filtered and concentrated in vacuo. The resulting oil was treated with a solution of Et₂NH in acetonitrile (1:1 v/v, 3.00 mL), stirred 0.3 hours, and then concentrated in vacuo. The resulting solid was used directly in the subsequent step.

### Part B—Preparation of 2-[(2-{[2-(4-{[N-((2S)-2-Amino-3-phenylpropanoylamino)-carbamoyl]methyl}piperidyl)-2-oxoethyl]{2-[bis(carboxymethyl)-amino]ethyl}-amino}ethyl)(carboxymethyl)amino]acetic Acid, Formic Acid Salt

A previously prepared solution of 2-{bis[2-(bis{[(tert-butyl)oxycarbonyl]-methyl}amino)-ethyl]amino}acetic acid (353 mg, 0.571 mmol) in acetonitrile (3.00 mL) containing HBTU (216 mg, 0.569 mmol) and Et₃N (217 μL, 1.56 mmol) was transferred to a flask containing the product of Part A (2.10×10² mg, 0.519 mmol). The resulting solution was maintained at 22° C. for 0.25 hours, then concentrated in vacuo. Global deprotection was then performed using TFA/CH₂Cl₂/Et₃SiH (90:8:2 v/v, 3.00 mL). After 2.5 hours at 22° C., the solution was concentrated in vacuo and purified by HPLC on a Phenomenex Luna C18 column (21.2×250 mm) using a 1.0%/min gradient of 0-20% acetonitrile containing 0.1% HCO₂H at a flow rate of 20 mL/min. The main product peak eluting at 12 minutes was lyophilized to a white solid (103 mg, 0.119 mmol; 23.0%). ¹H NMR (DMSO-d₆, 600 MHz): δ 10.08 (1H, br s), 7.34-7.29 (4H, m), 7.28-7.25 (1H, m), 4.29 (1H, br d, J=13.1 Hz), 3.93 (1H, br t, J=6.5 Hz), 3.87 (1H, br d, J=13.0 Hz), 3.79 (1H, br d, J=14.9 Hz), 3.68 (1H, br d, J=14.2 Hz), 3.38 (8H, s), 3.11 (1H, dd, J=14.0, 5.1 Hz), 2.95 (1H, br t, J=12.2 Hz), 2.90 (1H, dd, J=14.0, 8.0 Hz), 2.85 (8H, br s), 2.56 (1H, br t, J=12.2 Hz), 2.14 (2H, br d, J=6.6 Hz), 1.92 (1H, br s), 1.69 (2H, br d, J=11.7 Hz), 1.24-1.03 (2H, m). ¹³C NMR (DMSO-d₆, 151 MHz): δ172.7, 169.5, 168.2, 166.6, 135.6, 129.5, 128.4, 126.9, 56.2, 54.5, 52.9, 51.5, 50.5, 44.5, 38.0, 32.9, 31.6, 30.9. MS (ESI): 680.3 (100, M+H), 340.9 (98.2, M+2H). HRMS: Calcd for C₃₀H₄₃FeN₇O₁₁: 733.2364; found: 733.2360. The optical purity of the product was established by chiral GLC analysis; 99.3% D-phenylalanine.

### EXAMPLE 98

### Synthesis of 2-{[2-({[N-({4-[N-((2R)-2-Amino-4-phenylbutanoylamino)-carbamoyl]phenyl}methyl)carbamoyl]methyl}{2-[bis(carboxymethyl)amino]-ethyl}amino)ethyl](carboxymethyl)amino}acetic Acid, Trifluoroacetic Acid Salt

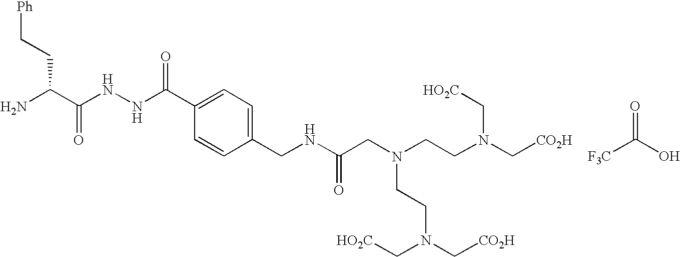

### Part A—Preparation of (2R)—N-Amino-2-[(tert-butoxy)carbonylamino]-4-phenylbutanamide

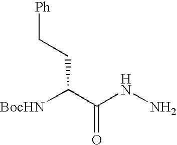

A solution of H-D-Hphe-OH (44.0 g, 245 mmol) in 1:1 H₂O/t-BuOH (350 mL) at 22° C. was treated with powdered NaOH (10.8 g, 270 mmol) followed by Boc₂O (58.9 g, 270 mmol) in three equal portions over 10 minutes. The resulting suspension was stirred 16 hours then diluted with H₂O (300 mL) and washed with Et₂O (3×200 mL). The remaining aqueous solution was acidified (pH 5) with AcOH then washed with ethyl acetate (3×250 mL). Note: during the ethyl acetate washes, additional AcOH was added to the aqueous layers to maintain pH. The combined organic extracts were washed with H₂O (250 mL) and saturated NaCl (250 mL) then dried over Na₂SO₄, filtered and concentrated in vacuo to a white solid (47.7 g, 171 mmol). The original Et₂O washes were acidified with aqueous AcOH in order to precipitate unreacted amino acid (6.8 g, 38 mmol). Additional Boc-protected material (8.51 g, 30.4 mmol) was recovered from the filtrate using the above extraction procedure; combined yield of 56.2 g (201 mmol, 82.0%). The product was used without further purification in subsequent reactions.

A solution of the Boc-D-Hphe-OH (47.7 g, 171 mmol) in 3:1 CH₂Cl₂/methanol (400 mL) was treated with (trimethylsilyl)diazomethane (205 mmol; 103 mL of a 2.0 M solution in Et₂O) dropwise over 10 minutes at 22° C. The resulting yellow solution was stirred an additional 15 minutes, then concentrated in vacuo. The crude material was redissolved in methanol (171 mL) and treated with hydrazine hydrate (33.2 mL, 684 mmol) at 22° C. Hydrazide formation was complete after 5 hours. All volatiles were removed in vacuo and the crude material recrystallized from hot ethyl acetate to afford a white powder (45.6 g, 155 mmol, 91.0%). ¹H NMR (DMSO-d₆, 600 MHz): δ 9.01 (1H, s), 7.27 (2H, dd, J=7.6, 7.5 Hz), 7.18-7.16 (3H, m), 6.94 (1H, d, J=8.2 Hz), 4.20 (2H, br s), 3.89 (1H, ddd, J=8.8, 5.5, 5.0 Hz), 2.60 (1H, ddd, J=15.1, 10.5, 5.2 Hz), 2.52-2.47 (1H, m), 1.86-1.74 (2H, m), 1.39 (9H, s). ¹³C NMR (DMSO-d₆, 151 MHz): δ 171.2, 155.2, 141.4, 128.2(2), 125.7, 77.9, 52.7, 34.0, 31.6, 28.2. MS (ESI): 238.4 (100, M-t-Bu), 194.4 (67.0, M-Boc), 177.4 (27.7). HRMS: Calcd for C₁₁H₁₆N₃O₃: 238.1186; found: 238.1186. The optical purity of the product was established by chiral GLC analysis (99.8% D-homophenylalanine).

### Part B—Preparation of (2R)—N-{[4-(Aminomethyl)phenyl]carbonylamino}-2-[(tert-butoxy)carbonylamino]-4-phenylbutanamide

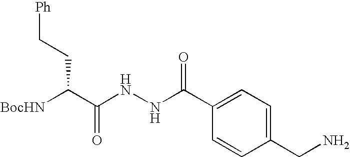

A solution of Boc-D-Hphe-OH (182 mg, 0.652 mmol) and HOBt (84.0 mg, 0.549 mmol) in dry DMF (5.00 mL) was successively treated with HBTU (209 mg, 0.551 mmol) and i-Pr₂NEt (349 μL, 2.00 mmol) then stirred 5 minutes at 22° C. The product of Example 29A was deprotected with a solution of TFA in CH₂Cl₂ (1:1 v/v) and the resulting salt (251 mg, 0.501 mmol) added in one portion to the preactivated solution followed by i-Pr₂NEt (80 μL, 0.459 mmol); additional DMF (2×1.00 mL) was used to wash down the sides of the reaction vessel. After 0.5 hours at 22° C., the crude reaction mixture was partitioned between ethyl acetate and 10% aqueous citric acid (50 mL each) and the layers separated. The ethyl acetate layer was washed with 10% aqueous citric acid (2×25 mL), saturated NaHCO₃ (3×25 mL), H₂O (25 mL) and saturated NaCl (25 mL), then dried over Na₂SO₄, filtered and concentrated in vacuo. The resulting oil was treated with a solution of Et₂NH in acetonitrile (1:1 v/v, 6.00 mL), stirred 0.3 hours, and then concentrated in vacuo. The crude product was used directly in the subsequent step. ¹H NMR (DMSO-d₆, 600 MHz): δ 7.82 (2H, AB, J_{AB}=8.3 Hz), 7.43 (2H, AB, J_{AB}=8.0 Hz), 7.29 (2H, dd, J=7.7, 7.4 Hz), 7.23 (2H, d, J=7.1 Hz), 7.19 (1H, dd, J=7.3, 7.3 Hz), 7.07 (1H, br d, J=8.1 Hz), 4.08 (1H, ddd, J=8.1, 6.1, 5.3 Hz), 3.77 (2H, s), 2.75-2.65 (2H, m), 2.01-1.95 (1H, m), 1.91-1.84 (1H, m), 1.41 (9H, s). MS (ESI): 853.4 (100, 2M+H), 427.4 (100, M+H), 371.3 (50.2, M-t-Bu), 327.4 (88.9).

### Part C—Preparation of 2-{[2-({[N-({4-[N-((2R)-2-Amino-4-phenylbutanoylamino)-carbamoyl]phenyl}methyl)carbamoyl]methyl}{2-[bis(carboxymethyl)amino]-ethyl}amino)ethyl](carboxymethyl)amino}acetic Acid, Trifluoroacetic Acid Salt

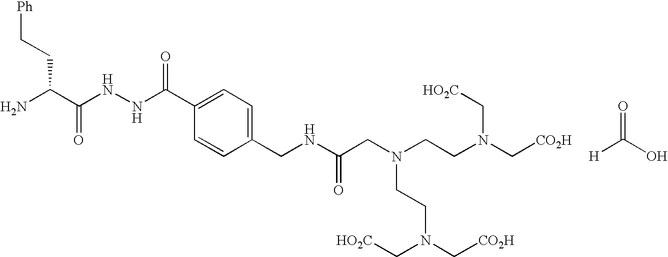

A previously prepared solution of 2-{bis[2-(bis{[(tert-butyl)oxycarbonyl]-methyl}amino)-ethyl]amino}acetic acid (309 mg, 0.500 mmol) in acetonitrile (5.00 mL) containing HBTU (1.90×10² mg, 0.501 mmol), HOBt (77.0 mg, 0.503 mmol) and Et₃N (279 □L, 2.00 mmol) was transferred to a solution of the product of Part B (213 mg, 0.501 mmol) in acetonitrile (2.00 mL); additional acetonitrile was (2×0.50 mL) was used to quantitate the transfer. The resulting solution was maintained at 22° C. for 0.5 hours, then concentrated in vacuo. The residue was redissolved in ethyl acetate (50 mL), washed with 10% aqueous citric acid (3×25 mL), saturated NaHCO₃ (3×25 mL) and saturated NaCl (25 mL), then dried over Na₂SO₄, filtered and concentrated in vacuo. The crude oil was treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 1.0%/min gradient of 0-30% acetonitrile containing 0.1% HCO₂H at a flow rate of 20 mL/min. The main product peak eluting at 19 minutes was lyophilized to a white solid (83.0 mg, 93.7 μmol; 18.7%); for the purpose of consistency of characterization, the TFA salt was prepared following repeated lyophilization in acetonitrile/H₂O (1:1 v/v) containing 0.1% TFA. ¹H NMR (DMSO-d₆, 600 MHz): δ 10.57 (1H, s), 10.53 (1H, s), 9.02 (1H, t, J=5.8 Hz), 8.39 (3H, br s), 7.88 (2H, AB, J_{AB}=8.3 Hz), 7.43 (2H, AB, J_{AB}=8.3 Hz), 7.33 (2H, dd, J=7.6, 7.1 Hz), 7.24 (3H, m), 4.44 (2H, d, J=5.6 Hz), 4.31 (2H, s), 4.03 (1H, br s), 3.52 (8H, s), 3.40 (4H, br t, J=5.5 Hz), 3.07 (4H, br t, J=5.6 Hz), 2.83-2.73 (2H, m), 2.14-2.07 (2H, m). ¹³C NMR (DMSO-d₆, 151 MHz): δ 172.7, 167.9, 165.3, 164.9, 157.9 (q, J=32.3 Hz), 142.6, 140.6, 130.8, 128.5, 128.1, 127.7, 127.2, 126.1, 116.7 (q, J=298 Hz), 54.3, 53.8, 52.2, 51.2, 48.6, 42.0, 33.3, 30.0. MS (ESI): 662.3 (9.7, M+Na), 640.4 (31.0, M+H), 320.9 (100, M+H). HRMS: Calcd for C₃₂H₄₁FeN₇O₁₁: 755.2208; found: 755.2200. The optical purity of the product was established by chiral GLC analysis; 99.7% D-homophenylalanine.

### EXAMPLE 99

### Synthesis of 2-({2-[({N-[(4-{N-[(2R)-2-Amino-3-(2,3,4,5,6-pentafluorophenyl)-propanoylamino]carbamoyl}phenyl)methyl]carbamoyl}methyl){2-[bis(carboxymethyl)amino]ethyl}amino]ethyl}(carboxymethyl)amino)acetic Acid, Formic Acid Salt

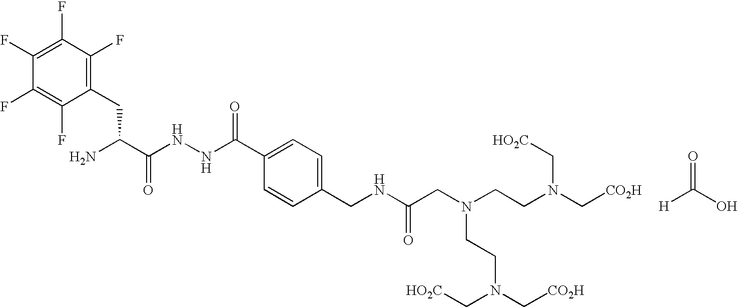

### Part A—Preparation of (2R)—N-{[4-(Aminomethyl)phenyl]carbonylamino}-2-[(tert-butoxy)carbonylamino]-3-(2,3,4,5,6-pentafluorophenyl)propanamide

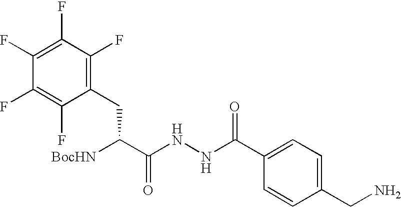

A solution of Boc-DPhe(F₅)—OH (231 mg, 0.650 mmol) and HOBt (84.0 mg, 0.549 mmol) in dry DMF (3.00 mL) was successively treated with HBTU (209 mg, 0.551 mmol) and i-Pr₂NEt (349 μL, 2.00 mmol) then stirred 5 minutes at 22° C. The product of Example 29A was deprotected with a solution of TFA in CH₂Cl₂ (1:1 v/v) and the resulting salt (251 mg, 0.501 mmol) added in one portion to the preactivated solution followed by i-Pr₂NEt (80.0 μL, 0.459 mmol); additional DMF (2×1.00 mL) was used to wash the sides of the reaction vessel. After 0.5 hours at 22° C., the crude reaction mixture was partitioned between ethyl acetate and 10% aqueous citric acid (50 mL each) and the layers separated. The ethyl acetate layer was washed with 10% aqueous citric acid (2×25 mL), saturated NaHCO₃ (3×25 mL), H₂O (25 mL) and saturated NaCl (25 mL), then dried over Na₂SO₄, filtered and concentrated in vacuo. The resulting oil was treated with a solution of Et₂NH in acetonitrile (1:1 v/v, 6.00 mL), stirred 0.3 hours, and then concentrated in vacuo. The crude material was used directly in the subsequent step.

### Part B—Preparation of 2-({2-[({N-[(4-{N-[(2R)-2-Amino-3-(2,3,4,5,6-pentafluorophenyl)-propanoylamino]carbamoyl}phenyl)methyl]carbamoyl}methyl){2-[bis(carboxymethyl)amino]ethyl}amino]ethyl}(carboxymethyl)amino)acetic Acid, Formic Acid Salt

A previously prepared solution of 2-{bis[2-(bis{[(tert-butyl)oxycarbonyl]-methyl}amino)-ethyl]amino}acetic acid (309 mg, 0.500 mmol) in acetonitrile (5.00 mL) containing HBTU (1.90×10² mg, 0.501 mmol), HOBt (77.0 mg, 0.503 mmol) and Et₃N (279 μL, 2.00 mmol) was transferred to a solution of the product of Part A (251 mg, 0.501 mmol) in acetonitrile (2.00 mL); additional acetonitrile was (2×0.50 mL) was used to quantitate the transfer. The resulting solution was maintained at 22° C. for 0.5 hours, then concentrated in vacuo. The residue was redissolved in ethyl acetate (50 mL), washed with 10% aqueous citric acid (3×25 mL), saturated NaHCO₃ (3×25 mL) and saturated NaCl (25 mL), then dried over Na₂SO₄, filtered and concentrated in vacuo. The crude oil was treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 1.0%/min gradient of 0-30% acetonitrile containing 0.1% HCO₂H at a flow rate of 20 mL/min. The main product peak eluting at 19 minutes was lyophilized to a white solid (1.00×10² mg, 0.104 mmol; 20.8%). ¹H NMR (DMSO-d₆, 600 MHz): δ 10.43 (1H, br s), 8.68 (1H, t, J=6.2 Hz), 7.79 (2H, AB, J_{AB}=8.2 Hz), 7.36 (2H, AB, J_{AB}=8.2 Hz), 4.36 (2H, br d, J=6.0 Hz), 3.69 (1H, br t, J=7.4 Hz), 3.38 (8H, s), 3.24 (2H, s), 3.08 (1H, dd, J=13.7, 7.0 Hz), 2.93 (1H, dd, J=13.4, 8.1 Hz), 2.80 (4H, br t, J=6.4 Hz), 2.68 (4H, br t, J=6.3 Hz). ¹³C NMR (DMSO-d₆, 151 MHz): δ 172.5, 170.6, 170.3, 165.1, 145.1 (d, J=246 Hz), 143.9, 139.2 (d, J=249 Hz), 136.8 (d, J=245 Hz), 130.7, 127.5, 126.9, 111.2 (t, J=15.9 Hz), 56.8, 55.7, 52.3, 52.0, 51.3, 41.7, 27.1. MS (ESI): 778.3 (51.0, M+H), 389.7 (100, M+2H). HRMS: Calcd for C₃₁H₃₄F₅FeN₇O₁₁: 831.1580; found: 831.1568.

### EXAMPLE 100

### Synthesis of 2-[(2-{[(N-{[4-(N-{(2R)-2-Amino-3-[4-(phenylcarbonylamino)phenyl]-propanoylamino}carbamoyl)phenyl]methyl}carbamoyl)methyl]{2-[bis(carboxymethyl)amino]ethyl}amino}ethyl)(carboxymethyl)amino]acetic Acid, Trifluoroacetic Acid Salt

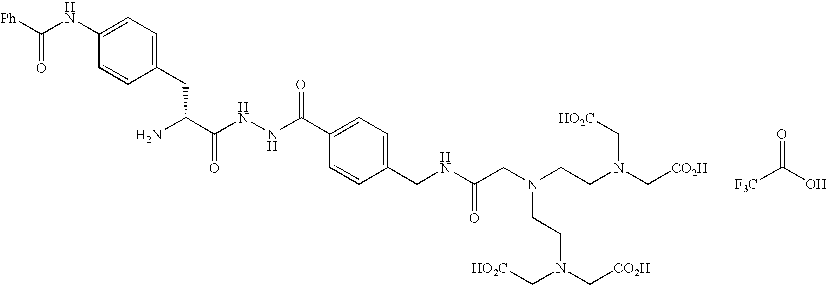

### Part A—Preparation of (2R)-2-[(tert-Butoxy)carbonylamino]-3-[4-(phenylcarbonylamino)phenyl]propanoic acid

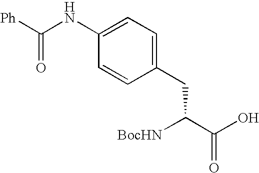

A solution of Boc-DPhe(4-NH₂)—OH•TFA (182 mg, 0.462 mmol) and Et₃N (212 μL, 1.52 mmol) in dry CH₂Cl₂ (4.00 mL) was treated with a solution of benzoyl chloride (0.510 mmol; 1.00 mL of a 0.51 M solution in CH₂Cl₂) dropwise over 10 minutes at 22° C. The resulting solution was heated to reflux and maintained for 3.5 hours. Upon cooling to 22° C., the resulting solution was diluted with CH₂Cl₂ (15 mL), washed with 2.5 M HCl (2×20 mL), H₂O (2×20 mL) and saturated NaCl (20 mL), then dried over Na₂SO₄, filtered and concentrated in vacuo to a white solid (48.0 mg, 0.125 mmol; 27.0%); small amounts of benzoic acid contaminate this material.

### Part B—Preparation of (2R)—N-{[4-(Aminomethyl)phenyl]carbonylamino}-2-[(tert-butoxy)carbonylamino]-3-[4-(phenylcarbonylamino)phenyl]propanamide, Formic Acid Salt

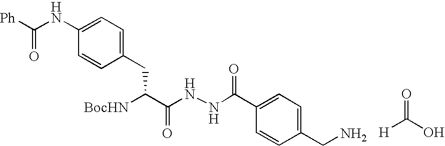

A solution of Part A (48.0 mg, 0.125 mmol) and HOBt (19.0 mg, 0.124 mmol) in dry DMF (3.00 mL) was successively treated with HBTU (47.0 mg, 0.124 mmol) and i-Pr₂NEt (88.0 μL, 0.505 mmol) then stirred 5 minutes at 22° C. The product of Example 29A was deprotected with a solution of TFA in CH₂Cl₂ (1:1 v/v) and the resulting salt (82.0 mg, 0.164 mmol) added in one portion to the preactivated solution. After 0.5 hours at 22° C., all volatiles were removed in vacuo and the resulting oil treated with a solution of Et₂NH in acetonitrile (1:1 v/v, 6.00 mL). The resulting solution was stirred 0.3 hours at 22° C., then concentrated in vacuo and purified by HPLC on a Phenomenex Luna C18 column (21.2×250 mm) using a 0.88%/min gradient of 10-45% acetonitrile containing 0.1% HCO₂H at a flow rate of 20 mL/min. The main product peak eluting at 25 minutes was lyophilized to a white solid (24.0 mg, 41.5 μmol; 33.3%).

### Part C—Preparation of 2-[(2-{[(N-{[4-(N-{(2R)-2-Amino-3-[4-(phenylcarbonylamino)-phenyl]propanoylamino}carbamoyl)phenyl]methyl}carbamoyl)methyl]{2-[bis(carboxymethyl)amino]ethyl}amino}ethyl)(carboxymethyl)amino]acetic Acid, Formic Acid Salt

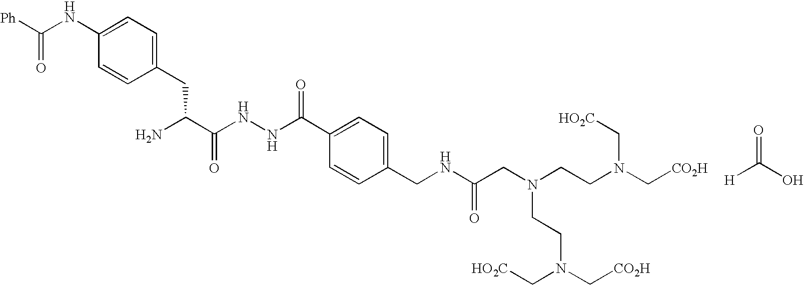

A previously prepared solution of 2-{bis[2-(bis{[(tert-butyl)oxycarbonyl]-methyl}amino)-ethyl]amino}acetic acid (46.0 mg, 74.5 μmol) in acetonitrile (1.50 mL) containing HBTU (28.0 mg, 73.8 μmol), HOBt (11.0 mg, 71.8 μmol) and Et₃N (41.0 μL, 0.294 mmol) was treated with a solution of the product of Part B (42.8 mg, 74.0 μmol) in acetonitrile (1.50 mL). The resulting solution was maintained at 22° C. for 0.5 hours, then concentrated in vacuo. The residue was redissolved in ethyl acetate (45 mL), washed with 10% aqueous citric acid (3×20 mL), saturated NaHCO₃ (3×20 mL) and saturated NaCl (20 mL), then dried over Na₂SO₄, filtered and concentrated in vacuo. The crude oil was treated with a solution of Et₃SiH in CH₂Cl₂ (4:1 v/v, 250 μL) followed by TFA (2.30 mL, 29.9 mmol). After stirring 1.5 hours at 22° C., the resulting solution was concentrated in vacuo and purified by HPLC on a Phenomenex Luna C18 column (21.2×250 mm) using a 1.0%/min gradient of 0-30% acetonitrile containing 0.1% HCO₂H at a flow rate of 20 mL/min. The main product peak eluting at 24 minutes was lyophilized to a white solid (28.0 mg, 28.3 μmol; 38.2%). ¹H NMR (DMSO-d₆, 600 MHz): δ 10.25 (1H, br s), 8.68 (1H, t, J=6.2 Hz), 7.96 (2H, AB, J_{AB}=7.1 Hz), 7.84 (2H, AB, J_{AB}=8.2 Hz), 7.74 (2H, AB, J_{AB}=8.4 Hz), 7.59 (1H, tt, J=7.3, 1.2 Hz), 7.53 (2H, dd, J=7.7, 7.2 Hz), 7.38 (2H, AB, J_{AB}=8.2 Hz), 7.32 (2H, AB, J_{AB}=8.3 Hz), 4.36 (2H, br d, J=6.1 Hz), 3.97 (1H, br s), 3.36 (8H, s), 3.18 (2H, s), 3.18-3.15 (1H, m), 2.94 (1H, dd, J=13.9, 7.8 Hz), 2.81 (4H, br t, J=6.5 Hz), 2.64 (4H, br t, J=6.4 Hz). ¹³C NMR (DMSO-d₆, 151 MHz): δ 172.5, 170.6, 169.2, 165.5, 165.4, 143.8, 138.0, 134.9, 131.5, 130.8, 130.7, 129.8, 128.3, 127.6, 127.5, 127.0, 120.3, 57.3, 56.6, 53.2, 52.3, 51.4, 41.7, 37.8. MS (ESI): 807.3 (81.1, M+H), 404.3 (100, M+2H). HRMS: Calcd for C₃₈H₄₄FeN₈O₁₂: 860.2423; found: 860.2420.

### EXAMPLE 101

### Synthesis of 2-[(2-{[(N-{[4-(N-{(2R)-2-Amino-3-[4-(phenylmethoxy)phenyl]-propanoylamino}carbamoyl)phenyl]methyl}carbamoyl)methyl]{2-[bis(carboxymethyl)amino]ethyl}amino}ethyl)(carboxymethyl)amino]acetic Acid, Trifluoroacetic Acid Salt

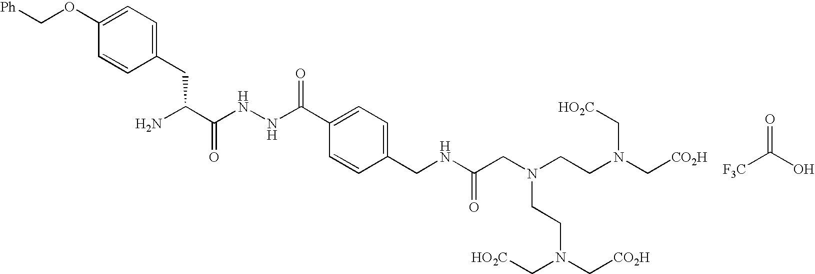

### Part A—Preparation of (2R)—N-{[4-(Aminomethyl)phenyl]carbonylamino}-2-[(tert-butoxy)carbonylamino]-3-[4-(phenylmethoxy)phenyl]propanamide

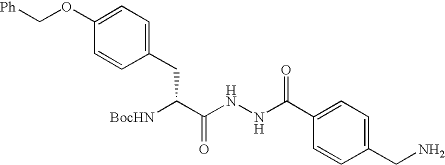

A solution of Boc-DTyr(Bn)-OH (241 mg, 0.649 mmol) and HOBt (84.0 mg, 0.549 mmol) in dry DMF (3.00 mL) was successively treated with HBTU (209 mg, 0.551 mmol) and i-Pr₂NEt (349 μL, 2.00 mmol) then stirred 5 minutes at 22° C. The product of Example 29A was deprotected with a solution of TFA in CH₂Cl₂ (1:1 v/v) and the resulting salt (251 mg, 0.501 mmol) added in one portion to the preactivated solution followed by i-Pr₂NEt (80.0 μL, 0.459 mmol); additional DMF (2×1.00 mL) was used to wash the sides of the reaction vessel. After 0.5 hours at 22° C., the crude reaction mixture was partitioned between ethyl acetate and 10% aqueous citric acid (50 mL each) and the layers separated. The ethyl acetate layer was washed with 10% aqueous citric acid (2×25 mL), saturated NaHCO₃ (3×25 mL) and saturated NaCl (25 mL), then dried over Na₂SO₄, filtered and concentrated in vacuo. The resulting solid was treated with a solution of Et₂NH in acetonitrile (1:1 v/v, 6.00 mL), stirred 0.3 hours, and then concentrated in vacuo. The crude material was used directly in the subsequent step.

### Part B—Preparation of 2-[(2-{[(N-{[4-(N-{(2R)-2-Amino-3-[4-(phenylmethoxy)phenyl]-propanoylamino}carbamoyl)phenyl]methyl}carbamoyl)methyl]{2-[bis(carboxymethyl)amino]ethyl}amino}ethyl)(carboxymethyl)amino]acetic Acid, Trifluoroacetic Acid Salt

A previously prepared solution of 2-{bis[2-(bis{[(tert-butyl)oxycarbonyl]-methyl}amino)-ethyl]amino}acetic acid (309 mg, 0.500 mmol) in acetonitrile (5.00 mL) containing HBTU (1.90×10² mg, 0.501 mmol), HOBt (77.0 mg, 0.503 mmol) and Et₃N (279 □L, 2.00 mmol) was transferred to a solution of the product of Part A (2.60×10² mg, 0.501 mmol) in acetonitrile (2.00 mL); additional acetonitrile was (2×0.50 mL) was used to quantitate the transfer. The resulting solution was maintained at 22° C. for 0.5 hours, then concentrated in vacuo. The residue was redissolved in ethyl acetate (50 mL), washed with 10% aqueous citric acid (3×25 mL), saturated NaHCO₃ (3×25 mL) and saturated NaCl (25 mL), then dried over Na₂SO₄, filtered and concentrated in vacuo. The crude oil was treated with a solution of Et₃SiH in CH₂Cl₂ (4:1 v/v, 1.00 mL) followed by TFA (9.00 mL, 117 mmol). After stirring 2 hours at 22° C., the resulting solution was concentrated in vacuo and purified by HPLC on a Phenomenex Luna C18 column (21.2×250 mm) using a 0.86%/min gradient of 5-35% acetonitrile containing 0.1% TFA and 10% H₂O at a flow rate of 20 mL/min. The main product peak eluting at 27 minutes was lyophilized to a white solid (66.0 mg, 52.8 μmol; 10.5%). ¹H NMR (DMSO-d₆, 600 MHz): δ 10.61 (1H, s), 9.02 (1H, br t, J=5.6 Hz), 8.21 (3H, br s), 7.89 (2H, AB, J_{AB}=8.2 Hz), 7.45 (2H, AB, J_{AB}=7.4 Hz), 7.43 (2H, AB, J_{AB}=8.3 Hz), 7.40 (2H, dd, J=7.7, 7.3 Hz), 7.33 (1H, dd, J=7.3, 7.2 Hz), 7.29 (2H, AB, J_{AB}=8.4 Hz), 7.00 (2H, AB, J_{AB}=8.5 Hz), 5.10 (2H, s), 4.44 (2H, br d, J=5.5 Hz), 4.30 (2H, br s), 4.10 (1H, br s), 3.52 (8H, s), 3.40 (4H, br t, J=5.6 Hz), 3.18 (1H, dd, J=14.0, 4.3 Hz), 3.07 (4H, br t, J=5.8 Hz), 2.98 (1H, dd, J=14.1, 8.2 Hz). ¹³C NMR (DMSO-d₆, 151 MHz): δ 172.7, 167.5, 165.1, 164.9, 157.8 (q, J=32.0 Hz), 157.7, 142.6, 137.1, 130.8, 128.4, 127.8, 127.7, 127.6, 127.1, 126.5, 117.8, 114.9, 69.2, 54.3, 53.9, 52.5, 52.2, 48.7, 42.0, 36.3. MS (ESI): 794.3 (68.6, M+H), 541.2 (23.9), 397.7 (100, M+2H). HRMS: Calcd for C₃₈H₄₅FeN₇O₁₂: 847.2470; found: 847.2469.

### EXAMPLE 102

### Synthesis of 2-({2-[({N-[(4-{N-[(2-Aminoindan-2-yl)carbonylamino]carbamoyl}-phenyl)methyl]carbamoyl}methyl){2-[bis(carboxymethyl)amino]ethyl}-amino]ethyl}(carboxymethyl)amino)acetic Acid, Trifluoroacetic Acid Salt

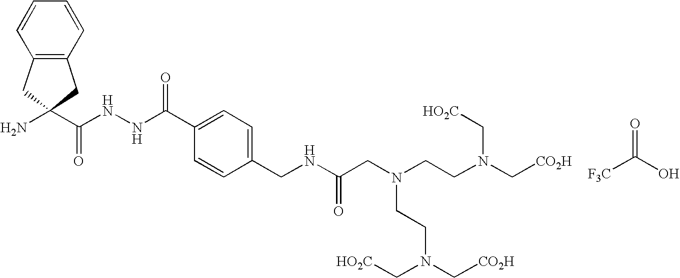

### Part A—Preparation of N-{[4-(Aminomethyl)phenyl]carbonylamino}{2-[(tert-butoxy)carbonylamino]indan-2-yl}carboxamide

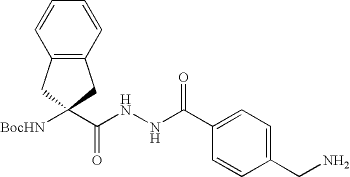

A solution of N-Boc-2-amino-ndane-2-carboxylic acid (166 mg, 0.599 mmol) and HOBt (84.0 mg, 0.549 mmol) in dry DMF (8.00 mL) was successively treated with HBTU (209 mg, 0.551 mmol) and i-Pr₂NEt (349 μL, 2.00 mmol) then stirred 5 minutes at 22° C. The product of Example 29A was deprotected with a solution of TFA in CH₂Cl₂ (1:1 v/v) and the resulting salt (251 mg, 0.501 mmol) added in one portion to the preactivated solution; additional DMF (2×1.00 mL) was used to wash the sides of the reaction vessel. After 0.5 hours at 22° C., the crude reaction mixture was diluted with ethyl acetate (100 mL), washed with 10% aqueous citric acid (3×30 mL), saturated NaHCO₃ (3×30 mL) and saturated NaCl (30 mL), then dried over Na₂SO₄, filtered and concentrated in vacuo. The resulting oil was treated with a solution of Et₂NH in acetonitrile (1:1 v/v, 10.0 mL), stirred 0.5 hours, and then concentrated in vacuo. The crude material was used directly in the subsequent step.

### Part B—Preparation of 2-({2-[({N-[(4-{N-[(2-Aminoindan-2-yl)carbonylamino]-carbamoyl}phenyl)methyl]carbamoyl}methyl){2-[bis(carboxymethyl)-amino]ethyl}-amino]ethyl}(carboxymethyl)amino)acetic Acid, Trifluoroacetic Acid Salt

A previously prepared solution of 2-{bis[2-(bis{[(tert-butyl)oxycarbonyl]-methyl}amino)-ethyl]amino}acetic acid (309 mg, 0.500 mmol) in acetonitrile (6.00 mL) containing HBTU (1.90×10² mg, 0.501 mmol), HOBt (77.0 mg, 0.503 mmol) and Et₃N (279 □L, 2.00 mmol) was transferred to a solution of the product of Part A (2.60×10² mg, 0.501 mmol) in acetonitrile (4.00 mL). The resulting solution was maintained at 22° C. for 0.5 hours, then concentrated in vacuo. The residue was redissolved in ethyl acetate (100 mL), washed with 10% aqueous citric acid (3×30 mL), saturated NaHCO₃ (3×30 mL) and saturated NaCl (30 mL), then dried over Na₂SO₄, filtered and concentrated in vacuo. The crude oil was treated with a solution of Et₃SiH in CH₂Cl₂ (1:1 v/v, 2.00 mL) followed by TFA (8.00 mL, 104 mmol). After stirring 2 hours at 22° C., the resulting solution was concentrated in vacuo and purified by HPLC on a Phenomenex Luna C18 column (21.2×250 mm) using a 0.86%/min gradient of 5-35% acetonitrile containing 0.1% TFA and 10% H₂O at a flow rate of 20 mL/min. The main product peak eluting at 10 minutes was lyophilized to a white solid (187 mg, 0.162 mmol; 32.3%). ¹H NMR (DMSO-d₆, 600 MHz): δ 10.53 (1H, s), 10.47 (1H, s), 9.02 (1H, br t, J=5.7 Hz), 8.54 (2H, br s), 7.86 (2H, AB, J_{AB}=8.2 Hz), 7.42 (2H, AB, J_{AB}=8.4 Hz), 7.39 (2H, dd, J=5.3, 3.3 Hz), 7.31 (2H, dd, J=5.5, 3.2 Hz), 4.44 (2H, br d, J=5.4 Hz), 4.30 (2H, br s), 3.79 (2H, AB, J_{AB}=17.5 Hz), 3.52 (8H, s), 3.40 (4H, br t, J=5.4 Hz), 3.31 (2H, AB, J_{AB}17.6 Hz), 3.07 (4H, br t, J=5.4 Hz). ¹³C NMR (DMSO-d₆, 151 MHz): δ 172.7, 171.0, 165.4, 164.9, 157.9 (q, J=33.1 Hz), 142.6, 138.5, 130.8, 127.6, 127.4, 127.2, 124.8, 116.6 (q, J=297 Hz), 64.1, 54.3, 53.9, 52.2, 48.7, 43.0, 42.0. MS (ESI): 700.3 (100, M+H), 541.2 (82.3), 350.6 (91.8, M+2H). HRMS: Calcd for C₃₂H₃₉FeN₇O₁₁: 753.2052; found: 753.2037.

### EXAMPLE 103

### Synthesis of 2-{[2-({[N-({4-[N-((2R)-2-Amino-3-indol-3-ylpropanoylamino)-carbamoyl]phenyl}methyl)carbamoyl]methyl}{2-[bis(carboxymethyl)-amino]ethyl}amino)ethyl](carboxymethyl)amino}acetic Acid, Trifluoroacetic Acid Salt

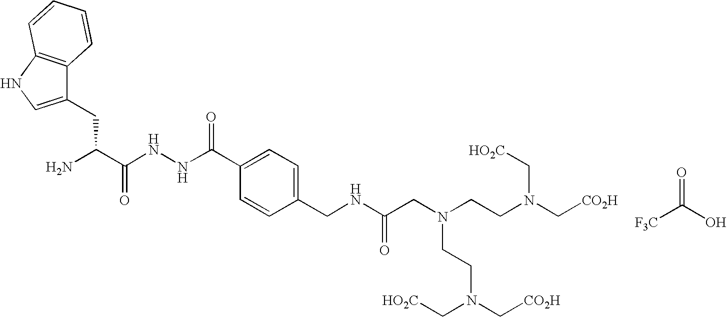

### Part A—Preparation of (2R)—N-{[4-(Aminomethyl)phenyl]carbonylamino}-2-[(tert-butoxy)carbonylamino]-3-indol-3-ylpropanamide, Trifluoroacetic Acid Salt

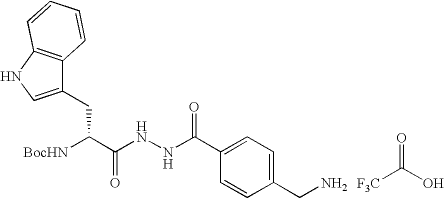

The product of Example 29A was deprotected with a solution of TFA in CH₂Cl₂ (1:1 v/v) and the resulting salt (251 mg, 0.501 mmol) dissolved in DMF (10.0 mL) and successively treated with Boc-DTrp-OH (183 mg, 0.601 mmol), HOBt (84.0 mg, 0.549 mmol), HBTU (209 mg, 0.551 mmol) and i-Pr₂NEt (349 μL, 2.00 mmol). After 0.5 hours at 22° C., the crude reaction mixture was diluted with ethyl acetate (100 mL), washed with 10% aqueous citric acid (3×30 mL), saturated NaHCO₃ (3×30 mL) and saturated NaCl (30 mL), then dried over Na₂SO₄, filtered and concentrated in vacuo. The resulting oil was treated with a solution of Et₂NH in acetonitrile (1:1 v/v, 10.0 mL), stirred 0.3 hours, then concentrated in vacuo and purified by HPLC on a Phenomenex Luna C18 column (21.2×250 mm) using a 1.2%/min gradient of 15-50% acetonitrile containing 0.1% TFA and 10% H₂O at a flow rate of 20 mL/min. The main product peak eluting at 17 minutes was lyophilized to a white solid (236 mg, 0.347 mmol; 69.4%). ¹H NMR (DMSO-d₆, 600 MHz): δ 10.82 (1H, s), 10.49 (1H, s), 10.18 (1H, s), 8.27 (3H, br s), 7.95 (2H, AB, J_{AB}=8.2 Hz), 7.69 (1H, d, J=7.9 Hz), 7.57 (2H, AB, J_{AB}=8.1 Hz), 7.33 (1H, d, J=8.1 Hz), 7.23 (1H, d, J=2.0 Hz), 7.07 (1H, dd, J=7.3, 7.3 Hz), 6.99 (1H, dd, J=7.5, 7.2 Hz), 6.82 (1H, d, J=8.4 Hz), 4.36 (1H, td, J=9.8, 4.1 Hz), 4.12 (2H, br d, J=4.5 Hz), 3.19 (1H, dd, J=14.5, 3.7 Hz), 2.97 (1H, dd, J=14.6, 10.2 Hz), 1.31 (9H, s). ¹³C NMR (DMSO-d₆, 151 MHz): δ 171.6, 164.8, 157.9 (q, J=32.0 Hz), 155.1, 137.6, 136.0, 132.5, 128.7, 127.7, 127.3, 123.8, 120.8, 118.5, 118.1, 116.9 (q, J=299 Hz), 111.2, 110.0, 77.9, 53.6, 41.9, 28.1. MS (ESI): 903.5 (66.9, 2M+H), 452.3 (100, M+H), 396.2 (30.0, M-t-Bu), 352.3 (32.7, M-Boc). HRMS: Calcd for C₂₄H₃₀N₅O₄: 452.2292; found: 452.2298.

### Part B—Preparation of 2-{[2-({[N-({4-[N-((2R)-2-Amino-3-indol-3-ylpropanoylamino)carbamoyl]phenyl}methyl)carbamoyl]methyl}{2-[bis(carboxymethyl)-amino]ethyl}amino)ethyl](carboxymethyl)amino}acetic Acid, Trifluoroacetic Acid Salt

A previously prepared solution of 2-{bis[2-(bis{[(tert-butyl)oxycarbonyl]-methyl}amino)-ethyl]amino}acetic acid (225 mg, 0.364 mmol) in acetonitrile (4.00 mL) containing HBTU (132 mg, 0.348 mmol), HOBt (53.0 mg, 0.346 mmol) and Et₃N (185 μL, 1.33 mmol) was transferred to a solution of the product of Part A (225 mg, 0.331 mmol) in acetonitrile (2.00 mL). The resulting solution was maintained at 22° C. for 0.5 hours, then concentrated in vacuo. The residue was redissolved in ethyl acetate (100 mL), washed with 10% aqueous citric acid (3×30 mL), saturated NaHCO₃ (3×30 mL) and saturated NaCl (30 mL), then dried over Na₂SO₄, filtered and concentrated in vacuo. The crude oil was treated with a solution of Et₃SiH in CH₂Cl₂ (1:1 v/v, 2.00 mL) followed by TFA (8.00 mL, 104 mmol). After stirring 2 hours at 22° C., the resulting solution was concentrated in vacuo and purified by HPLC on a Phenomenex Luna C18 column (21.2×250 mm) using a 0.8%/min gradient of 5-25% acetonitrile containing 0.1% TFA and 10% H₂O at a flow rate of 20 mL/min. The main product peak eluting at 21 minutes was lyophilized to a white solid (124 mg, 0.105 mmol; 31.7%). ¹H NMR (DMSO-d₆, 600 MHz): δ 11.06 (1H, s), 10.75 (1H, s), 10.65 (1H, s), 9.03 (1H, br t, J=5.8 Hz), 8.18 (3H, br s), 7.90 (2H, AB, J_{AB}=8.2 Hz), 7.78 (1H, d, J=7.9 Hz), 7.44 (2H, AB, J_{AB}=8.3 Hz), 7.39 (1H, d, J=8.1 Hz), 7.33 (1H, d, J=2.2 Hz), 7.12 (1H, td, J=7.0, 1.1 Hz), 7.05 (1H, td, J=7.0, 0.9 Hz), 4.45 (2H, br d, J=5.8 Hz), 4.31 (2H, s), 4.13 (1H, br s), 3.52 (8H, s), 3.41 (4H, br t, J=6.0 Hz), 3.38 (1H, dd, J=15.0, 4.6 Hz), 3.16 (1H, dd, J=15.0, 8.9 Hz), 3.07 (4H, br t, J=5.9 Hz). ¹³C NMR (DMSO-d₆, 151 MHz): δ 172.7, 167.9, 165.1, 164.9, 157.9 (q, J=32.7 Hz), 142.6, 136.3, 130.8, 127.7, 127.2, 127.0, 125.2, 121.2, 118.5, 118.4, 116.7 (q, J=298 Hz), 111.5, 106.5, 54.3, 53.9, 52.2, 51.6, 48.7, 42.0, 27.7. MS (ESI): 727.2 (83.7, M+H), 541.3 (21.0), 364.2 (61.9, M+2H), 355.7 (100). HRMS: Calcd for C₃₃H₄₀FeN8O₁₁: 780.2161; found: 780.2167.

### EXAMPLE 104

### Synthesis of 2-{[2-({[N-(4-{4-[N-((2R)-2-Amino-3-phenylpropanoylamino)-carbamoyl]phenyl}butyl)carbamoyl]methyl}{2-[bis(carboxymethyl)amino]-ethyl}amino)ethyl](carboxymethyl)amino}acetic Acid, Trifluoroacetic Acid Salt

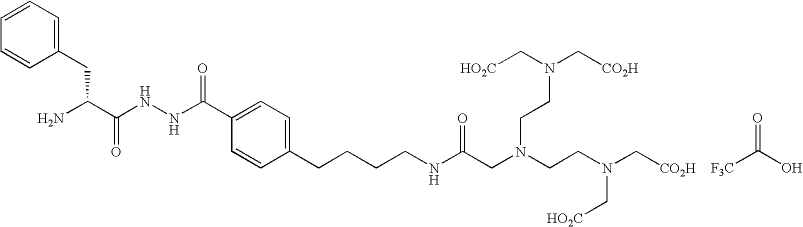

### Part A—Preparation of Methyl 4-[4-(1,3-Dioxoisoindolin-2-yl)butyl]benzoate

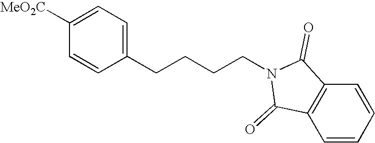

A solution of methyl 4-(4-hydroxybutyl)benzoate (2.10 g, 10.1 mmol) (Taylor, E. C.; Harrington, P. M. J. Org. Chem. 1990, 55, 3222), phthalimide (1.56 g, 10.6 mmol) and PPh₃ (2.78 g, 10.6 mmol) in THF (40.0 mL) at 0° C. was treated with diisopropyl azodicarboxylate (1.95 mL, 9.90 mmol) dropwise over 0.25 hours. The resulting solution was warmed slowly to 22° C. over 3 hours, then concentrated in vacuo and treated with Et₂O (75 mL) to induce precipitation of Ph₃P(O). The Et₂O was removed in vacuo and the resulting solid purified by chromatography on silica (1:3 ethyl acetate/pentane) afforded a white solid (2.40 g, 7.11 mmol; 71.8%). It should be noted that the purified product contained unreacted phthalimide (˜10% w/w). ¹H NMR (CDCl₃, 600 MHz): δ7.94 (2H, AA′XX′, J_{AX}=8.3 Hz, J_{AA′}=1.9 Hz), 7.85 (2H, dd, J=5.4, 3.0 Hz), 7.72 (2H, dd, J=5.5, 3.0 Hz), 7.24 (2H, AA′XX′, J_{AX}=8.4 Hz, J_{XX′}=1.9 Hz), 3.90 (3H, s), 3.73 (2H, t, J=6.9 Hz), 2.72 (2H, t, J=7.2 Hz), 1.77-1.67 (4H, m). ¹³C NMR (CDCl₃, 151 MHz): δ168.4, 167.1, 147.5, 133.9, 132.1, 129.7, 128.4, 127.9, 123.2, 51.9, 37.6, 35.3, 28.2, 28.1. MS (ESI): 360.2 (50.1, M+Na), 338.1 (9.8, M+H), 306.2 (100, M-methanol).

### Part B—Preparation of 2-{N-[4-(4-Carboxyphenyl)butyl]carbamoyl}benzoic Acid

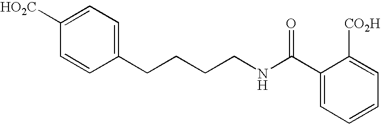

A solution of the product of Part A (1.00 g, 2.96 mmol) in THF/H₂O (4:1 v/v, 15.0 mL) at 22° C. was treated with LiOH.H₂O (0.500 g, 11.9 mmol) in one portion. The resulting solution was stirred 16 hours, then acidified with 0.5M HCl (pH=4-5). The resulting suspension was diluted with ethyl acetate (50 mL), washed with 5% aqueous citric acid and saturated NaCl (2×25 mL each), then filtered using a sintered glass funnel. The white powder thus obtained was set aside while the filtrate was dried over MgSO₄, filtered and concentrated in vacuo to afford a white powder; the collected solids were combined (957 mg, 2.80 mmol; 94.6%). ¹H NMR (CDCl₃, 600 MHz): δ7.43 (2H, AA′XX′, J_{AX}=8.2 Hz, J_{AA′}=1.8 Hz), 7.39 (1H, dd, J=7.7, 1.2Hz), 7.37 (1H, brt, J=5.8 Hz), 7.04 (1H, td, J=7.5, 1.4Hz), 6.97 (1H, td, J=7.6, 1.3 Hz), 6.94 (1H, dd, J=7.5, 1.3 Hz), 6.80 (2H, AA′XX′, J_{AX}=8.3 Hz, J_{XX′}=1.8 Hz), 2.88 (2H, td, J=7.0, 5.8 Hz), 2.24 (2H, t, J=7.6 Hz), 1.28-1.23 (2H, m), 1.18-1.13 (2H, m). MS (ESI): 342.2 (88.5, M+H), 324.2 (100, M-H₂O).

### Part C—Preparation of (2R)—N-{[4-(4-Aminobutyl)phenyl]carbonylamino}-2-[(tert-butoxy)carbonylamino]-3-phenylpropanamide, Trifluoroacetic Acid Salt

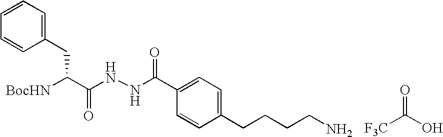

A solution of the product of Part B (183. mg, 0.536 mmol) in DMF (10.0 mL) containing HBTU (447 mg, 1.18 mmol), HOBt (182 mg, 1.19 mmol) and Et₃N (299 μL, 2.15 mmol) was treated with the product of Example 87A (329 mg, 1.18 mmol) in one portion at 22° C. After 0.25 hours, the solution was diluted with ethyl acetate (200 mL), washed with 5% aqueous citric acid (5×10 mL), 0.5 M NaOH (5×10 mL) and saturated NaCl (2×10 mL) then dried over MgSO₄, filtered and concentrated in vacuo. The resulting oil was directly treated with hydrazine hydrate (15.0 mL, 309 mmol), stirred 0.5 hours at 22° C., and then diluted with ethyl acetate (125 mL) with transfer to a separatory funnel. The ethyl acetate layer was washed with saturated solutions of NaHCO₃ (3×5 mL) and NaCl (2×5 mL), then dried over MgSO₄, filtered and concentrated in vacuo. The resulting solid was purified by HPLC on a Phenomenex Luna C18 column (41.2×250 num) using a 1.0%/min gradient of 20-50% acetonitrile containing 0.1% TFA and 10% H₂O at a flow rate of 80 mL/min. The main product peak eluting at 20 minutes was lyophilized to a white solid (93.7 mg, 0.165 mmol; 30.7%). MS (ESI): 909.45 (37.4, M+2H), 455.3 (100, M+H), 399.2 (18.8, M-t-Bu), 355.2 (64.0, M-Boc).

### Part D—Preparation of 2-{[2-({[N-(4-{4-[N-((2R)-2-Amino-3-phenylpropanoylamino)carbamoyl]phenyl}butyl)carbamoyl]methyl}{2-[bis(carboxymethyl)amino]ethyl}amino)ethyl](carboxymethyl)amino}acetic Acid, Trifluoroacetic Acid Salt

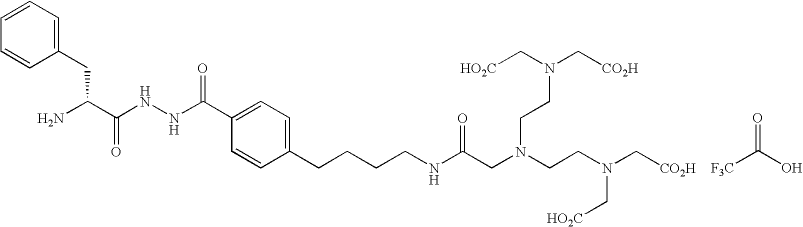

A previously prepared solution of 2-{bis[2-(bis{[(tert-butyl)oxycarbonyl]-methyl}amino)-ethyl]amino}acetic acid (80.5 mg, 0.130 mmol) in DMF (3.00 mL) containing HBTU (49.3 mg, 0.130 mmol) and Et₃N (36.3 μL, 0.260 mmol) was treated with the product of Part C (74.0 mg, 0.130 mmol) in one portion. The resulting solution was maintained at 22° C. for 0.75 hours, then concentrated in vacuo. The residue was redissolved in ethyl acetate (100 mL), washed with 5% aqueous citric acid (5×10 mL), 0.5 M NaOH (5×10 mL) and saturated NaCl (2×10 mL), then dried over MgSO₄, filtered and concentrated in vacuo. Global deprotection was then performed using TFA/CH₂Cl₂/Et₃SiH (90:8:2 v/v, 3.00 mL). After 1.5 hours at 22° C., the solution was concentrated in vacuo and purified by HPLC on a Phenomenex Luna C18 column (21.2×250 mm) using a 1.0%/min gradient of 0-30% acetonitrile containing 0.1% TFA and 10% H₂O at a flow rate of 20 mL/min. The main product peak eluting at 23 minutes was lyophilized to a white solid (90.3 mg, 76.1 μmol; 58.5%). ¹H NMR (DMSO-d₆, 600 MHz): δ 10.62 (1H, s), 10.56 (1H, s), 8.46 (1H, br t, J=5.3 Hz), 8.26 (3H, br s), 7.84 (2H, AB, J_{AB}=8.1 Hz), 7.37-7.34 (6H, m), 7.32-7.30 (1H, m), 4.16 (3H, br s), 3.50 (8H, s), 3.37 (4H, br t, J=5.6 Hz), 3.25 (1H, br d, J=12.5 Hz), 3.16 (2H, td, J=6.4, 6.0 Hz), 3.04 (4H, br t, J=5.7 Hz), 2.67 (2H, t, J=7.6 Hz), 1.62 (2H, tt, J=7.5, 7.3 Hz), 1.46 (2H, tt, J=7.4, 7.3 Hz). ¹³C NMR (DMSO-d₆, 151 MHz): δ 172.7, 167.5, 165.3, 164.4, 157.9 (q, J=32.5 Hz), 146.5, 134.5, 129.7, 129.6, 128.6, 128.4, 127.6, 127.2, 116.7 (q, J=298 Hz), 54.3, 53.8, 52.3, 52.1, 48.6, 38.5, 37.1, 34.5, 28.4, 27.9. MS (ESI): 730.4 (61.4, M+H), 365.8 (100, M+2H). HRMS: Calcd for C₃₄H₄₅FeN₇O₁₁: 783.2521; found: 782.2514. The optical purity of the product was established by chiral GLC analysis; 99.0% D-phenylalanine.

### EXAMPLE 105

### Synthesis of 2-{[2-({[N-({3-[N-((2R)-2-Amino-4-phenylbutanoylamino)carbamoyl]-phenyl}methyl)carbamoyl]methyl}{2-[bis(carboxymethyl)amino]ethyl}-amino)ethyl](carboxymethyl)amino}acetic Acid, Trifluoroacetic Acid Salt

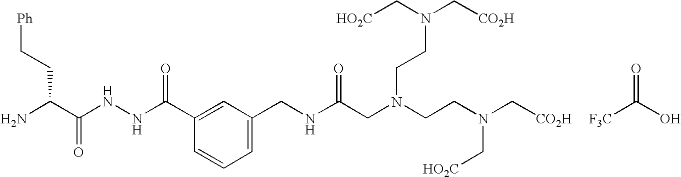

### Part A—Preparation of N-Amino(3-{[(fluoren-9-ylmethoxy)carbonylamino]-methyl}phenyl)carboxamide, Trifluoroacetic Acid Salt

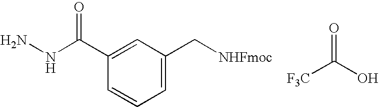

A solution of 3-{[(fluoren-9-ylmethoxy)carbonylamino]methyl}benzoic acid (2.50 g, 6.96 mmol) in dry DMF (15.0 mL) was successively treated with HBTU (2.51 g, 6.62 mmol) and collidine (2.50 mL, 18.9 mmol) then stirred 3 minutes at 22° C. tert-Butyl carbazate (0.840 g, 6.36 mmol) was added in one portion and the resulting solution stirred 0.5 hours at 22° C. The crude reaction mixture was diluted with ethyl acetate (350 mL), washed with 10% aqueous citric acid (5×10 mL), 0.5 M NaOH (5×10 mL) and saturated NaCl (2×20 mL), then dried over MgSO₄, filtered and concentrated in vacuo. The resulting solid was dissolved in CH₂Cl₂ (15.0 mL) and treated with TFA (15.0 mL, 0.190 mol). After 0.5 hours at 22° C., all volatiles were removed in vacuo and the residue redissolved in acetonitrile/H₂O (1:1 v/v) then lyophilized to an off white solid that was used without further purification in the subsequent step.

### Part B—Preparation of (2R)—N-{[3-(Aminomethyl)phenyl]carbonylamino}-2-[(tert-butoxy)carbonylamino]-4-phenylbutanamide

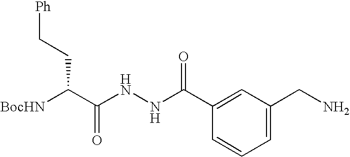

A solution Boc-D-Hphe-OH (159 mg, 0.569 mmol) in DMF (5.00 mL) containing HBTU (206 mg, 0.543 mmol), HOBt (83.9 mg, 0.548 mmol) and i-Pr₂NEt (181 μL, 1.04 mmol) was treated with the product of Part A (2.60×10² mg, 0.518 mmol) in one portion. After 0.5 hours at 22° C., the crude reaction mixture was diluted with ethyl acetate (125 mL), washed with 5% aqueous citric acid (5×10 mL), 0.5 M NaOH (5×10 mL) and saturated NaCl (2×10 mL), then dried over MgSO₄, filtered and concentrated in vacuo. The resulting oil was treated with a solution of Et₂NH in acetonitrile (1:1 v/v, 10.0 mL), stirred 0.3 hours, and then concentrated in vacuo. The resulting solid was used directly in the subsequent step.

### Part C—Preparation of 2-{[2-({[N-({3-[N-((2R)-2-Amino-4-phenylbutanoylamino)-carbamoyl]phenyl}methyl)carbamoyl]methyl}{2-[bis(carboxymethyl)-amino]ethyl}-amino)ethyl](carboxymethyl)amino}acetic Acid, Trifluoroacetic Acid Salt

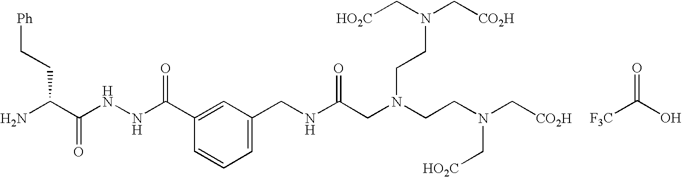

A previously prepared solution of 2-{bis[2-(bis{[(tert-butyl)oxycarbonyl]-methyl}amino)-ethyl]amino}acetic acid (327 mg, 0.529 mmol) in DMF (5.00 mL) containing HBTU (201 mg, 0.530 mmol) and Et₃N (141 μL, 1.01 mmol) was treated with the product of Part B (215 mg, 0.504 mmol). After 0.25 hours at 22° C., the crude reaction mixture was diluted with ethyl acetate (125 mL), washed with 5% aqueous citric acid (5×10 mL), 0.5 M NaOH (5×10 mL) and saturated NaCl (2×10 mL), then dried over MgSO₄, filtered and concentrated in vacuo. Global deprotection was then performed using TFA/CH₂Cl₂/Et₃SiH (90:8:2 v/v, 5.00 mL). After 1.5 hours at 22° C., the solution was concentrated in vacuo and purified by HPLC on a Phenomenex Luna C18 column (21.2×250 mm) using a 1.0%/min gradient of 10-35% acetonitrile containing 0.1% TFA at a flow rate of 20 mL/min. The main product peak eluting at 12 minutes was lyophilized to a white solid (206.3 mg, 0.178 mmol; 35.3%). ¹H NMR (DMSO-d₆, 600 MHz): δ 10.61 (1H, s), 10.57 (1H, s), 9.02 (1H, br t, J=5.7 Hz), 8.39 (3H, br s), 7.84 (1H, br s), 7.81 (1H, dt, J=7.3, 1.7 Hz), 7.53 (1H, dt, J=7.7, 1.6 Hz), 7.50 (1H, dd, J=7.7, 7.3 Hz), 7.34 (2H, dd, J=8.2, 6.9 Hz), 7.24 (2H, d, J=8.0 Hz), 7.23 (1H, tt, J=7.0, 1.3 Hz), 4.43 (2H, br d, J=5.7 Hz), 4.27 (2H, s), 4.03 (1H, br s), 3.51 (8H, s), 3.40 (4H, br t, J=5.6 Hz), 3.06 (4H, br t, J=5.8 Hz), 2.83-2.73 (2H, m), 2.14-2.06 (2H, m). ¹³C NMR (DMSO-d₆, 151 MHz): □172.6, 167.8, 165.4, 164.8, 157.8 (q, J=32.0 Hz), 140.6, 138.8, 132.2, 130.8, 128.6, 128.5, 128.0, 126.9, 126.1, 126.0, 116.9 (q, J=299 Hz), 54.3, 53.9, 52.2, 51.1, 48.6, 42.2, 33.3, 29.9. MS (ESI): 072.2 (100, M+H), 351.7 (79.0, M+2H). HRMS: Calcd for C₃₂H₄₁FeN₇O₁₁: 755.2208; found: 755.2216. The optical purity of the product was established by chiral GLC analysis; 98.0% D-homophenylalanine.

### EXAMPLE 106

### Synthesis of 2-[(2-{[(N-{5-[N-({4-[N-((2R)-2-Amino-4-phenylbutanoylamino)-carbamoyl]phenyl}methyl)carbamoyl]pentyl}carbamoyl)methyl]{2-[bis(carboxymethyl)amino]ethyl}amino}ethyl)(carboxymethyl)amino]acetic Acid, Trifluoroacetic Acid Salt

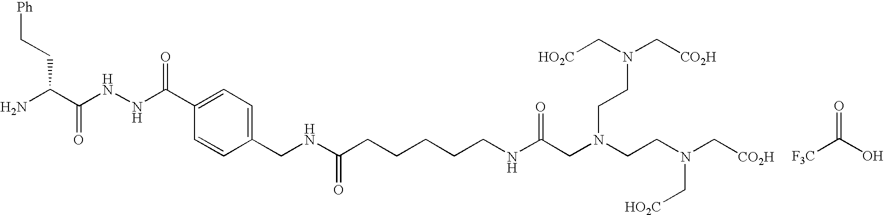

### Part A—Preparation of (2R)—N-{[4-(Aminomethyl)phenyl]carbonylamino}-2-[(tert-butoxy)carbonylamino]-4-phenylbutanamide

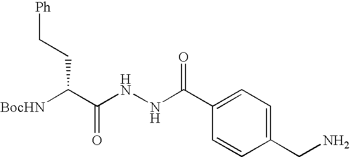

A solution Boc-D-Hphe-OH (159 mg, 0.569 mmol) in DMF (5.00 mL) containing HBTU (205 mg, 0.540 mmol), HOBt (83.5 mg, 0.545 mmol) and i-Pr₂NEt (1.80×10² μL, 1.03 mmol) was treated with the product of Example 29A (2.00×10² mg, 0.516 mmol) in one portion. After 0.75 hours at 22° C., the crude reaction mixture was diluted with ethyl acetate (125 mL), washed with 5% aqueous citric acid (5×10 mL), 0.5 M NaOH (5×10 mL) and saturated NaCl (2×10 mL), then dried over MgSO₄, filtered and concentrated in vacuo. The resulting oil was treated with a solution of Et₂NH in acetonitrile (1:1 v/v, 10.0 mL), stirred 0.3 hours, and then concentrated in vacuo. The resulting solid was used directly in the subsequent step.

### Part B—Preparation of N-{[4-(N-{(2R)-2-[(tert-Butoxy)carbonylamino]-4-phenylbutanoylamino}carbamoyl)phenyl]methyl}-6-aminohexanamide, Trifluoroacetic Acid Salt

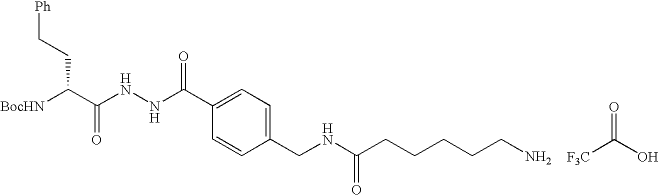

A solution of the product of Example 3A (201 mg, 0.569 mmol) in DMF (5.00 mL) containing HBTU (206 mg, 0.543 mmol) and i-Pr₂NEt (180 μL, 1.03 mmol) was treated with the product of Example 98B (2.20×10² mg, 0.516 mmol) in one portion. After 0.75 hours at 22° C., the crude reaction mixture was diluted with ethyl acetate (125 mL), washed with 5% aqueous citric acid (5×10 mL), 0.5 M NaOH (5×10 mL) and saturated NaCl (2×10 mL), then dried over MgSO₄, filtered and concentrated in vacuo. The resulting oil was treated with a solution of Et₂NH in acetonitrile (1:1 v/v, 10.0 mL), stirred 0.3 hours, and then concentrated in vacuo. The resulting solid was purified by HPLC on a Phenomenex Luna C18 column (21.2×250 mm) using a 1.0%/min gradient of 10-35% acetonitrile containing 0.1% TFA and 10% H₂O at a flow rate of 20 mL/min. The main product peak eluting at 18 minutes was lyophilized to a white solid (126 mg, 0.193 mmol; 37.5%).

### Part C—Preparation of 2-[(2-{[(N-{5-[N-({4-[N-((2R)-2-Amino-4-phenylbutanoylamino)carbamoyl]phenyl}methyl)carbamoyl]pentyl}carbamoyl)-methyl]{2-[bis(carboxymethyl)amino]ethyl}amino}ethyl)(carboxymethyl)amino]acetic Acid, Trifluoroacetic Acid Salt

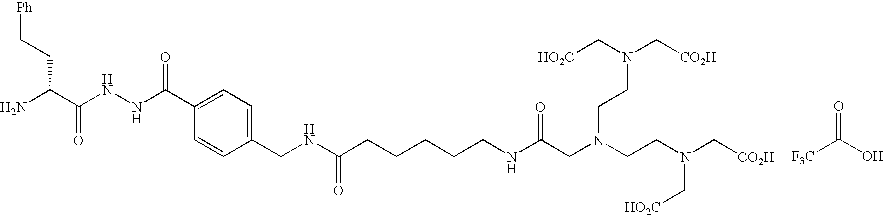

A previously prepared solution of 2-{bis[2-(bis{[(tert-butyl)oxycarbonyl]-methyl}amino)-ethyl]amino}acetic acid (119 mg, 0.193 mmol) in DMF (4.00 mL) containing HBTU (73.1 mg, 0.193 mmol) and Et₃N (51.2 δL, 0.367 mmol) was treated with the product of Part B (1.20×10² mg, 0.184 mmol). After 0.25 hours at 22° C., the crude reaction mixture was diluted with ethyl acetate (125 mL), washed with 5% aqueous citric acid (5×10 mL), 0.5 M NaOH (5×10 mL) and saturated NaCl (2×10 mL), then dried over MgSO₄, filtered and concentrated in vacuo. Global deprotection was then performed using TFA/CH₂Cl₂/Et₃SiH (90:8:2 v/v, 4.00 mL). After 1.5 hours at 22° C., the solution was concentrated in vacuo and purified by HPLC on a Phenomenex Luna C18 column (21.2×250 mm) using a 1.0%/min gradient of 10-35% acetonitrile containing 0.1% TFA at a flow rate of 20 mL/min. The main product peak eluting at 14 minutes was lyophilized to a white solid (131 mg, 0.103 mmol; 56.1%). ¹H NMR (DMSO-d₆, 600 MHz): δ 10.55 (1H, s), 10.52 (1H, s), 8.43 (1H, br t, J=5.4 Hz), 8.38 (3H, br s), 7.85 (2H, AB, J_{AB}=8.2 Hz), 7.37 (2H, AB, J_{AB}=8.3 Hz), 7.33 (2H, dd, J=7.7, 7.5 Hz), 7.24 (2H, d, J=7.6 Hz), 7.24-7.22 (1H, m), 4.33 (2H, br d, J=5.8 Hz), 4.16 (2H, s), 4.02 (1H, br s), 3.50 (8H, s), 3.37 (4H, br t, J=5.6 Hz), 3.12 (2H, td, J=6.8, 6.0 Hz), 3.04 (4H, br t, J=5.6 Hz), 2.83-2.72 (2H, m), 2.16 (2H, t, J=7.5 Hz), 2.13-2.07 (2H, m), 1.54 (2H, tt, J=7.7, 7.5 Hz), 1.45 (2H, tt, J=7.4, 7.0 Hz). ¹³C NMR (DMSO-d₆, 151 MHz): δ172.6, 172.1, 167.8, 165.3, 164.3, 157.9 (q, J=33.1 Hz), 144.0, 140.6, 130.5, 128.5, 128.0, 127.5, 127.0, 126.1, 116.5 (q, J=297 Hz), 54.3, 53.9, 52.2, 51.2, 48.6, 41.7, 38.6, 35.1, 33.2, 29.9, 28.4, 26.0, 24.8. MS (ESI): 815.3 (56.9, M+H), 654.2 (19.5), 408.2 (100, M+2H). HRMS: Calcd for C₃₈H₅₂FeN₈O₁₂: 868.3049; found: 868.3038. The optical purity of the product was established by chiral GLC analysis; 98.6% D-homophenylalanine.

### EXAMPLE 107

### Synthesis of 2-{[2-({[N-({4-[N-((2R)-2-Amino-3-(2-naphthyl)propanoylamino)carbamoyl]phenyl}methyl)carbamoyl]methyl}{2-[bis(carboxymethyl)amino]ethyl}amino)ethyl](carboxymethyl)amino}acetic Acid, Trifluoroacetic Acid Salt

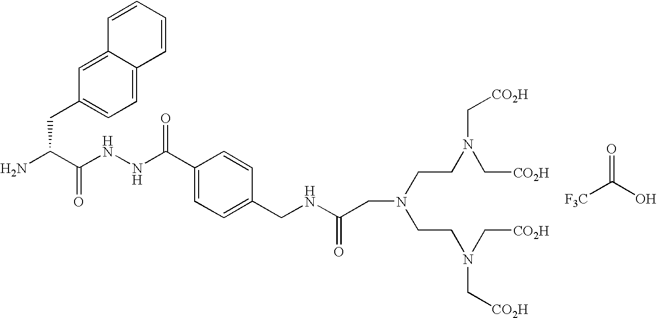

### Part A—Preparation of tert-Butyl 2-[(2-{[(N-{[4-(N-{(2R)-2-[(tert-butoxy)-carbonylamino]-3-(2-naphthyl)propanoylamino}carbamoyl)phenyl]methyl}-carbamoyl)methyl][2-(bis{[(tert-butyl)oxycarbonyl]methyl}amino)ethyl]-amino}ethyl){[(tert-butyl)oxycarbonyl]methyl}amino]acetate

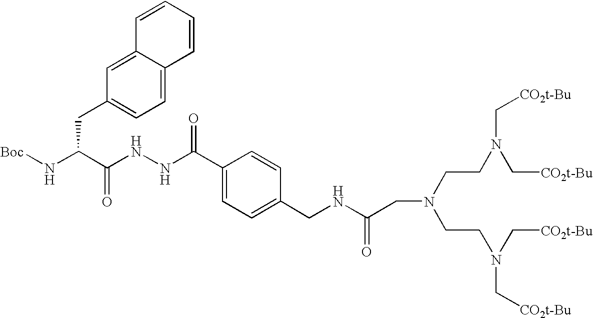

A solution of Boc-D-2-Nal-OH (250 mg, 0.793 mmol), the intermediate product of Example 29B (439 mg, 1.134 mmol), and DIEA (0.276 mL, 1.585 mmol) in DMF (2.0 mL) was treated with HBTU (361 mg, 0.951 mmol) and stirred at room temperature under nitrogen for 18 hours. The reaction mixture was diluted with ethyl acetate (100 mL), washed consecutively with 10% citric acid (3×100 mL), 1 N NaOH (3×100 mL), and saturated NaCl (100 mL), dried (Na₂SO₄), and concentrated under reduced pressure. The resulting residue was taken up in 50:50 TEA:acetonitrile (50 mL) and stirred at room temperature under nitrogen for 45 minutes. The volatiles were removed under vacuum and the resulting residue was triturated with cyclohexane (3×25 mL) to give a colorless solid (244 mg). MS (ESI): 925.5 (95, 2M+H), 463.4 (100, M+H).

A solution of the above solid (244 mg, 0.528 mmol), 2-{bis[2-(bis{[(tert-butyl)oxycarbonyl]methyl}amino)ethyl]amino}acetic acid (391 mg, 0.633 mmol), and DIEA (184 μL, 1.055 mmol) in DMF (2.0 mL) was treated with HBTU (240 mg, 0.633 mmol) and stirred at room temperature under nitrogen for 45 minutes. The reaction was diluted with ethyl acetate (80 mL), washed consecutively with 10% citric acid (3×50 mL), 1 N NaOH (3×50 mL), and saturated NaCl (80 mL), dried (MgSO₄), and concentrated under reduced vacuum. The resulting crude product was purified by HPLC on a Phenomenex Luna C18(2) column (41.4×250 mm) using a 0.9%/min gradient of 45 to 72% acetonitrile containing 0.1% TFA at a flow rate of 80 mL/min. The main product peak eluting at 26.6 minutes was lyophilized to give the title compound as a colorless solid (220 mg, 39%, HPLC purity 90%). MS (ESI): 1062.6 (100, M+H), 453.9 (10, M-Boc-tBu+2H).

### Part B—Preparation of 2-{[2-({[N-({4-[N-((2R)-2-Amino-3-(2-naphthyl)-propanoylamino)carbamoyl]phenyl}methyl)carbamoyl]methyl}{2-[bis(carboxymethyl)amino]ethyl}amino)ethyl](carboxymethyl)amino}acetic Acid, Trifluoroacetic Acid Salt

The product of Part A (60 mg, 0.056 mmol) was dissolved in 90:10:3 TFA:dichloromethane:TIS (10 mL) and stirred at room temperature under nitrogen for 4 hours. The solution was concentrated and the resulting residue was purified by HPLC on a Phenomenex Luna C18(2) column (21.2×250 mm) using a 0.9%/min gradient of 1.8 to 28.8% acetonitrile containing 0.1% TFA at a flow rate of 20 mL/min. The main product peak eluting at 25.6 minutes was lyophilized to give the title compound as a colorless solid (30 mg, 71%, HPLC purity 96%). ¹H NMR (DMSO-d₆): δ 7.91-7.85 (m, 3H), 7.81 (s, 1H), 7.73 (d, AA′ portion of AA′BB′ system, J=8.4 Hz, 2H), 7.53-7.47 (m, 2H), 7.47-7.42 (m, 1H), 7.39 (d, BB′ portion of AA′BB′ system, J=8.4 Hz, 2H), 4.40 (s, 2H), 4.36 (t, A portion of AXY system, J=7.8 Hz, 1H), 3.82 (s, 2H), 3.66 (s, 8H), 3.41 (dd, X portion of AXY system Jₐₓ=7.8 Hz, J_{xy}=15 Hz, 1H), 3.33 (dd, Y portion of AXY system Jₐₓ=7.8 Hz, J_{xy}=15 Hz, 1H), 3.23-3.13 (m, 8H); ¹³C NMR (1:1 CD₃C:D₂O): δ 173.31, 169.10, 168.92, 168.31, 162.35 (q, J=34.4 Hz), 144.10, 134.47, 133.76, 132.41, 131.27, 129.80, 128.99, 128.87, 128.74, 128.39, 127.60, 127.41, 56.31, 55.97, 54.26, 53.22, 51.47, 43.73, 37.88. MS (ESI): 738.3 (100, M+H), 369.9 (40, M+2H); HRMS: Calcd for C₃₅H₄₁FeN₇O₁₁ (M+Fe-2H): 791.2208; Found: 791.2216.

### EXAMPLE 108

### Synthesis of 2-{[2-({[N-(2-{2-[(N-{(1R)-1-[N-((2R)-2-Amino-4-phenylbutanoylamino)carbamoyl]-2-phenylethyl}carbamoyl)methoxy]ethoxy}ethyl)carbamoyl]methyl}{2-[bis(carboxymethyl)amino]ethyl}amino)ethyl](carboxymethyl)amino}acetic Acid, Trifluoroacetic Acid Salt

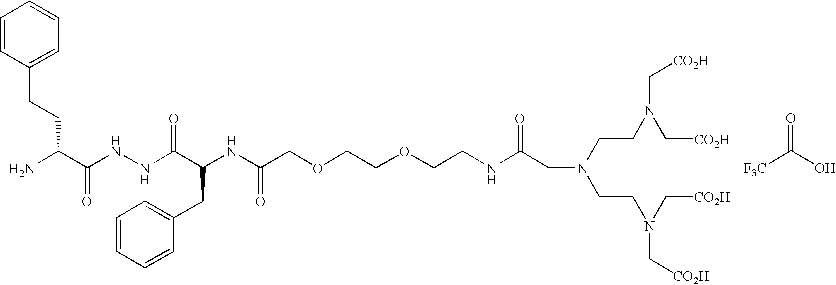

### Part A—Preparation of tert-Butyl 2-[(2-{[(N-{2-[2-({N-[(1R)-1-(N-{(2R)-2-[(tert-Butoxy)carbonylamino]-4-phenylbutanoylamino}carbamoyl)-2-phenylethyl]carbamoyl}methoxy)ethoxy]ethyl}carbamoyl)methyl][2-(bis{[(tert-butyl)oxycarbonyl]methyl}amino)ethyl]amino}ethyl){[(tert-butyl)oxycarbonyl]methyl}amino]acetate

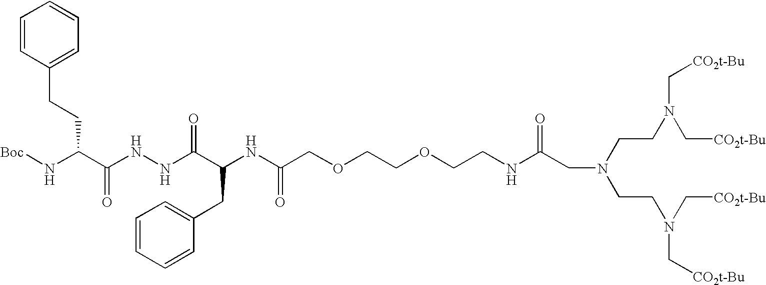

A solution of Fmoc-Phe-OH (697 mg, 1.80 mmol), HBTU (625 mg, 1.65 mmol), HOBt (253 g, 1.65 mmol), and DIEA (650 μL, 3.73 mmol) in DMF (10 mL) was stirred at room temperature under nitrogen for 20 minutes. The solution was treated with the product of Example 98A (440 mg, 1.50 mmol, 60% pure) and sufficient DIEA (350 μL, 2.00 mmol) to raise the pH to 10. The solution was stirred an additional 18 hours and concentrated under vacuum. The resulting residue was dissolved in ethyl acetate (50 mL) and washed consecutively with 10% citric acid (2×50 mL), 1 N NaOH 2×50 mL), and saturated NaCl (50 mL). The ethyl acetate layer was dried (MgSO₄) and concentrated to give a light brown solid (340 mg). MS (ESI): 563.3 (100, M-Boc+H), 685.3 (10, M+Na).

A solution of the above solid in 50:50 DEA:acetonitrile was stirred under nitrogen at room temperature for 30 minutes and concentrated using reduced pressure. The resulting solid residue was triturated with cyclohexane (3×30 mL) and collected by vacuum filtration to give a fine yellow powder. This solid was added to a previously prepared solution of 2-(2-{2-[(fluoren-9-ylmethoxy)carbonylamino]ethoxy}ethoxy)acetic acid (286 mg, 0.90 mmol), HBTU (286 mg, 0.76 mmol), HOBt (32 mg, 0.21 mmol), and DIEA (250 μL, 1.50 mmol) in DMF (6.0 mL) and the solution was stirred at room temperature under nitrogen for 20 hours. The solution was concentrated using reduced pressure and the resulting oily mixture was dissolved in ethyl acetate (50 mL). The solution was washed consecutively with 10% citric acid (50 mL), 1 N NaOH (50 mL), and saturated NaCl (50 mL). The ethyl acetate layer was dried (MgSO₄) and concentrated to give a tan solid (230 mg). MS (ESI): 708.3 (100, M+H−Boc), 830.4 (5, M+Na).

The above solid (202 mg, 0.250 mmol) was dissolved in 50:50 DEA:acetonitrile (1.0 mL) and stirred under nitrogen at room temperature for 30 minutes. The solution was concentrated and the resulting solid was triturated with cyclohexane (3×30 mL) to give a fine yellow powder. This solid was added to a previously prepared solution of 2-{bis[2-(bis{[(tert-butyl)oxycarbonyl]methyl}-amino)ethyl]amino}acetic acid (185 mg, 0.300 mmol), HBTU (104 mg, 0.275 mmol), and DIEA (174 μL, 1.00 mmol) in DMF (1.0 mL) and stirring was continued at room temperature under nitrogen for 18 hours. The volatiles were removed under reduced pressure and the resulting residue was purified by HPLC on a Phenomenex Luna C18(2) column (41.4×250 mm) using a 0.9%/min gradient of 45 to 72% acetonitrile containing 0.1% TFA at a flow rate of 80 mL/min. The main product peak eluting at 27.0 minutes was lyophilized to give the title compound as a colorless powder (32 mg, 11%, HPLC purity 100%). MS (ESI): 1185.5 (100, M+H).

### Part B—Preparation of 2-{[2-({[N-(2-{2-[(N-{(1R)-1-[N-((2R)-2-Amino-4-phenylbutanoylamino)carbamoyl]-2-phenylethyl}carbamoyl)methoxy]ethoxy}-ethyl)carbamoyl]methyl}{2-[bis(carboxymethyl)amino]ethyl}amino)ethyl]-(carboxymethyl)amino}acetic Acid, Trifluoroacetic Acid Salt

The product of Part B (32.0 mg, 0.027 mmol) was dissolved in 90:7:3 TFA:dichloromethane:TIS (5.0 mL), stirred at room temperature under nitrogen for 3 hours, and concentrated under reduced pressure. The crude product was purified by HPLC on a Phenomenex Luna C18(2) column (41.4×250 mm) using a 0.9%/min gradient of 9 to 36% acetonitrile containing 0.1% TFA at a flow rate of 80 mL/min. The product peak eluting at 18 minutes was lyophilized to give the title compound as a colorless solid (24 mg, 91%, HPLC purity 90%). MS (ESI): 861.3 (40, M+H), 431.3 (100, M+2H). HRMS: Calcd for C₃₉H₅₄FeN₈O₁₄ (M−2H+Fe): 914.3103; Found: 914.3111; Chiral Analysis: 99.7% D-Hphe, 97.3% L-Phe.

### EXAMPLES 109-117

### Synthesis of Complexes [¹⁵⁷Gd(H-D-Amino Acid-Hydrazide-DTPA)]

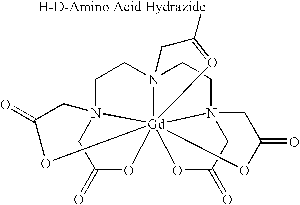

Example 114 was prepared as follows: A solution of the product of Example 19 (3.97 g, 3.64 mmol) in Milli-Q H₂O (18.2 mL) was treated with GdCl₃ (1.44 g, 5.46 mmol) in one portion at 22° C. The pH of the solution was adjusted to ˜7 with aqueous NaOH (25 mmol); direct HPLC analysis of the reaction mixture using a pH 7 mobile phase indicated complexation was complete. The reaction mixture was directly purified by HPLC on a Phenomenex Luna C18 column (21.2×250 mm) using a 1.0%/minute gradient of 0-20% acetonitrile at a flow rate of 20 mL/min; 15 mM NH₄OAc was employed as the aqueous component. The main product peak eluting at 12 minutes was lyophilized to give the title compound as a colorless solid (3.01 g, 3.82 mmol; >98%). The remaining examples were prepared in an analogous manner. Yield and characterization data are shown in Table 1.

TABLE 1
Characterization data for Examples 109-117

| | Starting | Yield | | HRMS (calcd; |
|---|---|---|---|---|
| Example # | Ligand | (%) | LRMS (ESI) | found) |
| 109 | 50 | 51 | 857.3 (85, M + H), 429.2 (100, M + 2H) | C₃₂H₄₁GdN₇O₁₁: |
| | | | | 857.2100; 857.2098 |
| 110 | 53 | 61 | 869.2 (100, M + H), 435.1 (60, | na |
| | | | M + 2H) | |
| 111 | 57 | 71 | 837.2 (100, M + H), 419.2 (75, | C₃₀H₄₅GdN₇O₁₁: |
| | | | M + 2H) | 837.2413; 837.2419 |
| 112 | 62 | 75 | 934.2 (100, M + H), 468.5 (85, | C₃₅H₅₄GdN₈O₁₂: |
| | | | M + 2H) | 936.3097; 936.311 |
| 113 | 69 | 75 | 893.3 (75, M + H), 447.2 (100, | C₃₅H₄₁GdN₇O₁₁: |
| | | | M + 2H) | 893.2100; 893.2091 |
| 114 | 19 | >98 | 789.3 (100, M + H), 394.9 (36.6, | C₂₆H₄₅GdN₇O₁₁: |
| | | | M + 2H) | 789.2413; |
| | | | | 789.2418 |
| 115 | 91 | 89.5 | 849.1 (47.0, M + H), 696.2 (34.6), | C₃₁H₄₄GdN₇O₁₁: |
| | | | 447.4 (100), 425.2 (59.8, | 849.2413; |
| | | | M + 2H) | 849.2397 |
| 116 | 98 | >98 | 857.0 (100, M + H), 696.1 (28.3), | C₃₂H₄₁GdN₇O₁₁: |
| | | | 428.9 (48, M + 2H) | 857.2100; |
| | | | | 857.2108 |
| 117 | 103 | 53.0 | 881.1 (100, M + H), 441.4 (51.2, | C₃₃H₄₀GdN₈O₁₁: |
| | | | M + 2H) | 882.2052; |
| | | | | 882.2037 |

### EXAMPLES 118 & 119

### Synthesis of Complexes [^{99m}Tc(H-D-Amino Acid-Hydrazide-HYNIC)(tricine)(TPPTS)]

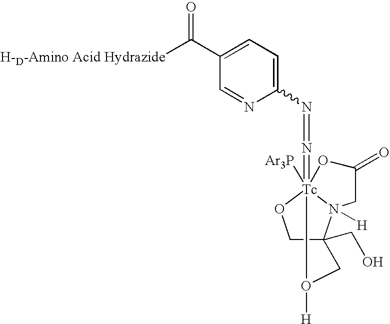

To a lead shielded lyophilized vial containing TPPTS (4.48 mg), tricine (6.3 mg), mannitol (40 mg), succinic acid buffer, pH 4.8, and 0.1% Pluronic F-64 surfactant, was added sterile water for injection (1.1 mL), the appropriate H-D-amino acid-hydrazide-HYNIC-conjugate (20 μg) in deionized water or 50% aqueous ethanol (0.2 mL), and ^{99m}TcO₄⁻ (50±5 mCi) in saline (0.2 mL). The reconstituted kit was heated in a 95° C. water bath for 10 minutes, and allowed to cool 5 minutes at room temperature. A sample of the reaction mixture was analyzed by HPLC. The RCP results are listed in Table 2.

HPLC Method

| Detector: | INUS β-Ram, UV at 220 nm | | | | | |
|---|---|---|---|---|---|---|
| Column: | Zorbax Rx C18, 25 cm × 4.6 mm | | | | | |
| Guard: | Zorbax C18 | | | | | |
| Temperature: | Ambient | | | | | |
| Flow: | 1.0 mL/min | | | | | |
| Solvent A: | 25 mM ammonium acetate (no pH adjustment) | | | | | |
| Solvent B: | 100% Acetonitrile | | | | | |
| Gradient: | | | | | | |
| t (min) | 0 | 20 | 21 | 25 | 26 | 32 |
| % Solvent B | 10 | 40 | 60 | 60 | 10 | 10 |

TABLE 2
Yield Data for ^{99m}Tc Complexes 118 and 119

| Cold | | |
|---|---|---|
| Example # | Radiolabeled Product Example # | % RCP |
| 1 | 118 | 100.0 |
| 2 | 119 | 72.0 |

### EXAMPLES 120-152

### Synthesis of [¹⁴C]H-D-Amino Acid-Hydrazide-Acetyl Conjugates

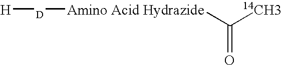

### Part A—Preparation of [¹⁴C]Sodium Acetate Solutions

Two hundred fifty millicuries of [1-¹⁴C]acetic acid, sodium salt, solid 50-60 mCi/mmole specific activity was obtained from General Electric Health Care (formerly Amersham Biosciences). The [1-¹⁴C]acetic acid, sodium salt, solid was dissolved in anhydrous acetonitrile (25 mL) to prepare a [¹⁴C]sodium acetate stock solution. The solution was vortex mixed for 10 minutes. Aliquots were removed for radioassay using liquid scintillation counter (LSC) method. The LSC radioassays were conducted by distributing a measured aliquot of the radioactive solution into a 10 mL glass scintillation vial containing Perkin Elmer Ultima Gold™ scintillation fluid (5 mL) and subsequently measuring the radioactive content using either a Packard model 2500TR or 1600TR LSC. Subsequent ten fold dilutions were made from this stock solution to prepare solutions used in the reactions. Prior to each reaction LSC radioassays were conducted on the reagent solution.

### Part B—Conjugation of [¹⁴C]Sodium Acetate to Amino Acid-Hydrazides

Acetylation of the Boc-amino acid-hydrazides were performed by the coupling of amine with the ¹⁴C-containing sodium acetate in a solution of HBTU and DIEA in DMF at ambient temperature (25° C.). The contents were combined in a 5 mL conical interior Wheaton™ thick walled reaction vial, and allowed to react for 1 hour.

### Part C—Deprotection and Final Purification

Side chain protecting groups were removed using one of the following methods.
Method A: 50:50 TFA:dichloromethane at RT for 15 minutes.
Method B: 95:2.5:2.5 TFA:Anisole:water at RT for 45 minutes.
Method C: 2 mol % Pd(OAc)₂, 4 mol % TPPTS, Et₂NH in 2:1 acetonitrile:H₂O

The crude reaction mixtures were analyzed using a HPLC interfaced with a mass spectrometer (LC/MS) on a Zorbax Eclipse XDB C-18 (4.6 mm×250 mm) column. The solutions were concentrated under reduced pressure and the crude product was purified by HPLC on a Phenomenex™ LUNA C18(2) column (10 mm×250 mm) using H₂O:acetonitrile gradients containing 0.1% trifluoroacetic acid at a flow rate of 5 mL/min. Product fractions were concentrated under reduced pressure and analzyed by LC/MS on a Zorbax Eclipse XDB C-18 column (4.6 mm×250 mm) using H₂O:acetonitrile gradients containing 0.1% formic acid. LCMS and HPLC interfaced with a radioactivity detector was used to confirm RCP. Purity data are shown in Table 3.

TABLE 3
Yield Data for [¹⁴C]H-D-Amino Acid-Hydrazide-Acetyl Conjugates

| | Radiolabeled Product | |
|---|---|---|
| Cold Ex # | Example # | % RCP |
| 3 | 120 | 99.0 |
| 4 | 121 | 99.0 |
| 5 | 122 | 100.0 |
| 6 | 123 | 100.0 |
| 7 | 124 | 95.0 |
| 8 | 125 | 100.0 |
| 9 | 126 | 94.0 |
| 13 | 127 | 100.0 |
| 14 | 128 | 100.0 |
| 20 | 129 | 100.0 |
| 21 | 130 | 100.0 |
| 22 | 131 | 100.0 |
| 23 | 132 | 90.0 |
| 24 | 133 | 100.0 |
| 25 | 134 | 100.0 |
| 26 | 135 | 91.0 |
| 27 | 136 | 100.0 |
| 28 | 137 | 100.0 |
| 29 | 138 | 100.0 |
| 30 | 139 | 100.0 |
| 39 | 140 | 100.0 |
| 40 | 141 | 100.0 |
| 45 | 142 | 95.0 |
| 46 | 143 | 100.0 |
| 74 | 144 | 92.0 |
| 75 | 145 | 99.0 |
| 76 | 146 | 98.0 |
| 77 | 147 | 100.0 |
| 78 | 148 | 100.0 |
| 79 | 149 | 91.0 |
| 80 | 150 | 100.0 |
| 83 | 151 | 100.0 |
| 85 | 152 | 100.0 |

### EXAMPLES 153-213

### Synthesis of ¹¹¹In-Labeled DTPA and DOTA Conjugates

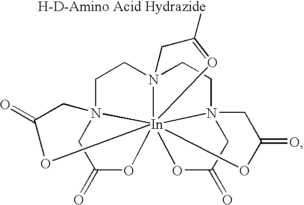

A solution of the H-D-amino acid-hydrazide-DTPA conjugate (50 μg) in 0.5 M pH 6.0 ammonium acetate (1.0 mL) was treated with ¹¹¹InCl₃ stock solution (2.0 mCi) in 0.05 N HCl. The resulting solution was heated in a boiling water bath for 20 minutes. The crude reaction mixtures were analyzed using a HPLC interfaced with a mass spectrometer (LC/MS) on a Zorbax Eclipse XDB C-18 (4.6 mm×250 mm) column using H₂O:acetonitrile gradients containing 25 mM NH₄OAc (pH 6.8). Product was purified by HPLC on a Phenomenex™ LUNA C18(2) column (10 mm×250 mm) using H₂O:acetonitrile gradients containing 25 mM NH₄OAc (pH 6.8). Product fractions were concentrated under reduced pressure and analzyed using a HPLC interfaced with a radioactivity detector on a Zorbax Eclipse XDB C-18 (4.6 mm×250 mm) column using H₂O:acetonitrile gradients containing 25 mM NH₄OAc (pH 6.8). Purity data are shown in Table 4.

TABLE 4
Purity Data for ¹¹¹In-Labeled DTPA and DOTA Conjugates

| Cold Ex # | Radiolabeled Product Example # | % RCP |
|---|---|---|
| 10 | 153 | 100.0 |
| 11 | 154 | |
| 19 | 155 | 100.0 |
| 31 | 156 | 99.0 |
| 38 | 157 | 100.0 |
| 41 | 158 | 97.0 |
| 42 | 159 | 99.0 |
| 43 | 160 | 98.0 |
| 47 | 161 | 80.0 |
| 48 | 162 | 97.0 |
| 49 | 163 | 100.0 |
| 50 | 164 | 100.0 |
| 51 | 165 | 100.0 |
| 52 | 166 | 100.0 |
| 53 | 167 | 100.0 |
| 54 | 168 | 95.0 |
| 55 | 169 | 100.0 |
| 56 | 170 | 96.0 |
| 57 | 171 | 97.0 |
| 58 | 172 | 95.0 |
| 59 | 173 | 100.0 |
| 60 | 174 | 99.0 |
| 61 | 175 | 98.0 |
| 62 | 176 | 96.0 |
| 63 | 177 | 100.0 |
| 64 | 178 | 100.0 |
| 65 | 179 | 99.0 |
| 66 | 180 | 99.0 |
| 67 | 181 | 98.0 |
| 68 | 182 | 100.0 |
| 69 | 183 | 98.0 |
| 70 | 184 | 100.0 |
| 71 | 185 | 99.0 |
| 72 | 186 | 100.0 |
| 73 | 187 | 93.0 |
| 81 | 188 | 100.0 |
| 82 | 189 | 94.0 |
| 84 | 190 | 100.0 |
| 86 | 191 | 100.0 |
| 87 | 192 | 87.0 |
| 88 | 193 | 100.0 |
| 89 | 194 | 100.0 |
| 90 | 195 | 100.0 |
| 91 | 196 | 100.0 |
| 92 | 197 | 95.0 |
| 93 | 198 | 96.0 |
| 94 | 199 | 96.0 |
| 95 | 200 | 70.0 |
| 96 | 201 | 78.0 |
| 97 | 202 | 87.0 |
| 98 | 203 | 100.0 |
| 99 | 204 | 100.0 |
| 100 | 205 | 100.0 |
| 101 | 206 | 100.0 |
| 102 | 207 | 84.0 |
| 103 | 208 | 100.0 |
| 104 | 209 | 95.0 |
| 105 | 210 | 100.0 |
| 106 | 211 | 100.0 |
| 107 | 212 | 100.0 |
| 108 | 213 | 100.0 |

### EXAMPLES 214-216

### Synthesis of ⁶⁸Ga-Labeled DTPA and DOTA Conjugates

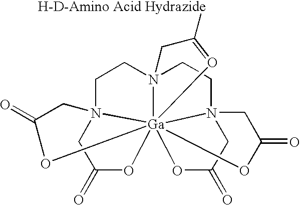

A solution of the H-d-amino acid-hydrazide-chelator conjugate (350 μg) in NH₄OAc buffer (0.5 M, pH 6.0, 450 μL) was treated with ⁶⁸GaCl₃ (18 mCi, 33 μmole) and heated at 100° C. for 30 minutes. The reaction mixture was checked by HPLC interfaced with a Berthold X and γ-ray detector which indicated ˜100% purity, as shown in Table 5.

TABLE 5
⁶⁸Ga Complexes

| | Radiolabeled Product | |
|---|---|---|
| Cold Ex # | Example # | % RCP |
| 19 | 214 | 100.0 |
| 11 | 215 | 100.0 |
| 84 | 216 | 100.0 |

### EXAMPLE 219

### Ex Vivo Blood Vessel Binding Assay

Normal aorta was obtained from New Zealand white rabbits. Aorta bearing atherosclerotic plaque was obtained from New Zealand white rabbits balloon stripped along the abdominal aorta and placed on a high fat diet (0.5% cholesterol) for 16-22 weeks. Vascular injury was produced with a 3-F Fogarty catheter along the abdominal aorta and left iliofemoral artery. This procedure generates an accelerated complex lesion development with a lipid rich core covered by a fibrous cap in rabbits. Rabbit plaque aorta or normal rabbit aorta (0.5 cm) was incubated with 10 nCi of ¹⁴C labeled compound or 1.5 μCi of ^{99m} Tc labeled compound or 0.135 μCi of ¹¹¹In labeled compound or 0.135 μCi of ⁶⁸Ga labeled compound or 0.135 82 Ci of ¹⁵³Gd labeled compound diluted in phosphate buffered saline (450 μL) for 2 hours at 37° C. The supernatant from this sample was collected and analyzed by HPLC to determine compound stability in presence of blood vessels. The blood vessel tissue was then washed with phosphate buffered saline (3×10 mL). Phosphate buffered saline (10 ml) was added to the tissue and incubated at 37° C. for 1 hour. The tissue was then washed with phosphate buffered saline (3×10 mL). The washed tissue was oxidized in a tissue oxidizer for C-14 labeled compounds. The counts in the oxidized tissue were determined on a beta counter. Blood vessels incubated with ^{99m} Tc labeled compound or ¹¹¹In labeled compound or ⁶⁸Ga labeled compound or ¹⁵³Gd labeled compound were counted on a gamma counter. The amount of compound bound to the tissue was determined as a percentage of the incubated compound. This was calculated using the following formula:

$$\% Tissue Uptake = \frac{Counts bound to tissue}{Total counts in test tube} \times 100$$

The data are shown in Table 7.

TABLE 7
Ex Vivo Blood Vessel Binding Data

| Cold | Radiolabeled | | Tissue |
|---|---|---|---|
| Ex # | Product Ex # | Structure | Binding % |
| 1 | 118 | H-d-Leu-Ahxh-Hynic | 15.74 |
| 2 | 119 | H-NLeu-Ahxh-Hynic | 3.33 |
| 3 | 120 | Boc-d-Leu-Ahxh-H | 14.09 |
| 4 | 121 | Boc-d-Leu-Apah-H | 30.55 |
| 5 | 122 | Boc-d-Leu-NHNH2 | 13.99 |
| 6 | 123 | Boc-d-Leu-NHNH—CO-(PEG)4-NH2 | 9.74 |
| 7 | 124 | Boc-d-Leu-NHNH—CO—NHNH2 | 5.85 |
| 8 | 125 | N,N-Me2-d-Leu-Ahxh-H | 4.63 |
| 9 | 126 | Boc-d-Leu-N(Me)-NH-Ahx-NH2 | 2.97 |
| 10 | 155 | H-d-Leu-Ahxh-DOTA | 3.49 |
| 13 | 127 | Boc-Leu-PABA-CO-d-Leu-Ahxh-H | 24.22 |
| 14 | 128 | Ac-PL-NLys(Boc)˜LL-PABA-CO-d- | 5.77 |
| | | Leu-Ahxh-H | |
| 19 | 157 | H-d-Leu-Ahxh-DTPA | 5.98 |
| 20 | 129 | Boc-d-Phe-Ahxh-H | 14.42 |
| 21 | 130 | Boc-Aib-Ahxh-H | 3.33 |
| 22 | 131 | Boc-d-Arg(Pmc)-Ahxh-H | 5.19 |
| 23 | 132 | Boc-d-Glu(tBu)-Ahxh-H | 2.47 |
| 24 | 133 | Boc-cLeu-Ahxh-H | 9.10 |
| 25 | 134 | Boc-d-Ala-Ahxh-H | 5.72 |
| 26 | 135 | Boc-βLeu-Ahxh-H | 2.30 |
| 27 | 136 | Boc-d-Lys(e-Leu(Boc))-Ahxh-H | 9.15 |
| 28 | 137 | H-Ahxh-d-Leu-Boc | 1.43 |
| 29 | 138 | Boc-d-Leu-Ambh-H | 21.11 |
| 30 | 139 | Boc-d-Leu-Inph-H | 18.36 |
| 31 | 158 | H-d-Leu-Inph-DTPA | 4.35 |
| 38 | 157 | H-D-Phe-Ahxh-DTPA | 9.23 |
| 39 | 140 | Boc-d-Leu-Gly-NH-Butyl-NH2 | 0.52 |
| 40 | 141 | Boc-d-Leu-N(Me)-NH-Ahx-H | 0.75 |
| 41 | 158 | H-d-Leu-Ambh-DTPA | 5.29 |
| 42 | 159 | H-d-Leu-Apah-DTPA | 10.10 |
| 43 | 160 | H-d-Leu-Apah-DOTA | 9.59 |
| 47 | 161 | H-d-Phe-Ambh-DTPA | 7.75 |
| 48 | 162 | H-d-Bip-NHNH-DTPA | 6.39 |
| 49 | 163 | H-Pro-Ahxh-DTPA | 2.61 |
| 50 | 164 | H-d-Phe-Apah-DTPA | 13.88 |
| 51 | 165 | H-d-Pro-Ahxh-DTPA | 5.51 |
| 52 | 166 | H-d-Ser(Bzl)-Ahxh-DTPA | 5.91 |
| 53 | 167 | H-d-Cys(Bzl)-Ahxh-DTPA | 9.74 |
| 54 | 168 | H-d-Hphe-Ahxh-DTPA | 9.17 |
| 55 | 169 | H-d-Tyr(Et)-Apah-DTPA | 13.53 |
| 56 | 170 | H-d-Cha-Apah-DTPA | 7.55 |
| 57 | 171 | H-d-Leu-Apph-DTPA | 16.56 |
| 58 | 172 | H-d-His-Ahxh-DTPA | 4.22 |
| 59 | 173 | H-d-Cys(Bzl)-Apah-DTPA | 13.82 |
| 60 | 174 | H-d-Phe-Apph-DTPA | 17.15 |
| 61 | 175 | H-d-Bip-Ahxh-DTPA | 32.09 |
| 62 | 176 | H-d-Leu-Apah-Ahx-DTPA | 25.21 |
| 63 | 177 | H-d-Phe(CF3)-Apah-DTPA | 13.53 |
| 64 | 178 | H-d-Tic-Apah-DTPA | 6.00 |
| 65 | 179 | H-d-Cys(Bzl)-Ambh-DTPA | 9.67 |
| 66 | 180 | H-d-Hphe-Apph-DTPA | 18.24 |
| 67 | 181 | H-d-Cys(Bzl)-Apph-DTPA | 13.91 |
| 68 | 182 | H-d-Phe-Apph-Ahx-DTPA | 12.02 |
| 69 | 183 | H-d-1-Nal-Ambh-DTPA | 24.41 |
| 70 | 184 | H-d-Trp-Apph-ETPA | 13.08 |
| 71 | 185 | H-d-H2phe-Ambh-DTPA | 10.64 |
| 72 | 186 | [H-d-Cys(Bzl)-Apph]2EDTA[APEEPA- | 7.78 |
| | | DTPA]2 | |
| 73 | 187 | H-d-Stya-Ambh-DTPA | 13.66 |
| 77 | 147 | Boc-d-Phe[p-(Ac-PL-NLys(Boc)˜Hphe- | 44.73 |
| | | L-NH)]-Ahxh-H | |
| 80 | 150 | Boc-L-d-Leu-Ahxh-H | 5.36 |
| 81 | 188 | H-cLeu-Ahxh-DTPA | 3.91 |
| 82 | 189 | H-d-Leu-NHNH-DTPA | 1.08 |
| 83 | 151 | Boc-d-Aphe(ε-Leu(Boc))-Ahxh-H | 26.92 |
| 86 | 191 | H-d-Val-Ahxh-DTPA | 3.02 |
| 87 | 192 | H-d-Phe-NHNH-DTPA | 1.78 |
| 88 | 193 | H-d-Aphe-Ahxh-DTPA | 8.69 |
| 89 | 194 | H-d-Cha-Ahxh-DTPA | 16.33 |
| 90 | 195 | H-cLeu-Ambh-DTPA | 9.52 |
| 91 | 196 | H-d-Cha-Ambh-DTPA | 8.25 |
| 92 | 197 | H-d-Cha-Inph-DTPA | 6.97 |
| 93 | 198 | H-d-Val-Ambh-DTPA | 5.26 |
| 94 | 199 | H-d-Phe-Inph-DTPA | 3.36 |
| 95 | 200 | H-d-Nle-Ahxh-DTPA | 2.72 |
| 96 | 201 | H-d-Leu-XInph-DTPA | 2.94 |
| 97 | 202 | H-d-Phe-XInph-DTPA | 5.44 |
| 98 | 203 | H-d-Hphe-Ambh-DTPA | 17.96 |
| 99 | 204 | H-d-Phe(F5)-Ambh-DTPA | 16.11 |
| 100 | 205 | H-d-Phe(4-NHBz)-Ambh-DTPA | 8.83 |
| 101 | 206 | H-d-Tyr(Bzl)-Ambh-DTPA | 19.62 |
| 102 | 207 | H-Aic-Ambh-DTPA | 5.26 |
| 103 | 208 | H-d-Trp-Ambh-DTPA | 14.91 |
| 104 | 209 | H-d-Phe-Bbh-DTPA | 20.9 |
| 105 | 210 | H-d-Hphe-mAmbh-DTPA | 13.13 |
| 106 | 211 | H-d-Hphe-Ambh-Ahx-DTPA | 18.09 |
| 107 | 212 | H-d-2-Nal-Ambh-DTPA | 18.78 |
| 108 | 213 | H-d-Hphe-Pheh-AEEA-DTPA | 26.68 |
| 11 | 215 | H-d-Leu-Ahxh-Apa-DOTA | 11.54 |

### EXAMPLE 220

### Aminopeptidase N Cleavage of Test Substrates

### Part A—Preparation of Substrates

The test compounds were dissolved in 50:50 TFA:dichloromethane and allowed to stand at ambient temperature under nitrogen for 10 minutes to remove the Boc protecting group from the N-terminus amino acid. The solutions were concentrated and the resulting oily residue was dissolved in 50:50 acetonitrile:water and lyophilized. The resulting flocculent solids were used directly in the APN assay.

### Part B—Enzyme Assay

Aminopeptidase N cleaves amino acids at the N-terminus of proteins and peptides attached to another amino acid. A stock solution of test substrates was prepared in 100% DMSO at a concentration of 8 mM. The stock solution (4.7 μL) was added to buffer (50 mM Hepes/pH 7.5, 10 mM CaCl₂, 0.1% Brij) for a final concentration of 0.5 mM test substrate in the reaction. To this reaction solution 0.02 U of the enzyme (APN) was added, the solution was mixed, and immediately 30 μL of the mix was transferred to HPLC vials containing acetic acid (15 μL) for t=0 minutes measurement. The rest of the mix in the test tube was incubated at 37° C. for 25 minutes. At the 25 minutes time point 30 μL of the mix was transferred to an HPLC vial containing acetic acid (15 μl) for the t=25 minute measurement. The test substrates and products were separated by reversed phase HPLC on a Zorbax SB-C18 column (4.6×150 mm, 5 micron) using a water:acetonitrile gradient containing 0.1% TFA at a flow rate of 1.0 mL/min and with UV detection. The peak areas were integrated and the substrate peak area was used to determine rate constant k in the following equation:
K={(% hydrolyzed/100)*[S]}/[E]*[time]
where S=test substrate concentration in μmoles

E=aminopeptidase N concentration in units/ml

K=μmoles of substrate hydrolyzed/minute/unit enzyme

The rate of hydrolysis of the test substrates is shown in Table 8.

TABLE 8
Rate of APN Hydrolysis of Test Substrates

| | μmoles/ |
|---|---|
| Compound # | minute/U |
| 13 | 0.33 |
| 80 | 0.00 |
| 83 | 0.43 |

### EXAMPLE 221

### Kinetic Measurements of MMP-2 and MMP-9 Mediated Hydrolysis of MMP Substrates

### Part A—Activation and Active Site Titration of MMP-2 and MMP-9

Purified MMP-2 (10 μg) or MMP-9 (10 μg) were reconstituted in 100 μL of TCN buffer (50 mM Tris (pH 7.5), 10 mM CaCl₂, 150 mM NaCl). Purified human MMP-9 was activated by incubation with 1 mM aminophenyl mercuric acetate (APMA) for 16 hours at 37° C. Pro-MMP-2 was activated by incubation with 1 mM APMA for 2 hours at 37° C. At the end of incubation 100% glycerol (100 μL) was added to active MMP-2 and active MMP-9 (final concentration 50% glycerol). Active MMP-2 and active MMP-9 were aliquoted and stored at −20° C. and −20° C., respectively.

### Part B—Active Site Titration of MMP-2/MMP-9

The level of active protease was quantified by active site titration studies prior to kinetic studies. The active site of MMP-9 and MMP-2 was titrated using the GM6001 dissolved in 100% DMSO at a stock concentration of 2.5 mM. Dilutions (1:2) of GM6001 were prepared in TCN buffer to give a final concentration of 5 nM to 0.08 nM GM6001 in the active site titration assay. Activated MMP-2 or activated MMP-9 (2 nM) was incubated with increasing concentrations of GM6001 at 37° C. in 96 well black microtiter plates and fluorescent substrate I (Mca-P-L-G-L-Dpa-A-R—NH₂) (150 μL) in assay buffer (50 mM tricine/pH 7.5, 100 mM CaCl₂, 0.2% NaN₃) was added to each well. The plate was shaken vigorously for 1 minute at room temperature and incubated at 27° C. for 1 hour. The reaction was stopped with 20 μL of 0.5 M EDTA. Plates were read on a fluorescence spectrophotometer at an excitation wavelength of 320 nm and an emission wavelength of 395 nm. The concentration of the active enzyme was determined using the Morrison equation and Kaleidagraph software (Reading, Pa.).

### Part C—Kinetic Measurements of Substrate Hydrolysis

The kinetic parameters of substrate hydrolysis by active MMP-2 and active MMP-9 were determined using a radio HPLC assay. A stock solution of different test substrates (1 mM) was prepared in 100% DMSO. Stock solutions of the test substrates were diluted 66.6 fold (15 nM) in buffer (50 mM Hepes/pH 7.5, 10 mM CaCl₂, 0.1% Brij) to give working stock solution. Working stock solution of the test substrate (10 μl) was added to buffer (115 μL) in a test tube and warmed at 37° C. for 2 minutes. To this solution 15 μl of working stock of active MMP-2 (final concentration 10 nM) or active MMP-9 (final concentration 2 nM) was added. Finally, 10 μCi of radiolabeled test substrate was added and the solution was mixed and immediately 67.5 μl of the mix was transferred to HPLC vials containing 7.5 μl of 0.5 M EDTA for a t=0 minutes measurement. The rest of the mix in the test tube was incubated at 37° C. for 60 minutes. At the 60 minutes time point 67.5 μl of the mix was transferred to the HPLC vial containing 7.5 μL of 0.5 M EDTA for the t=60 minutes measurement. The radiolabeled substrates and products were separated by reversed phase HPLC on a Zorbax Rx-C18 column (4.6×250 mm) maintained at a column temperature of 25° C. with a 1 ml/min flow rate and 60 μL sample size. Mobile phase A (MPA) was 25 mM ammonium acetate and mobile phase B (MPB) was 100% acetonitrile. A step gradient of 2% MPB at 3 minutes, 40% MPB at 13 minutes, 80% MPB at 18 minutes was used for separation of products and substrate. The radiolabel was detected by a IN/US beta ram detector. The peak areas were integrated and the substrate peak area was used to determine rate constant k in the following equation:
k=(−ln(St/So))/t
where St=Substrate peak at 60 min

So=Substrate peak at 0 min

T=3600 seconds.

In this reaction substrate concentration is much lower than Km therefore
Kcat/Km=k/[Et](M⁻¹S⁻¹)

The Kcat/Km values of various test substrates are presented in Table 9.

TABLE 9
Rate of MMP-2 and MMP-9 Hydrolysis of Test Substrates

| | |
|---|---|
| Example | Kcat/Km (M⁻¹s⁻¹) |

| Number | MMP-2 | MMP-9 |
|---|---|---|
| 128 | 295,168 | 499,578 |
| 142 | >500,000 | >500,000 |
| 143 | >500,000 | >500,000 |
| 144 | 0 | 0 |
| 145 | 1,625 | 542 |
| 146 | 25,139 | 10,591 |
| 147 | >500,000 | >500,000 |
| 148 | 11,401 | 9,432 |

### EXAMPLE 222

### In Vivo ApoE Mouse Aorta Uptake Studies

The apolipoprotein E (apoE) knockout mouse is a model of hypercholesterolemia that develops atherosclerotic lesions in the brachiocephalic artery, the aortic arch and the abdominal aorta. Mice were fed a high-fat diet to accelerate plaque formation and compounds were tested in the mice between 37-41 weeks on diet. Test compounds were radiolabeled (¹⁴C, ^{99m}Tc, or ¹¹¹In as described above) and administered at 0.02-4.0 mCi/kg to anesthetized mice in a single, bolus injection via the tail vein. Blood samples were collected via the tail between 0-30 minutes postinjection for pharmacokinetic analysis and mice were euthanized by CO₂ at 60 minutes for tissue harvesting. The aorta were flushed with saline through the left ventricle exiting via the femoral vein. The aorta were then removed from the heart to the renal bifurcation and additional tissue samples collected (blood, muscle, liver, kidney, heart, lung, spleen and innominate artery). All samples were weighed and assayed for radioactivity. Tissue uptake is expressed as percent of injected dose per gram of tissue (% ID/g). The aorta to blood ratios were calculated from the % ID/g data. Data are shown in Table 10.

TABLE 10
Aorta Plaque Uptake and Aorta:Blood Ratio of Radiolabeled
Compounds in the ApoE Mouse at 60 minutes Postinjection.

ApoE Mouse Data,
60 min

| Radiolabeled | Aorta | Aorta/Blood |
|---|---|---|
| Example # | (% ID/g) | Ratio |
| 118 | 7.0 | 2.6 |
| 120 | 8.9 | 2.2 |
| 121 | 9.3 | 4.6 |
| 122 | 4.0 | 3.0 |
| 126 | 1.3 | 0.6 |
| 128 | 4.1 | 2.3 |
| 129 | 8.1 | 3.3 |
| 133 | 5.7 | 2.9 |
| 138 | 8.6 | 6.7 |
| 139 | 17.2 | 5.0 |
| 140 | 1.3 | 0.8 |
| 142 | 2.1 | 1.9 |
| 145 | 2.0 | 1.4 |
| 146 | 3.5 | 1.0 |
| 147 | 11.0 | 8.8 |
| 150 | 2.1 | 1.2 |
| 151 | 9.0 | 2.9 |
| 155 | 5.1 | 2.0 |
| 156 | 3.5 | 2.5 |
| 157 | 5.1 | 3.3 |
| 158 | 11.4 | 3.6 |
| 159 | 9.5 | 3.0 |
| 161 | 8.7 | 4.9 |
| 162 | 7.1 | 4.7 |
| 163 | 6.5 | 0.9 |
| 164 | 8.2 | 7.5 |
| 166 | 4.0 | 3.3 |
| 167 | 8.0 | 4.9 |
| 168 | 7.5 | 5.8 |
| 169 | 6.1 | 5.1 |
| 170 | 7.0 | 5.4 |
| 171 | 6.7 | 8.8 |
| 172 | 3.7 | 1.2 |
| 173 | 9.5 | 5.8 |
| 174 | 8.7 | 11.0 |
| 175 | 7.9 | 5.9 |
| 176 | 12.2 | 13.3 |
| 177 | 7.9 | 8.7 |
| 178 | 8.3 | 4.2 |
| 180 | 8.3 | 13.2 |
| 181 | 9.8 | 7.3 |
| 182 | 11.0 | 4.5 |
| 183 | 15.7 | 11.7 |
| 184 | 12.5 | 11.3 |
| 185 | 11.2 | 8.1 |
| 186 | 7.0 | 1.5 |
| 187 | 6.1 | 5.4 |
| 188 | 2.8 | 2.2 |
| 189 | 3.3 | 1.0 |
| 190 | 4.9 | 2.1 |
| 191 | 5.3 | 1.5 |
| 192 | 2.3 | 1.9 |
| 193 | 6.2 | 3.0 |
| 194 | 6.8 | 3.3 |
| 195 | 4.6 | 1.9 |
| 196 | 9.8 | 7.8 |
| 200 | 5.1 | 2.2 |
| 201 | 4.3 | 2.1 |
| 202 | 4.3 | 4.0 |
| 203 | 12.9 | 8.7 |
| 204 | 6.7 | 5.3 |
| 205 | 7.8 | 6.3 |
| 206 | 5.8 | 3.2 |
| 207 | 3.8 | 2.1 |
| 208 | 14.8 | 8.7 |
| 209 | 10.8 | 9.6 |
| 210 | 10.5 | 11.2 |
| 211 | 9.9 | 3.9 |
| 212 | 12.6 | 10.8 |
| 213 | 8.4 | 9.8 |
| 214 | 6.1 | 3.1 |

### EXAMPLE 223

### In Vivo Rabbit Aorta Uptake Studies

Atherosclerosis was induced in New Zealand White male rabbits (3 kg) with aortic balloon endothelial injury followed by feeding a 0.5% cholesterol diet for 22 weeks. Test compounds were radiolabeled(¹⁴C, ^{99m}Tc, or ¹¹¹In as described above) and administered at 0.02-1.0 mCi/kg to anesthetized rabbits in a single, bolus injection vis the marginal ear vein. Blood samples were collected from the central ear artery at 0, 2, 5, 7, 10, 15, 30, and 60 minutes post injection. Rabbits were euthanized at 60 minutes post injection for tissue harvesting (blood, muscle, bile, urine, kidney, liver, spleen, heart, lung, colon, small intestine, stomach, testes). Abdominal aorta (upper, middle, and lower) and left and right femoral arteries were collected. Both plaque bearing and non-plaque bearing rabbits were administered compounds to compare aorta uptake. All samples were weighed and assayed for radioactivity. Tissue uptake is expressed as percent of injected dose per gram of tissue (% ID/g). The aorta to blood ratios were calculated from the % ID/g data. Data are shown in Table 11.

TABLE 11
Aorta Plaque Uptake and Aorta:Blood Ratio of Radiolabeled Compounds
in the Atherosclerotic Rabbit Model and Normal Rabbit at 60 minutes
Postinjection.

| | Plaque Rabbit | | Normal Rabbit | |
|---|---|---|---|---|
| | Aorta | | Aorta | |
| Radiolabeled | Uptake, | | Uptake, | |
| Example # | % ID/g | Aorta/Blood | % ID/g | Aorta/Blood |
| 118 | 0.099 | 5.54 | 0.095 | 6.14 |
| 120 | 0.088 | 3.98 | | |
| 128 | 0.076 | 1.93 | | |
| 139 | 0.096 | 2.92 | | |
| 147 | 0.24 | 9.05 | | |
| 155 | 0.080 | 1.67 | 0.065 | 2.21 |
| 159 | 0.083 | 4.08 | | |
| 164 | 0.085 | 3.56 | | |
| 171 | 0.168 | 2.89 | 0.143 | 4.49 |
| 174 | | | 0.111 | 5.42 |
| 176 | | | 0.123 | 4.95 |
| 183 | | | 0.146 | 4.72 |
| 203 | 0.127 | 2.76 | 0.129 | 4.78 |
| 208 | | | 0.135 | 5.58 |
| 215 | 0.092 | 1.31 | | |

### EXAMPLE 224

### In Vivo ApoE Mouse Aorta MRI Imaging

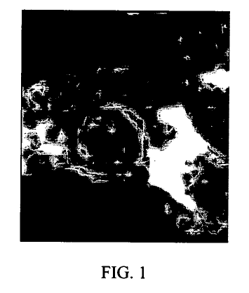

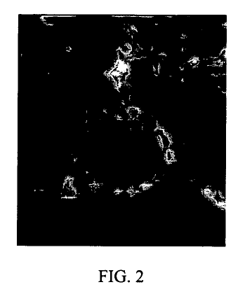

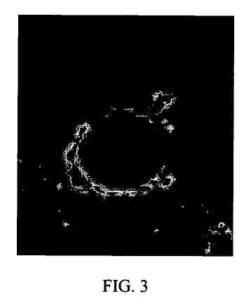

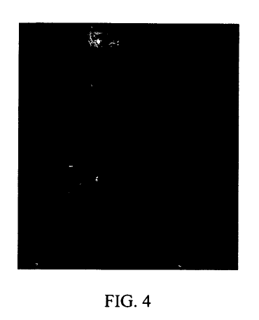

Shown in FIG. 1, FIG. 2, and FIG. 3 are examples of magnetic resonance images of the abdominal aorta of ApoE knockout mice administered one of compounds in this series. Mice were fed a high-fat diet to accelerate plaque formation and compounds were tested in the mice between 37-41 weeks on diet. Test compounds were administered at 0.05 mmol/kg (except for Example 114, administered at 0.1 mmol/kg) to anesthetized mice in a single, bolus injection via the tail vein. The images were acquired 1 hour post injection at 4.7 tesla using a 2.5 cm field of view (approximately 90 microns resolution) with a black blood, flow-suppressed spin-echo method. FIG. 4 is a similar image derived from administration of Magnevist® (gadopentetate dimeglumine), a DTPA chelate of gadolinium.

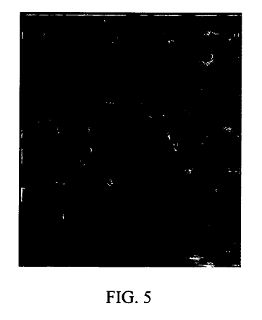

It can be seen that although the background image intensity varies, all of the compounds in the series yield significantly increased relative image intensity in the aorta, (seen as a ring-shaped structure in these transaxial images) compared with gadopentetate dimuglumine. For reference, an image obtained using the same method prior to the injection of contrast is shown in FIG. 5. This shows little or no contrast with surrounding muscular tissue and decreased signal-to-noise compared with the images obtained using contrast agent.

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A compound of formula (I) 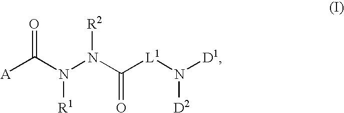
or a pharmaceutically acceptable salt thereof; wherein
A is a D-amino acid residue or a peptide consisting of a D-amino acid residue and a second D-amino acid;
D¹ and D² are independently selected from hydrogen, a chelator, and an imaging moiety;
L¹ is a linker; or
L¹ and D², together with the nitrogen atom to which they are attached, form a five- to seven-membered ring; and
R¹ and R² are independently selected from hydrogen and alkyl.

2. A compound of claim 1 wherein at least one of D¹ and D² is an imaging moiety.

3. A compound of claim 2 wherein the imaging moiety comprises of a non-metallic isotope.

4. A compound of claim 3 wherein the non-metallic isotope is ¹⁴C, ¹³N, ¹⁸F, ¹²³I, or ¹²⁵I.

5. A compound of claim 1 wherein L¹ is a linker selected from alkylene, alkenylene, arylene, heteroalkylene, arylalkylene, and heterocyclylene.

6. A compound of claim 5 wherein L¹ is alkylene.

7. A compound of claim 5 wherein L¹ is arylalkylene.

8. A compound of claim 1 wherein A is a D-amino acid residue.

9. A compound of claim 8 wherein A is 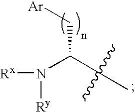
wherein
n is 0-6;
Ar is an aryl group; and
R^{x} and R^{y} are independently selected from hydrogen, alkenyl, alkoxycarbonyl, alkylcarbonyl, alkyl, aryl, and arylalkyl.

10. A compound of claim 9 wherein
n is 2;
Ar is phenyl; and
R^{x} and R^{y} are hydrogen.

11. A compound of claim 1 wherein one of D¹ and D² is a hydrogen and the other is a chelator.

12. A compound of claim 11 further comprising an imaging agent.

13. A compound of claim 11 wherein one of D¹ and D² is hydrogen and the other is a chelator of formula (II) 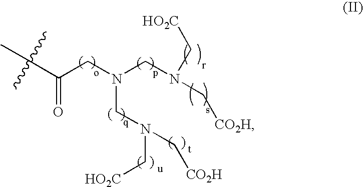
wherein
o, p, q, r, s, t, and u are each independently 1-6.

14. A compound of claim 13 wherein
o, r, s, t, and u are each 1; and
p and q are each 2.

15. A diagnostic agent comprising:
a. a compound of formula (III) 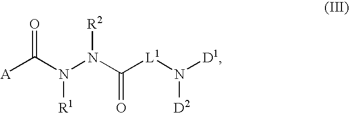
or a pharmaceutically acceptable salt thereof, wherein
A is a D-amino acid residue or a peptide consisting of a D-amino acid residue and a second D-amino acid;
D¹ and D² are independently selected from hydrogen and a chelator;
L¹ is a linker; or
L¹ and D², together with the nitrogen atom to which they are attached, form a five- to seven-membered ring; and
R¹ and R² are independently selected from hydrogen and alkyl; and
b. an imaging agent.

16. A diagnostic agent of claim 15 wherein the imaging agent is an echogenic substance, an optical reporter, a boron neutron absorber, a paramagnetic metal ion, a ferromagnetic metal, a gamma-emitting radioisotope, a positron-emitting radioisotope, or an x-ray absorber.

17. A diagnostic agent of claim 16 wherein the imaging agent is a paramagnetic metal ion.

18. A diagnostic agent of claim 17 wherein the paramagnetic metal ion is Gd(III).

19. A diagnostic agent of claim 16 wherein the imaging agent is a gamma-emitting radioisotope or positron-emitting radioisotope selected from ^{99m}Tc, ⁹⁵Tc, ¹¹¹In, ⁶²Cu, ⁶⁴Cu, ⁶⁷Ga, ⁶⁸Ga, and ¹⁵³Gd.

20. A diagnostic agent of claim 19 wherein the gamma-emitting radioisotope is ^{99m}Tc.

21. A diagnostic agent of claim 19 wherein the gamma-emitting radioisotope is ¹¹¹In.

22. A compound which is 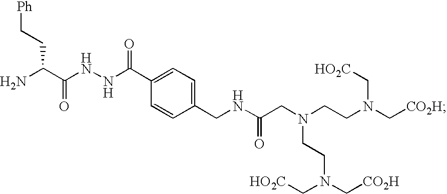
or a pharmaceutically acceptable salt thereof.

23. A diagnostic agent which is 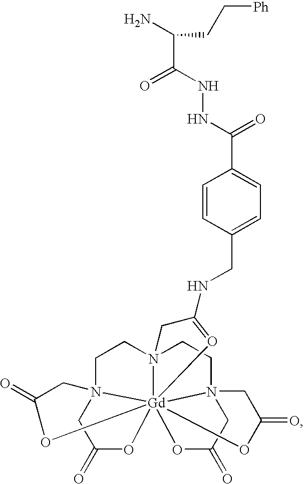
or a pharmaceutically acceptable salt thereof.

24. A diagnostic agent which is 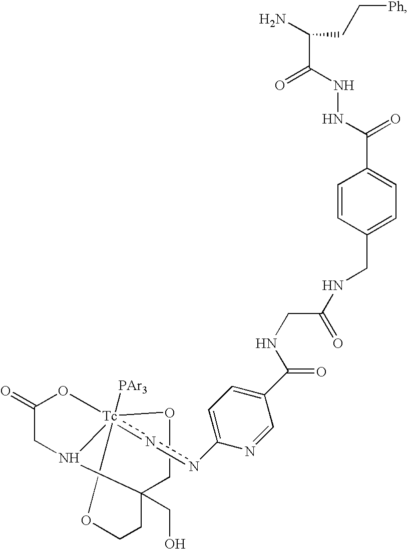
or a pharmaceutically acceptable salt thereof, wherein Ar is selected from phenyl, m-phenylsulfonic acid, or p-phenylsulfonic acid.

25. A diagnostic agent which is 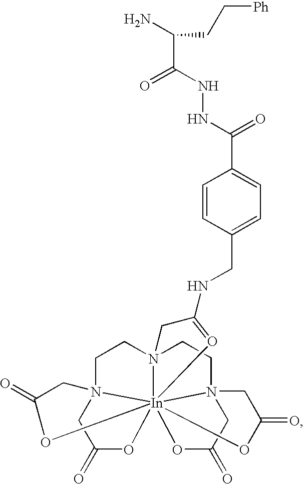
or a pharmaceutically acceptable salt thereof.

26. A composition comprising:
(a) a compound of claim 1; and
(b) a pharmaceutically acceptable carrier.

27. A composition comprising:
(a) a diagnostic agent of claim 15; and
(b) a pharmaceutically acceptable carrier.

28. A kit for detecting, imaging, and/or monitoring the presence of coronary plaque, carotid plaque, aortic plaque, plaquie of any arterial vessel, aneurism, vasculitis, and/or other diseases of the arterial wall in a patient comprising:
a. a compound of formula (III) 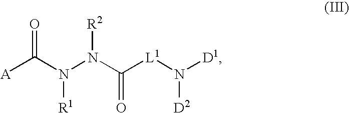
or a pharmaceutically acceptable salt thereof, wherein
A is a D-amino acid residue or a peptide consisting of a D-amino acid residue and a second D-amino acid;
D¹ and D² are independently selected from hydrogen and a chelator;
L¹ is a linker; or
L¹ and D², together with the nitrogen atom to which they are attached, form a five- to seven-membered ring; and
R¹ and R² are independently selected from hydrogen and alkyl;
b. an imaging agent;
c. a pharmaceutically acceptable carrier; and
d. instructions for preparing a composition comprising a diagnostic agent for detecting, imaging, and/or monitoring the presence of coronary plaque, carotid plaque, aortic plaque, plaquie of any arterial vessel, aneurism, vasculitis, and/or other diseases of the arterial wall in a patient.

29. A method of detecting, imaging, and/or monitoring the presence of coronary plaque, carotid plaque, aortic plaque, plaquie of any arterial vessel, aneurism, vasculitis, and/or other diseases of the arterial wall in a patient comprising the steps of:
a. administering to the patient a compound of claim 1; and
b. acquiring an image of a site of concentration of the compound in the patient by a diagnostic imaging technique.

30. A method of detecting, imaging, and/or monitoring the presence of coronary plaque, carotid plaque, aortic plaque, plaquie of any arterial vessel, aneurism, vasculitis, and/or other diseases of the arterial wall in a patient comprising the steps of:
a. administering to the patient a diagnostic agent of claim 15; and
b. acquiring an image of a site of concentration of the compound in the patient by a diagnostic imaging technique.
